(19) United States
(12) Patent Application Publication
(10) Pub. No.: US 2025/0127917 A1
(43) Pub. Date: Apr. 24, 2025

(54) COMPOSITIONS AND METHODS FOR TREATING CANCERS

(71) Applicants: Yale University, New Haven, CT (US); Gennao Bio, Inc., Pennington, NJ (US)

(72) Inventors: Elias Quijano, Durham, CT (US); Peter Glazer, Guilford, CT (US); Bruce C. Turner, Bryn Mawr, PA (US); Dale Ludwig, Pennington, NJ (US); Venugopalareddy Bommireddy Venkata, Pennington, NJ (US); Luisa Escobar-Hoyos, New Haven, CT (US)

(21) Appl. No.: 18/687,041

(22) PCT Filed: Aug. 31, 2022
(86) PCT No.: PCT/US2022/075762

§ 371 Date: Feb. 27, 2024

Related U.S. Application Data
(60) Provisional application No. 63/297,542, filed on Jan. 7, 2022.
(60) Provisional application No. 63/239,372, filed on Aug. 31, 2021.

(51) Int. Cl. A61K 47/68; A61K 31/7105; A61K 38/46; A61K 39/00; A61P 35/00; C12N 15/115

(52) U.S. Cl. CPC A61K 47/6843; A61K 31/7105; A61K 38/465; A61K 39/0011; A61K 39/001119; A61K 39/001186; A61K 39/001189; A61K 47/6807; A61K 47/6813; A61P 35/00; C12N 15/115; C12N 2310/113; C12N 2310/12; C12N 2310/14; C12N 2310/141

(57) ABSTRACT

Compositions and methods are provided for treating cancer by administering a complex formed between a therapeutic polynucleotide and a 3E10 antibody or variant thereof, or antigen-binding fragment thereof. In some instances, the complexes are stabilized through a molar ratio of 3E10 antibody or variant thereof, or antigen-binding fragment thereof to therapeutic polynucleotide of at least about 2:1.

### STATEMENT REGARDING FEDERALLY SPONSORED RESEARCH

This invention was made with government support under R35CA197574 awarded by National Institutes of Health. The Government has certain rights in the invention.

### TECHNICAL FIELD

The present disclosure relates generally to compositions and methods for treating cancers.

### BACKGROUND

Cancers are a diverse group of hyper-proliferative diseases having a wide range of etiologies and clinical manifestations. Because of the significant differences across cancer biology, no single therapeutic strategy—let alone particular therapy—is sufficient for treatment of every cancer type. Rather, different therapeutic approaches have been developed to treat different types of cancer. Among these various therapeutic strategies, several classes of polynucleotide-based cancer therapies have been developed.

Among the polynucleotide-based cancer therapies, many different strategies have evolved. For instance, immunostimulant polynucleotides have been used to agonize mediators of proinflammatory cytokines, such as pattern recognition receptors, in various cancer immunotherapies. Gene-regulating polynucleotides, e.g., siRNA, miRNA, ASO, etc., have been used to silence targeted genes, regulating signaling pathways involved in cancer progression. Polynucleotides encoding therapeutic proteins, e.g., mRNA or plasmids encoding antigens or cancer immunotherapeutic proteins have also been used therapeutically. Functional nucleic acids, such as aptamers, have also been used in a similar fashion to antibody-based cancer therapies, e.g., by binding and blocking key oncology targets, such as PD-1. Gene editing polynucleotides have also been used, in an analogous fashion as gene-regulating polynucleotides, to silence expression of cancer mediators. For a review of various polynucleotide-based cancer therapies see, for example, Medicine in Drug Discovery, 6 (2020) 100023 and Hager et al., Cells, 9(9):2061 (2020), the contents of which are incorporated herein by reference.

Although these polynucleotide-based cancer therapies have met with some success in preclinical studies, they have fallen short of expectations when evaluated for therapeutic efficacy in clinical trials (Lopes et al., Cancer DNA vaccines: Current preclinical and clinical developments and future perspectives. J. Exp. Clin. Cancer Res., 2019, 38, 146; Dörrie et al., Therapeutic Cancer Vaccination with ex vivo RNA-Transfected Dendritic Cells—An Update. Pharmaceutics, 2020, 12, 92). One major hurdle has been the lack of appropriate delivery systems required to prevent degradation of pDNA/mRNA, and to enable cell type-specific delivery (Angell et al., DNA Nanotechnology for Precise Control over Drug Delivery and Gene Therapy. Small (Weinheim an der Bergstrasse, Germany), 2016, 12, 1117-1132; Das et al., Gene Therapies for Cancer: Strategies, Challenges and Successes. J. Cell. Physiol., 2015, 230, 259-271).

Nucleic acids do not readily cross the cell membrane. Conventional approaches to overcoming this obstacle include packaging nucleic acids in liposomal-based delivery vehicles, which presents immunological challenges as seen in DNA-based therapies. Further, nucleic acids are readily degraded by extracellular nucleases present in skin, tissues, and blood. Kowalski P S et al., Mol Ther., 27(4):710-28 (2019), the content of which is incorporated by reference herein.

### SUMMARY

Given the background above, there is a need in the art for improved methods of treating cancers with therapeutic polynucleotides. Polynucleotide-based cancer therapies present a promising path for cancer therapy because of their versatility to encode any polypeptide, the availability of highly reproducible manufacturing methods, the ability to make simple and precise adjustments to polynucleotide sequences, their inexpensive nature, their ability to specifically target and/or edit any genetic sequence, etc. However, the delivery of polynucleotide therapeutics to specific tissues in vivo has posed many challenges, including the rapid degradation of foreign nucleic acids in the body and immunogenicity caused by common delivery vehicles, such as liposomes and viral vectors. See, for example, Zhou et al., Medicine in Drug Discovery, 6 (2020) 100023 and Dahlman et al., Nature Nanotechnol. 9(8):648-655 (2014), the contents of which are incorporated herein by reference. Advantageously, the present disclosure provides compositions and methods for polynucleotide-based cancer therapy that are not reliant upon liposomal or viral vector based nucleic acid delivery.

In some aspects, these compositions and methods are based on, at least in part, on the discovery that 3E10 antibodies or variants thereof, or antigen-binding fragments thereof can be used to efficiently deliver therapeutic polynucleotides to cancerous tissue in vivo. For instance, as described in the Examples, 3E10 mediates delivery of polynucleotides in vivo to various cancers following systematic administration in murine models, including mammary cancer (Examples 1 and 2), brain cancer (Example 7), skin cancer (Examples 16, 18, and 21), pancreatic cancer (Example 17), and colon cancer (Example 22).

Accordingly, in one aspect, the disclosure provides a method for treating a cancer in a subject in need thereof by administering to the subject a therapeutically effective amount of a composition comprising a complex formed between (i) a therapeutic polynucleotide, and (ii) a 3E10 antibody or variant thereof, or antigen-binding fragment thereof.

In another aspect, the present disclosure provides pharmaceutical compositions of a complex formed between (i) a therapeutic polynucleotide, and (ii) a 3E10 antibody or variant thereof, or antigen-binding fragment thereof.

In some embodiments of the methods and compositions described herein, the therapeutic polynucleotide is, or encodes for, a polynucleotide immunostimulant, e.g., polynucleotide ligand capable of stimulating a pattern recognition receptor (PRR), cyclic-GMP-AMP-synthase (cGAS), or Stimulator of interferon genes (STING). In some embodiments of the methods and compositions described herein, the therapeutic polynucleotide encodes a protein or peptide for cancer therapy, e.g., a tumor antigen or proinflammatory cytokine. In some embodiments of the methods and compositions described herein, the therapeutic polynucleotide is, or encodes for, a gene-regulating polynucleotide, e.g., an siRNA, miRNA, saRNA, antagomir, antisense oligonucleotide, or decoy oligonucleotide. In some embodiments of the methods and compositions described herein, the therapeutic polynucleotide encodes a genome editing effector, e.g., a zinc-finger nuclease, a transcription activator-like effector nuclease (TALEN), or a CRISPR system comprising a Cas protein and a guide RNA. In some embodiments of the methods and compositions described herein, the therapeutic polynucleotide is, or encodes for, an effector polynucleotide, e.g., an aptamer or ribozyme.

Advantageously, this ability of 3E10-PRR agonist complexes to mediate cell death is exploited in the compositions and methods described herein for the treatment of various cancers.

Accordingly, one aspect of the present disclosure provides methods for treating a cancer in a subject in need thereof. In some embodiments, the method includes administering to the subject, a therapeutically effective amount of a composition comprising a complex formed between (i) a polynucleotide ligand capable of stimulating a pattern recognition receptor (PRR), and (ii) a 3E10 antibody or variant thereof, or antigen-binding fragment thereof.

In some embodiments, the advantageous properties of the compositions and methods described herein are based, at least in part, on the discovery that 3E10 antibodies or variants thereof, or antigen-binding fragments thereof, as described below, readily cross the blood-brain barrier. For instance, as described in Example 6 and illustrated in FIG. 13, 3E10-D31N accumulated and was retained in the CNS of mice bearing human medulloblastoma tumors for at least 8 days, both in the brain and at the spinal cord. Advantageously, the ability of 3E10 to cross the blood-brain barrier is exploited in the compositions and methods described herein to deliver polynucleotide PRR agonists across the BBB and into the CNS for the treatment of various cancers.

In some embodiments, the advantageous properties of the compositions and methods described herein are based, at least in part, on the discovery that complexes of polynucleotide PRR agonists and 3E10 antibodies or variants thereof, or antigen-binding fragments thereof, as described below, significantly reduce tumor burden of a CNS cancer following system administration. For instance, as described in Example 7 and illustrated in FIG. 14, and particularly in FIG. 14D, a single dose of GMAB^{D31N}/3pRNA, but not the PBS control, significantly reduced tumor burden of a human medulloblastoma in the brain of mice following intravenous administration. Advantageously, the ability of these complexes to reduce CNS tumor burden following systemic administration is exploited in the compositions and methods described herein to provide relatively simple and effective therapy of CNS cancers.

In some embodiments, the advantageous properties of the compositions and methods described herein are based, at least in part, on the discovery that complexes of polynucleotide PRR agonists and 3E10 antibodies or variants thereof, or antigen-binding fragments thereof, as described below, significantly prevent metastasis of brain tumors to the spinal cord following system administration. For instance, as described in Example 7 and illustrated in FIG. 14, and particularly in FIG. 14E, a single dose of GMAB^{D31N}/3pRNA, but not the PBS control, significantly prevented metastasis of a human medulloblastoma in the brain of mice following intravenous administration. Advantageously, the ability of these complexes to prevent or reduce metastasis of brain tumors following systemic administration is exploited in the compositions and methods described herein to provide relatively simple and effective therapy of CNS cancers.

In some embodiments, the advantageous properties of the compositions and methods described herein are based, at least in part, on the discovery that use of higher molar ratios of 3E10 antibody or variant thereof, or antigen-binding fragment thereof to polynucleotides result in greater protection of the polynucleotide from RNA degradation. For instance, as described in Example 6 and illustrated in FIGS. 13A and 13B, while parental 3E10 and 3E10 (D31N) variant antibodies protected mRNA from RNAse A-mediated RNA degradation at molar ratios of 2:1 and 20:1, the protection afforded by the 20:1 molar ratio exceeded the protection afforded at 2:1. Advantageously, the increased protection afforded polynucleotide at higher 3E10 antibody or variant thereof, or antigen-binding fragment thereof concentrations is exploited in the compositions and methods described herein to improve the pharmacokinetic properties of therapeutic compositions delivering polynucleotides in vivo.

Accordingly, one aspect of the present disclosure provides pharmaceutical compositions of a complex formed between (i) a polynucleotide ligand capable of stimulating a pattern recognition receptor (PRR), and (ii) a 3E10 antibody or variant thereof, or antigen-binding fragment thereof, for the treatment of various cancers. In some embodiments, the pharmaceutical composition has a molar ratio of 3E10 antibody or variant thereof, or antigen-binding fragment thereof to polynucleotide ligand of at least 2:1.

In some embodiments of the methods and compositions described herein, the 3E10 antibody or variant thereof, or antigen-binding fragment thereof includes (a) a light chain variable region (VL) complementarity determining region (CDR) 1 comprising the amino acid sequence of 3E10-VL-CDR1 (SEQ ID NO: XX), (b) a VL CDR2 comprising the amino acid sequence of 3E10-VL-CDR2 (SEQ ID NO: XX), (c) a VL CDR3 comprising the amino acid sequence of 3E10-VL-CDR3 (SEQ ID NO: XX), (d) a heavy chain variable region (VH) CDR1 comprising the amino acid sequence of 3E10-VH-CDR1a (SEQ ID NO: XX), (e) a VH CDR2 comprising the amino acid sequence of 3E10-VH-CDR2 (SEQ ID NO: XX), and (f) a VH CDR3 comprising the amino acid sequence of 3E10-VH-CDR3 (SEQ ID NO: XX).

In some embodiments of the methods and compositions described herein, the 3E10 antibody or variant thereof, or antigen-binding fragment thereof includes (a) a light chain variable region (VL) complementarity determining region (CDR) 1 comprising an amino acid sequence having no more than two amino acid substitutions relative to 3E10-VL-CDR1 (SEQ ID NO: XX), (b) a VL CDR2 comprising an amino acid sequence having no more than two amino acid substitutions relative to 3E10-VL-CDR2 (SEQ ID NO: XX), (c) a VL CDR3 comprising an amino acid sequence having no more than two amino acid substitutions relative to 3E10-VL-CDR3 (SEQ ID NO: XX), (d) a heavy chain variable region (VH) CDR1 comprising an amino acid sequence having no more than two amino acid substitutions relative to 3E10-VH-CDR1a (SEQ ID NO: XX), (e) a VH CDR2 comprising an amino acid sequence having no more than two amino acid substitutions relative to 3E10-VH-CDR2 (SEQ ID NO: XX), and (f) a VH CDR3 comprising an amino acid sequence having no more than two amino acid substitutions relative to 3E10-VH-CDR3 (SEQ ID NO: XX).

In some embodiments of the methods and compositions described herein, the 3E10 antibody or variant thereof, or antigen-binding fragment thereof includes (a) a light chain variable region (VL) complementarity determining region (CDR) 1 comprising the amino acid sequence of 3E10-VL-CDR1m (SEQ ID NO: XX), (b) a VL CDR2 comprising the amino acid sequence of 3E10-VL-CDR2m (SEQ ID NO: XX), (c) a VL CDR3 comprising the amino acid sequence of 3E10-VL-CDR3m (SEQ ID NO: XX), (d) a heavy chain variable region (VH) CDR1 comprising the amino acid sequence of 3E10-VH-CDR1m (SEQ ID NO: XX), (e) a VH CDR2 comprising the amino acid sequence of 3E10-VH-CDR2m (SEQ ID NO: XX), and (f) a VH CDR3 comprising the amino acid sequence of 3E10-VH-CDR3m (SEQ ID NO: XX).

In some embodiments of the methods and compositions described herein, the 3E10 antibody or variant thereof, or antigen-binding fragment thereof is a bispecific antibody that includes a binding sequence that targets a cell type, tissue, or organ of interest, e.g., a cancerous tissue or cell.

### BRIEF DESCRIPTION OF THE DRAWINGS

FIG. 1 illustrates amino acid sequences for the parent 3E10 monoclonal antibody.

FIGS. 2A, 2B, and 2C illustrate amino acid sequences for the D31N variant (FIG. 2A), other CDR variants (FIG. 2B), and additionally contemplated CDR variants (FIG. 2C) of the 3E10 monoclonal antibody, in accordance with some embodiments of the present disclosure.

FIG. 3 illustrates example charge-conserved CDR variants of the 3E10 monoclonal antibody, in accordance with various embodiments of the present disclosure.

FIG. 4 illustrates example CDR variants containing a combination of amino acid substitutions, charged-conserved amino acid substitutions, and rationally designed amino acid substitutions of the 3E10 monoclonal antibody, in accordance with various embodiments of the present disclosure.

FIG. 5 illustrates a sequence alignment of examples of humanized 3E10 heavy chain variable regions, with CDRs underlined as indicated.

FIG. 6 illustrates a sequence alignment of examples of humanized 3E10 light chain variable regions, with CDRs and putative nuclear localization signals (NLS) underlined as indicated.

FIGS. 7A, 7B, 7C, 7D, and 7E collectively illustrate a sequence alignment of example of humanized di-scFv constructs of the 3E10 monoclonal antibody.

FIG. 8A is a bar graph showing accumulation in tumors of fluorescently labeled siRNA mixed with increasing doses of 3E10 (0.25, 0.5, and 1 mg) for 15 minutes at room temperature prior to systemic injection in mice.

FIG. 8B is a bar graph showing accumulation in tumors of fluorescently labeled siRNA mixed with 1 mg 3E10 or 0.1 mg D31N variant 3E10 for 15 minutes at room temperature prior to systemic injection in mice. All tumors were analyzed 24 hours after injection.

FIGS. 9A, 9B, and 9C collectively show in vivo tumor localization of 3E10-D31N following intravenous administration. FIGS. 9A and 9B are images showing control (FIG. 9A) and distribution of IV Injected 3E10-D31N to syngeneic colon tumors (CT26) (FIG. 9B), imaged by IVIS (Perkin Elmer) 24 hours after injection. FIG. 9C is a bar graph quantifying the fluorescence in the IVIS images.

FIGS. 10A, 10B, 10C, and 10D collectively show in vivo tumor localization of ssDNA following intravenous administration with and without 3E10-D31N. FIGS. 10A, 10B, and 10C are images showing control (FIG. 10A), and distribution of IV Injected naked single stranded DNA (ssDNA) (FIG. 10B) and 3E10-D31N+ssDNA (FIG. 10C) syngeneic colon tumors (CT26), imaged by IVIS (Perkin Elmer) 24 hours after injection. FIG. 10D is a bar graph quantifying the fluorescence in the IVIS images.

FIG. 11 is a bar graph showing 3E10-mediated delivery and stimulation of RIG-I.

FIGS. 12A and 12B are bar graphs showing the impact of wildtype (12A) or D31N 3E10 (12B)+Poly (I:C) in a melanoma cell viability.

FIGS. 13A and 13B collectively show that intravenous injection of fluorescently labeled 3E10-D31N in a mouse model of human medulloblastoma (DAYO) results in antibody accumulation and retention in the CNS of mice, both in the brain and at the spinal cord. FIG. 13A shows IVIS imaging of 3E10-D31N fluorescence in vivo over 8 days following administration. FIG. 13B illustrates quantitation of the fluorescence in treated (open circles) and un-treated control (closed circles) mice.

FIGS. 13C and 13D collectively illustrate that cD31N/3p-hpRNA targets orthoptic medulloblastoma tumors and suppresses tumor growth. FIG. 13C shows IVIS visualization, and FIG. 13D shows quantification, of tumor growth in mice bearing luciferase-expressing orthotopic medulloblastoma tumors (DAOY) treated with vehicle control (PBS), 3p-hpRNA, cD31N, cD31N/3p-hpRNA, or temozolomide. Data from n=4 per group, with statistical analysis performed by student's t-test for individual comparison, with p values indicated.

FIGS. 14A, 14B, 14C, 14D, and 14E collectively show that systemic administration of a single dose of a RIG-I polynucleotide agonist and 3E10-D31N complex reduces tumor burden and prevents metastasis in a mouse model of a human medulloblastoma tumor. FIG. 14A shows images of luciferase expression in DAYO cells 1, 3, 4, 8, and 9 days post intravenous administration of PBS control (top row), 3E10-D31N alone (middle row), or a complex of 3E10-D31N and 3pRNA RIG-I agonist (bottom row). FIG. 14B shows images of luciferase expression in DAYO cells 14 days post intravenous administration of PBS control (top row) or a complex of 3E10-D31N and 3pRNA RIG-I agonist (bottom row). FIG. 14C illustrates quantitation of medulloblastoma growth over two weeks post intravenous administration of PBS control (black), 3E10-D31N alone (gray), or a complex of 3E10-D31N and 3pRNA RIG-I agonist (blue). FIG. 14D shows images of luciferase expression in DAYO cells in the brain of mice 14 days post intravenous administration of PBS control (left panel) or a complex of 3E10-D31N and 3pRNA RIG-I agonist (right panel). FIG. 14E shows images of luciferase expression in DAYO cells at the spinal cord of mice 14 days post intravenous administration of PBS control (left panel) or a complex of 3E10-D31N and 3pRNA RIG-I agonist (right panel).

FIGS. 15A and 15B illustrate electrostatic surface potential renderings of a molecular model of a 3E10-scFv construct, revealing a putative Nucleic Acid Binding pocket (NAB1). FIG. 15A additionally shows predicted structural and electrostatic potential changes induced by amino acid substitutions at residue HC CDR1 residue 31. FIG. 15B is an illustration of molecular modeling of 3E10-scFv (Pymol) with NAB1 amino acid residues highlighted by punctate dots.

FIG. 15C illustrates mapping of the putative nucleic acid binding pocket, as identified by the molecular modeling shown in FIGS. 15A and 15B, onto the amino acid sequence of the 3E10-scFv construct.

FIGS. 16A and 16B show gel electrophoresis of mRNA protection assays performed with complexes formed between 3E10 and a 720 nucleotide mRNA molecule prepared at 20:1 (FIG. 16A) and 2:1 (FIG. 16B) (3E10:mRNA) molar ratios, as described in Example 9.

FIG. 17 shows gel electrophoresis analysis of mRNA protection assays performed with complexes formed between 3E10 and a 14 kb mRNA molecule prepared at 1:1, 2:1, 5:1, 10:1 and 100:1 (3E10:mRNA) molar ratios, as described in Example 10.

FIGS. 18A, 18B, and 18C illustrate histograms collectively showing a 3-day time course of the type-1 IFN response in THP-1 monocytes following exposure to PBS (control), 3p-hpRNA alone, increasing amounts of 3E10-D31N (GMAB) alone, and 3E10-D31N/3p-hpRNA complexes prepared in increasing molar ratios (3E10:3p-hpRNA), as described in Example 11.

FIGS. 19A and 19B illustrate histograms showing viability of human brain tumor cells following exposure to PBS (control), 3p-hpRNA alone, 3E10-D31N (GMAB) alone, and 3E10-D31N/3p-hpRNA complexes, as described in Example 12.

FIGS. 20A and 20B illustrate histograms showing cell necrosis of human brain tumor cells following exposure to PBS (control), 3p-hpRNA alone, 3E10-D31N (GMAB) alone, and 3E10-D31N/3p-hpRNA complexes, as described in Example 13.

FIGS. 21A, 21B, 21C, and 21D illustrate histograms showing cell necrosis of human (21A and 21B) and murine (21C and 21D) colorectal cancer cells following exposure to PBS (control), 3p-hpRNA alone, 3E10-D31N (GMAB) alone, and 3E10-D31N/3p-hpRNA complexes, as described in Example 14.

FIG. 22 illustrates a histogram showing cell necrosis of human breast cancer cells following exposure to PBS (control), 3p-hpRNA alone, 3E10-D31N (GMAB) alone, and 3E10-D31N/3p-hpRNA complexes, as described in Example 15.

FIG. 23 illustrates a histogram showing IL-10 concentration following exposure of B16 melanoma cells to PBS (control), 3E10-D31N (GMAB) alone, 3p-hpRNA alone, 3E10-D31N/3p-hpRNA complexes, or an anti-CTLA-4 antibody, as described in Example 16.

FIG. 24 illustrates a histogram showing IL-6 concentration following exposure of B16 melanoma cells to PBS (control), 3E10-D31N (GMAB) alone, 3p-hpRNA alone, 3E10-D31N/3p-hpRNA complexes, or anti-CTLA-4, as described in Example 16.

FIGS. 25A and 25B show in vivo images of bioluminescence of a luciferase reporter in cancerous tissue (FIG. 25A) and fluorescence of labeled 3E10-D31N (FIG. 25B) in mice having orthotopic pancreatic tumors following intravenous administration of PBS (left panels) or 3E10-D31N/3p-hpRNA complexes, as described in Example 17.

FIGS. 26A and 26B show bioluminescence of a luciferase reporter in cancerous tissue (FIG. 26A) and fluorescence of labeled 3E10-D31N (FIG. 26B) in resected orthotopic pancreatic tumors following intravenous administration of PBS (left panels), 3E10-D31N alone (middle panels), or 3E10-D31N/3p-hpRNA complexes (right panels), as described in Example 17.

FIG. 26C illustrates fluorescence of labeled 3E10-D31N in resected organs from representative mice having orthotopic pancreatic tumors following intravenous administration of PBS (left panel) or 3E10-D31N/3p-hpRNA complexes, as described in Example 17.

FIG. 26D shows a histogram of average radiant efficiency (fluorescence) of labeled 3E10-D31N in resected organs from mice in cohorts treated with PBS (control; left plot in each group), 3E10-D31N (GMAB) alone (middle plot in each group), and 3E10-D31N/3p-hpRNA complexes (right plot in each group), as described in Example 17.

FIG. 26E shows bioluminescence of a luciferase reporter in cancerous tissue (left column) and fluorescence of labeled 3E10-D31N (right column) in resected organs of all mice in the cohort treated with 3E10-D31N/3p-hpRNA complexes, as described in Example 17.

FIGS. 27A, 27B, 27C, 27D, 27E, 27F, 27G, 27H, 27I, 27J, 27K, 27L, 27M, and 27N collectively show cytokine concentration and TIL tumor infiltration following exposure of a murine melanoma model to PBS (control), 3E10-D31N (GMAB) alone, 3p-hpRNA alone, 3E10-D31N/3p-hpRNA complexes, or an anti-CTLA-4 antibody, as described in Example 18.

FIG. 28 illustrates the Type I IFN response elicited by 3E10-D31N mediated delivery of 3p-hpRNA, as described in Example 19.

FIG. 29 illustrates cell death caused by 3E10-D31N mediated delivery of 3p-hpRNA, as described in Example 20.

FIGS. 30A, 30B, 30C, 30D, 30E, 30F, 30G, 30H, 30I, 30J, and 30K collectively show that intravenous administration of 3E10-D31N/3p-hpRNA complexes suppresses tumor growth in a mouse model of melanoma. FIG. 30A illustrates average melanoma tumor volume in a murine model following systemic administration of PBS (control; ●), 3E10-D31N (GMAB; ▪) alone, 3p-hpRNA alone (▴), 3E10-D31N/3p-hpRNA complexes (▾), or an anti-CTLA-4 antibody (♦) at days 9, 11, 13, and 15 post tumor-transplantation, as described in Example 21. The tumor volume (mm³) for each of five mice per cohort, and an image of a representative tumor in the cohort at day 23 post-transplantation, following systemic administration of PBS (control; 30B and 30C), 3E10-D31N (GMAB; 30D and 30E) alone, 3p-hpRNA alone (30F and 30G), 3E10-D31N/3p-hpRNA complexes (30H and 30I), or an anti-CTLA-4 antibody (30J and 30K) at days 9, 11, 13, and 15 post tumor-transplantation are also shown.

FIGS. 31A, 31B, 31C, 31D, 31E, 31F, 31G, 31H, 31I, 31J, 31K, 31L, and 31M collectively show that intravenous administration of 3E10-D31N/3p-hpRNA complexes synergize with anti-PD-1 antibody therapy to suppress tumor growth in a mouse model of colon cancer. FIG. 31A illustrates average colon tumor volume in a murine model following systemic administration of PBS (control; ●), 3E10-D31N/3p-hpRNA complexes (▾), 3E10-D31N/3p-hpRNA complexes+anti-PD-1 (♦), or anti-PD-1 alone (∘) at days 8, 11, and 14 post cancer cell injection, as described in Example 22. The tumor volume (mm³) for each of five mice per cohort, and an image of a representative tumor in the cohort at day 21 post-injection, following systemic administration of PBS (control; 31B and 31C), 3E10-D31N alone (GMAB; 31D and 31E), 3p-hpRNA alone (31F and 31G), 3E10-D31N/3p-hpRNA complexes (31H and 31I), an anti-PD-1 antibody (31J and 31K), or 3E10-D31N/3p-hpRNA complexes+anti-PD-1 antibody (31L and 31M) at days 8, 11, and 14 post cancer cell injection are also shown.

FIGS. 32A, 32B, 32C, 32D, 32E, 32F, 32G, and 32H collectively show that intravenous administration of 3E10(D31N)/3p-hpRNA complexes synergize with anti-PD-1 antibody therapy to suppress tumor growth in a mouse model of breast cancer. FIG. 32A illustrates average breast tumor volume in a murine model following systemic administration of PBS (control; e), 3E10(D31N)/3p-hpRNA complexes (▾), 3E10(D31N)/3p-hpRNA complexes+anti-PD-1 (4), or anti-PD-1 alone (∘) at days 8, 11, and 14 post cancer cell injection, as described in Example 32. The tumor volume (mm³) for each of five mice per cohort following systemic administration of PBS (control 32B), 3E10(D31N) alone (GMAB; 32C), 3p-hpRNA alone (32D), 3E10(D31N)/3p-hpRNA complexes (32E), an anti-PD-1 antibody (32F), or 3E10(D31N)/3p-hpRNA complexes+anti-PD-1 antibody (32G) at days 8, 11, and 14 post cancer cell injection are also shown. FIG. 32H illustrates that intravenous administration of 3E10(D31N)/3p-hpRNA complexes synergize with anti-PD-1 antibody therapy to generate immunological memory.

FIGS. 33A, 33B, 33C, 33D, 33E, and 33F collectively show that intravenous administration of 3E10(D31N)/3p-hpRNA complexes reduce tumor volume in a mouse model of medulloblastoma. FIGS. 33A-33E show representative images of luciferase expression in DAYO cells 10 days post intravenous administration of PBS control (33A), temozolomide (33B), 3E10-D31N alone (GMAB; 33C), 3p-hpRNA alone (33D), and 3E10-D31N/3p-hpRNA complexes (33E). FIG. 33F illustrates average normalized tumor volumes across mice in each cohort, as described in Example 7.

FIG. 34 shows a histogram of cytosolic, membrane, nuclear protein, and gDNA fractions after administration of ⁸⁹Zr labeled isotype control, 3E10-WT, and 3E10-D31N antibodies, as described in Example 24.

FIGS. 35A, 35B, 35C, 35D, 35E collectively demonstrate that chimeric 3E10-D31N antibody (cD31N) non-covalently binds to RNA and distributes to tumors in vivo. FIG. 35A shows quantification of binding of the cD31N antibody and the related chimeric 3E10 antibody (cWT; without the D31N substitution) to single-stranded DNA by ELISA. FIG. 35B shows quantification of cD31N binding to single-stranded RNA by ELISA with cWT as a comparison. FIG. 35C illustrates Bio-Layer Interferometry (BLI) measurement of cD31N affinity for 89 nucleotide 3p-hpRNA as a function of increasing RNA concentration. Derived values for the dissociation constant (KD) and related parameters are indicated in the accompanying table. FIG. 35D shows measurement of hydrodynamic radii of cD31N/3p-hpRNA antibody RNA complexes (3p-ARCs) as compared to cD31N alone using dynamic light scattering. Data are mean±s.e.m. from n=16. P values determined by two-tailed unpaired t-test. FIG. 35E illustrates (Left) penetration of fluorescently labeled cD31N into U2OS human osteosarcoma cells compared to an isotype control, and (Right) delivery of fluorescently labeled 3p-hpRNA into U2OS cells when cells are treated with 3p-hpRNA complexed with cD31N compared to labeled 3p-hpRNA incubated with an isotype control. FIG. 35F illustrates IVIS visualization of tumor and normal tissue distribution of IV administered fluorescently labeled cD31N in mice bearing EMT6 flank tumors 24 hours after administration (n=1). FIG. 35G illustrates IVIS visualization of tumor and normal tissue distribution of IV administered siGLO (fluorescently labeled siRNA) complexed to cD31N in mice bearing EMT6 flank tumors relative to the siRNA alone as a control 24 hours after administration (n=1).

FIGS. 36A, 36B, 36C, 36D, and 36E collectively illustrate that chimeric 3E10-D31N antibody (cD31N)/3p-hpRNA antibody/RNA complexes induce a RIG-I-dependent interferon response in cells and mediate tumor growth suppression in a mouse model of melanoma. FIG. 36A shows quantification of type 1 IFN induction in THP-1 monocytes or THP-1 RIG-I KO monocytes both containing an IFN-response luciferase reporter construct. Cells were treated with vehicle control (PBS), 3p-hpRNA, cD31N, or cD31N/3p-hpRNA. Data are mean±s.e.m. from n=4 with statistical analysis performed by ANOVA, with p values indicated. FIG. 36B illustrates a schematic representation of in vivo dosing schedule by retro-orbital IV injection in immune competent C57Bl/6 mice bearing B16.F10.OVA flank tumors for tumor growth study.

FIG. 36C shows tumor growth curves in mice bearing B16.F10.OVA flank tumors treated with vehicle control (PBS), cD31N, 3p-hpRNA, cD31N/3p-hpRNA, or anti-CTLA-4. Data from n=5-6 per group with statistical analysis performed by student's t-test for individual comparison, with p values indicated. FIG. 36D illustrates body weights of treated mice bearing B16.F10.OVA flank tumors over the course of the experiment. FIG. 36E illustrates terminal bone marrow cell counts following treatment in B16.F10.OVA flank tumor bearing mice. Data from n=5-6 per group with statistical analysis performed by ANOVA, with p values indicated.

FIGS. 37A and 37B collectively illustrate that cD31N/3p-hpRNA antibody/RNA complexes induce tumor infiltrating leukocytes in melanoma flank tumors. Percentages of CD45+, CD8+, NK, NKT, and CD19+ cells in tumors (FIG. 37A) and in splenic controls (FIG. 37B) harvested from treated C57Bl/6 mice bearing B16.F10.OVA tumors 24 hours after two doses of indicated treatments were administered (on days 10 and 13 post-implantation). Data are mean±s.e.m. from n=5 treated mice with statistical analysis performed by ANOVA, with p values indicated.

FIGS. 38A, 38B, 38C, 38D, 38E, 38F, 38G, 38H, 38I, 38J, 38K, 38L, 38M, 38N, 38O, 38P collectively show that cD31N/3p-hpRNA complexes penetrate orthotopic pancreatic tumors, enhance survival, induce tumor necrosis, and promote immunogenicity. FIG. 38A illustrates a schematic representation of in vivo dosing schedule by retro-orbital IV injection in mice bearing orthotopic KPC tumors. FIG. 38B shows RT-PCR quantification of 3p-hpRNA delivery to tumor cells compared to CD45+ cells isolated from KPC orthotopic tumors by flow sorting one day following the last of three doses of 3p-hpRNA/cD31N. Data from n=5 per group with statistical analysis performed by student's t-test for individual comparison, with p values indicated. FIG. 38C illustrates survival curves in control or cD31N/3p-hpRNA treated mice. Data from n=6-7 with statistical analysis performed by Log-rank (Mantel-Cox) test, with indicated p values. FIG. 38D illustrates quantification of histological analysis of tumor cell necrosis. Data from n=4 with statistical analysis performed by student's t-test for individual comparison, with p values indicated. FIG. 38E shows H&E staining images of tumor sections from control and cD31N/3p-hpRNA treated mice at two magnifications illustrative of tumor cell necrosis in cD31N/3p-hpRNA treated mice relative to controls. FIG. 38F shows percentages of CD4+ cells isolated from tumors and quantified by flow cytometry. FIG. 38G shows percentages of CD8+ cells isolated from tumors and quantified by flow cytometry. FIG. 38H and FIG. 38I show 3E10-D31N efficiently targeted orthotopic KPC tumors in mice, as demonstrated by robust co-localization of the labeled antibody with the luciferase-expressing tumors. FIG. 38J shows complexation of labeled 3E10-D31N with 3p-hpRNA did not compromise the antibody's ability to target orthotopic pancreatic tumors. FIG. 38K shows isolated tumors and flow-sorted single cell suspensions into EPCAM+ epithelial (tumor) or CD45+ populations. FIG. 38L shows 3p-hpRNA delivery specifically into KPC pancreatic tumor cells relative to CD45+ immune cells within the same tumors (p<0.0001). FIG. 38M illustrates survival curves in control or cD31N/3p-hpRNA treated mice. FIG. 38N shows treatment with 3E10-D31N/3p-hpRNA RIG-I agonist led to robust increases in tumor cell necrosis. FIG. 38O shows 3E10-D31N/3p-hpRNA RIG-I agonist treatment reducing the proportion of mice with detectable metastases in the organs (liver, lung, and kidney). FIG. 38P shows RT-PCR data quantifying CD3, CD4 and CD8 mRNA expression after 3E10-D31N/3p-hpRNA RIG-I agonist administration in a murine KPC model of pancreatic ductal adenocarcinoma.

FIGS. 39A and 39B collectively illustrate that cD31N binds multiple species of RNA. Bio-Layer Interferometry (BLI) measurement of cD31N affinity for (FIG. 39A) GFP-mRNA and (FIG. 39B) a KRAS-targeting siRNA. Derived values for the dissociation constants (KD) and related parameters are indicated in the corresponding tables.

FIG. 40 illustrates that cD31N cellular penetration is ENT2 dependent. U2OS human osteosarcoma cells were pre-treated with dipyridamole or NBMPR (6-S-[(4-Nitrophenyl)methyl]-6-thioinosine) for 30 minutes prior to incubation with cD31N. Following 30 minutes of treatment with cD31N, uptake was quantified by immunofluorescence normalized to DAPI (4′,6-diamidino-2-phenylindole) staining of nuclei. Statistical analysis performed by student's t-test for individual comparison, with p values indicated.

FIGS. 41A and 41B collectively illustrate that ENT2 is highly expressed in multiple tumor models. Western blot analysis (FIG. 41A) and quantification (FIG. 41B) of ENT2 protein expression in cell lysates prepared from murine tissues (C57Bl/6) and indicated tumor cell lines.

### DETAILED DESCRIPTION

Advantageously, methods and compositions were developed for targeting therapeutic polynucleotides to various cancer tissues in vivo and facilitating delivery of these therapeutic polynucleotides into diseased cells, e.g., cancer cells displaying high levels of ENT2 on their cell surface. Further, these methods protect the therapeutic polynucleotides from degradation by extracellular and intracellular nucleases, without blocking access to the polynucleotides once internalized within the cell. Thus, the present disclosure provides compositions and methods for delivering therapeutic polynucleotides to cancerous tissue. In some embodiments, the methods and compositions find particular use for the treatment of cancers. For instance, compositions comprising a complex formed between (i) a therapeutic polynucleotide and (ii) a 3E10 antibody or variant thereof, or antigen-binding fragment thereof, as well as methods for using such compositions for the treatment of cancers, are described.

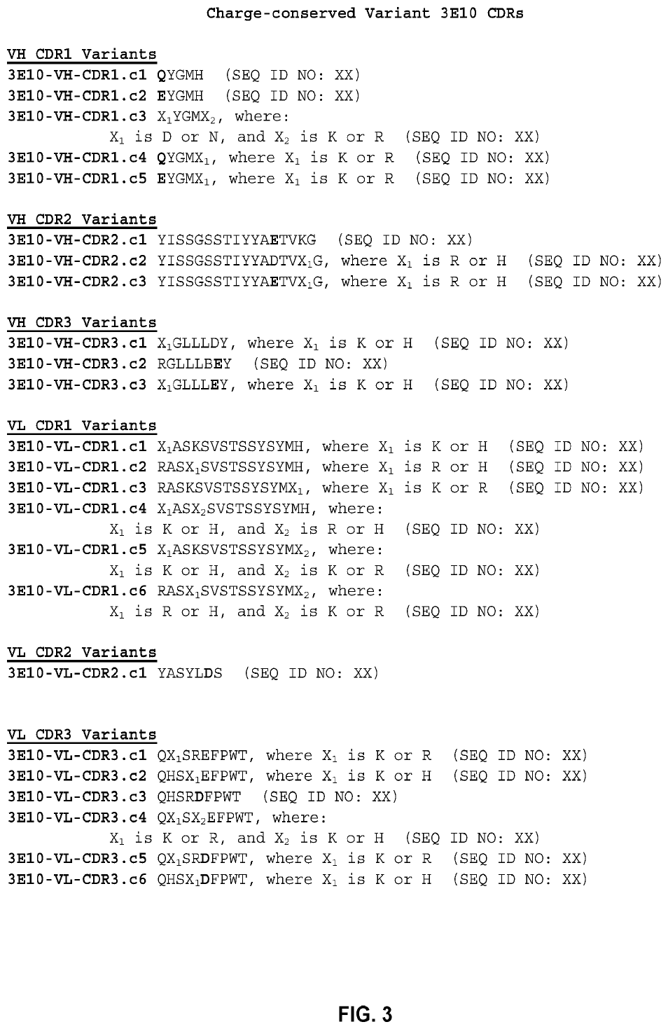

In some embodiments, the advantageous properties of the compositions and methods described herein are based, at least in part, on the discovery that 3E10 antibodies or variants thereof, or antigen-binding fragments thereof, as described below, localize to cancerous tissue in vivo following systemic administration. For instance, as described in Examples 1 and 2 and illustrated in FIGS. 8 and 9, 3E10 and 3E10-D31N variant antibodies localized to EMT6 flank tumors in mice following intravenous administration. Advantageously, this tropism for cancerous tissues is exploited in the compositions and methods described herein to target such tissues for delivery of therapeutic polynucleotides for the treatment of various cancers, e.g., cancers displaying ENT2.

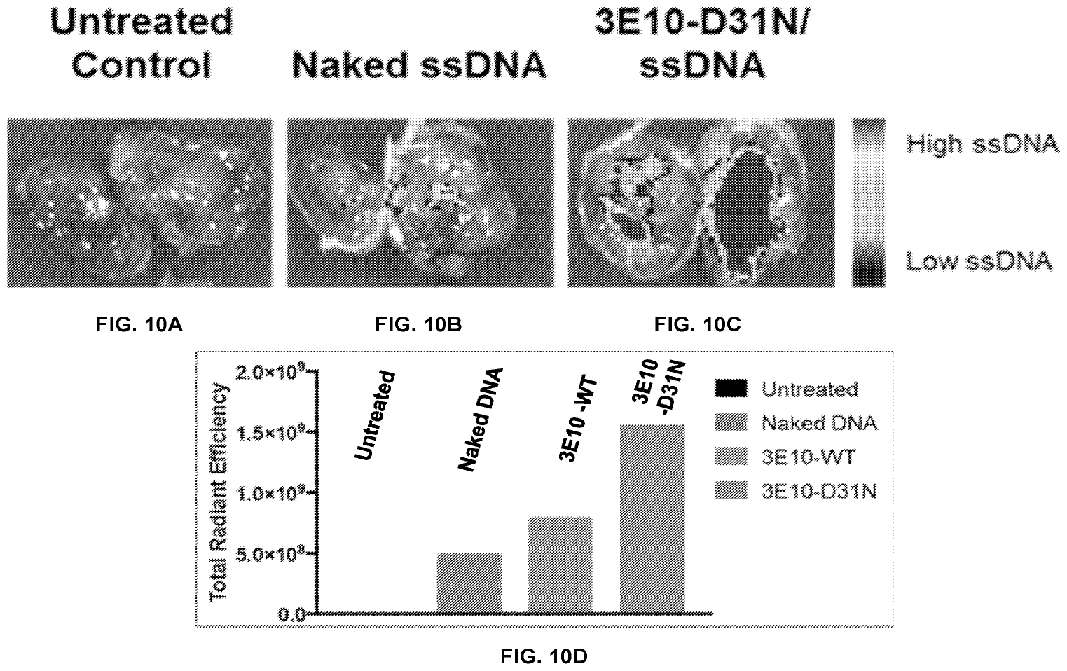

In some embodiments, the advantageous properties of the compositions and methods described herein are based, at least in part, on the discovery that 3E10 antibodies or variants thereof, or antigen-binding fragments thereof, as described below, deliver polynucleotides to cancerous tissues in vivo following systemic administration, and facilitate transport of the polynucleotides into cancer cells, through non-covalent interactions. In some other embodiments, compositions and methods described herein are based, at least in part, on the discovery that 3E10 antibodies or variants thereof, or antigen-binding fragments thereof, as described below, deliver polynucleotides across the blood brain barrier and facilitate delivery into tumor cells in the CNS. For instance, as described in Example 3 and illustrated in FIG. 10, 3E10 and 3E10-D31N variant antibodies localized to flank syngeneic colon tumors (CT26) in mice following intravenous administration. Advantageously, this ability to deliver and transport polynucleotides into cancerous tissues in vivo is exploited in the compositions and methods described herein to deliver therapeutic polynucleotides into various cancers, e.g., cancers displaying ENT2.

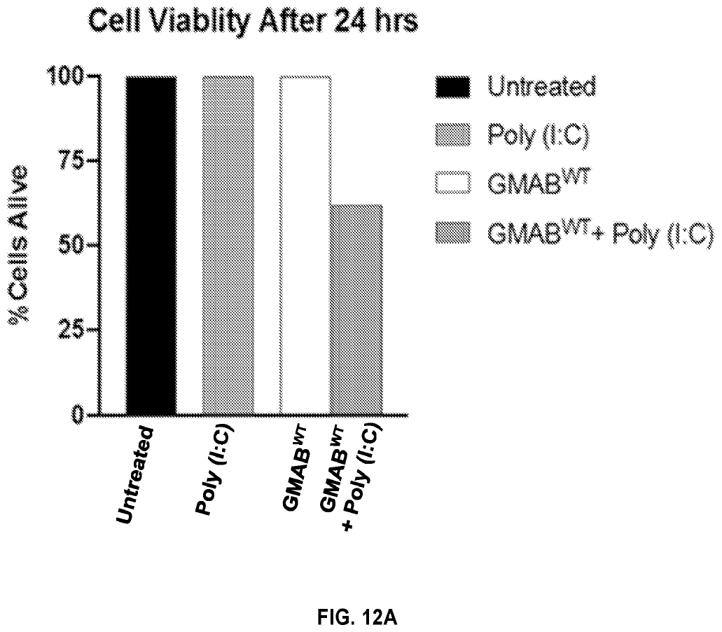

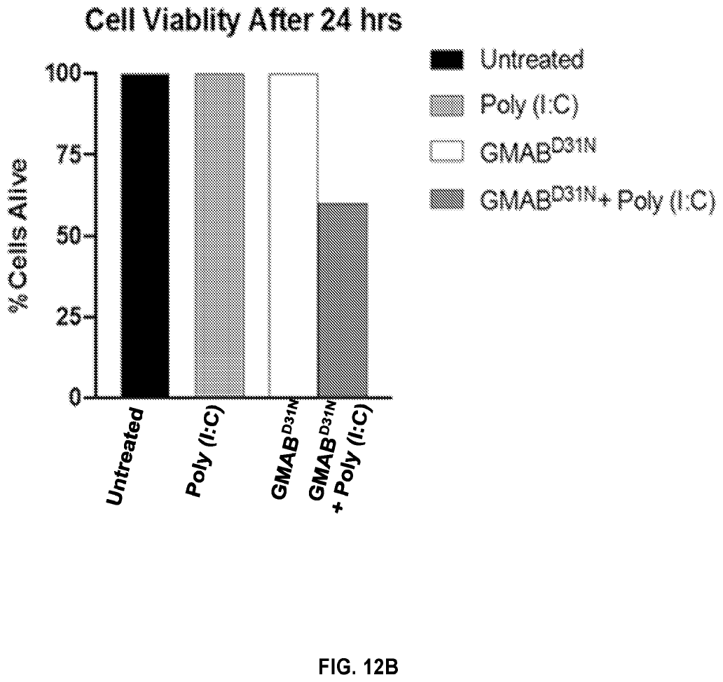

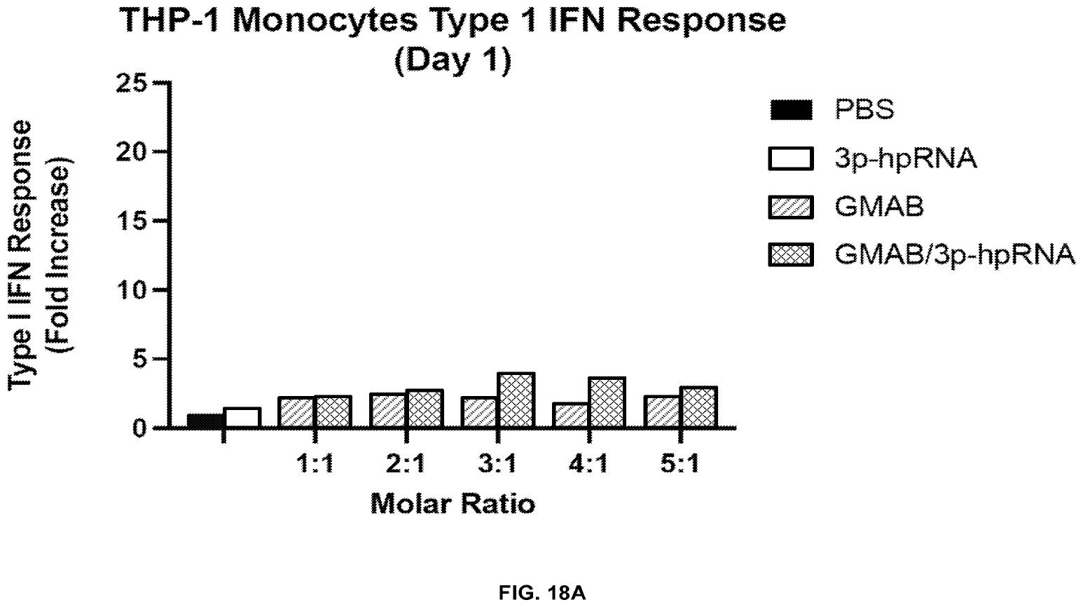

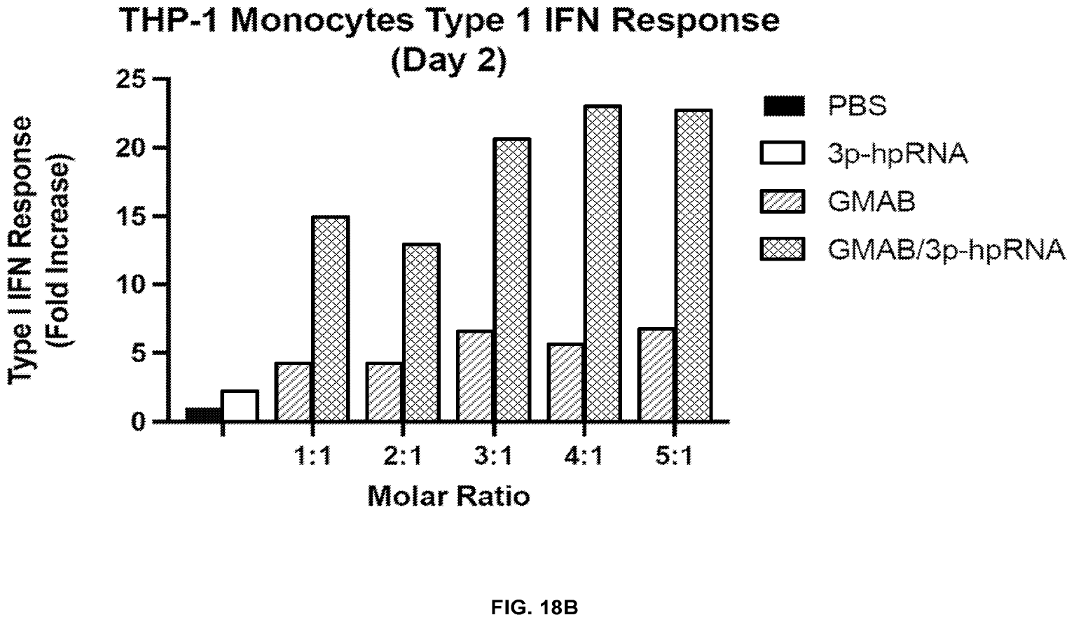

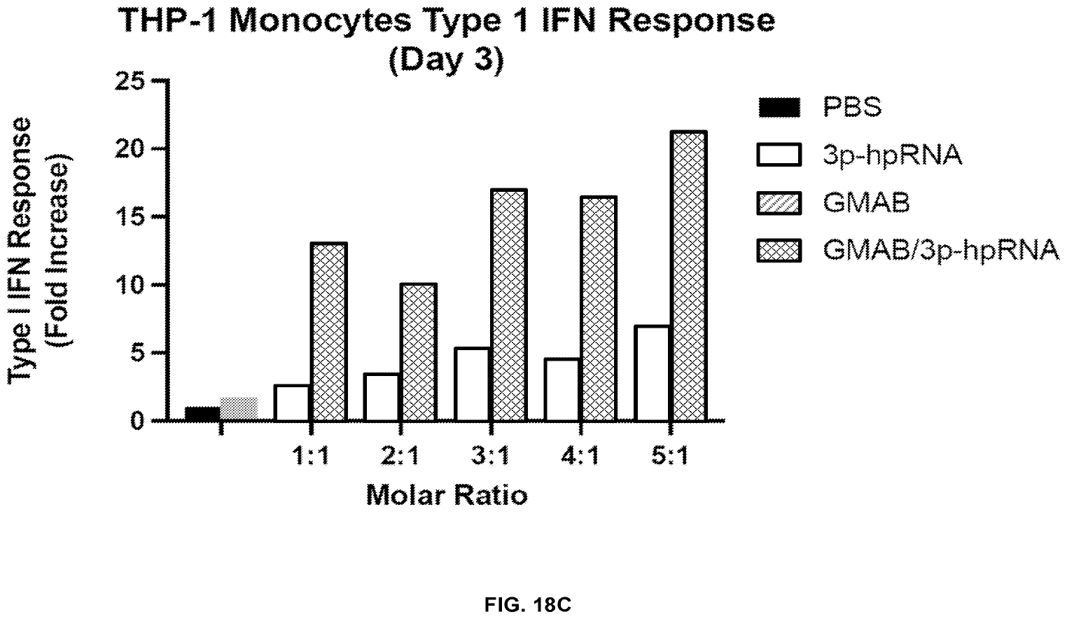

In some embodiments, the advantageous properties of the compositions and methods described herein are based, at least in part, on the discovery that therapeutic polynucleotides delivered into tumor cells by 3E10 antibodies or variants thereof, or antigen-binding fragments thereof, as described below, retain their ability to mediate cell death in cancer cells. For instance, as described in Example 5 and illustrated in FIG. 12, 3E10 and 3E10-D31N variant antibody mediated delivery of poly(I:C) into melanoma cells resulted into reduced cell viability within 24 hours. Similarly, as described in Example 11, and illustrated in FIG. 18, 3E10-D31N mediated delivery of a RIG-I stimulating ligand into monocytic cells derived from an acute monocytic leukemia effectively induces a Type-I IFN response characteristic of immunotherapy over at least three days, suggesting that the therapeutic polynucleotide was released over time from the 3E10 carrier after integration into the cell. Advantageously, this ability to deliver and integrate therapeutic polynucleotides into cancerous cells, while still making the polynucleotides available to function within the cell, is exploited in the compositions and methods described herein for the treatment of various cancers.

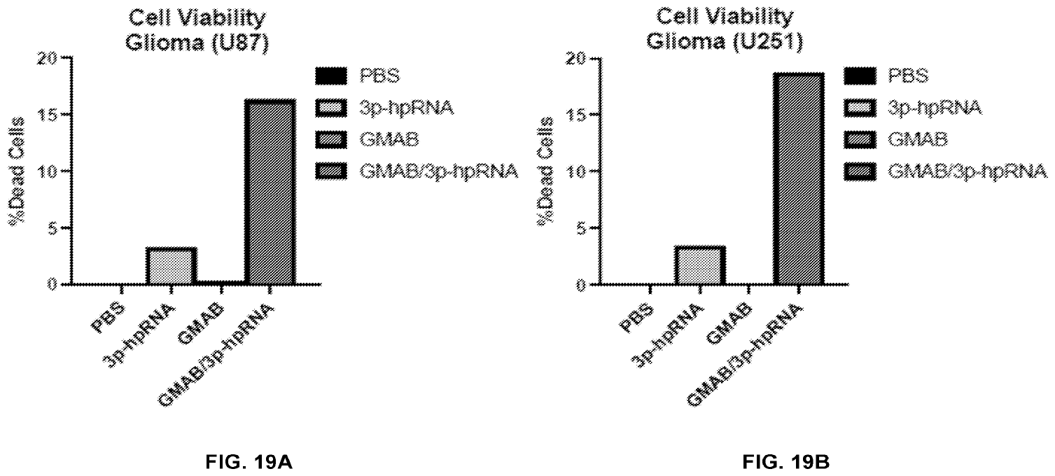

In some embodiments, the advantageous properties of the compositions and methods described herein are based, at least in part, on the discovery that 3E10 antibodies or variants thereof, or antigen-binding fragments thereof, as described below, facilitate delivery of therapeutic polynucleotide into tumor cells, and that the delivered therapeutic polynucleotides retain their ability to mediate cell death in cancer cells. For instance, as described in Examples 12-15, and illustrated in FIGS. 19-22, exposure of cell lines derived from brain tumors, colorectal cancers, and breast cancer to a complex formed between an 3E10-D31N antibody and a RIG-I stimulating ligand resulted in significant cell death in all tumor types. Advantageously, the ability to cause cell death in cancer cells by treatment with such complexes is exploited in the compositions and methods described herein for the treatment of various cancers.

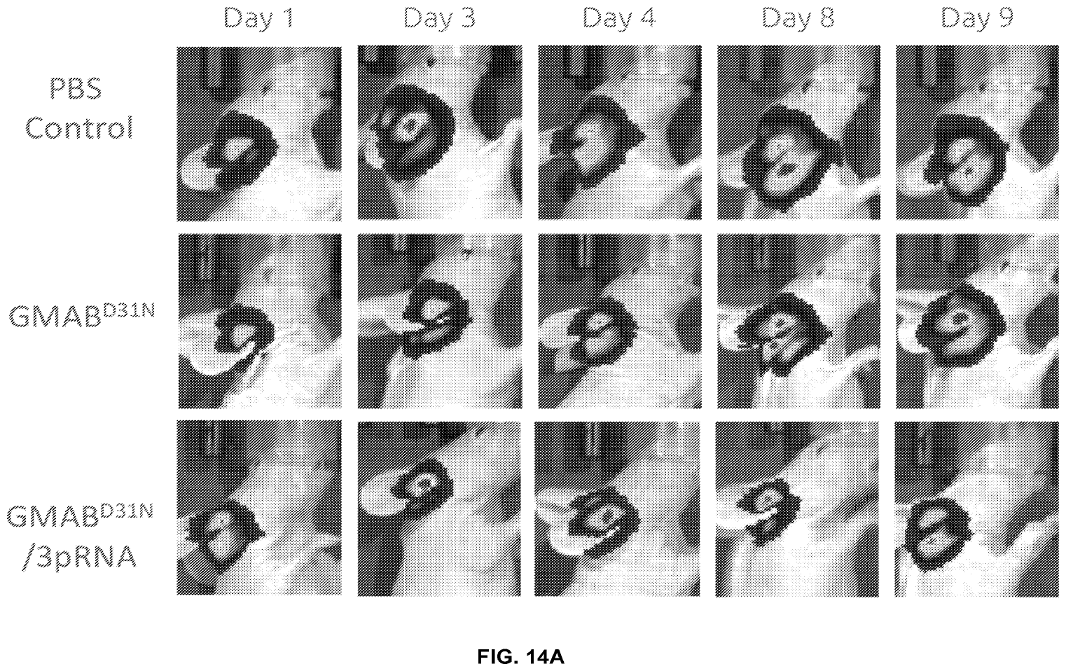

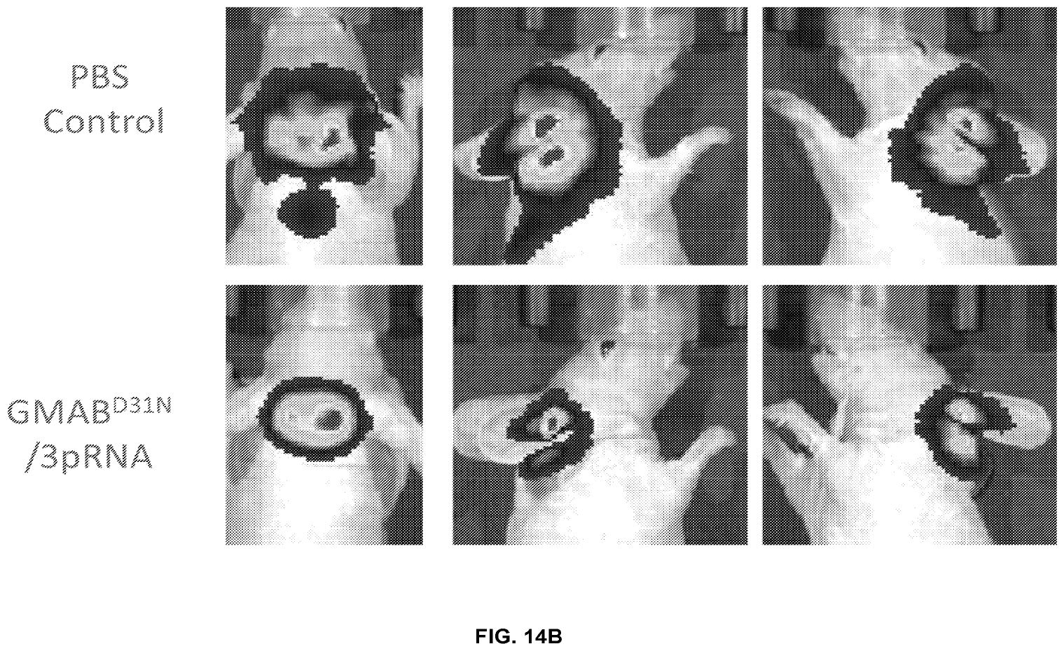

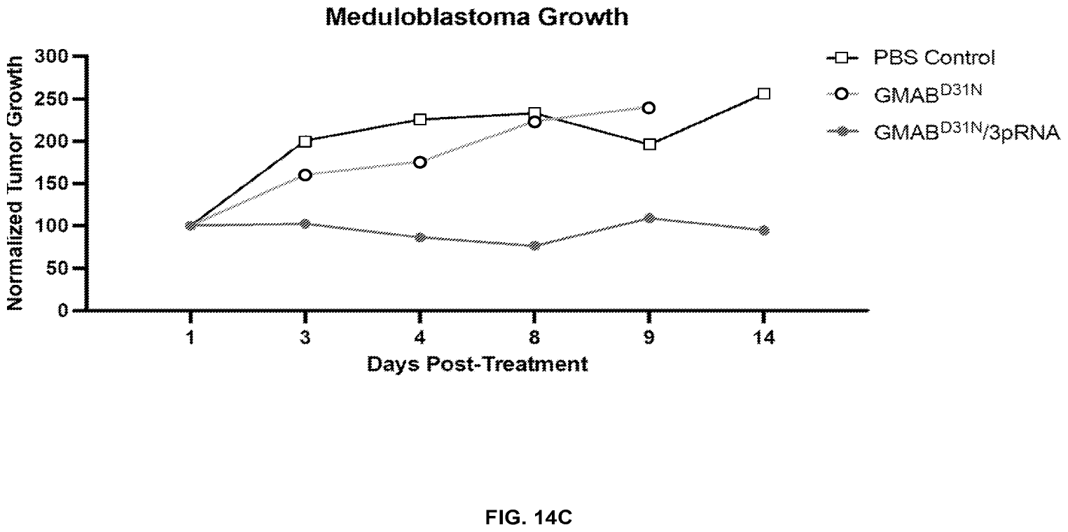

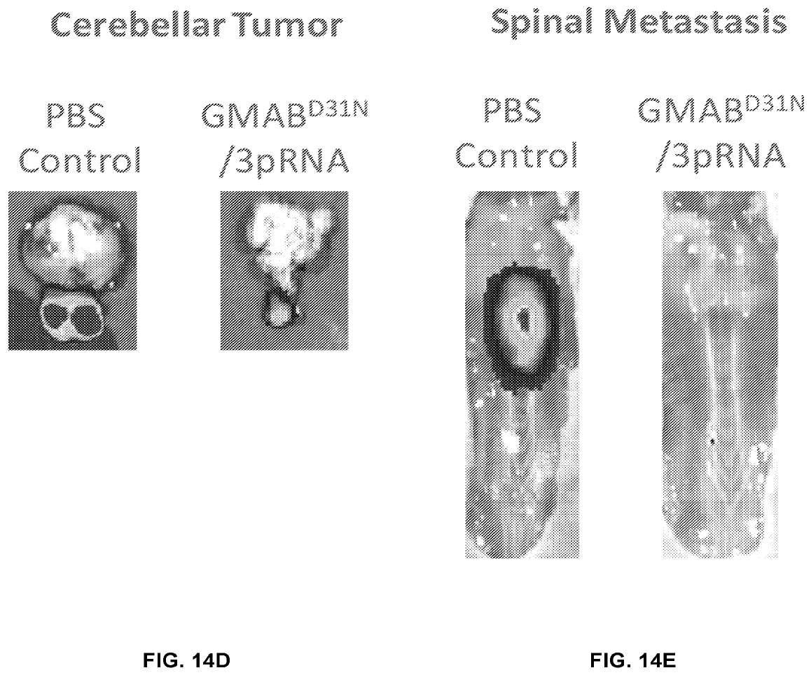

In some embodiments, the advantageous properties of the compositions and methods described herein are based, at least in part, on the discovery that complexes of 3E10 antibodies or variants thereof, or antigen-binding fragments thereof and therapeutic polynucleotides, as described below, significantly reduce tumor burden following system administration. For instance, as described in Example 7 and illustrated in FIG. 14, and particularly in FIG. 14D, a single dose of GMABD^{D31N}/3pRNA, but not the PBS control, significantly reduced tumor burden of a human medulloblastoma in the brain of mice following intravenous administration. Advantageously, the ability of these complexes to reduce tumor burden following systemic administration is exploited in the compositions and methods described herein for the treatment of various cancers.

Accordingly, one aspect of the present disclosure provides methods for treating a cancer in a subject, e.g., a cancer displaying ENT2, in need thereof. In some embodiments, the method includes administering to the subject, a therapeutically effective amount of a composition comprising a complex formed between (i) a therapeutic polynucleotide for treating cancer, and (ii) a 3E10 antibody or variant thereof, or antigen-binding fragment thereof.

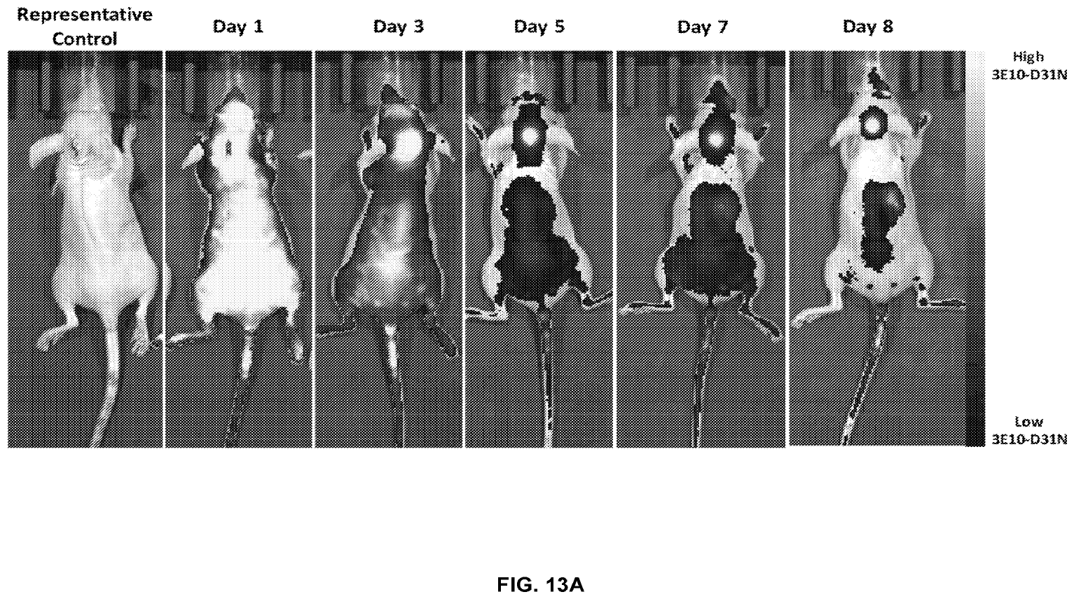

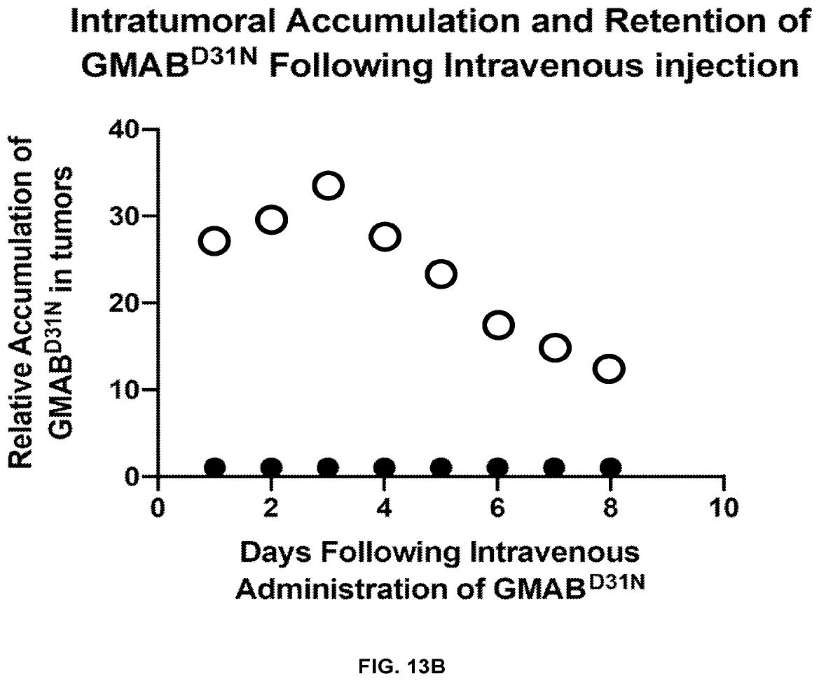

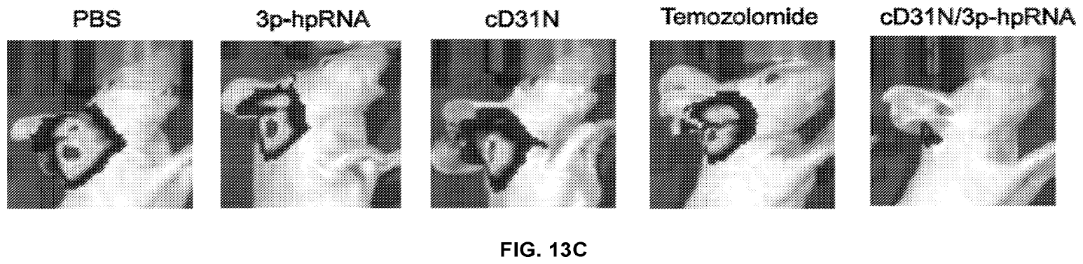

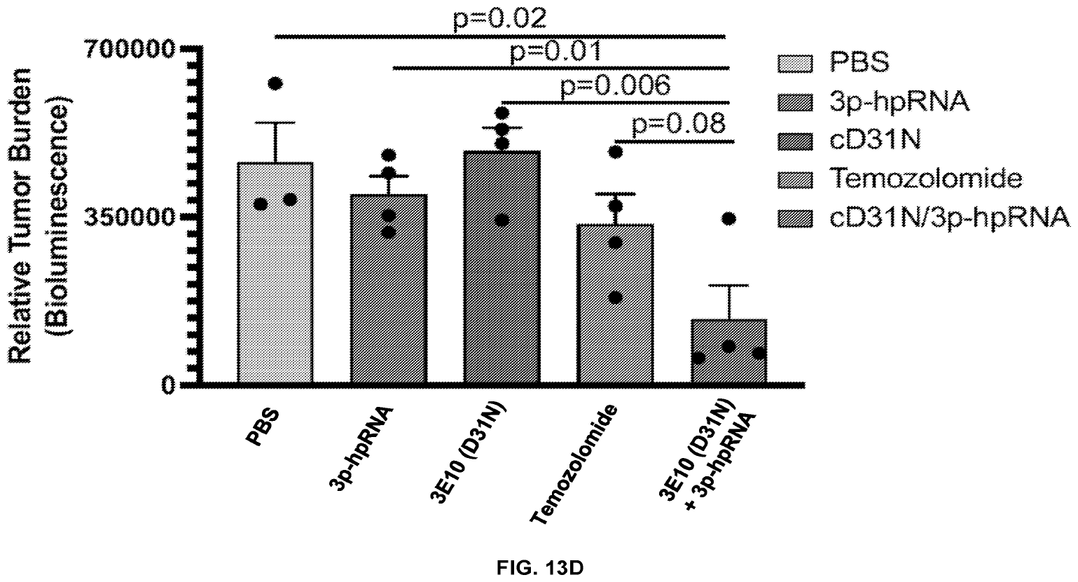

In some embodiments, the advantageous properties of the compositions and methods described herein are based, at least in part, on the discovery that 3E10 antibodies or variants thereof, or antigen-binding fragments thereof, as described below, readily cross the blood-brain barrier. For instance, as described in Example 6 and illustrated in FIG. 13, 3E10-D31N accumulated and was retained in the central nervous system (CNS) of mice bearing human medulloblastoma tumors for at least 8 days, both in the brain and at the spinal cord. Advantageously, the ability of 3E10 to cross the blood-brain barrier is exploited in the compositions and methods described herein to deliver therapeutic polynucleotides across the blood brain barrier (BBB) and into the CNS for the treatment of various cancers.

Accordingly, one aspect of the present disclosure provides methods for treating a cancer of the central nervous system, e.g., a cancer displaying ENT2, in a subject in need thereof.

### Definitions

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. Unless the context requires otherwise, it will be further understood that the terms “includes,” “comprising,” or any variation thereof, when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%.

### Antibodies and Variants Thereof

By “antigen binding domain” or “ABD” herein is meant a set of six Complementary Determining Regions (CDRs) that, when present as part of a polypeptide sequence or sequences, specifically binds a target antigen as discussed herein. Thus, a “nucleic acid binding domain” binds a nucleic acid antigen as outlined herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. Thus, in some cases, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of the hinge in the second monomer.

For all positions discussed in the present disclosure that relate to antibodies, unless otherwise noted, amino acid position numbering is according to the EU index. The EU index or EU index as in Kabat or EU numbering scheme refers to the numbering of the EU antibody. Kabat et al. collected numerous primary sequences of the variable regions of heavy chains and light chains. Based on the degree of conservation of the sequences, they classified individual primary sequences into the CDR and the framework and made a list thereof. See, SEQUENCES OF IMMUNOLOGICAL INTEREST, 5th edition, NIH publication, No. 91-3242, E. A. Kabat et al.; Edelman et al., 1969, Proc Natl Acad Sci USA 63:78-85, the contents of which are incorporated herein by reference. The modification can be an addition, deletion, or substitution.

By “target antigen” as used herein is meant the molecule that is bound specifically by the antigen binding domain comprising the variable regions of a given antibody. As discussed below, in the present case the target antigens are nucleic acids.

As described below, in some embodiments a parent polypeptide, for example an Fc parent polypeptide, is a human wild type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 75% identity with a parent protein sequence, or at least about 80% identity with a parent protein sequence, and most preferably at least about 90% identity, more preferably at least about 95%, or at least about 98%, or at least about 99% sequence identity. In some embodiments, the protein variant sequence herein has at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity with a parent protein sequence. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2, IgG3, or IgG4.

By “isotype” as used herein is meant any of the subclasses of immunoglobulins defined by the chemical and antigenic characteristics of their constant regions. It should be understood that therapeutic antibodies can also comprise hybrids of isotypes and/or subclasses.

By “Fab” or “Fab region” as used herein is meant a polypeptide that comprises the VH, CH1, VL, and CL immunoglobulin domains, generally on two different polypeptide chains (e.g. VH-CH1 on one chain and VL-CL on the other). Fab may refer to this region in isolation, or this region in the context of an antibody of the disclosure. In the context of a Fab, the Fab comprises an Fv region in addition to the CH1 and CL domains.

By “Fv” or “Fv fragment” or “Fv region” as used herein is meant a polypeptide that comprises the VL and VH domains of an ABD. Fv regions can be formatted as both Fabs (as discussed above, generally two different polypeptides that also include the constant regions as outlined above) and scFvs, where the vl and vh domains are combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g. H.X_L.Y means N- to C-terminal is vh-linker-vl, and L.Y_H.X is vl-linker-vh.

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc domain” includes both amino acids 231-447 (CH2-CH3) or 216-447 (hinge-CH2-CH3), or fragments thereof. An “Fe fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.) Human IgG Fe domains are of particular use in the present disclosure, and can be the Fc domain from human IgG1, IgG2 or IgG4.

A “variant Fc domain” contains amino acid modifications as compared to a parental Fc domain. Thus, a “variant human IgG1 Fc domain” is one that contains amino acid modifications (generally amino acid substitutions, although in the case of ablation variants, amino acid deletions are included) as compared to the human IgG1 Fc domain. In general, variant Fc domains have at least about 80, about 85, about 90, about 95, about 97, about 98 or about 99 percent identity to the corresponding parental human IgG Fc domain (using the identity algorithms discussed below, with one embodiment utilizing the BLAST algorithm as is known in the art, using default parameters). Alternatively, the variant Fe domains can have from 1 to about 20 (e.g., 1, 2, 3, 4, 5, 6, 7, 8, 9, 10, 11, 12, 13, 14, 15, 16, 17, 18, 19 or 20) amino acid modifications as compared to the parental Fc domain. Additionally, as discussed herein, the variant Fc domains herein still retain the ability to form a dimer with another Fc domain as measured using known techniques as described herein, such as non-denaturing gel electrophoresis.

By “heavy chain constant region” herein is meant the CH1-hinge-CH2-CH3 portion of an antibody (or fragments thereof), excluding the variable heavy domain; in EU numbering of human IgG1 this is amino acids 118-447 By “heavy chain constant region fragment” herein is meant a heavy chain constant region that contains fewer amino acids from either or both of the N- and C-termini but still retains the ability to form a dimer with another heavy chain constant region.

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

The antibodies of the present disclosure are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogenous host cells, and they can be isolated as well.

As used herein, the term “cell-penetrating antibody” refers to an immunoglobulin protein, fragment, variant thereof, or fusion protein based thereon that is transported into the cytoplasm and/or nucleus of living mammalian cells. The “cell-penetrating anti-DNA antibody” specifically binds DNA (e.g., single-stranded and/or double-stranded DNA). In some embodiments, the antibody is transported into the cytoplasm of the cells without the aid of a carrier or conjugate. In other embodiments, the antibody is conjugated to a cell-penetrating moiety, such as a cell penetrating peptide. In some embodiments, the cell-penetrating antibody is transported in the nucleus with or without a carrier or conjugate.

In some embodiments, the present disclosure provides bispecific (heterodimeric) antibodies that rely on the use of two different heavy chain variant Fc sequences, that will self-assemble to form heterodimeric Fc domains and heterodimeric antibodies. Accordingly, in some embodiments, the disclosure provides heterodimeric antibodies that bind to more than one antigen or ligand, e.g., to allow for bispecific binding to a therapeutic polynucleotide and to an antigen present on the surface of a cell or tissue of interest, such as a cancer antigen.

The heterodimeric antibody constructs are based on the self-assembling nature of the two Fc domains of the heavy chains of antibodies, e.g., two “monomers” that assemble into a “dimer”. Heterodimeric antibodies are made by altering the amino acid sequence of each monomer as more fully discussed below. Thus, in some embodiments, the disclosure includes a heterodimeric 3E10 antibody or variant thereof, or antigen-binding fragment thereof, for the treatment of cancers. In some embodiments, these heterodimeric antibodies rely on amino acid variants in the constant regions that are different on each chain to promote heterodimeric formation and/or allow for ease of purification of heterodimers over homodimers.

Thus, in some embodiments, the present disclosure provides bispecific antibodies. An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in purifying the heterodimeric antibodies away from the homodimeric antibodies and/or biasing the formation of the heterodimer over the formation of the homodimers.

There are a number of mechanisms that can be used to generate the heterodimers of the present invention. In addition, as will be appreciated by those in the art, these mechanisms can be combined to ensure high heterodimerization. Thus, amino acid variants that lead to the production of heterodimers are referred to as “heterodimerization variants”. As discussed below, heterodimerization variants can include steric variants (e.g., the “knobs and holes” or “skew” variants described below and the “charge pairs” variants described below) as well as “pI variants”, which allows purification of homodimers away from heterodimers. As is generally described in WO2014/145806, hereby incorporated by reference in its entirety and specifically as below for the discussion of “heterodimerization variants”, useful mechanisms for heterodimerization include “knobs and holes” (“KIH”; sometimes herein as “skew” variants (see discussion in WO2014/145806), “electrostatic steering” or “charge pairs” as described in WO2014/145806, pI variants as described in WO2014/145806, and general additional Fc variants as outlined in WO2014/145806 and below.

Several basic mechanisms can lead to ease of purifying heterodimeric antibodies, for example, one strategy uses pI variants, such that each monomer has a different pI, thus allowing the isoelectric purification of A-A, A-B and B-B dimeric proteins. Alternatively, some scaffold formats, such as the “triple F” format, also allows separation on the basis of size. It is also possible to “skew” the formation of heterodimers over homodimers. Thus, a combination of steric heterodimerization variants and pI or charge pair variants find particular use in the invention.

The bispecific antibodies described herein have two different antigen binding domains (ABDs) that bind to two different target antigens (“target pairs”), in either bivalent, bispecific formats or trivalent, bispecific formats. In the present disclosure, the bispecific heterodimeric antibodies have a first binding domain containing CDRs from a 3E10 antibody or variant thereof, or antigen-binding fragment thereof that binds a therapeutic polynucleotide as described herein, on one side and a second binding domain that binds a second antigen on the other side, such as a cell-surface antigen (e.g., a tumor antigen) or an effector antigen internal to the cell, such as a checkpoint protein/complex.

In some embodiments, the bispecific antibodies provided herein have two different antigen binding domains (ABDs) that bind to two different target antigens (“target pairs”), e.g., in bivalent, bispecific formats or trivalent, bispecific formats. In the present disclosure, the bispecific heterodimeric antibodies bind polynucleotide PRR agonists on one side, via CDR sequences from a 3E10 antibody or variant thereof, and a second antigen on the other side selected from EGF, Ras, Myc, HER2/Neu, p53, CD19, CD20, and PSMA. Accordingly, in some embodiments, the disclosure provides a bispecific antibody that binds polynucleotide PRR agonists, via CDR sequences from a 3E10 antibody or variant thereof, and EGF.

In some embodiments, the disclosure provides a bispecific antibody that binds polynucleotide PRR agonists, via CDR sequences from a 3E10 antibody or variant thereof, and Ras.

In some embodiments, the disclosure provides a bispecific antibody that binds polynucleotide PRR agonists, via CDR sequences from a 3E10 antibody or variant thereof, and Myc.

In some embodiments, the disclosure provides a bispecific antibody that binds polynucleotide PRR agonists, via CDR sequences from a 3E10 antibody or variant thereof, and HER2/Neu.

In some embodiments, the disclosure provides a bispecific antibody that binds polynucleotide PRR agonists, via CDR sequences from a 3E10 antibody or variant thereof, and p53.

In some embodiments, the disclosure provides a bispecific antibody that binds polynucleotide PRR agonists, via CDR sequences from a 3E10 antibody or variant thereof, and CD19.

In some embodiments, the disclosure provides a bispecific antibody that binds polynucleotide PRR agonists, via CDR sequences from a 3E10 antibody or variant thereof, and CD20.

In some embodiments, the disclosure provides a bispecific antibody that binds polynucleotide PRR agonists, via CDR sequences from a 3E10 antibody or variant thereof, and PSMA.

Bispecific antibodies and other binding proteins having a first heavy chain and a first light chain from 3E10 and a second heavy chain and a second light chain from a monoclonal antibody that specifically binds a second target are discussed in Weisbart, et al., Mol. Cancer Ther., 11(10):2169-73 (2012), and Weisbart, et al., Int. J. Oncology, 25.1113-8 (2004), and U.S. Patent Application No. 2013/0266570, which are specifically incorporated by reference in their entireties. In some embodiments, the second target is specific for a target cell-type, tissue, organ etc. Thus, the second heavy chain and second light chain can serve as a targeting moiety that targets the complex to the target cell-type, tissue, organ, e.g., to a cancerous tissue. In some embodiments, the second heavy chain and second light chain target an effector antigen internal of a cell-type, tissue, organ, etc., for example a cell-cycle checkpoint protein targeted by cancer immunotherapy.

### Proteins, Polypeptides, and Variants

By “polynucleotide PRR agonist” herein is meant a polynucleotide that is recognized by, and capable of stimulating, a pattern recognition receptor. Non-limiting examples of pattern recognition receptors include Toll-like receptors (TLRs), C-type lectin receptors (CLRs), RIG-I-like receptors (RLRs), Nucleotide-binding Oligomerization Domain (NOD)-like receptors (NLRs), and cytosolic DNA sensors (CDS). For a review of pattern recognition receptors, and their targeting for immunotherapy of various cancers, see, for example, Bai L., et al., “Promising targets based on pattern recognition receptors for cancer immunotherapy,” Pharmacological Research, 159 (2020) 105017, the content of which is incorporated herein by reference, in its entirety, for all purposes.

By “modification” herein is meant an amino acid substitution, insertion, and/or deletion in a polypeptide sequence.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the parent protein, using the alignment programs described below, such as BLAST.

Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol., 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see the webpage located at URL blast.ncbi.nlm.nih.gov/Blast.cgi. When using any of the aforementioned algorithms, the default parameters (for Window length, gap penalty, etc.) are used. Unless specifically stated otherwise, sequence identity is determined using the BLAST algorithm, using default parameters

### Subjects and Treatment of Cancer

As used herein, the term “subject” means any individual who is the target of administration. The subject can be a vertebrate, for example, a mammal. Thus, the subject can be a human. The term does not denote a particular age or sex.

As used herein, the term “pharmaceutically effective amount” means that the amount of the composition used is of sufficient quantity to ameliorate one or more causes or symptoms of a disease or disorder. Such amelioration only requires a reduction or alteration, not necessarily elimination. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “carrier” or “excipient” refers to an organic or inorganic ingredient, natural or synthetic inactive ingredient in a formulation, with which one or more active ingredients are combined. The carrier or excipient would naturally be selected to minimize degradation of the active ingredient or to minimize adverse side effects in the subject, as would be well known to one of skill in the art.

As used herein, the term “treat” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “cancer” refers to an abnormal growth of cells from any tissue. Cancers include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the presently claimed 3E10 antibody and therapeutic polynucleotide complexes, include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, meningioma, neuroblastoma, retinoblastoma and brain metastases).

As used herein, the term “CNS cancer” or “cancer of the central nervous system” refers to abnormal growth of cells from any tissue of the central nervous system, including the brain, spinal cord, meninges, or hematopoietic tissue of the primary CNS of a subject. Non-limited examples of CNS cancers include neuroepithelial cancers (such as gliomas, mature neuron cancers, primitive neuroectodermal tumors, and primitive brain cancers), meningeal cancers, and primary central nervous system hematopoietic cancers. Further examples of CNS cancers are provided

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### 3E10 Antibodies, Variants, and Fragments Thereof

In some aspects, the present disclosure relates to the use of 3E10 antibodies, and derivatives thereof, for delivering therapeutic polynucleotide to treat a cancer. As is discussed below, the term antibody is used generally. Antibodies that find use in the present disclosure take on a number of formats as described herein, including traditional antibodies as well as antibody derivatives, fragments, and mimetics, described herein in various embodiments.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present disclosure is directed to antibodies that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M).

The light chain generally comprises two domains, the variable light domain (containing the light chain CDRs and together with the variable heavy domains forming the Fv region), and a constant light chain region (often referred to as CL or Cκ). The heavy chain comprises a variable heavy domain and a constant domain, which includes a CH1-optional hinge-Fc domain comprising a CH2-CH3.

The hypervariable region of an antibody generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs useful for the compositions and methods described herein are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is described in Lafranc et al., Dev. Comp. Immunol. 27(1):55-77 (2003).

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present disclosure provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as nucleic acids, amino acids, or sugar side chains and usually have specific structural characteristics, as well as specific charge characteristics. A single antigen may have more than one epitope. The antibodies described herein bind to nucleic acid epitopes in a partially sequence-independent manner. That is, while the antibodies described herein bind to some polynucleotide structures and sequences with greater affinity than other nucleic acid structures and sequences, they have some general affinity for polynucleotides.

The “Fc domain” of the heavy chain includes the —CH2-CH3 domain, and optionally a hinge domain (—H—CH2-CH3). For IgG, the Fc domain comprises immunoglobulin domains CH2 and CH3 (Cγ2 and Cγ3) and the lower hinge region between CH1 (Cγ1) and CH2 (Cγ2). Although the boundaries of the Fc region may vary, the human IgG heavy chain Fc region is usually defined to include residues C226 or P230 to its carboxyl-terminus, wherein the numbering is according to the EU index as in Kabat. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. Thus, the “Fc domain” includes the —CH2-CH3 domain, and optionally a hinge domain (hinge-CH2-CH3). In the embodiments herein, when a scFv is attached to an Fc domain, it is generally the C-terminus of the scFv construct that is attached to all or part of the hinge of the Fc domain; for example, it is generally attached to the sequence EPKS which is the beginning of the hinge. In some embodiments, as is more fully described below, amino acid modifications are made to the Fc region, for example to alter binding to one or more FcγR receptors or to the FcRn receptor, and to enable heterodimer formation and purification, as outlined herein.

Another part of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain.

An scFv comprises a variable heavy chain, an scFv linker, and a variable light domain. In most of the constructs and sequences outlined herein, the C-terminus of the variable heavy chain is attached to the N-terminus of the scFv linker, the C-terminus of which is attached to the N-terminus of a variable light chain (N-vh-linker-vl-C) although that can be switched (N-vl-linker-vh-C).

Thus, the present disclosure relates to different antibody domains. As described herein and known in the art, the heterodimeric antibodies described in certain embodiments of the disclosure comprise different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

In certain embodiments, the antibodies of the disclosure comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence, e.g., that of the 3E10 antibody. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene. In certain cases, the humanized antibody may display no more than 5, or even no more than 4, 3, 2, or 1 amino acid difference from the amino acid sequence encoded by the germline immunoglobulin gene.

In one embodiment, the parent antibody has been affinity matured, as is known in the art. Structure-based methods may be employed for humanization and affinity maturation, for example as described in U.S. application Ser. No. 11/004,590, which is incorporated herein by reference. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all of which are incorporated herein by reference. Other humanization methods may involve the grafting of only parts of the CDRs, including but not limited to methods described in U.S. application Ser. No. 09/810,510; Tan et al., 2002, J. Immunol. 169:1119-1125; De Pascalis et al., 2002, J. Immunol. 169:3076-3084, all of which are incorporated herein by reference.

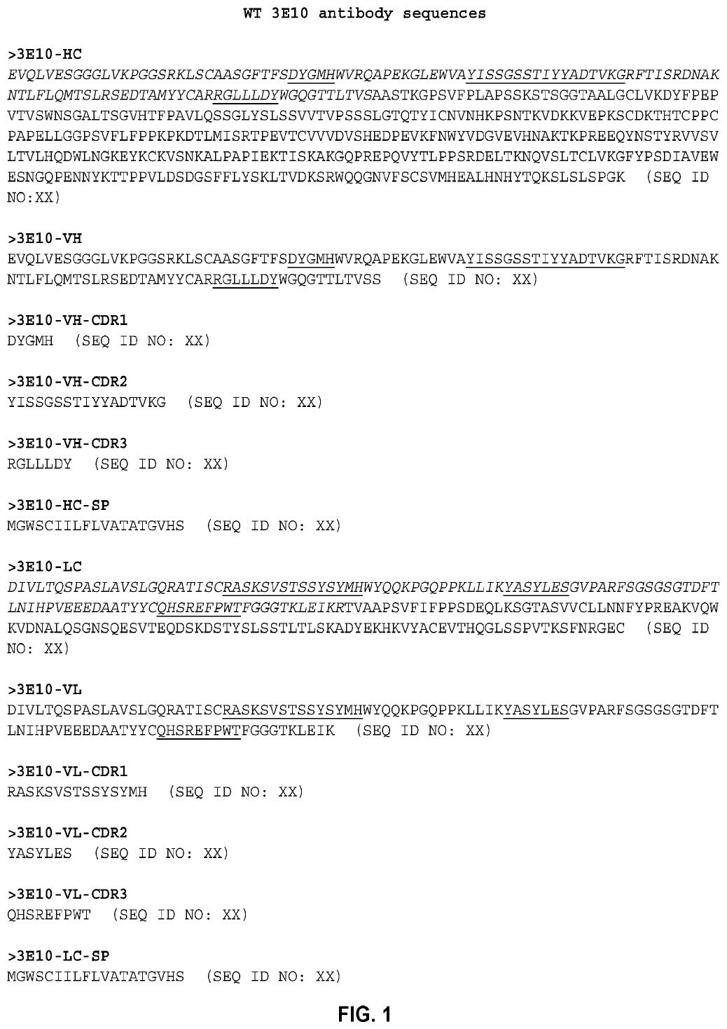

In some aspects, the disclosure relates to the use of antigen binding domains (ABDs) that bind to nucleic acids, and specifically that bind to therapeutic polynucleotides used to treat cancer, derived from the 3E10 antibody. The amino acid sequence of the heavy and light chains of the parent 3E10 antibody are shown in FIG. 1. Accordingly, in some embodiments, the compositions described herein include a 3E10 antibody or variant thereof, or antigen-binding fragment thereof.

In some embodiments, a 3E10 antibody or variant thereof, or antigen-binding fragment thereof described herein includes CDR sequences corresponding to the parent 3E10 antibody, shown in FIG. 1. Accordingly, in some embodiments, the a 3E10 antibody or variant thereof, or antigen-binding fragment thereof includes a light chain variable region (VL) complementarity determining region (CDR) 1 comprising the amino acid sequence of 3E10-VL-CDR1 (SEQ ID NO: XX), a VL CDR2 comprising the amino acid sequence of 3E10-VL-CDR2 (SEQ ID NO: XX), a VL CDR3 comprising the amino acid sequence of 3E10-VL-CDR3 (SEQ ID NO: XX), a heavy chain variable region (VH) CDR1 comprising the amino acid sequence of 3E10-VH-CDR1 (SEQ ID NO: XX), a VH CDR2 comprising the amino acid sequence of 3E10-VH-CDR2 (SEQ ID NO: XX), and a VH CDR3 comprising the amino acid sequence of 3E10-VH-CDR3 (SEQ ID NO: XX).

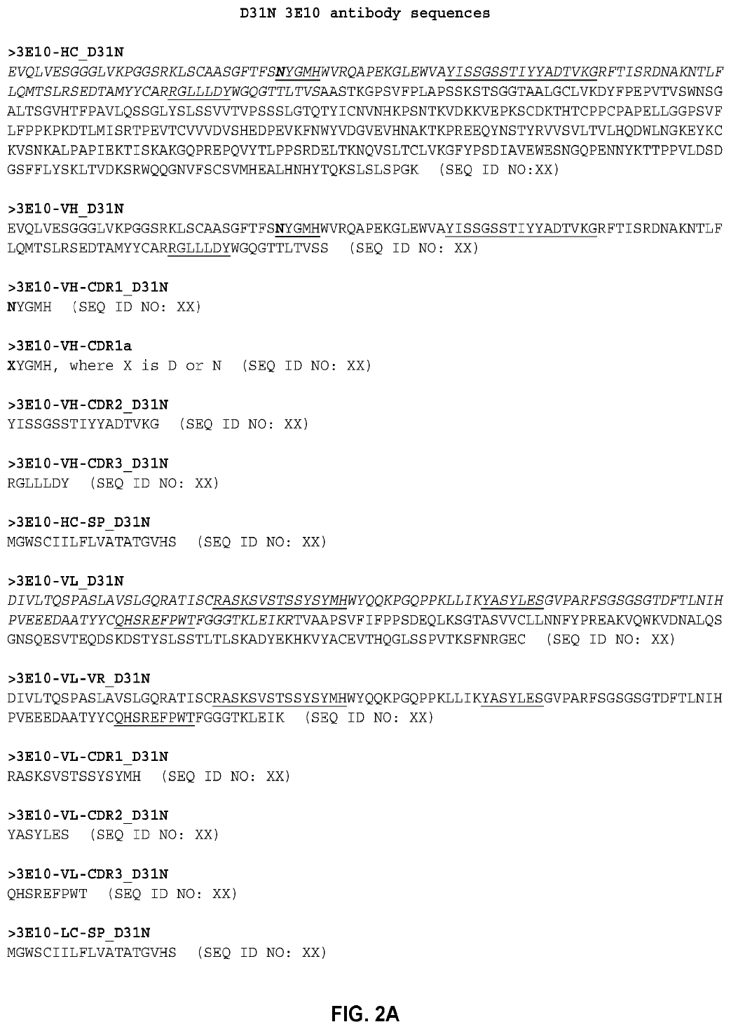

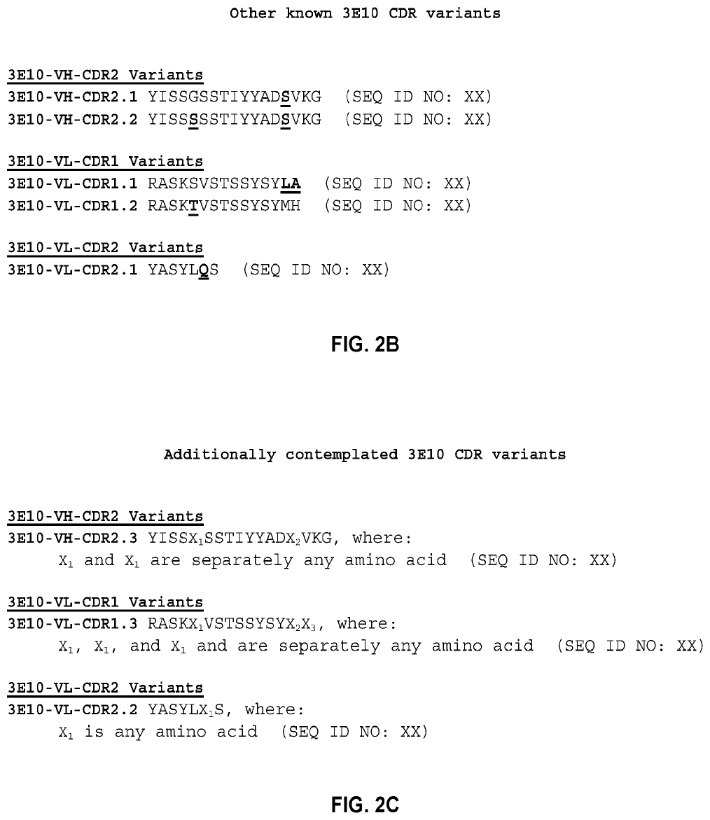

In some embodiments, a 3E10 antibody or variant thereof, or antigen-binding fragment thereof described herein includes CDR sequences from a variant 3E10 antibody that includes a D31N amino acid substitution in the VH CDR1, as shown in FIG. 2. Accordingly, in some embodiments, the a 3E10 antibody or variant thereof, or antigen-binding fragment thereof includes a light chain variable region (VL) complementarity determining region (CDR) 1 comprising the amino acid sequence of 3E10-VL-CDR1_D31N (SEQ ID NO: XX), a VL CDR2 comprising the amino acid sequence of 3E10-VL-CDR2_D31N (SEQ ID NO: XX), a VL CDR3 comprising the amino acid sequence of 3E10-VL-CDR3_D31N (SEQ ID NO: XX), a heavy chain variable region (VH) CDR1 comprising the amino acid sequence of 3E10-VH-CDR1_D31N (SEQ ID NO: XX), a VH CDR2 comprising the amino acid sequence of 3E10-VH-CDR2_D31N (SEQ ID NO: XX), and a VH CDR3 comprising the amino acid sequence of 3E10-VH-CDR3_D31N (SEQ ID NO: XX).

In some embodiments, a 3E10 antibody or variant thereof, or antigen-binding fragment thereof described herein refers to CDR sequences corresponding to the parent 3E10 antibody, shown in FIG. 1, optionally including a D31N amino acid substitution in the VH CDR1. Accordingly, in some embodiments, a 3E10 antibody or variant thereof, or antigen-binding fragment thereof includes a light chain variable region (VL) complementarity determining region (CDR) 1 comprising the amino acid sequence of 3E10-VL-CDR1 (SEQ ID NO: XX), a VL CDR2 comprising the amino acid sequence of 3E10-VL-CDR2 (SEQ ID NO: XX), a VL CDR3 comprising the amino acid sequence of 3E10-VL-CDR3 (SEQ ID NO: XX), a heavy chain variable region (VH) CDR1 comprising the amino acid sequence of 3E10-VH-CDR1a (SEQ ID NO: XX), a VH CDR2 comprising the amino acid sequence of 3E10-VH-CDR2 (SEQ ID NO: XX), and a VH CDR3 comprising the amino acid sequence of 3E10-VH-CDR3 (SEQ ID NO: XX).

In some embodiments, a 3E10 antibody or variant thereof, or antigen-binding fragment thereof described herein includes CDR sequences corresponding to the parent 3E10 antibody, shown in FIG. 1, with a known amino acid substitution in one or more CDR. For example, FIG. 2B shows the amino acid sequence of several known VH CDR2, VL CDR1, and VL CDR2 amino acid sequences. Accordingly, in some embodiments, a 3E10 antibody or variant thereof, or antigen-binding fragment thereof described herein includes one or more amino acid substitution, relative to the CDR sequences of the parent 3E10 (shown in FIG. 1) or 3E10-D31N variant (shown in FIG. 2), selected from a G to S substitution at position 5 of VH CDR2, a T to S substitution at position 14 of VH CDR2, an S to T substitution at position 5 of VL CDR1, an M to L substitution at position 14 of VL CDR1, an H to A substitution at position 15 of VL CDR1, and an E to Q substitution at position 6 of VL CDR2.

Accordingly, in some embodiments, a 3E10 antibody or variant thereof, or antigen-binding fragment thereof includes VH CDR2 comprising the amino acid sequence of 3E10-VH-CDR2.1 (SEQ ID NO: XX) or 3E10-VH-CDR2.2 (SEQ ID NO: XX). In some embodiments, the 3E10 antibody or variant thereof, or antigen-binding fragment thereof further includes VL CDRs 1-3, and VH CRDs 1 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 according to the 3E10-D31N variant (as shown in FIG. 2A). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1) or relative to the 3E10-D31N variant (as shown in FIG. 2A).

Similarly, in some embodiments, a 3E10 antibody or variant thereof, or antigen-binding fragment thereof includes VL CDR1 comprising the amino acid sequence of 3E10-VL-CDR1.1 (SEQ ID NO: XX) or 3E10-VL-CDR1.2 (SEQ ID NO: XX). In some embodiments, the 3E10 antibody or variant thereof, or antigen-binding fragment thereof further includes VL CDRs 2 and 3, and VH CRDs 1-3 according to the parent 3E10 antibody (as shown in FIG. 1). In some embodiments, the 3E10 antibody or variant thereof, or antigen-binding fragment thereof further includes VL CDRs 2 and 3, and VH CRDs 1-3 according to the 3E10-D31N variant (as shown in FIG. 2A). In some embodiments, the 3E10 antibody or variant thereof, or antigen-binding fragment thereof further includes VL CDRs 2 and 3, and VH CRDs 1-3 having one or more amino acid substitutions relative to the CDRs of the parent 3E10 antibody (as shown in FIG. 1) or relative to the 3E10-D31N variant (as shown in FIG. 2A).

Similarly, in some embodiments, a 3E10 antibody or variant thereof, or antigen-binding fragment thereof includes VL CDR2 comprising the amino acid sequence of 3E10-VL-CDR2.1 (SEQ ID NO: XX). In some embodiments, the 3E10 antibody or variant thereof, or antigen-binding fragment thereof further includes VL CDRs 1 and 3, and VH CRDs 1-3 according to the parent 3E10 antibody (as shown in FIG. 1). In some embodiments, the 3E10 antibody or variant thereof, or antigen-binding fragment thereof further includes VL CDRs 1 and 3, and VH CRDs 1-3 according to the 3E10-D31N variant (as shown in FIG. 2A). In some embodiments, the 3E10 antibody or variant thereof, or antigen-binding fragment thereof further includes VL CDRs 1 and 3, and VH CRDs 1-3 having one or more amino acid substitutions relative to the CDRs of the parent 3E10 antibody (as shown in FIG. 1) or relative to the 3E10-D31N variant (as shown in FIG. 2A).

While some of the amino acid substitutions described above are fairly conservative substitutions—e.g., an S to T substitution at position 5 of VL CDR1—other substitutions are to amino acids that have vastly different properties—e.g., an M to L substitution at position 14 of VL CDR1, an H to A substitution at position 15 of VL CDR1, and an E to Q substitution at position 6 of VL CDR2. This suggests, without being bound by theory, that at least these positions within the 3E10 CDR framework are tolerant to other amino acid substitutions.

Accordingly, in some embodiments, a 3E10 antibody or variant thereof, or antigen-binding fragment thereof includes VH CDR2 comprising the amino acid sequence of 3E10-VH-CDR2.3 (SEQ ID NO: XX). In some embodiments, the 3E10 antibody or variant thereof, or antigen-binding fragment thereof further includes VL CDRs 1-3, and VH CRDs 1 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 according to the 3E10-D31N variant (as shown in FIG. 2A). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1) or relative to the 3E10-D31N variant (as shown in FIG. 2A), e.g., as described herein.

Similarly, in some embodiments, a 3E10 antibody or variant thereof, or antigen-binding fragment thereof includes VL CDR1 comprising the amino acid sequence of 3E10-VL-CDR1.3 (SEQ ID NO: XX). In some embodiments, the 3E10 antibody or variant thereof, or antigen-binding fragment thereof further includes VL CDRs 2 and 3, and VH CRDs 1-3 according to the parent 3E10 antibody (as shown in FIG. 1). In some embodiments, the 3E10 antibody or variant thereof, or antigen-binding fragment thereof further includes VL CDRs 2 and 3, and VH CRDs 1-3 according to the 3E10-D31N variant (as shown in FIG. 2A). In some embodiments, the 3E10 antibody or variant thereof, or antigen-binding fragment thereof further includes VL CDRs 2 and 3, and VH CRDs 1-3 having one or more amino acid substitutions relative to the CDRs of the parent 3E10 antibody (as shown in FIG. 1) or relative to the 3E10-D31N variant (as shown in FIG. 2A), e.g., as described herein.

Similarly, in some embodiments, a 3E10 antibody or variant thereof, or antigen-binding fragment thereof, includes VL CDR2 comprising the amino acid sequence of 3E10-VL-CDR2.2 (SEQ ID NO: XX). In some embodiments, the 3E10 antibody or variant thereof, or antigen-binding fragment thereof further includes VL CDRs 1 and 3, and VH CRDs 1-3 according to the parent 3E10 antibody (as shown in FIG. 1). In some embodiments, the 3E10 antibody or variant thereof, or antigen-binding fragment thereof further includes VL CDRs 1 and 3, and VH CRDs 1-3 according to the 3E10-D31N variant (as shown in FIG. 2A). In some embodiments, the 3E10 antibody or variant thereof, or antigen-binding fragment thereof further includes VL CDRs 1 and 3, and VH CRDs 1-3 having one or more amino acid substitutions relative to the CDRs of the parent 3E10 antibody (as shown in FIG. 1) or relative to the 3E10-D31N variant (as shown in FIG. 2A), e.g., as described herein.

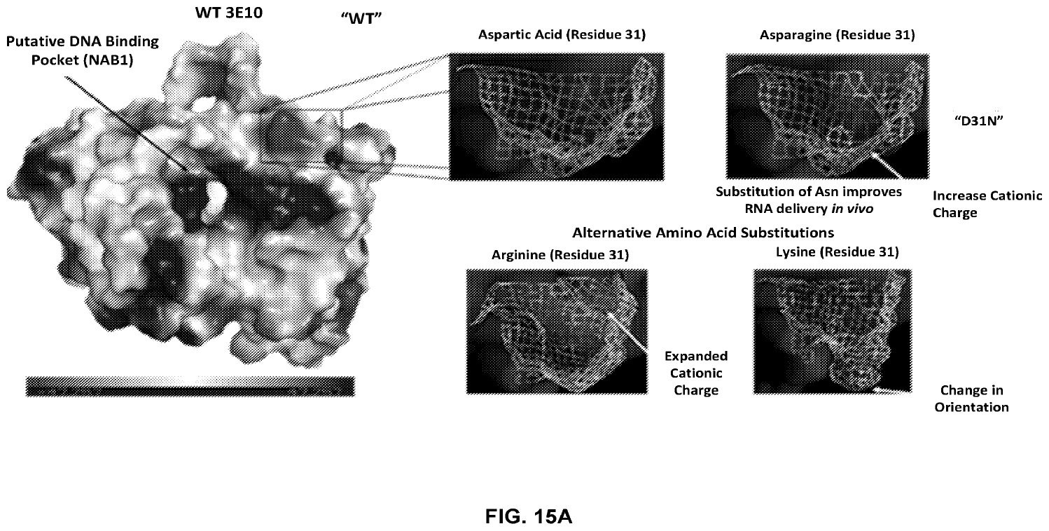

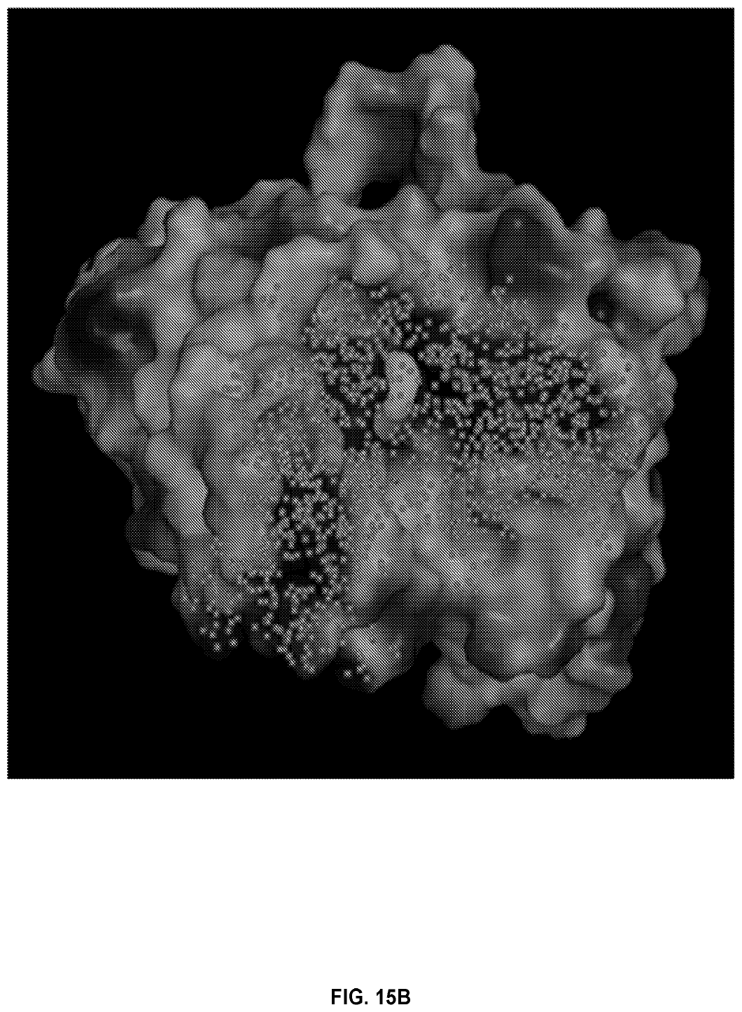

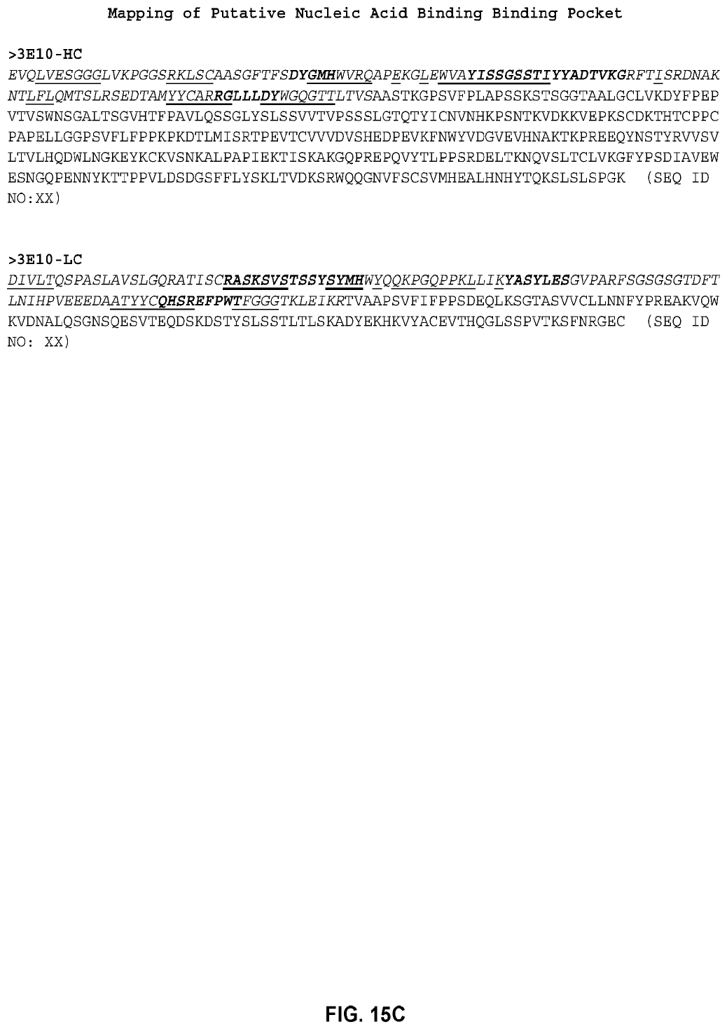

Further, because 3E10 antibodies or variants thereof, or antigen-binding fragments thereof, bind to nucleic acid in a partially sequence-independent manner, and without being bound by theory, it was contemplated that the interaction may be mediated by electrostatic interactions with the nucleotide backbone. To investigate this theory, electrostatic surface potential renderings of a molecular model of a 3E10-scFv construct—the amino acid sequence of which is illustrated in FIG. 15C—were generated, as shown in FIGS. 15A and 15B. These models revealed a putative Nucleic Acid Binding pocket (NAB1) corresponding to a large basic region on the surface of the molecule, as illustrated in FIG. 15A. The position of the non-hydrogen atoms of the amino acids contributing to the putative Nucleic Acid Binding pocket in the model are superposed in FIG. 15B, and the amino acid residues are mapped onto the sequence of the construct in FIG. 15C.

Thus, it is contemplated that amino acid substitutions within the CDRs of a 3E10 antibody or variant thereof, or antigen-binding fragment thereof, as described herein, that maintain the electrostatic character of this putative Nucleic Acid Binding pocket will also retain the nucleic acid binding properties of the construct. Accordingly, in some embodiments, a 3E10 antibody or variant thereof, or antigen-binding fragment thereof, includes one or more amino acid substitution of a first basic amino acid to a second basic amino acid (e.g., K, R, or H). Similarly, in some embodiments, a 3E10 antibody or variant thereof, or antigen-binding fragment thereof, includes one or more amino acid substitution of a first acidic amino acid to a second acidic amino acid (e.g., D or E). Examples of such charge-conserved variant 3E10 CDRs are shown in FIG. 3.

Accordingly, in some embodiments, a 3E10 antibody or variant thereof, or antigen-binding fragment thereof includes VH CDR1 comprising the amino acid sequence of 3E10-VH-CDR1.c1 (SEQ ID NO: XX), 3E10-VH-CDR1.c2 (SEQ ID NO: XX), 3E10-VH-CDR1.c3 (SEQ ID NO: XX), 3E10-VH-CDR1.c4 (SEQ ID NO: XX), or 3E10-VH-CDR1.c5 (SEQ ID NO: XX). In some embodiments, the 3E10 antibody or variant thereof, or antigen-binding fragment thereof further includes VL CDRs 1-3, and VH CRDs 2 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR2 comprising the amino acid sequence of 3E10-VH-CDR2.c1 (SEQ ID NO: XX), 3E10-VH-CDR2.c2 (SEQ ID NO: XX), or 3E10-VH-CDR2.c3 (SEQ ID NO: XX). In some embodiments, the 3E10 antibody or variant thereof, or antigen-binding fragment thereof further includes VL CDRs 1-3, and VH CRDs 1 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 according to the 3E10-D31N variant (as shown in FIG. 2A). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR3 comprising the amino acid sequence of 3E10-VH-CDR3.c1 (SEQ ID NO: XX), 3E10-VH-CDR3.c2 (SEQ ID NO: XX), or 3E10-VH-CDR3.c3 (SEQ ID NO: XX). In some embodiments, the 3E10 antibody or variant thereof, or antigen-binding fragment thereof further includes VL CDRs 1-3, and VH CRDs 1 and 2 according to the parent 3E10 antibody (as shown in FIG. 1). In some embodiments, the 3E10 antibody or variant thereof, or antigen-binding fragment thereof further includes VL CDRs 1-3, and VH CRDs 1 and 2 according to the 3E10-D31N variant (as shown in FIG. 2A). In some embodiments, the 3E10 antibody or variant thereof, or antigen-binding fragment thereof further includes VL CDRs 1-3, and VH CRDs 1 and 2 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR1 comprising the amino acid sequence of 3E10-VL-CDR1.c1 (SEQ ID NO: XX), 3E10-VL-CDR1.c2 (SEQ ID NO: XX), 3E10-VL-CDR1.c3 (SEQ ID NO: XX), 3E10-VL-CDR1.c4 (SEQ ID NO: XX), 3E10-VL-CDR1.c5 (SEQ ID NO: XX), or 3E10-VL-CDR1.c6 (SEQ ID NO: XX). In some embodiments, the 3E10 antibody or variant thereof, or antigen-binding fragment thereof further includes VL CDRs 2 and 3, and VH CRDs 1-3 according to the parent 3E10 antibody (as shown in FIG. 1). In some embodiments, the 3E10 antibody or variant thereof, or antigen-binding fragment thereof further includes VL CDRs 2 and 3, and VH CRDs 1-3 according to the 3E10-D31N variant (as shown in FIG. 2A). In some embodiments, the 3E10 antibody or variant thereof, or antigen-binding fragment thereof further includes VL CDRs 2 and 3, and VH CRDs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR2 comprising the amino acid sequence of 3E10-VL-CDR2.c1 (SEQ ID NO: XX). In some embodiments, the 3E10 antibody or variant thereof, or antigen-binding fragment thereof further includes VL CDRs 1 and 3, and VH CRDs 1-3 according to the parent 3E10 antibody (as shown in FIG. 1). In some embodiments, the 3E10 antibody or variant thereof, or antigen-binding fragment thereof further includes VL CDRs 1 and 3, and VH CRDs 1-3 according to the 3E10-D31N variant (as shown in FIG. 2A). In some embodiments, the 3E10 antibody or variant thereof, or antigen-binding fragment thereof further includes VL CDRs 1 and 3, and VH CRDs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR3 comprising the amino acid sequence of 3E10-VL-CDR3.c1 (SEQ ID NO: XX), 3E10-VL-CDR3.c2 (SEQ ID NO: XX), 3E10-VL-CDR3.c3 (SEQ ID NO: XX), 3E10-VL-CDR3.c4 (SEQ ID NO: XX), 3E10-VL-CDR3.c5 (SEQ ID NO: XX), or 3E10-VL-CDR3.c6 (SEQ ID NO: XX). In some embodiments, the 3E10 antibody or variant thereof, or antigen-binding fragment thereof further includes VL CDRs 1 and 2, and VH CRDs 1-3 according to the parent 3E10 antibody (as shown in FIG. 1). In some embodiments, the 3E10 antibody or variant thereof, or antigen-binding fragment thereof further includes VL CDRs 1 and 2, and VH CRDs 1-3 according to the 3E10-D31N variant (as shown in FIG. 2A). In some embodiments, the 3E10 antibody or variant thereof, or antigen-binding fragment thereof further includes VL CDRs 1 and 2, and VH CRDs 1-3 having one or more amino acid substitutions relative to the CDRs of the parent 3E10 antibody (as shown in FIG. 1), e.g., as described herein.

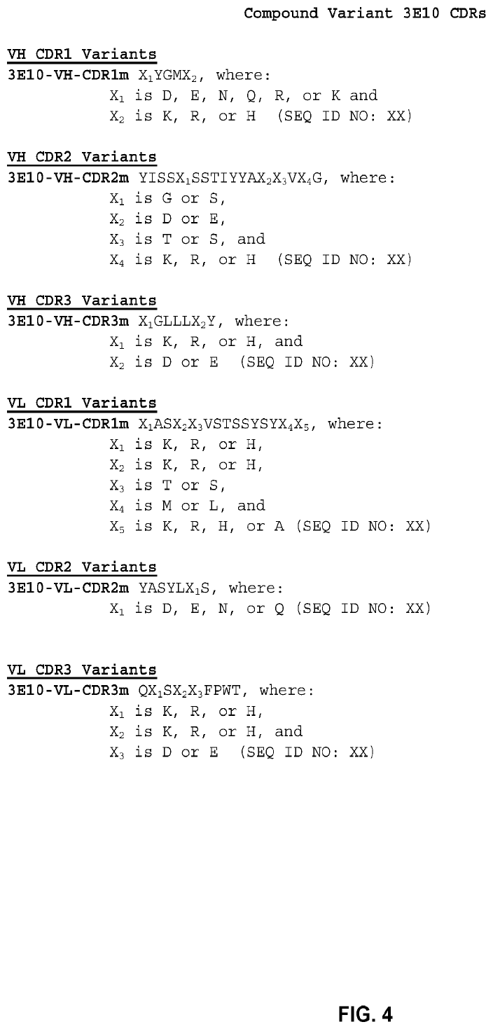

It is also contemplated that a 3E10 antibody or variant thereof, or antigen-binding fragment thereof, as described herein, includes any combination of the 3E10 CDR amino acid substitutions described above. Examples of 3E10 variant CDR sequences that incorporate one or more of the amino acid substitutions described herein are shown in FIG. 4.

Accordingly, in some embodiments, a 3E10 antibody or variant thereof, or antigen-binding fragment thereof includes VH CDR1 comprising the amino acid sequence of 3E10-VH-CDR1m (SEQ ID NO: XX). In some embodiments, the 3E10 antibody or variant thereof, or antigen-binding fragment thereof further includes VL CDRs 1-3, and VH CRDs 2 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR2 comprising the amino acid sequence of 3E10-VH-CDR2m (SEQ ID NO: XX). In some embodiments, the 3E10 antibody or variant thereof, or antigen-binding fragment thereof further includes VL CDRs 1-3, and VH CRDs 1 and 3 according to the parent 3E10 antibody (as shown in FIG. 1). In some embodiments, the 3E10 antibody or variant thereof, or antigen-binding fragment thereof further includes VL CDRs 1-3, and VH CRDs 1 and 3 according to the 3E10-D31N variant (as shown in FIG. 2A). In some embodiments, the 3E10 antibody or variant thereof, or antigen-binding fragment thereof further includes VL CDRs 1-3, and VH CRDs 1 and 3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VH CDR3 comprising the amino acid sequence of 3E10-VH-CDR3m (SEQ ID NO: XX). In some embodiments, the 3E10 antibody or variant thereof, or antigen-binding fragment thereof further includes VL CDRs 1-3, and VH CRDs 1 and 2 according to the parent 3E10 antibody (as shown in FIG. 1). In some embodiments, the 3E10 antibody or variant thereof, or antigen-binding fragment thereof further includes VL CDRs 1-3, and VH CRDs 1 and 2 according to the 3E10-D31N variant (as shown in FIG. 2A). In some embodiments, the 3E10 antibody or variant thereof, or antigen-binding fragment thereof further includes VL CDRs 1-3, and VH CRDs 1 and 2 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR1 comprising the amino acid sequence of 3E10-VL-CDR1m (SEQ ID NO: XX). In some embodiments, the 3E10 antibody or variant thereof, or antigen-binding fragment thereof further includes VL CDRs 2 and 3, and VH CRDs 1-3 according to the parent 3E10 antibody (as shown in FIG. 1). In some embodiments, the 3E10 antibody or variant thereof, or antigen-binding fragment thereof further includes VL CDRs 2 and 3, and VH CRDs 1-3 according to the 3E10-D31N variant (as shown in FIG. 2A). In some embodiments, the 3E10 antibody or variant thereof, or antigen-binding fragment thereof further includes VL CDRs 2 and 3, and VH CRDs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR2 comprising the amino acid sequence of 3E10-VL-CDR2m (SEQ ID NO: XX). In some embodiments, the 3E10 antibody or variant thereof, or antigen-binding fragment thereof further includes VL CDRs 1 and 3, and VH CRDs 1-3 according to the parent 3E10 antibody (as shown in FIG. 1). In some embodiments, the 3E10 antibody or variant thereof, or antigen-binding fragment thereof further includes VL CDRs 1 and 3, and VH CRDs 1-3 according to the 3E10-D31N variant (as shown in FIG. 2A). In some embodiments, the 3E10 antibody or variant thereof, or antigen-binding fragment thereof further includes VL CDRs 1 and 3, and VH CRDs 1-3 having one or more amino acid substitutions relative to the CDRs of the parent 3E10 antibody (as shown in FIG. 1), e.g., as described herein.

Similarly, in some embodiments, a 3E10 antibody or variant thereof, or antigen-binding fragment thereof includes VL CDR3 comprising the amino acid sequence of 3E10-VL-CDR3m (SEQ ID NO: XX). In some embodiments, the 3E10 antibody or variant thereof, or antigen-binding fragment thereof further includes VL CDRs 1 and 2, and VH CRDs 1-3 according to the parent 3E10 antibody (as shown in FIG. 1). In some embodiments, the 3E10 antibody or variant thereof, or antigen-binding fragment thereof further includes VL CDRs 1 and 2, and VH CRDs 1-3 according to the 3E10-D31N variant (as shown in FIG. 2A). In some embodiments, the 3E10 antibody or variant thereof, or antigen-binding fragment thereof further includes VL CDRs 1 and 2, and VH CRDs 1-3 having one or more amino acid substitutions relative to the CDRs of the parent 3E10 antibody (as shown in FIG. 1), e.g., as described herein.

In some embodiments, a 3E10 antibody or variant thereof, or antigen-binding fragment thereof described herein includes a light chain variable region (VL) complementarity determining region (CDR) 1 comprising the amino acid sequence of 3E10-VL-CDR1m (SEQ ID NO: XX), a VL CDR2 comprising the amino acid sequence of 3E10-VL-CDR2m (SEQ ID NO: XX), a VL CDR3 comprising the amino acid sequence of 3E10-VL-CDR3m (SEQ ID NO: XX), a heavy chain variable region (VH) CDR1 comprising the amino acid sequence of 3E10-VH-CDR1m (SEQ ID NO: XX), a VH CDR2 comprising the amino acid sequence of 3E10-VH-CDR2m (SEQ ID NO: XX), and a VH CDR3 comprising the amino acid sequence of 3E10-VH-CDR3m (SEQ ID NO: XX).

In some embodiments, a 3E10 antibody or variant thereof, or antigen-binding fragment thereof described herein refers to CDR sequences having no more than one amino acid substitution relative to the parent 3E10 antibody, shown in FIG. 1, optionally including a D31N amino acid substitution in the VH CDR1. Accordingly, in some embodiments, a 3E10 antibody or variant thereof, or antigen-binding fragment thereof includes a light chain variable region (VL) complementarity determining region (CDR) 1 comprising an amino acid sequence having no more than one amino acid substitution relative to 3E10-VL-CDR1 (SEQ ID NO: XX), a VL CDR2 comprising an amino acid sequence having no more than one amino acid substitution relative to 3E10-VL-CDR2 (SEQ ID NO: XX), a VL CDR3 comprising an amino acid sequence having no more than one amino acid substitution relative to 3E10-VL-CDR3 (SEQ ID NO: XX), a heavy chain variable region (VH) CDR1 comprising an amino acid sequence having no more than one amino acid substitution relative to 3E10-VH-CDR1a (SEQ ID NO: XX), a VH CDR2 comprising an amino acid sequence having no more than one amino acid substitution relative to 3E10-VH-CDR2 (SEQ ID NO: XX), and a VH CDR3 comprising an amino acid sequence having no more than one amino acid substitution relative to 3E10-VH-CDR3 (SEQ ID NO: XX).

In some embodiments, a 3E10 antibody or variant thereof, or antigen-binding fragment thereof described herein refers to CDR sequences having no more than two amino acid substitution relative to the parent 3E10 antibody, shown in FIG. 1, optionally including a D31N amino acid substitution in the VH CDR1. Accordingly, in some embodiments, a 3E10 antibody or variant thereof, or antigen-binding fragment thereof includes a light chain variable region (VL) complementarity determining region (CDR) 1 comprising an amino acid sequence having no more than two amino acid substitutions relative to 3E10-VL-CDR1 (SEQ ID NO: XX), a VL CDR2 comprising an amino acid sequence having no more than two amino acid substitutions relative to 3E10-VL-CDR2 (SEQ ID NO: XX), a VL CDR3 comprising an amino acid sequence having no more than two amino acid substitutions relative to 3E10-VL-CDR3 (SEQ ID NO: XX), a heavy chain variable region (VH) CDR1 comprising an amino acid sequence having no more than two amino acid substitutions relative to 3E10-VH-CDR1a (SEQ ID NO: XX), a VH CDR2 comprising an amino acid sequence having no more than two amino acid substitutions relative to 3E10-VH-CDR2 (SEQ ID NO: XX), and a VH CDR3 comprising an amino acid sequence having no more than two amino acid substitutions relative to 3E10-VH-CDR3 (SEQ ID NO: XX).

Other variants of a 3E10 antibody or variant thereof, or antigen-binding fragment thereof are also known in the art, as disclosed for example, in Zack, et al., J. Immunol., 157(5):2082-8 (1996). For example, amino acid position 31 of the heavy chain variable region of 3E10 has been determined to be influential in the ability of the antibody and fragments thereof to penetrate nuclei and bind to DNA (bolded in SEQ ID NOs: 1, 2 and 13). A D31N mutation (bolded in SEQ ID NOs: 2 and 13) in CDR1 penetrates nuclei and binds DNA with much greater efficiency than the original antibody (Zack, et al., Immunology and Cell Biology, 72.513-520 (1994), Weisbart, et al., J. Autoimmun., 11, 539-546 (1998); Weisbart, Int. J. Oncol., 25, 1867-1873 (2004)). In some embodiments, the antibody has the D31N substitution.

Although generally referred to herein as “3E10” or “3E10 antibodies,” it will be appreciated that fragments and binding proteins, including antigen-binding fragments, variants, and fusion proteins such as scFv, di-scFv, tr-scFv, and other single chain variable fragments, and other cell-penetrating, nucleic acid transporting molecules disclosed herein are encompassed by the phrase are also expressly provided for use in compositions and methods disclosed herein. Thus, the antibodies and other binding proteins are also referred to herein as cell-penetrating.

In preferred embodiments, the 3E10 antibody is transported into the cytoplasm and/or nucleus of the cells without the aid of a carrier or conjugate. For example, the monoclonal antibody 3E10 and active fragments thereof that are transported in vivo to the nucleus of mammalian cells without cytotoxic effect are disclosed in U.S. Pat. Nos. 4,812,397 and 7,189,396 to Richard Weisbart.

Antibodies useful in the compositions and methods described herein include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the antibodies typically contain at least the CDRs necessary to maintain DNA binding and/or interfere with DNA repair.

The 3E10 antibody is typically a monoclonal 3E10, or a variant, derivative, fragment, fusion, or humanized form thereof that binds the same or different epitope(s) as 3E10.

A deposit according to the terms of the Budapest Treaty of a hybridoma cell line producing monoclonal antibody 3E10 was received on Sep. 6, 2000, and accepted by, American Type Culture Collection (ATCC), 10801 University Blvd., Manassas, VA 20110-2209, USA, and given Patent Deposit Number PTA-2439.

Thus, the antibody may have the same or different epitope specificity as monoclonal antibody 3E10 produced by ATCC No. PTA 2439 hybridoma. The antibody can have the paratope of monoclonal antibody 3E10. The antibody can be a single chain variable fragment of 3E10, or a variant, e.g., a conservative variant thereof. For example, the antibody can be a single chain variable fragment of 3E10 (3E10 Fv), or a variant thereof.

Additionally, or alternatively, the heavy chain complementarity determining regions (CDRs) can be defined according to the IMGT system. The complementarity determining regions (CDRs) as identified by the IMGT system include CDR H1.3 (original sequence): GFTFSDYG (SEQ ID NO: XX); CDR H1.4 (with D31N mutation): GFTFSNYG (SEQ ID NO: XX); CDR H2.2: ISSGSSTI (SEQ ID NO: XX) and variant ISSSSSTI (SEQ ID NO: XX); CDR H3.2: ARRGLLLDY (SEQ ID NO: XX).

Additionally, or alternatively, the light chain complementarity determining regions (CDRs) can be defined according to the IMGT system. The complementarity determining regions (CDRs) as identified by the IMGT system include CDR L1.2 KSVSTSSYSY (SEQ ID NO: XX) and variant KTVSTSSYSY (SEQ ID NO: XX); CDR L2.2: YAS (SEQ ID NO: XX); CDR L3.2: QHSREFPWT (SEQ ID NO: XX).

In some embodiments, the antibody is a humanized antibody. Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

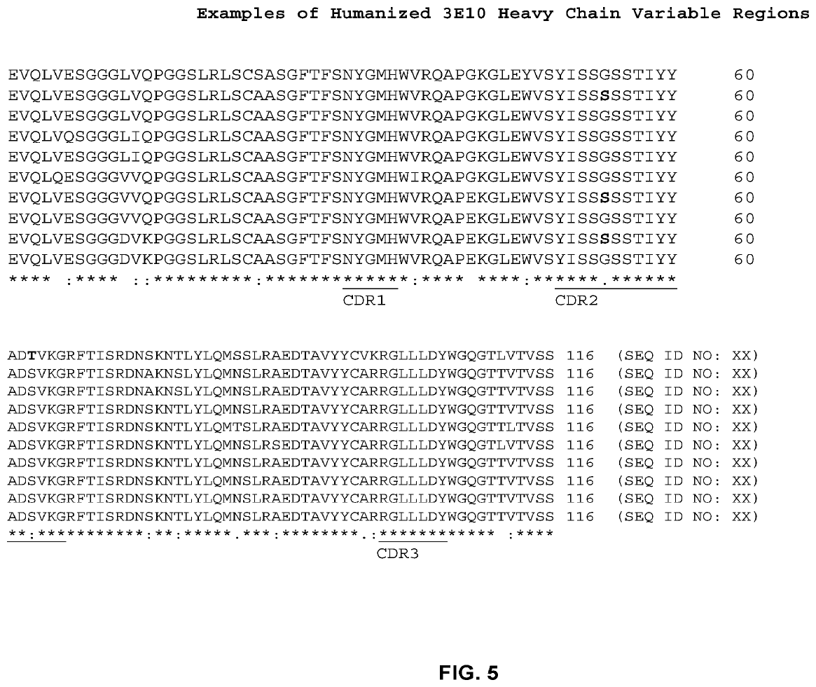

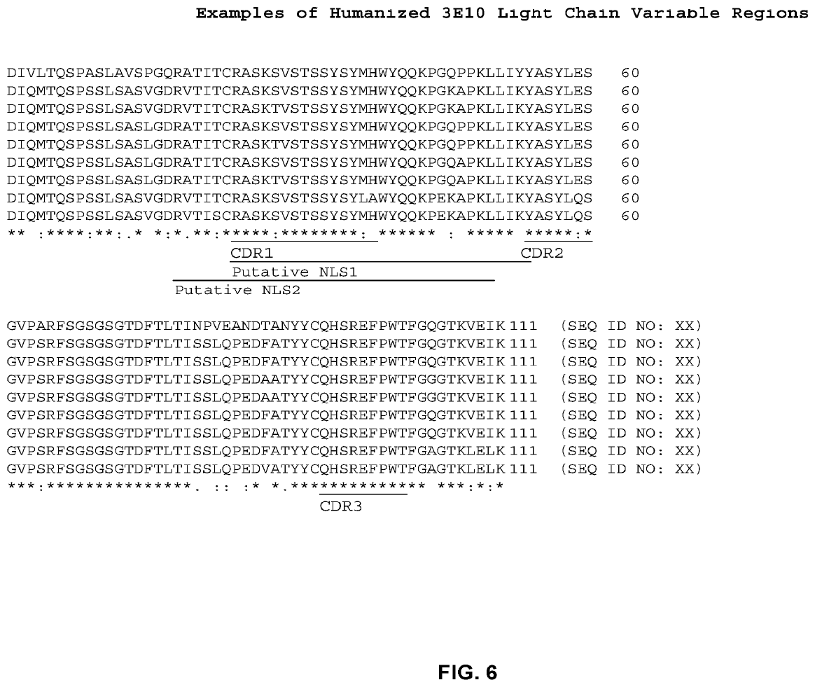

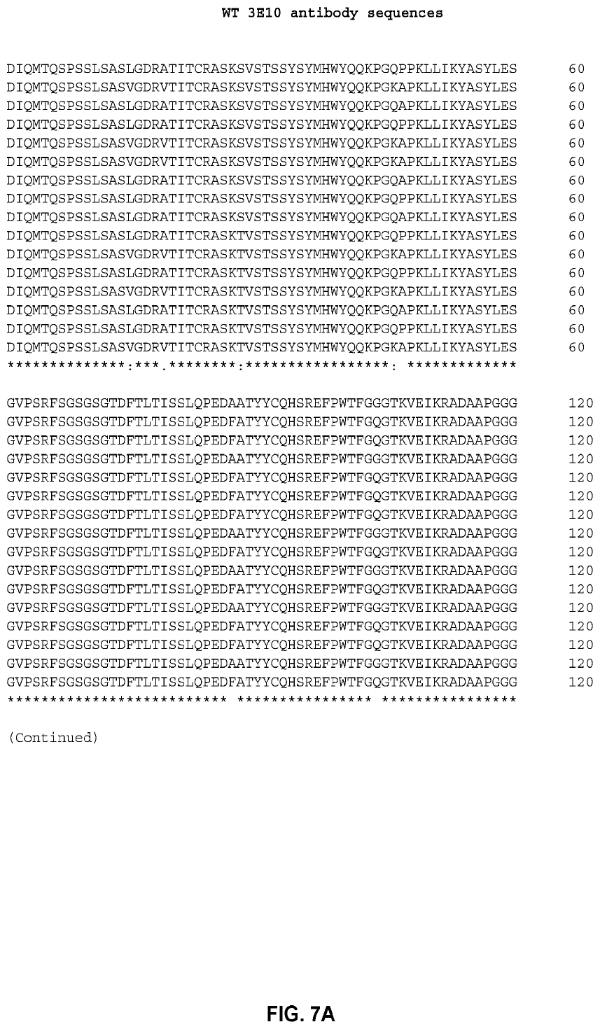

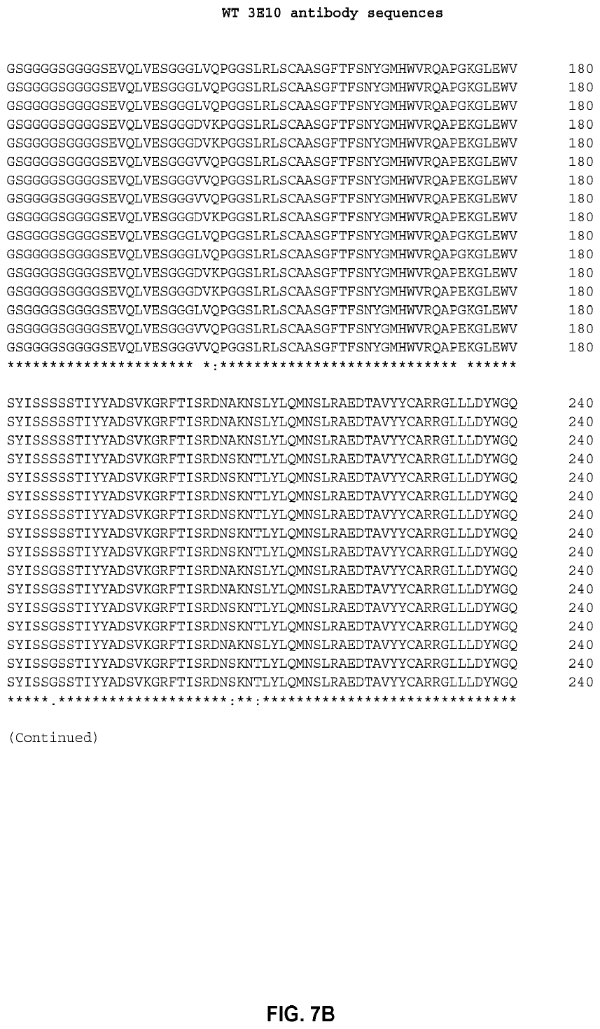

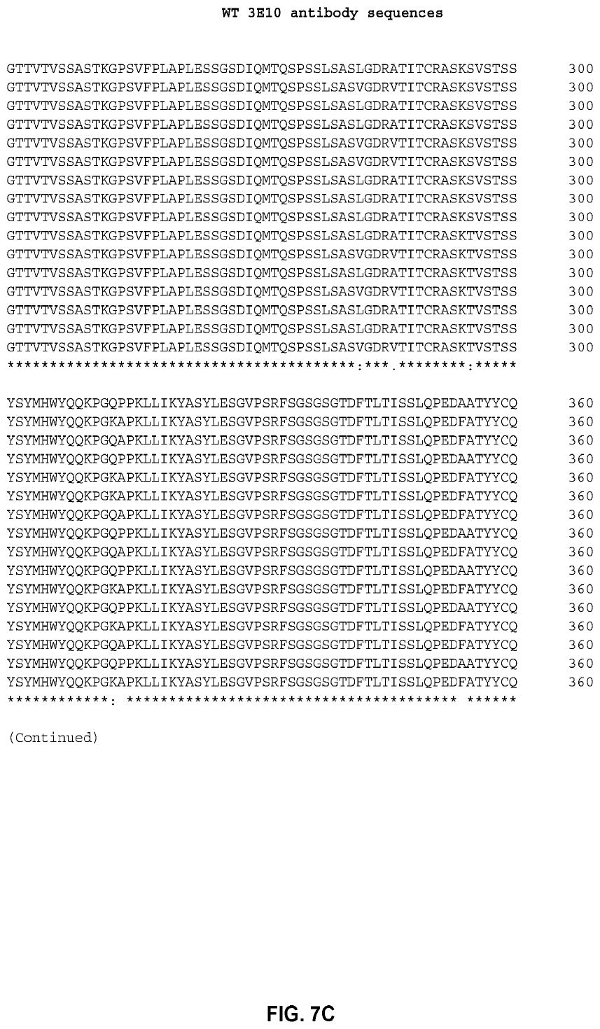

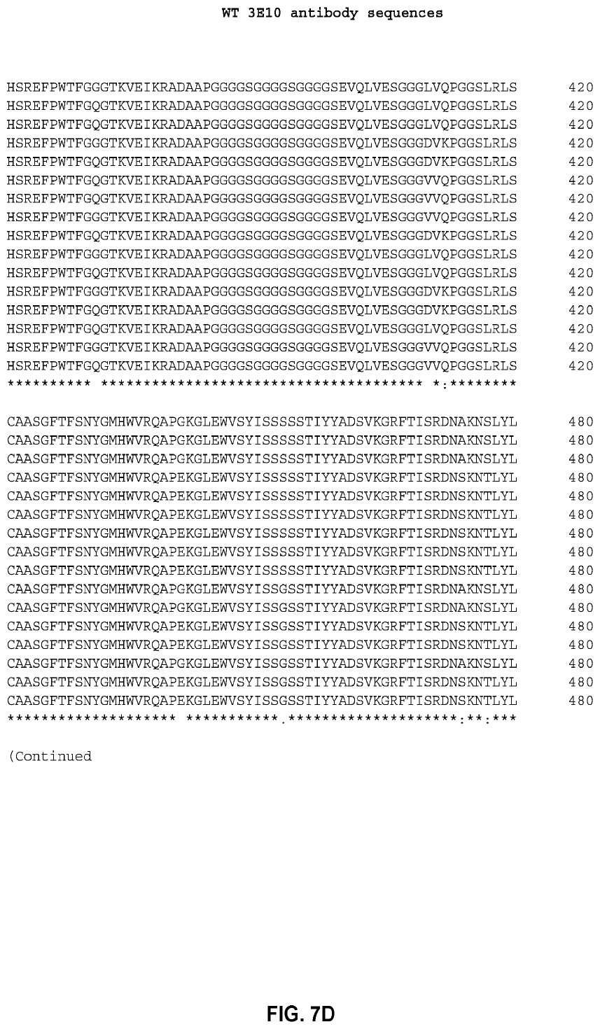

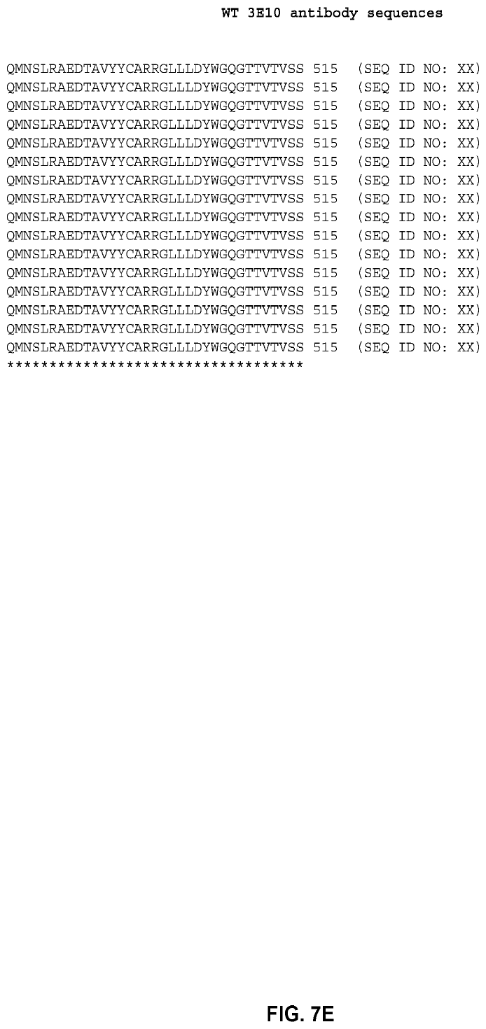

Examples of 3E10 humanized sequences are discussed in WO 2015/106290, WO 2016/033324, WO 2019/018426, and WO/2019/018428, the disclosures of which are incorporated herein by reference in their entireties for all purposes, and specifically for their disclosure of humanized 3E10 sequences and methods for generating the same. Other examples of humanized 3E10 heavy chain variable regions (SEQ ID NOS: XX-YY) and humanized 3E10 light chain variable regions (SEQ ID NOS: XX-YY) are shown in FIGS. 5 and 6, respectively.

The disclosed compositions and methods typically utilize 3E10 antibodies that maintain the ability to penetrate cells, and optionally nuclei.

The mechanisms of cellular internalization by autoantibodies are diverse. Some are taken into cells through electrostatic interactions or FcR-mediated endocytosis, while others utilize mechanisms based on association with cell surface myosin or calreticulin, followed by endocytosis (Ying-Chyi et al., Eur. J. Immunol. 38, 3178-3190 (2008), Yanase et al., J. Clin. Invest. 100, 25-31 (1997)).

3E10 penetrates cells in an Fc-independent mechanism (as evidenced by the ability of 3E10 fragments lacking an Fc to penetrate cells) that involves the presence of the nucleoside transporter ENT2 (Weisbart et al., Sci Rep 5:12022. doi: 10.1038/srep12022. (2015), Zack et al., J. Immunol. 157, 2082-2088 (1996), Hansen et al., J. Biol. Chem. 282, 20790-20793 (2007)). Thus, in some embodiments, the 3E10 antibodies or variants thereof, or antigen-binding fragments thereof utilized in the disclosed compositions and methods penetrate cells in an Fc-independent mechanism. Similarly, in some embodiments, the 3E10 antibodies or variants thereof, or antigen-binding fragments thereof utilized in the disclosed compositions and methods penetrate cells in an ENT2-dependent fashion. The disclosed compositions and methods use 3E10 antibodies or variants thereof, or antigen-binding fragments thereof that maintain the ability to bind polynucleotides, e.g., therapeutic polynucleotides for treating cancer.

Example 8 describes molecular modeling of 3E10 and additional 3E10 variants. Molecular modeling of 3E10 (Pymol) revealed a putative Nucleic Acid Binding pocket (NAB1) (see, e.g., FIGS. 15A and 15B), and illustrated with underlining in FIG. 15C. NAB1 amino acids predicted from molecular modeling have been underlined in the heavy and light chain sequences in FIG. 15C. FIG. 15B is an illustration showing molecular modeling of 3E10-scFv (Pymol) with NAB1 amino acid residues illustrated with punctate dots.

In some embodiments, the disclosed 3E10 antibodies or variants thereof, or antigen-binding fragments thereof include some or all of the underlined NAB1 sequences. In some embodiments, the 3E10 antibodies or variants thereof, or antigen-binding fragments thereof include a variant sequence that has an altered ability of bind nucleic acids. In some embodiments, the mutations (e.g., substitutions, insertions, and/or deletions) in the NAB1 improve binding of the antibody to nucleic acids such as RNA. In some embodiments, the mutations are conservative substitutions. In some embodiments, the mutations increase the cationic charge of the NAB1 pocket.

As discussed and exemplified herein, mutation of aspartic acid at residue 31 of CDR1 to asparagine increased the cationic charge of this residue and enhanced nucleic acid binding and delivery in vivo (3E10-D31N). Mutation of aspartic acid at residue 31 of CDR1 to asparagine increased the cationic charge of this residue and enhanced nucleic acid binding and delivery in vivo (3E10-D31N). Mutation of aspartic acid at residue 31 of CDR1 to arginine (3E10-D31R), further expanded the cationic charge while mutation to lysine (3E10-D31K) changed charge orientation (FIG. 15A).

Accordingly, in some embodiments, the 3E10 antibodies or variants thereof, or antigen-binding fragments thereof include a substitution of aspartic acid at residue 31 of CDR1 to arginine (3E10-D31R), which modeling indicates expands cationic charge, or lysine (3E10-D31K) which modeling indicates changes charge orientation. Thus, in some embodiments, the 3E10 binding protein includes a D31R or D31K substitution. Accordingly, it is contemplated that all sequences disclosed herein having the residue corresponding to 3E10 D31 or N31 may include a D31R or D31K substitution therein.

Mutations in 3E10 that interfere with its ability to bind DNA may render the antibody incapable of nuclear penetration. Thus, typically the disclosed variants and humanized forms of the antibody maintain the ability to bind nucleic acids, particularly DNA. In addition, 3E10 scFv has previously been shown capable of penetrating into living cells and nucleic in an ENT2-dependent manner, with efficiency of uptake impaired in ENT2-deficient cells (Hansen, et al., J. Biol. Chem. 282, 20790-20793 (2007)). Thus, in some embodiments, the disclosed variants and humanized forms of the antibody maintain the ability penetrate into cells in an ENT-dependent, preferably ENT2-dependent manner.

### Immunostimulatory Oligonucleotides

Macromolecular stimulators of the innate immune system, particularly polynucleotide agonists of pattern recognition receptors (PRRs), hold great promise for the treatment of cancer. Pattern recognition receptors (PRRs) recognize pathogen-associated as well as endogenous damage-associated molecular patterns. Once ligand binding occurs, signaling cascades develop within the cells to activate effector molecules, resulting in the recruitment and activation of antitumor immune cells and release of inflammatory cytokines. As such, PRR agonists have been used with success as immunotherapies for the treatment of a wide range of cancers. For a review of PRRs and the use of PRR agonists in cancer immunotherapies see, for example, Bai L., et al., “Promising targets based on pattern recognition receptors for cancer immunotherapy,” Pharmacological Research, 159 (2020) 105017, the content of which is incorporated herein by reference, in its entirety, for all purposes.

Accordingly, in some embodiments, a 3E10 antibody or variant thereof, or antigen-binding fragment thereof is complexed with a polynucleotide immunostimulant, e.g., a polynucleotide capable of stimulating a pattern recognition receptor (PRR).

### Pattern Recognition Receptor (PRR) Agonists

In one aspect, the present disclosure relates to compositions and methods for treating a cancer in a subject by administering to the subject a therapeutically effective amount of a composition including a complex formed between (i) a polynucleotide ligand capable of stimulating a pattern recognition receptor (PRR) and (ii) a 3E10 antibody or variant thereof, or antigen-binding fragment thereof, as described herein. As recognized in the art, stimulation of the innate immune system, e.g., through activation of pattern recognition receptors, represents a promising therapeutic avenue for treating cancer. Generally, PRRs stimulate the innate immune system following recognition of pathogen-associated patterns (PAMPs) and/or damage-associated patterns (DAMPs). Conventionally, PRRs are grouped into five categories: Toll-like receptors (TLRs), C-type lectin receptors (CLRs), RIG-I-like receptors (RLRs), Nucleotide-binding Oligomerization Domain (NOD)-like receptors (NLRs), and cytosolic DNA sensors (CDS). The method and compositions described herein act through any of these classes of PRRs the recognize, and are activated by, a polynucleotide antigen.

Accordingly, in one aspect, compositions and methods are provided for treating a cancer by administering to the subject a therapeutically effective amount of a composition including a complex formed between (i) a polynucleotide ligand capable of stimulating a Toll-like receptor (TLR), and (ii) a 3E10 antibody or variant thereof, or antigen-binding fragment thereof. At least 13 Toll-like receptors have been identified, including TLR1, TLR2, TLR3, TLR4, TLR5, TLR6, TLR7, TLR8, TLR9, TLR10, TLR11, TLR12, and TLR13. Each of these Toll-like receptors has affinity for a different antigen. In accordance with various embodiments of the present disclosure, the methods and compositions described herein include use a polynucleotide agonist of a TLR. For example, each of TLR3, TLR7, TLR8, and TLR9 have affinity for, and are activated by, various polynucleotides. Accordingly, in some embodiments, the agonists used in the methods and compositions described herein are capable of stimulating TLR3, TLR7, TLR8, or TLR9.

For example, unmethylated CpG sites can be detected by TLR9 on plasmacytoid dendritic cells and B cells in humans (Zaida, et al., Infection and Immunity, 76(5):2123-2129, (2008)). Therefore, the sequence of oligonucleotide can include one or more unmethylated cytosine-guanine (CG or CpG, used interchangeably) dinucleotide motifs. The ‘p’ refers to the phosphodiester backbone of DNA, however, in some embodiments, oligonucleotides including CG can have a modified backbone, for example a phosphorothioate (PS) backbone.

In some embodiments, an oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, AM, Advanced Drug Delivery Reviews 61(3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, GL, PNAS USA 94(20): 10833-7 (1997); Dalpke, A H, Immunology 106(1): 102-12 (2002); Hartmann, G, J of Immun. 164(3):1617-2 (2000), each of which is incorporated herein by reference).

RIG-I like receptors (RLRs) are a family of RNA helicases that function as cytoplasmic sensors of pathogen-associated molecular patterns (PAMPs) within viral RNA. Accordingly, in another aspect, compositions and methods are provided for treating a cancer by administering to the subject a therapeutically effective amount of a composition including a complex formed between (i) a polynucleotide ligand capable of stimulating a RIG-I-like receptor (RLR), and (ii) a 3E10 antibody or variant thereof, or antigen-binding fragment thereof. Identified RLRs include RIG-I (retinoic acid-inducible gene I), MDA5 (melanoma differentiation associated factor 5), and LGP2 (laboratory of genetics and physiology 2).

Exemplary RIG-I ligands include, but are not limited to, 5′ppp-dsRNA, a specific agonist of RIG-I; 3p-hpRNA, a specific agonist of RIG-I; Poly(I:C)/LyoVec complexes that are recognized by RIG-I and/or MDA-5 depending of the size of poly(I:C); Poly(dA:dT)/LyoVec complexes that are indirectly recognized by RIG-I. In some embodiments, the 3p-hpRNA is a 5′ triphosphate hairpin RNA that was generated by in vitro transcription of a sequence from the influenza A (H1N1). In some embodiments, the 3p-hpRNA is an RNA oligonucleotide that contains an uncapped 5′ triphosphate extremity and a double-strand fragment. In some embodiments, the 3p-hpRNA is about 50 bp, about 55 bp, about 60 bp, about 65 bp, about 70 bp, about 75 bp, about 80 bp, about 85 bp, about 90 bp, about 100 bp, or more. In some embodiments, the 3p-hpRNA is 89 bp long.

In one embodiment, compositions and methods are provided for treating a cancer by administering to the subject a therapeutically effective amount of a composition including a complex formed between (i) a polynucleotide ligand capable of stimulating RIG-I, and (ii) a 3E10 antibody or variant thereof, or antigen-binding fragment thereof. RIG-I is a helical ATP-dependent DExD/H box RNA helicase, with preference for short (e.g., less than 300 base pairs) dsRNA with a 5′ triphosphate moiety. Several studies have also suggested that RIG-I has a preference for RNAs containing poly-uracil (polyU) regions, e.g., of at least 5, preferably at least 8 consecutive uracil residues.

Accordingly, in some embodiments, compositions and methods are provided for treating a cancer by administering to the subject a therapeutically effective amount of a composition including a complex formed between (i) an RNA molecule that is at least partially double-stranded and is capable of stimulating RIG-I, and (ii) a 3E10 antibody or variant thereof, or antigen-binding fragment thereof. In some embodiments, the at least partially double-stranded RNA molecule comprises two separate RNA strands that anneal to form a double-stranded portion of the molecule. In other embodiments, the at least partially double-stranded RNA molecule is a single RNA strand with self-complementarity, such that under physiological conditions it anneals to itself to form a double-stranded portion of the molecule, e.g., thereby forming one or more hairpin structures.

Similarly, in some embodiments, compositions and methods are provided for treating a cancer by administering to the subject a therapeutically effective amount of a composition including a complex formed between (i) a polynucleotide that is at least partially double-stranded, contains at least one 5′ triphosphate moiety, and is capable of stimulating RIG-I, and (ii) a 3E10 antibody or variant thereof, or antigen-binding fragment thereof. In some embodiments, the at least partially double-stranded RNA molecule comprises two separate RNA strands that anneal to form a double-stranded portion of the molecule. In other embodiments, the at least partially double-stranded RNA molecule is a single RNA strand with self-complementarity, such that under physiological conditions it anneals to itself to form a double-stranded portion of the molecule, e.g., thereby forming one or more hairpin structures. Examples of polynucleotide RIG-I agonists are provided in the literature. Generally, any one of these polynucleotide RIG-I agonists finds use in the methods and compositions described herein.

For instance, PCT Application Publication No. WO 2008/017473, the content of which is incorporated herein by reference, in its entirety, for all purposes, describes examples of polynucleotide RIG-I agonists having 5′ triphosphate groups. As described in WO 2008/017473, in some embodiments, the RIG-I agonist having a 5′ triphosphate group is free of any 5′ cap or other modification. Accordingly, in some embodiments, compositions and methods are provided for treating a cancer by parenterally administering to the periphery of the subject a therapeutically effective amount of a composition including a complex formed between (i) a polynucleotide that is at least partially double-stranded, contains at least one 5′ triphosphate moiety, is free of any 5′ cap or other modification, and is capable of stimulating RIG-I, and (ii) a 3E10 antibody or variant thereof, or antigen-binding fragment thereof. In some embodiments, the RIG-I agonist contains at least 1 ribonucleotide at a 5′ end of the polynucleotide. In some embodiments, the RIG-I agonist contains at least 2, 3, 4, 5, 6, 7, 8, 9, 10, or more ribonucleotides at a 5′ end of the polynucleotide. In some embodiments, the RIG-I agonist is a polyribonucleotide molecule, or a modified version thereof. In some embodiments, the RIG-I agonist is a polynucleotide disclosed or described in WO 2008/017473.

As described in WO 2008/017473, in some embodiments, the polynucleotide RIG-I agonist includes a 2′-methyl-dNTP and/or 2′-fluorine-dNTP modification. Accordingly, in some embodiments, compositions and methods are provided for treating a cancer by parenterally administering to the periphery of the subject a therapeutically effective amount of a composition including a complex formed between (i) a polynucleotide that is at least partially double-stranded, contains at least one 5′ triphosphate moiety, contains a 2′-methyl-dNTP and/or 2′-fluorine-dNTP modification, and is capable of stimulating RIG-I, and (ii) a 3E10 antibody or variant thereof, or antigen-binding fragment thereof. In some embodiments, the RNA molecule is free of any 5′ cap or other modification. In some embodiments, the RIG-I agonist contains at least 1 ribonucleotide at a 5′ end of the polynucleotide. In some embodiments, the RIG-I agonist contains at least 2, 3, 4, 5, 6, 7, 8, 9, 10, or more ribonucleotides at a 5′ end of the polynucleotide. In some embodiments, the RIG-I agonist is a polyribonucleotide molecule, or a modified version thereof.

As described in WO 2008/017473, in some embodiments, the polynucleotide RIG-I agonist has no overhangs at the 3′ end of one strand. Accordingly, in some embodiments, compositions and methods are provided for treating a cancer by parenterally administering to the periphery of the subject a therapeutically effective amount of a composition including a complex formed between (i) a polynucleotide that is at least partially double-stranded, contains at least one 5′ triphosphate moiety, has no overhangs at the 3′ end of one strand, and is capable of stimulating RIG-I, and (ii) a 3E10 antibody or variant thereof, or antigen-binding fragment thereof. In some embodiments, the RNA molecule is free of any 5′ cap or other modification. In some embodiments, the RIG-I agonist contains at least 1 ribonucleotide at a 5′ end of the polynucleotide. In some embodiments, the RIG-I agonist contains at least 2, 3, 4, 5, 6, 7, 8, 9, 10, or more ribonucleotides at a 5′ end of the polynucleotide. In some embodiments, the RIG-I agonist is a polyribonucleotide molecule, or a modified version thereof. In some embodiments, the RIG-I agonist contains a 2′-methyl-dNTP and/or 2′-fluorine-dNTP modification.

As described in WO 2008/017473, in some embodiments, the polynucleotide RIG-I agonist has a single nucleotide overhang at the 3′ end of one strand. Accordingly, in some embodiments, compositions and methods are provided for treating a cancer by parenterally administering to the periphery of the subject a therapeutically effective amount of a composition including a complex formed between (i) a polynucleotide that is at least partially double-stranded, contains at least one 5′ triphosphate moiety, has a single nucleotide overhang at the 3′ end of one strand, and is capable of stimulating RIG-I, and (ii) a 3E10 antibody or variant thereof, or antigen-binding fragment thereof. In some embodiments, the RNA molecule is free of any 5′ cap or other modification. In some embodiments, the RIG-I agonist contains at least 1 ribonucleotide at a 5′ end of the polynucleotide. In some embodiments, the RIG-I agonist contains at least 2, 3, 4, 5, 6, 7, 8, 9, 10, or more ribonucleotides at a 5′ end of the polynucleotide. In some embodiments, the RIG-I agonist is a polyribonucleotide molecule, or a modified version thereof. In some embodiments, the RIG-I agonist contains a 2′-methyl-dNTP and/or 2′-fluorine-dNTP modification.

As described in WO 2008/017473, in some embodiments, the polynucleotide RIG-I agonist has a two-nucleotide overhang at the 3′ end of one strand. Accordingly, in some embodiments, compositions and methods are provided for treating a cancer by parenterally administering to the periphery of the subject a therapeutically effective amount of a composition including a complex formed between (i) a polynucleotide that is at least partially double-stranded, contains at least one 5′ triphosphate moiety, has a two-nucleotide overhang at the 3′ end of one strand, and is capable of stimulating RIG-I, and (ii) a 3E10 antibody or variant thereof, or antigen-binding fragment thereof. In some embodiments, the RNA molecule is free of any 5′ cap or other modification. In some embodiments, the RIG-I agonist contains at least 1 ribonucleotide at a 5′ end of the polynucleotide. In some embodiments, the RIG-I agonist contains at least 2, 3, 4, 5, 6, 7, 8, 9, 10, or more ribonucleotides at a 5′ end of the polynucleotide. In some embodiments, the RIG-I agonist is a polyribonucleotide molecule, or a modified version thereof. In some embodiments, the RIG-I agonist contains a 2′-methyl-dNTP and/or 2′-fluorine-dNTP modification.

Similarly, PCT Application Publication Nos. WO 2009/141146 and WO 2010/002851, the contents of which are incorporated herein by reference, in their entireties, for all purposes, describes examples of polynucleotide RIG-I agonists having 5′ triphosphate groups. As described in WO 2008/017473, in some embodiments, the RIG-I agonist having a 5′ triphosphate group has a blunt end. Accordingly, in some embodiments, compositions and methods are provided for treating a cancer by parenterally administering to the periphery of the subject a therapeutically effective amount of a composition including a complex formed between (i) a polynucleotide that is at least partially double-stranded, contains at least one 5′ triphosphate moiety, has one blunt end, and is capable of stimulating RIG-I, and (ii) a 3E10 antibody or variant thereof, or antigen-binding fragment thereof. In some embodiments, the RIG-I agonist contains at least 1 ribonucleotide at a 5′ end of the polynucleotide. In some embodiments, the RIG-I agonist contains at least 2, 3, 4, 5, 6, 7, 8, 9, 10, or more ribonucleotides at a 5′ end of the polynucleotide. In some embodiments, the RIG-I agonist is free of any 5′ cap or other modification. In some embodiments, the RIG-I agonist has a single polynucleotide chain. In some embodiments, the RIG-I agonist has two polynucleotide chains. In some embodiments, the RIG-I agonist is a polyribonucleotide molecule, or a modified version thereof. In some embodiments, the RIG-I agonist is a polynucleotide disclosed or described in WO 2009/141146 or WO 2010/002851.

As described in WO 2009/141146 and WO 2010/002851, in some embodiments, the RIG-I agonist having a 5′ triphosphate group has two blunt ends. Accordingly, in some embodiments, compositions and methods are provided for treating a cancer by parenterally administering to the periphery of the subject a therapeutically effective amount of a composition including a complex formed between (i) a polynucleotide that is at least partially double-stranded, contains at least one 5′ triphosphate moiety, has two blunt ends, and is capable of stimulating RIG-I, and (ii) a 3E10 antibody or variant thereof, or antigen-binding fragment thereof. In some embodiments, the RIG-I agonist contains at least 1 ribonucleotide at a 5′ end of the polynucleotide. In some embodiments, the RIG-I agonist contains at least 2, 3, 4, 5, 6, 7, 8, 9, 10, or more ribonucleotides at a 5′ end of the polynucleotide. In some embodiments, the RIG-I agonist is free of any 5′ cap or other modification. In some embodiments, the RIG-I agonist is a polyribonucleotide molecule, or a modified version thereof.

PCT Application Publication No. WO 2014/049079, the content of which is incorporated herein by reference, in its entirety, for all purposes, describes examples of polynucleotide RIG-I agonists having 5′ triphosphate groups that are chemically modified to improve their stability. As described in WO 2014/049079, in some embodiments, the RIG-I agonist having a 5′ triphosphate group includes at least one 2′-O-methylated nucleotide. Accordingly, in some embodiments, compositions and methods are provided for treating a cancer by parenterally administering to the periphery of the subject a therapeutically effective amount of a composition including a complex formed between (i) a polynucleotide that is at least partially double-stranded, contains at least one 5′ triphosphate moiety, contains at least one 2′-O-methylated nucleotide, and is capable of stimulating RIG-I, and (ii) a 3E10 antibody or variant thereof, or antigen-binding fragment thereof. In some embodiments, the RIG-I agonist contains at least 1 ribonucleotide at a 5′ end of the polynucleotide. In some embodiments, the RIG-I agonist contains at least 2, 3, 4, 5, 6, 7, 8, 9, 10, or more ribonucleotides at a 5′ end of the polynucleotide. In some embodiments, the RIG-I agonist is free of any 5′ cap or other modification. In some embodiments, the RIG-I agonist is a polyribonucleotide molecule, or a modified version thereof. In some embodiments, the RIG-I agonist is a polynucleotide disclosed or described in WO 2014/049079.

As described in WO 2014/049079, in some embodiments, the RIG-I agonist having a 5′ triphosphate group includes at least one 2′-fluoro nucleotide. Accordingly, in some embodiments, compositions and methods are provided for treating a cancer by parenterally administering to the periphery of the subject a therapeutically effective amount of a composition including a complex formed between (i) a polynucleotide that is at least partially double-stranded, contains at least one 5′ triphosphate moiety, contains at least one 2′-fluoro nucleotide, and is capable of stimulating RIG-I, and (ii) a 3E10 antibody or variant thereof, or antigen-binding fragment thereof. In some embodiments, the RIG-I agonist contains at least 1 ribonucleotide at a 5′ end of the polynucleotide. In some embodiments, the RIG-I agonist contains at least 2, 3, 4, 5, 6, 7, 8, 9, 10, or more ribonucleotides at a 5′ end of the polynucleotide. In some embodiments, the RIG-I agonist is free of any 5′ cap or other modification. In some embodiments, the RIG-I agonist is a polyribonucleotide molecule, or a modified version thereof. In some embodiments, the RIG-I agonist is a polynucleotide disclosed or described in WO 2014/049079.

As described in WO 2014/049079, in some embodiments, the RIG-I agonist having a 5′ triphosphate group includes at least one dinucleotide linked by a phosphorothioate bond. Accordingly, in some embodiments, compositions and methods are provided for treating a cancer by parenterally administering to the periphery of the subject a therapeutically effective amount of a composition including a complex formed between (i) a polynucleotide that is at least partially double-stranded, contains at least one 5′ triphosphate moiety, contains at least one dinucleotide linked by a phosphorothioate bond, and is capable of stimulating RIG-I, and (ii) a 3E10 antibody or variant thereof, or antigen-binding fragment thereof. In some embodiments, the RIG-I agonist contains at least 1 ribonucleotide at a 5′ end of the polynucleotide. In some embodiments, the RIG-I agonist contains at least 2, 3, 4, 5, 6, 7, 8, 9, 10, or more ribonucleotides at a 5′ end of the polynucleotide. In some embodiments, the RIG-I agonist is free of any 5′ cap or other modification. In some embodiments, the RIG-I agonist is a polyribonucleotide molecule, or a modified version thereof. In some embodiments, the RIG-I agonist is a polynucleotide disclosed or described in WO 2014/049079.

In some embodiments, as described in WO 2014/049079, the RIG-I agonist includes a sense strand and an antisense strand, where the sense strand includes the nucleotide sequence 5′ GACGCUGfACCCUGAAmGUUCAUCPTOUfPTOU 3′ (SEQ ID NO:XX), and the antisense strand includes the nucleotide sequence 3′ CUGmCGACUGGGACfUUCAAGUAGPTOAPTOA 5′ (SEQ ID NO:XX), where a nucleotide indexed m is 2′-O-methylated, a nucleotide indexed f is 2′-fluoro; and the index PTO between two nucleotides indicates that said two nucleotides are linked by a phosphorothioate bond.

In some embodiments, as described in WO 2014/049079, the RIG-I agonist includes a sense strand and an antisense strand, where the sense strand includes the nucleotide sequence 5′ GPTOAPTOCGCUGfACCCUGAAmGUUCAUCPTOUfPTOU 3′ (SEQ ID NO:XX), and the antisense strand includes the nucleotide sequence 3′ CUGpmCGACUGGGACfUUCAAGUAGPTOAPTOA 5′ (SEQ ID NO:XX), where a nucleotide indexed m is 2′-O-methylated, a nucleotide indexed f is 2′-fluoro; and the index PTO between two nucleotides indicates that said two nucleotides are linked by a phosphorothioate bond.

PCT Application Publication No. WO 2017/001702, the content of which is incorporated herein by reference, in its entirety, for all purposes, describes examples of discontinuous oligonucleotide RIG-I agonists. As described in WO 2017/001702, in some embodiments, the RIG-I agonist has the structure:

RNA₁-B-RNA₂

RNA₃ RNA₄

where RNA₁ represents a first strand of a ribonucleic acid or an analog or derivative thereof of at least six nucleotides in length, RNA₂ represents a second strand of a ribonucleic acid or an analog or derivative thereof of at least six nucleotides in length, RNA₃ represents a third strand of a ribonucleic acid or an analog or derivative thereof of at least six nucleotides in length, which forms at least five complementary base pairs with RNA₁, RNA₄ represents a fourth strand of a ribonucleic acid or an analog or derivative thereof of at least six nucleotides in length, which forms at least five complementary base pairs with RNA₂, B represents a bivalent linker that covalently bonds the 5′ terminus of RNA₁ to the 5′ terminus of RNA₂ or the 3′ terminus of RNA₁ to the 3′ terminus of RNA₂, and RNA₃ and RNA₄ are not covalently bonded to one another.

Accordingly, in some embodiments, compositions and methods are provided for treating a cancer by parenterally administering to the periphery of the subject a therapeutically effective amount of a composition including a complex formed between (i) a discontinuous oligonucleotide having the structure

RNA₁-B-RNA₂

RNA₃ RNA₄

where RNA₁ represents a first strand of a ribonucleic acid or an analog or derivative thereof of at least six nucleotides in length, RNA₂ represents a second strand of a ribonucleic acid or an analog or derivative thereof of at least six nucleotides in length, RNA₃ represents a third strand of a ribonucleic acid or an analog or derivative thereof of at least six nucleotides in length, which forms at least five complementary base pairs with RNA₁, RNA₄ represents a fourth strand of a ribonucleic acid or an analog or derivative thereof of at least six nucleotides in length, which forms at least five complementary base pairs with RNA₂, B represents a bivalent linker that covalently bonds the 5′ terminus of RNA₁ to the 5′ terminus of RNA₂ or the 3′ terminus of RNA₁ to the 3′ terminus of RNA₂, and RNA₃ and RNA₄ are not covalently bonded to one another, and (ii) a 3E10 antibody or variant thereof, or antigen-binding fragment thereof. In some embodiments, the RIG-I agonist is a polyribonucleotide molecule, or a modified version thereof. In some embodiments, the RIG-I agonist is a polynucleotide disclosed or described in WO 2017/001702.

PCT Application Publication No. WO 2018/172546, the content of which is incorporated herein by reference, in its entirety, for all purposes, describes examples of polynucleotide RIG-I agonists containing a preferred binding motif. As described in WO 2017/001702, in some embodiments, the polynucleotide RIG-I agonist has a sequence motif on the 5′ end of a nucleotide strand selected from:

(SEQ ID NO: XX)
5′-gbucndnwnnnnnnnnwnsnn-3′,
(SEQ ID NO: XX)
5′-gucuadnwnnnnnnnnwnsnn-3′,
(SEQ ID NO: XX)
5′-guagudnwnnnnnnnnwnsnn-3′,
(SEQ ID NO: XX)
5′-gguaadnwnnnnnnnnwnsnn-3′,
(SEQ ID NO: XX)
5′-ggcagdnwnnnnnnnnwnsnn-3′,
(SEQ ID NO: XX)
5′-gcuucdnwnnnnnnnnwnsnn-3′,
(SEQ ID NO: XX)
5′-gcccadnwnnnnnnnnwnsnn-3′,
and
(SEQ ID NO: XX)
5′-gcgcudnwnnnnnnnnwnsnn-3′.

Accordingly, in some embodiments, compositions and methods are provided for treating a cancer by parenterally administering to the periphery of the subject a therapeutically effective amount of a composition including a complex formed between (i) a polynucleotide RIG-I agonist having a sequence motif on the 5′ end of a nucleotide strand selected from:

(SEQ ID NO: XX)
5′-gbucndnwnnnnnnnnwnsnn-3′,
(SEQ ID NO: XX)
5′-gucuadnwnnnnnnnnwnsnn-3′,
(SEQ ID NO: XX)
5′-guagudnwnnnnnnnnwnsnn-3′,
(SEQ ID NO: XX)
5′-gguaadnwnnnnnnnnwnsnn-3′,
(SEQ ID NO: XX)
5′-ggcagdnwnnnnnnnnwnsnn-3′,
(SEQ ID NO: XX)
5′-gcuucdnwnnnnnnnnwnsnn-3′,
(SEQ ID NO: XX)
5′-gcccadnwnnnnnnnnwnsnn-3′,
and
(SEQ ID NO: XX)
5′-gcgcudnwnnnnnnnnwnsnn-3′,

and (ii) a 3E10 antibody or variant thereof, or antigen-binding fragment thereof. In some embodiments, the RIG-I agonist is a polyribonucleotide molecule, or a modified version thereof. In some embodiments, the RIG-I agonist is a polynucleotide disclosed or described in WO 2018/172546.

PCT Application Publication No. WO 2019/204179, the content of which is incorporated herein by reference, in its entirety, for all purposes, describes examples of modified polynucleotide RIG-I agonists that improve RIG-I selectivity and/or boost or abrogate RIG-I driven immunity. Accordingly, in some embodiments, the RIG-I agonist used in the methods and compositions described herein is an agonist disclosed or described in WO 2019/204179.

PCT Application Publication No. WO 2020/260547, the content of which is incorporated herein by reference, in its entirety, for all purposes, describes examples of modified polynucleotide RIG-I agonists that improve RIG-I selectivity and/or boost or abrogate RIG-I driven immunity. Specifically, WO 2019/204179 discloses polynucleotide RIG-I agonists with specific 2′-0-methylation and/or 2′-fluorination modification patterns. Accordingly, in some embodiments, the RIG-I agonist used in the methods and compositions described herein is an agonist disclosed or described in WO 2020/260547.

PCT Application Publication No. WO 2019/246450, the content of which is incorporated herein by reference, in its entirety, for all purposes, describes examples of short hairpin polynucleotide RIG-I agonists. As described in WO 2019/246450, in some embodiments, the RIG-I agonist is a double-stranded duplex or a single chain polynucleotide containing a loop or hairpin structure and a double stranded portion containing fewer than 25 base pairs. In some embodiments, the double stranded portion contains fewer than 30, fewer than 25, fewer than 20, fewer than 19, fewer than 18, fewer than 17, fewer than 16, fewer than 15, fewer than 14, fewer than 13, fewer than 12, fewer than 11, fewer than 10, fewer than 9, fewer than 8, or less base pairs. Accordingly, in some embodiments, compositions and methods are provided for treating a cancer by parenterally administering to the periphery of the subject a therapeutically effective amount of a composition including a complex formed between (i) a short hairpin polynucleotide capable of stimulating RIG-I, and (ii) a 3E10 antibody or variant thereof, or antigen-binding fragment thereof. In some embodiments, the RIG-I agonist used in the methods and compositions described herein is an agonist disclosed or described in WO 2019/246450.

PCT Application Publication No. WO 2009/095226, the content of which is incorporated herein by reference, in its entirety, for all purposes, describes examples of polynucleotide RIG-I agonists having one or more poly-U sequence elements. In some embodiments, the poly-U element is at least 5 consecutive uracil residues. In some embodiments, the poly-U element is at least 6, 7, 8, 9, 10, 11, 12, 13, 14, 15, or more consecutive uracil residues. As described in WO 2009/095226, in some embodiments, the RIG-I agonist contains the sequence motif (NuGlXmGnNv)a, where:
G is guanosine (guanine), uridine (uracil) or an analogue of guanosine (guanine) or uridine (uracil), preferably guanosine (guanine) or an analogue thereof,
X is guanosine (guanine), uridine (uracil), adenosine (adenine), thymidine (thymine), cytidine (cytosine), or an analogue of these nucleotides (nucleosides), preferably uridine (uracil) or an analogue thereof;
N is a nucleic acid sequence having a length of about 4 to 50, preferably of about 4 to 40, more preferably of about 4 to 30 or 4 to 20 nucleic acids, each N independently being selected from guanosine (guanine), uridine (uracil), adenosine (adenine), thymidine (thymine), cytidine (cytosine) or an analogue of these nucleotides (nucleosides);
a is an integer from 1 to 20, preferably from 1 to 15, most preferably from 1 to 10;
I is an integer from 1 to 40, wherein when I=1, G is guanosine (guanine) or an analogue thereof, when I>1, at least 50% of these nucleotides (nucleosides) are guanosine (guanine) or an analogue thereof,
m is an integer and is at least 3;
wherein when m=3, X is uridine (uracil) or an analogue thereof, and when m>3, at least 3 successive uridines (uracils) or analogues of uridine (uracil) occur;
n is an integer from 1 to 40, wherein when n=1, G is guanosine (guanine) or an analogue thereof, when n>1, at least 50% of these nucleotides (nucleosides) are guanosine (guanine) or an analogue thereof;
u, v may be, independently from each other, an integer from 0 to 50, preferably wherein when u=0, v>1, or when v=0, u>1;
wherein the nucleic acid molecule has a length of at least 50 nucleotides, preferably of at least 100 nucleotides, more preferably of at least 150 nucleotides, even more preferably of at least 200 nucleotides and most preferably of at least 250 nucleotides.

Accordingly, in some embodiments, compositions and methods are provided for treating a cancer by parenterally administering to the periphery of the subject a therapeutically effective amount of a composition including a complex formed between (i) a polynucleotide containing the sequence motif contains the sequence motif (NuGlXmGnNv)a that is capable of stimulating RIG-I, and (ii) a 3E10 antibody or variant thereof, or antigen-binding fragment thereof. In some embodiments, the polynucleotide agonist further includes a poly-U element. In some embodiments, the RIG-I agonist used in the methods and compositions described herein is an agonist disclosed or described in WO 2009/095226.

PCT Application Publication No. WO 2013/064584, the content of which is incorporated herein by reference, in its entirety, for all purposes, describes examples of short double-stranded polynucleotide RIG-I agonists. As described in WO 2013/064584, in some embodiments, the RIG-I agonist has the structure:

5′-(G/C)x(N)y(G/C)z-3′

3′-(G/C)x(N)y(G/C)z-5′
where G/C is a G or C ribonucleotide with the proviso that when a G ribonucleotide is in a position in one strand the other strand has a C ribonucleotide in the opposite position and vice versa; N is any ribonucleotide with the proviso that the sequences of both strands are essentially complementary; each x is an integer of from 3 to 10, in particular 3, 4, 5, 6, 7, 8, 9, or 10, each y is typically an integer of 30 to 200 or more such as 500 or 600, preferably, 30 to 150, more preferably 30 to 100, even more preferred 30 to 50, and each z is an integer of from 3 to 10, with the proviso that x in one strand is equal to the x in the other strand, y in one strand is equal to the y in the other strand, and z in one strand is equal to the z in the other strand; and with the further proviso that the length of said double-stranded structure is at least 45 bp, typically 45 to 520 bp or more such as 620 bp, preferably 45 to 220 bp, more preferably 45 to 150 bp, particularly preferred 45 to 90 bp, even more preferred 45 to 70 bp. Accordingly, in some embodiments, compositions and methods are provided for treating a cancer by parenterally administering to the periphery of the subject a therapeutically effective amount of a composition including a complex formed between (i) a double-stranded polynucleotide having the structure:

5′-(G/C)x(N)y(G/C)z-3′

3′-(G/C)x(N)y(G/C)z-5′

capable of stimulating RIG-I, and (ii) a 3E10 antibody or variant thereof, or antigen-binding fragment thereof. In some embodiments, the RIG-I agonist used in the methods and compositions described herein is an agonist disclosed or described in WO 2013/064584.

PCT Application Publication No. WO 2014/169049, the content of which is incorporated herein by reference, in its entirety, for all purposes, describes examples of RIG-I agonists having a 5′ triphosphate group. As described in WO 2014/169049, in some embodiments, the RIG-I agonist is a non-linear single stranded RNA at least 17 nucleotides long that includes a 2′ fluoro-modified pyrimidine. In some embodiments, the non-linear single stranded RNA is at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, or more nucleotides long. Accordingly, in some embodiments, compositions and methods are provided for treating a cancer by parenterally administering to the periphery of the subject a therapeutically effective amount of a composition including a complex formed between (i) a non-linear single stranded RNA at least 17 nucleotides long having a 5′ triphosphate group and a 2′ fluoro-modified pyrimidine capable of stimulating RIG-I, and (ii) a 3E10 antibody or variant thereof, or antigen-binding fragment thereof. In some embodiments, the RIG-I agonist used in the methods and compositions described herein is an agonist disclosed or described in WO 2014/169049.

U.S. Pat. No. 9,775,894 and PCT Application Publication No. WO 2019/152884, the contents of which are incorporated herein by reference, in their entireties, for all purposes, describe examples of polynucleotide RIG-I agonists having one or more poly-U sequence elements. In some embodiments, the poly-U element is at least 5 consecutive uracil residues. In some embodiments, the poly-U element is at least 6, 7, 8, 9, 10, 11, 12, 13, 14, 15, or more consecutive uracil residues. Accordingly, in some embodiments, compositions and methods are provided for treating a cancer by parenterally administering to the periphery of the subject a therapeutically effective amount of a composition including a complex formed between (i) a polynucleotide containing a poly-U element capable of stimulating RIG-I, and (ii) a 3E10 antibody or variant thereof, or antigen-binding fragment thereof. In some embodiments, the RIG-I agonist used in the methods and compositions described herein is an agonist disclosed or described in U.S. Pat. No. 9,775,894 or PCT Application Publication No. WO 2019/152884.

PCT Application Publication No. WO 2017/173427, the content of which is incorporated herein by reference, in its entirety, for all purposes, describes examples of RIG-I agonists with a central hairpin and an internal loop, which produce no interferon response. Accordingly, in some embodiments, compositions and methods are provided for treating a cancer by parenterally administering to the periphery of the subject a therapeutically effective amount of a composition including a complex formed between (i) a polynucleotide with a central hairpin and an internal loop, which produces no interferon response capable of stimulating RIG-I, and (ii) a 3E10 antibody or variant thereof, or antigen-binding fragment thereof. In some embodiments, the RIG-I agonist used in the methods and compositions described herein is an agonist disclosed or described in WO 2017/173427.

PCT Application Publication No. WO 2019/143297, the content of which is incorporated herein by reference, in its entirety, for all purposes, describes examples of short hairpin RNA agonists of RIG-I with a kink in the stem structure. In some embodiments, the short hairpin RNA has no more than 100 nucleotides. In some embodiments, the short hairpin RNA has no more than 75, no more than 50, no more than 40, no more than 35, no more than 30, no more than 25, no more than 20, no more than 19, no more than 18, no more than 17, no more than 16, no more than 15, or fewer nucleotides. Accordingly, in some embodiments, compositions and methods are provided for treating a cancer by parenterally administering to the periphery of the subject a therapeutically effective amount of a composition including a complex formed between (i) a short hairpin RNA with a kink in the stem structure capable of stimulating RIG-I, and (ii) a 3E10 antibody or variant thereof, or antigen-binding fragment thereof. In some embodiments, the RIG-I agonist used in the methods and compositions described herein is an agonist disclosed or described in WO 2019/143297.

PCT Application Publication No. WO 2019/204743, the content of which is incorporated herein by reference, in its entirety, for all purposes, describes examples of short blunt-ended, hairpin RNA with a 5′ di- or ti-phosphate with a sequence motif that improves RLR-mediated activity. In some embodiments, the short hairpin RNA has no more than 100 nucleotides. In some embodiments, the short hairpin RNA has no more than 75, no more than 50, no more than 40, no more than 35, no more than 30, no more than 25, no more than 20, no more than 19, no more than 18, no more than 17, no more than 16, no more than 15, or fewer nucleotides. Accordingly, in some embodiments, compositions and methods are provided for treating a cancer by parenterally administering to the periphery of the subject a therapeutically effective amount of a composition including a complex formed between (i) a short blunt-ended, hairpin RNA with a 5′ di- or tri-phosphate capable of stimulating RIG-I, and (ii) a 3E10 antibody or variant thereof, or antigen-binding fragment thereof. In some embodiments, the RIG-I agonist used in the methods and compositions described herein is an agonist disclosed or described in WO 2019/204743.

PCT Application Publication No. WO 2021/076713, the content of which is incorporated herein by reference, in its entirety, for all purposes, describes examples of polynucleotide RIG-I agonists containing modified nucleotides. As described in WO 2021/076713, in some embodiments, the RIG-I agonist includes the sequence motif 5′-PEPSZV-3′ (SEQ ID NO:XX), where each P is independently, and for each occurrence, a C-5 modified pyrimidine; E is a C-5 modified pyrimidine, A or G; S is a G or C; Z is a C-5 modified pyrimidine or A; and V is a C, A, or G. Accordingly, in some embodiments, compositions and methods are provided for treating a cancer by parenterally administering to the periphery of the subject a therapeutically effective amount of a composition including a complex formed between (i) a polynucleotide containing the sequence motif contains the sequence motif 5′-PEPSZV-3′ (SEQ ID NO:XX) that is capable of stimulating RIG-I, and (ii) a 3E10 antibody or variant thereof, or antigen-binding fragment thereof. As described in WO 2021/076713, in some embodiments, the RIG-I agonist includes the sequence motif 5′-PEPSFP-3′ (SEQ ID NO:XX), wherein each P is independently, and for each occurrence, a C-5 modified pyrimidine; E is a C-5 modified pyrimidine, A or G; S is a G or C; and F is a C-5 modified pyrimidine, unmodified C, G, or A. Accordingly, in some embodiments, compositions and methods are provided for treating a cancer by parenterally administering to the periphery of the subject a therapeutically effective amount of a composition including a complex formed between (i) a polynucleotide containing the sequence motif contains the sequence motif 5′-PEPSFP-3′ (SEQ ID NO:XX) that is capable of stimulating RIG-I, and (ii) a 3E10 antibody or variant thereof, or antigen-binding fragment thereof. In some embodiments, the RIG-I agonist used in the methods and compositions described herein is an agonist disclosed or described in WO 2021/076713.

In some embodiments, the RIG-I agonist is a 5′ triphosphate hairpin RNA that was generated by in vitro transcription of a sequence from the influenza A (H1N1) virus, a single-stranded negative-sense RNA virus (3p-hpRNA) having the sequence 5′-pppGGAGCAAAAGCAGGGUGACAAAGACAUAAUGGAUCCAA ACACUGUGUCAAGCUUUCAGGUAGAUUGCUUUCUUU GGCAUGUCCGCAAAC-3′ (SEQ ID NO:XX), or a highly conserved nucleotide sequence thereto. See, for example, Rehwinkel J. et al., Cell, 140:397-408 (2010) and Liu G. et al., J Virol. 89(11):6067-79 (2015), the contents of which are incorporated herein by reference, in their entireties, for all purposes. Accordingly, in some embodiments, compositions and methods are provided for treating a cancer by administering to the subject a therapeutically effective amount of a composition including a complex formed between (i) a polynucleotide having the sequence of 3p-hpRNA (SEQ ID NO:XX) that is capable of stimulating RIG-I, and (ii) a 3E10 antibody or variant thereof, or antigen-binding fragment thereof.

In some embodiments, the RIG-I agonist has a sequence that is at least 80% identical to the sequence of 3p-hpRNA (SEQ ID NO:XX). In some embodiments, the RIG-I agonist has a sequence that is at least 85% identical to the sequence of 3p-hpRNA (SEQ ID NO:XX). In some embodiments, the RIG-I agonist has a sequence that is at least 90% identical to the sequence of 3p-hpRNA (SEQ ID NO:XX). In some embodiments, the RIG-I agonist has a sequence that is at least 95% identical to the sequence of 3p-hpRNA (SEQ ID NO:XX). In some embodiments, the RIG-I agonist has a sequence that is at least 96% identical to the sequence of 3p-hpRNA (SEQ ID NO:XX). In some embodiments, the RIG-I agonist has a sequence that is at least 97% identical to the sequence of 3p-hpRNA (SEQ ID NO:XX). In some embodiments, the RIG-I agonist has a sequence that is at least 98% identical to the sequence of 3p-hpRNA (SEQ ID NO:XX). In some embodiments, the RIG-I agonist has a sequence that is at least 99% identical to the sequence of 3p-hpRNA (SEQ ID NO:XX). Accordingly, in some embodiments, compositions and methods are provided for treating a cancer by administering to the subject a therapeutically effective amount of a composition including a complex formed between (i) a polynucleotide having a sequence that is at least 80%, at least 85%, at least 90%, at least 95%, at least 96%, at least 97%, at least 98%, or at least 99% identical to the nucleotide sequence of 3p-hpRNA (SEQ ID NO:XX) that is capable of stimulating RIG-I, and (ii) a 3E10 antibody or variant thereof, or antigen-binding fragment thereof.

In some embodiments, the polynucleotide RIG-I agonist is a single-stranded polynucleotide of at least 20 nucleotides in length. In some embodiments, the polynucleotide RIG-I agonist is a single-stranded polynucleotide of at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, or more nucleotides in length.

In some embodiments, the polynucleotide RIG-I agonist is poly(dA:dT). In some embodiments, the polynucleotide RIG-I agonist is poly(I:C).

In some embodiments, the polynucleotide RIG-I agonist is a single-stranded polynucleotide of no more than 500 nucleotides in length. In some embodiments, the polynucleotide RIG-I agonist is a single-stranded polynucleotide of no more than 400, no more than 350, no more than 300, no more than 250, no more than 200, no more than 150, no more than 125, no more than 100, no more than 75, no more than 50, or fewer nucleotides in length.

In some embodiments, the polynucleotide RIG-I agonist is a single-stranded polynucleotide of from 20 to 500 nucleotides in length. In some embodiments, the polynucleotide RIG-I agonist is a single-stranded polynucleotide of from 20 to 400 nucleotides in length. In some embodiments, the polynucleotide RIG-I agonist is a single-stranded polynucleotide of from 20 to 300 nucleotides in length. In some embodiments, the polynucleotide RIG-I agonist is a single-stranded polynucleotide of from 20 to 250 nucleotides in length. In some embodiments, the polynucleotide RIG-I agonist is a single-stranded polynucleotide of from 20 to 200 nucleotides in length. In some embodiments, the polynucleotide RIG-I agonist is a single-stranded polynucleotide of from 20 to 150 nucleotides in length. In some embodiments, the polynucleotide RIG-I agonist is a single-stranded polynucleotide of from 20 to 125 nucleotides in length. In some embodiments, the polynucleotide RIG-I agonist is a single-stranded polynucleotide of from 20 to 100 nucleotides in length. In some embodiments, the polynucleotide RIG-I agonist is a single-stranded polynucleotide of from 20 to 75 nucleotides in length. In some embodiments, the polynucleotide RIG-I agonist is a single-stranded polynucleotide of from 20 to 50 nucleotides in length.

In some embodiments, the polynucleotide RIG-I agonist is a single-stranded polynucleotide of from 35 to 500 nucleotides in length. In some embodiments, the polynucleotide RIG-I agonist is a single-stranded polynucleotide of from 35 to 400 nucleotides in length. In some embodiments, the polynucleotide RIG-I agonist is a single-stranded polynucleotide of from 35 to 300 nucleotides in length. In some embodiments, the polynucleotide RIG-I agonist is a single-stranded polynucleotide of from 35 to 250 nucleotides in length. In some embodiments, the polynucleotide RIG-I agonist is a single-stranded polynucleotide of from 35 to 200 nucleotides in length. In some embodiments, the polynucleotide RIG-I agonist is a single-stranded polynucleotide of from 35 to 150 nucleotides in length. In some embodiments, the polynucleotide RIG-I agonist is a single-stranded polynucleotide of from 35 to 125 nucleotides in length. In some embodiments, the polynucleotide RIG-I agonist is a single-stranded polynucleotide of from 35 to 100 nucleotides in length. In some embodiments, the polynucleotide RIG-I agonist is a single-stranded polynucleotide of from 35 to 75 nucleotides in length. In some embodiments, the polynucleotide RIG-I agonist is a single-stranded polynucleotide of from 35 to 50 nucleotides in length.

In some embodiments, the polynucleotide RIG-I agonist is a single-stranded polynucleotide of from 50 to 500 nucleotides in length. In some embodiments, the polynucleotide RIG-I agonist is a single-stranded polynucleotide of from 50 to 400 nucleotides in length. In some embodiments, the polynucleotide RIG-I agonist is a single-stranded polynucleotide of from 50 to 300 nucleotides in length. In some embodiments, the polynucleotide RIG-I agonist is a single-stranded polynucleotide of from 50 to 250 nucleotides in length. In some embodiments, the polynucleotide RIG-I agonist is a single-stranded polynucleotide of from 50 to 200 nucleotides in length. In some embodiments, the polynucleotide RIG-I agonist is a single-stranded polynucleotide of from 50 to 150 nucleotides in length. In some embodiments, the polynucleotide RIG-I agonist is a single-stranded polynucleotide of from 50 to 125 nucleotides in length. In some embodiments, the polynucleotide RIG-I agonist is a single-stranded polynucleotide of from 50 to 100 nucleotides in length. In some embodiments, the polynucleotide RIG-I agonist is a single-stranded polynucleotide of from 50 to 75 nucleotides in length.

In some embodiments, the polynucleotide RIG-I agonist is a single-stranded polynucleotide of from 75 to 500 nucleotides in length. In some embodiments, the polynucleotide RIG-I agonist is a single-stranded polynucleotide of from 75 to 400 nucleotides in length. In some embodiments, the polynucleotide RIG-I agonist is a single-stranded polynucleotide of from 75 to 300 nucleotides in length. In some embodiments, the polynucleotide RIG-I agonist is a single-stranded polynucleotide of from 75 to 250 nucleotides in length. In some embodiments, the polynucleotide RIG-I agonist is a single-stranded polynucleotide of from 75 to 200 nucleotides in length. In some embodiments, the polynucleotide RIG-I agonist is a single-stranded polynucleotide of from 75 to 150 nucleotides in length. In some embodiments, the polynucleotide RIG-I agonist is a single-stranded polynucleotide of from 75 to 125 nucleotides in length. In some embodiments, the polynucleotide RIG-I agonist is a single-stranded polynucleotide of from 75 to 100 nucleotides in length.

In some embodiments, the polynucleotide RIG-I agonist is a single-stranded polynucleotide of from 100 to 500 nucleotides in length. In some embodiments, the polynucleotide RIG-I agonist is a single-stranded polynucleotide of from 100 to 400 nucleotides in length. In some embodiments, the polynucleotide RIG-I agonist is a single-stranded polynucleotide of from 100 to 300 nucleotides in length. In some embodiments, the polynucleotide RIG-I agonist is a single-stranded polynucleotide of from 100 to 250 nucleotides in length. In some embodiments, the polynucleotide RIG-I agonist is a single-stranded polynucleotide of from 100 to 200 nucleotides in length. In some embodiments, the polynucleotide RIG-I agonist is a single-stranded polynucleotide of from 100 to 150 nucleotides in length. In some embodiments, the polynucleotide RIG-I agonist is a single-stranded polynucleotide of from 100 to 125 nucleotides in length.

In some embodiments, the polynucleotide RIG-I agonist is a single-stranded polynucleotide of from 125 to 500 nucleotides in length. In some embodiments, the polynucleotide RIG-I agonist is a single-stranded polynucleotide of from 125 to 400 nucleotides in length. In some embodiments, the polynucleotide RIG-I agonist is a single-stranded polynucleotide of from 125 to 300 nucleotides in length. In some embodiments, the polynucleotide RIG-I agonist is a single-stranded polynucleotide of from 125 to 250 nucleotides in length. In some embodiments, the polynucleotide RIG-I agonist is a single-stranded polynucleotide of from 125 to 200 nucleotides in length. In some embodiments, the polynucleotide RIG-I agonist is a single-stranded polynucleotide of from 125 to 150 nucleotides in length.

In some embodiments, the polynucleotide RIG-I agonist is a single-stranded polynucleotide of from 150 to 500 nucleotides in length. In some embodiments, the polynucleotide RIG-I agonist is a single-stranded polynucleotide of from 150 to 400 nucleotides in length. In some embodiments, the polynucleotide RIG-I agonist is a single-stranded polynucleotide of from 150 to 300 nucleotides in length. In some embodiments, the polynucleotide RIG-I agonist is a single-stranded polynucleotide of from 150 to 250 nucleotides in length. In some embodiments, the polynucleotide RIG-I agonist is a single-stranded polynucleotide of from 150 to 200 nucleotides in length. In some embodiments, the polynucleotide RIG-I agonist is a single-stranded polynucleotide of from 150 to 150 nucleotides in length.

### Polynucleotides Encoding Effector Polypeptides

In some embodiments, a 3E10 antibody or variant thereof, or antigen-binding fragment thereof described herein is used to deliver a polynucleotide that encodes an effector polypeptide to a cancerous tissue. An effector polypeptide is any polypeptide that effects treatment of a cancer, either directly or indirectly, for example by slowing down progression of the cancer, inducing cellular death of cancer cells, inducing senescence of cancer cells, and the like. For example, in some embodiments, an effector polypeptide stimulates immune cells to upregulate the production of cytokines, causing targeting of cancerous cells resulting in cell death. In other embodiments, an effector polypeptide expresses, or stimulates tumor cells to upregulate expression, of tumor antigens which are markers for immune cells to identify tumor cells. For a review of effector polypeptides see, for example, Esensten et al., “CD28 costimulation: from mechanism to therapy,” Immunity Review, 44, (2016) 973; Immunity, 2016, 44, 973-988; Smolle et al., Noncoding RNAs and immune checkpoints, FEBS Journal, 2017, 284, 1952-1966; Chen et al., Anti-PD-1-PD-L1 therapy of human cancer past, present, and future, Journal of Clinical Investigation, 2015, Volume 125, 9, 3384-3391; Rowshanravan et al., CTLA-4 a moving target in immunotherapy, Blood, 2018, Volume 131, 1, 58-67; and Dougall et al., TIGIT and CD96 New checkpoint receptor targets for cancer immunotherapy, Immunological Reviews, 2017, 276, 112-120, the content of each of which is incorporated herein by references, in its entirety, for all purposes.

Accordingly, in one aspect, the present disclosure relates to compositions and methods for treating a cancer in a subject by administering to the subject a therapeutically effective amount of a composition including a complex formed between (i) a therapeutic polynucleotide that encodes an effector polypeptide, and (ii) a 3E10 antibody or variant thereof, or antigen-binding fragment thereof, as described herein.

### Tumor Antigens

In some embodiments, the present disclosure relates to compositions and methods for treating a cancer in a subject by administering to the subject a therapeutically effective amount of a composition including a complex formed between (i) a therapeutic polynucleotide encoding a tumor associated antigen, and (ii) a 3E10 antibody or variant thereof, or antigen-binding fragment thereof, as described herein.

Tumor antigens are peptides that are presented almost exclusively on the cell surface of cancerous cells, thereby distinguishing cancerous cells from non-cancerous cells that do not display the tumor antigen. When cancerous cells die, these tumor antigens are released into the tumor microenvironment and can be recognized by the immune system as foreign peptides, altered self-peptides, or self-peptides. Upon release of a sufficient amount of a tumor antigen, the immune system can generate antitumor immunity by generating an immune response to the tumor antigen. Specifically, the immune system targets and destroys cancerous cells displaying the tumor antigen that was used to generate the immune response.

Several classes of antigens have been exploited experimentally to generate antitumor immunity. Specifically, tumor antigens are exogenously administered, as either a peptide or a nucleic acid encoding the antigen, to a patient with a cancer displaying the tumor antigen on its cell surface. In turn, the immune system is presented with sufficient amounts of the antigen to generate an immune response against the tumor antigen, resulting in antitumor immunity. Examples of classes of tumor antigens include oncoviral protein antigens, neoantigens, and antigens derived from a cancer-germline gene. Often, the tumor antigen, or polynucleotide encoding the tumor antigen, is co-administered with an adjuvant that activated dendritic cells, or with dendritic cells themselves, to promote generation of the antitumor immunity. For review, see, for example, Haen et al., “Towards new horizons Characterization, classification and implications of the tumor antigenic repertoire,” Nature Reviews-Clinical Oncology, 2020, Volume 17, 595-610; Saxena M. et al., Nat. Rev. Cancer 21, 360-378 (2021), the contents of which are incorporated herein by reference, in their entireties, for all purposes.

In some embodiments, the present disclosure relates to compositions and methods for treating a cancer in a subject by administering to the subject a therapeutically effective amount of a composition including a complex formed between (i) a therapeutic polynucleotide encoding an oncoviral protein antigen, and (ii) a 3E10 antibody or variant thereof, or antigen-binding fragment thereof, as described herein. An oncoviral protein antigen is an antigen presented on the cell surface of a cancer that is derived from an oncogenic virus associated with the cancer. For instance, a vast majority of cervical cancers are associated, if not caused by, HPV infection. Accordingly, an antigen derived from HPV that is presented on the cell surface of a cervical cancer cell represents an oncoviral protein antigen. Non-limiting examples of oncoviral protein antigens, and an example of the cancers these antigens have been associated with are presented in Table 1 below.

TABLE 1
Examples of oncoviral proteins and associated cancer types.

| Virus | Associated cancer types | Protein Antigens |
|---|---|---|
| Human | HPV types 16 and 18 are | E6/E7-associated protein |
| papillomaviruses | associated with cancers of cervix, | |
| (HPV) | anus, penis, vulva, vagina, | |
| | and HPV-positive oropharyngeal | |
| | cancers. | |
| Epstein-Barr | Burkitt’s lymphoma, Hodgkin’s | EBV nuclear antigens |
| virus (EBV) | lymphoma, post-transplant | (EBNAs) 1, 2, 3a, 3b, 3c |
| | lymphoproliferative | and LP, BARF1, latent |
| | disease, nasopharyngeal | membrane proteins |
| | carcinoma and a subtype | (LMPs) 1, 2a, and 2b, |
| | of stomach cancer. | BamHI |
| | | A rightward transcripts |
| | | (BARTs), EBV-encoded |
| | | RNAs EBERs). |

Accordingly, in some embodiments, the present disclosure relates to compositions and methods for treating a cancer in a subject by administering to the subject a therapeutically effective amount of a composition including a complex formed between (i) a therapeutic polynucleotide encoding an oncoviral protein antigen derived from a viral protein listed in Table 1, and (ii) a 3E10 antibody or variant thereof, or antigen-binding fragment thereof, as described herein. In some embodiments, the therapeutically effective amount of the composition is co-administered with an adjuvant. In some embodiments, the therapeutically effective amount of the composition is co-administered with dendritic cells.

In some embodiments, the present disclosure relates to compositions and methods for treating a cancer in a subject by administering to the subject a therapeutically effective amount of a composition including a complex formed between (i) a therapeutic polynucleotide encoding a respective oncoviral protein antigen derived from a viral protein listed in Table 1, and (ii) a 3E10 antibody or variant thereof, or antigen-binding fragment thereof, as described herein, where the cancer is a cancer associated with the respective oncoviral protein antigen within Table 1. In some embodiments, the therapeutically effective amount of the composition is co-administered with an adjuvant. In some embodiments, the therapeutically effective amount of the composition is co-administered with dendritic cells.

In some embodiments, the present disclosure relates to compositions and methods for treating a cancer in a subject by administering to the subject a therapeutically effective amount of a composition including a complex formed between (i) a therapeutic polynucleotide encoding a neoantigen, and (ii) a 3E10 antibody or variant thereof, or antigen-binding fragment thereof, as described herein. Neoantigens are peptides, presented on the surface of a cancer cell, having an amino acid sequence that is novel to a cancerous tissue. That is, the neoantigen has an amino acid sequence that is not present in the germline (wildtype) human genome. Neoantigens are created by mutation of the genome during or after development of the cancer and, in this fashion, are specific to an individual patient.

Deep-sequencing technologies can be used to identify mutations present within the exome of an individual tumor to predict neoantigens. This is done by identifying neoantigens that can be recognized by T cells. For example, in some embodiments, tumor material is analyzed for nonsynonymous somatic mutations. RNA sequencing data are used to focus on mutations in expressed genes. Peptide stretches containing any of the identified nonsynonymous mutations are generated in silico and are either left unfiltered, filtered using a predictive algorithm, or used to identify MHC-associated neoantigens in mass spectrometry data generated from the patient's cancerous tissue. Modeling of the effect of mutations on the resulting peptide-MHC complex may be used as an additional filter, to identify particularly promising neoantigens. Resulting epitope sets can also be used to identify physiologically occurring neoantigen-specific T cell responses by MHC multimer-based screens. (See, e.g., Science, 3 Apr. 2015: Vol. 348, Issue 6230, pp. 69-74). However, other techniques, including exomic analysis and proteomic analysis can also be used to identify novel genomic or novel peptide sequences corresponding to a neoantigen, respectively. Non-limiting examples of neoantigens identified from individual cancers, using transcriptomic, exomic, and proteomic analyses are described, for example, in Haen et al., Towards new horizons Characterization, classification and implications of the tumor antigenic repertoire, Nature Reviews-Clinical Oncology, 2020, Volume 17, 595-610.

Thus, in some embodiments, the present disclosure provides a method for treating a cancer in a subject by first identifying a neoantigen of the cancer in the subject, and second administering to the subject a therapeutically effective amount of a composition including a complex formed between (i) a therapeutic polynucleotide encoding the identified neoantigen, and (ii) a 3E10 antibody or variant thereof, or antigen-binding fragment thereof, as described herein.

In some embodiments, the present disclosure relates to compositions and methods for treating a cancer in a subject by administering to the subject a therapeutically effective amount of a composition including a complex formed between (i) a therapeutic polynucleotide encoding a cancer germline antigen, and (ii) a 3E10 antibody or variant thereof, or antigen-binding fragment thereof, as described herein. Cancer germline antigens are a class of immunogenic tumor antigens encoded by genes expressed in gametogenic cells of the testis and/or ovary and in human cancer. Examples of cancer germline antigens include, but are not limited to, antigens derived from synovial sarcoma X-2 (SSX-2), New York-esophageal squamous cell carcinoma-1 (NY-ESO-1), melanoma associated antigen 1 (MAGA1), and melanoma associated antigen 3 (MAGA3), each which are over-expressed in different human cancers such as in melanoma and lung cancer.

Accordingly, in some embodiments, the present disclosure relates to compositions and methods for treating a cancer in a subject by administering to the subject a therapeutically effective amount of a composition including a complex formed between (i) a therapeutic polynucleotide encoding a cancer germline antigen derived from an SSX-2, NY-ESO-1, MAGA1, or MAGA3 protein and (ii) a 3E10 antibody or variant thereof, or antigen-binding fragment thereof, as described herein. In some embodiments, the cancer is melanoma. In some embodiments, the cancer is a lung cancer.

In some embodiments, the present disclosure relates to compositions and methods for treating a cancer in a subject by administering to the subject a therapeutically effective amount of a composition including a complex formed between (i) a therapeutic polynucleotide encoding a tumor-associated antigen (TAA), and (ii) a 3E10 antibody or variant thereof, or antigen-binding fragment thereof, as described herein. In some embodiments, the therapeutically effective amount of the composition is co-administered with an adjuvant. In some embodiments, the therapeutically effective amount of the composition is co-administered with dendritic cells.

Tumor-associated antigens are peptides derived from wild-type protein sequences that are expressed primarily in cancerous tissue. TAAs are primarily generated by genetic amplification or post-translational modifications, that cause the underlying protein to be differentially expressed within cancer cells, relative to non-cancerous cells. Non-limiting examples of tumor associated antigens that have been identified are presented in Table 2.

TABLE 2
Examples of tumor associated antigens and associated cancers.

| Tumor Associated Antigen | Cancers |
|---|---|
| Alphafetoprotein (AFP) | Germ cell tumors |
| | Hepatocellular carcinoma |
| Carcinoembryonic | Bowel cancers |
| antigen (CEA) | |
| CA-125 | Ovarian cancer |
| MUC-1 | Breast cancer |
| Epithelial tumor antigen (ETA) | Breast cancer |
| Tyrosinase | Malignant melanoma |

Accordingly, in some embodiments, the present disclosure relates to compositions and methods for treating a cancer in a subject by administering to the subject a therapeutically effective amount of a composition including a complex formed between (i) a therapeutic polynucleotide encoding a tumor-associated antigen derived from a protein listed in Table 2, and (ii) a 3E10 antibody or variant thereof, or antigen-binding fragment thereof, as described herein. In some embodiments, the therapeutically effective amount of the composition is co-administered with an adjuvant. In some embodiments, the therapeutically effective amount of the composition is co-administered with dendritic cells.

In some embodiments, the present disclosure relates to compositions and methods for treating a cancer in a subject by administering to the subject a therapeutically effective amount of a composition including a complex formed between (i) a therapeutic polynucleotide encoding a respective tumor-associated antigen derived from a protein listed in Table 2, and (ii) a 3E10 antibody or variant thereof, or antigen-binding fragment thereof, as described herein, where the cancer is a cancer associated with the respective tumor-associated antigen within Table 2. In some embodiments, the therapeutically effective amount of the composition is co-administered with an adjuvant. In some embodiments, the therapeutically effective amount of the composition is co-administered with dendritic cells.

CD28 is a member of a subfamily of costimulatory molecules characterized by an extracellular variable immunoglobulin-like domain. Human CD28 is composed of four exons encoding a protein of 220 amino acids that is expressed on the cell surface as a glycosylated, disulfide-linked homodimer of 44 kDa. Members of the CD28 family share a number of common features such as, for example, paired V-set immunoglobulin superfamily (IgSF) domains attached to single transmembrane domains and cytoplasmic domains that contain critical signaling motifs. (Esensten et al., Immunity Review (2016)). CD28 has been reported to regulate T-cell activation via interaction with the signaling motifs. For example, tyrosine phosphorylation of CD28 plays a role in the early signaling events that characterize CD28 costimulation and consequent regulation of T-cell activation. Thus, in some embodiments, the compositions for treating a cancer provided herein include a complex formed between (i) a polynucleotide encoding a signaling motif of a costimulatory molecule having paired V-set immunoglobulin superfamily (IgSF) domains attached to single transmembrane domains and cytoplasmic domains, and (ii) a 3E10 antibody or variant thereof, or antigen-binding fragment thereof.

### Proinflammatory Cytokines

Proinflammatory cytokines limit tumor cell growth by a direct anti-proliferative or pro-apoptotic activity, or indirectly by stimulating the cytotoxic activity of immune cells against tumor cells. Thus, proinflammatory cytokines can improve antigen priming, increase the number of effector immune cells in the tumor microenvironment and/or enhance their cytolytic activity. Examples of proinflammatory cytokine include, but are not limited to, IL-2, IL-15, IL-21, IL-12, IFN-alpha, granulocyte-macrophage colony-stimulating factor (GM-CSF, CSF-2), and TGF-beta.

IL-2 was approved for the treatment of advanced renal cell carcinoma (RCC) and metastatic melanoma, and IFN-alpha was approved for the treatment of hairy cell leukemia, follicular non-Hodgkin lymphoma, melanoma and AIDS-related Kaposi's sarcoma (Berraondo et al., Cytokines in clinical cancer immunotherapy. British Journal of Cancer, 2019, 120, 6-15; Fyfe et al., Results of treatment of 255 patients with metastatic renal cell carcinoma who received high-dose recombinant interleukin-2 therapy. J. Clin. Oncol., 1995, 13, 688-696; Atkins et al., High-dose recombinant interleukin 2 therapy for patients with metastatic melanoma: analysis of 270 patients treated between 1985 and 1993. J. Clin. Oncol., 1999, 17, 2105-2116; Golomb et al., Alpha-2 interferon therapy of hairy-cell leukemia: a multi-center study of 64 patients. J. Clin. Oncol., 1986, 4, 900-905; Solal-Celigny et al., Recombinant interferon alfa-2b combined with a regimen containing doxorubicin in patients with advanced follicular lymphoma. Groupe d'Etude des Lymphomes de l'Adulte. New Engl. J. Med., 1993, 329, 1608-1614; Kirkwood et al., Interferon alfa-2b adjuvant therapy of high-risk resected cutaneous melanoma: the Eastern Cooperative Oncology Group Trial EST 1684. J. Clin. Oncol., 1996, 14, 7-17; Groopman et al., Recombinant alpha-2 interferon therapy for Kaposi's sarcoma associated with the acquired immunodeficiency syndrome. Ann. Intern. Med., 1984, 100, 671-676).

Accordingly, in some embodiments, the composition for treatment of cancer described herein includes a 3E10 antibody or variant thereof, or antigen-binding fragment thereof and a polynucleotide that encodes a proinflammatory cytokine.

In some embodiments, the composition for treatment of cancer described herein includes a complex formed between (i) a polynucleotide encoding an agonist for a proinflammatory cytokine and (ii) a 3E10 antibody or variant thereof, or antigen-binding fragment thereof, as described herein.

### Gene Regulating Polynucleotides

In some embodiments, a 3E10 antibody or variant thereof, or antigen-binding fragment thereof described herein is used to deliver a gene-regulating polynucleotide that reduces or silences expression a gene product that promotes cancer growth and/or progression, e.g., by targeting the gene or a transcript thereof. Non-limiting examples of gene-regulating polynucleotides include siRNA, miRNA, saRNA, antagomirs, antisense oligonucleotides, and decoy oligonucleotides. For a review of the various types of gene-regulating polynucleotides that have been researched for therapeutic capability see, for example, Roberts T C, Langer R, Wood M J A, “Advances in oligonucleotide drug delivery,” Nat. Rev. Drug Discov., 19(10):673-94 (2020), the content of which is incorporated herein by reference, in its entirety, for all purposes.

Accordingly, in one aspect, the present disclosure relates to compositions and methods for treating a cancer in a subject by administering to the subject a therapeutically effective amount of a composition including a complex formed between (i) a gene-regulating polynucleotide, and (ii) a 3E10 antibody or variant thereof, or antigen-binding fragment thereof, as described herein.

siRNAs

In some embodiments, the composition for treatment of cancer described herein includes a complex formed between (i) an siRNA, and (ii) a 3E10 antibody or variant thereof, or antigen-binding fragment thereof, as described herein. Small interfering RNA (siRNA), also known as short interfering RNA or silencing RNA, is a class of double-stranded RNA non-coding RNA molecules, typically 20-27 base pairs in length, and operating within the RNA interference (RNAi) pathway. Gene-regulating nucleic acid drugs such as siRNA can regulate post-transcriptional gene expression, and silence targeted genes, further regulating intracellular signaling pathway involved in cancer progression (Zhou et al., Delivery of nucleic acid therapeutics for cancer immunotherapy, Medicine in Drug Discovery, Mar. 24, 2020; Dahlman et al., In vivo endothelial siRNA delivery using polymeric nanoparticles with low molecular weight, Nature Nanotechnol. 2014; 9(8):648-655).

siRNAs can, thus, be used for modulating the expression of immune checkpoint molecules, such as those described herein, by regulating the post-translational gene expression and/or silencing corresponding genes. Similarly, siRNAs can be used for indirectly regulating the activity of immune checkpoint molecules by modulating the expression of agonists or inhibitors of immune checkpoint molecules. Accordingly, in some embodiments, the composition for treatment of cancer described herein includes a complex formed between (i) an siRNA targeting an mRNA transcript from a gene encoding an immune checkpoint molecule, and (ii) a 3E10 antibody or variant thereof, or antigen-binding fragment thereof, as described herein.

PD-1 and certain homologs thereof such as, for example, PD-L1, suppress T-cell responses, especially in the tumor microenvironment. Thus, inhibitors of PD-1 and/or PD-L1 may improve efficacy of T-cells in attacking and killing tumor cells. Suppression of PD-1 and/or PD-L1 activity can be accomplished, for example, by inhibiting the production of PD-1 and/or PD-L1 within the cells. Accordingly, in some embodiments, the compositions for a cancer provided herein include a complex formed between (i) siRNA targeting an mRNA transcript for PD-1 or PD-L1, and (ii) a 3E10 antibody or variant thereof, or antigen-binding fragment thereof.

Suppression of Cytotoxic T-lymphocyte-associated antigen 4 (CTLA-4) activity is known to result in rapid infiltration of T cells. Thus, inhibitors of CTLA-4 may result in promoting T-cell responses. Accordingly, in some embodiments, the compositions for treating a cancer provided herein include a complex formed between (i) siRNA targeting an mRNA transcript for CTLA-4, and (ii) a 3E10 antibody or variant thereof, or antigen-binding fragment thereof.

siRNAs can similarly be used for silencing genes regulating tumor growth or angiogenesis. For example, siRNAs have been used to target vascular endothelial growth factor (VEGF) and kinesin spindle protein (KSP) (for solid tumors, e.g., liver metastasis from colon cancer). Other genetic targets that can be silenced using siRNAs include, but are not limited to, genes encoding protein kinase N3 (PKN3) (e.g., for metastatic pancreatic cancer), M2 subunit of ribonucleotide reductase (RRM2) (e.g., for solid tumors), Myc oncoprotein (e.g., for hepatocellular carcinoma), ephrin type-A receptor 2 (EphA2) (e.g., for advanced cancers), and KRAS G12D mutation (e.g., for advanced pancreatic cancers). See, e.g., International Journal of Nanomedicine 2019:14 3111-3128. Accordingly, in some embodiments, the compositions for treating a cancer provided herein include a complex formed between (i) siRNA targeting an mRNA transcript for VEGF, KSP, PKN3, RRM2, EphA2, or KRAS, and (ii) a 3E10 antibody or variant thereof, or antigen-binding fragment thereof.

Other examples of siRNA that find use in the methods and compositions described herein include, but are not limited to, siRNA targeting an mRNA transcript from a gene encoding CD25 (IL-2 receptor) to downregulate IL-2 signaling in CD8+ T-cells.

Other non-limiting examples of siRNA and associated cancer types being studied are provided in Table 2 below (See, e.g., Int. J Mol. Sci. 22 (2021) 3295):

TABLE 2
Example siRNA and associated cancer types.

| Drug/Therapeutic | Target | Cancer |
|---|---|---|
| KRAS G12D siRNA | KRASG12D | Pancreatic cancer |
| EphA2-targeting DOPC- | EPHA2 | Solid tumors |
| encapsulated siRNA | | |
| APN401 | CBLB | Brain cancer, melanoma, |
| | | pancreatic cancer, |
| | | renal cell cancer |
| Proteosome siRNA and | LMP2, | Melanoma |
| tumor antigen RNA- | LMP7, | |
| transfected dendritic cells | MECL1 | |
| TKM-080301 | PLK1 | Cancer with hepatic |
| | | metastases, liver cancer, |
| | | hepatocellular cancer, |
| | | adrenocortical cancer |
| Atu027 | PNK3 | Solid tumors, |
| | | pancreatic cancer |
| DCR-MYC | MYC | Solid tumors, |
| | | hepatocellular cancer |
| CALAA-01 | M2 subunit of | Solid tumors |
| | ribonucleotide | |
| | reductase 2 | |
| siG12D LODER | KRASG12D | Pancreatic cancer |
| ARO-HIF2 | HIF2A | Clear cell renal |
| | | cell carcinoma |
| SV40 vectors | Unknown | Chronic myeloid |
| carrying siRNA | | leukemia |

Accordingly, in some embodiments, the present disclosure relates to compositions and methods for treating a cancer in a subject by administering to the subject a therapeutically effective amount of a composition including a complex formed between (i) an siRNA listed in Table 2, and (ii) a 3E10 antibody or variant thereof, or antigen-binding fragment thereof, as described herein.

In some embodiments, the present disclosure relates to compositions and methods for treating a cancer in a subject by administering to the subject a therapeutically effective amount of a composition including a complex formed between (i) a respective siRNA listed in Table 2, and (ii) a 3E10 antibody or variant thereof, or antigen-binding fragment thereof, as described herein, where the cancer is a cancer associated with the respective siRNA in Table 2.

In some embodiments, the present disclosure relates to compositions and methods for treating a cancer in a subject by administering to the subject a therapeutically effective amount of a composition including a complex formed between (i) an siRNA targeting a transcript from a gene listed in Table 2, and (ii) a 3E10 antibody or variant thereof, or antigen-binding fragment thereof, as described herein.

In some embodiments, the present disclosure relates to compositions and methods for treating a cancer in a subject by administering to the subject a therapeutically effective amount of a composition including a complex formed between (i) an siRNA targeting a transcript from a respective gene listed in Table 2, and (ii) a 3E10 antibody or variant thereof, or antigen-binding fragment thereof, as described herein, where the cancer is a cancer associated with the respective gene in Table 2.

miRNAs

In some embodiments, the composition for treatment of cancer described herein includes a complex formed between (i) an miRNA, and (ii) a 3E10 antibody or variant thereof, or antigen-binding fragment thereof, as described herein. MicroRNAs (miRNAs) are a class of non-coding RNAs that play important roles in regulating gene expression. miRNA is endogenous small non-coding RNA of about 18-24 nt in length that can regulate target gene expression by a mechanism similar to siRNA (Zhou et al., Delivery of nucleic acid therapeutics for cancer immunotherapy, Medicine in Drug Discovery, Mar. 24, 2020; Xiao et al., MicroRNA control in the immune system; basic principles, Cell, 2009; 136(1):26-36). One main challenge of miRNA delivery is to deliver them into tumor tissue with deep tissue penetration efficiently. Moreover, the complexation of tumor microenvironment also prevents miRNA from efficient intracellular delivery into target tumor cells (Rupaimoole et al., MiRNA deregulation in cancer cells and the tumor microenvironment. Cancer Discov. 2016; 6(3):235-46).

Advantageously, however, expression of miRNAs is specific to distinct tumors, and miRNAs are involved in early regulation of immune responses. One approach to treating cancer is to modulate the expression of immune checkpoint molecules, such as those described herein, by modulating levels of miRNAs. Examples of miRNAs regulating immune checkpoint-related processes include, but are not limited to, miR-15a, -15b, -16, -195, -424, 497, -503, which regulate the expression of PD-L1 and CD80. Another example of miRNA with tumor-suppressive function is miR-28, which inhibits the expression of TIM3, BTLA, and PD-1 in T-cells by binding to their respective 3′ UTRs. Yet another example of miRNA is miR-138 which inhibits the expression of PD-1 and CTLA-4 on the surface of both effector and regulatory T-cells. miR-34 family which includes miR-34a, -34b and -34c, inhibits expression of PD-L1.

Expression of miR-138-5p is known to impede proliferation of CRC cell, block their transition from G1 to S phase of the cell cycle and directly inhibit PD-L1 expression.

Other miRNAs such as, for example, miR-20b, -21, and 130b, which are overexpressed in certain types of cancer cells may be effective in indirectly mitigating T-cell activation in tumor microenvironment by expression of PTEN.

Accordingly, the composition for treatment of cancer described herein includes a complex formed between (i) an miRNA which directly or indirectly modulates the expression of immune checkpoint molecules, and (ii) a 3E10 antibody or variant thereof, or antigen-binding fragment thereof, as described herein.

In some embodiments, the composition for treatment of cancer described herein includes a complex formed between (i) an miRNA mimicking molecules which directly or indirectly modulates the expression of immune checkpoint molecules, and (ii) a 3E10 antibody or variant thereof, or antigen-binding fragment thereof, as described herein. The miRNAs may be double-stranded synthetic RNAs that mimic endogenous miRNAs because of the same sequence.

In some embodiments, the composition for treatment of cancer described herein includes a complex formed between (i) an miRNA expression vector encoding an miRNA which directly or indirectly modulates the expression of immune checkpoint molecules, and (ii) a 3E10 antibody or variant thereof, or antigen-binding fragment thereof, as described herein.

In some embodiments, the composition for treatment of cancer described herein includes a complex formed between (i) an LNA-modified antisense oligodeoxyribonucleotide (ASO) targeting an miRNA which directly or indirectly modulates the expression of immune checkpoint molecules, and (ii) a 3E10 antibody or variant thereof, or antigen-binding fragment thereof, as described herein. An LNA is a bicyclic RNA analog with the ribose locked in C3′-endo conformation by the introduction of a 2′-O, 4′-C methylene bridge.

In some embodiments, the composition for treatment of cancer described herein includes a complex formed between (i) an antagomir targeting an miRNA which directly or indirectly modulates the expression of immune checkpoint molecules, and (ii) a 3E10 antibody or variant thereof, or antigen-binding fragment thereof, as described herein. An antagomir may be a single-stranded 23-nucleotide RNA molecule complementary to the targeted miRNA that has been modified with a partial phosphorothioate backbone in addition to 2′-O-methoxyethyl. This is known to increase the stability of miRNA by protecting it from degradation.

In some embodiments, the composition for treatment of cancer described herein includes a complex formed between (i) an antisense oligodeoxyribonucleotide (ASO) targeting an miRNA which directly or indirectly modulates the expression of immune checkpoint molecules, and (ii) a 3E10 antibody or variant thereof, or antigen-binding fragment thereof, as described herein.

In some embodiments, composition for treatment of cancer described herein includes a complex formed between (i) an miRNA sponge which directly or indirectly modulates the expression of immune checkpoint molecules, and (ii) a 3E10 antibody or variant thereof, or antigen-binding fragment thereof, as described herein. An miRNA sponge may be an RNA containing multiple tandem binding sites for the miRNA of interest transcribed from expression vectors.

Similarly, miRNA modulating the expression of proteins that are associated with tumor growth or angiogenesis may also be delivered by complexing with 3E10 antibody or variant thereof, or antigen-binding fragment thereof, as described herein. Non-limiting Examples of miRNA and cancer they are associated with are given in Table 3.

TABLE 3
Example miRNAs being studied for treatment of cancers (see, e.g.,
Journal of the International Federation of Clinical Chemistry and
Laboratory Medicine (2019) Vol. 30, No. 2, pp. 114-127).

| miRNA | Cancer |
|---|---|
| miR-122 | HCV |
| miR-155 | Lymphoma and leukemia |
| miR-16 | Mesothelioma |
| miR-34 | Renal cell carcinoma, acral melanoma, |
| | hepatocellular carcinoma |
| MRX34 | Liver cancer, lung cancer, lymphoma, |
| | melanoma, multiple myeloma, |
| | renal cell cancer |
| INT-1B3 | Solid tumor |
| TargomiRs | Malignant pleural mesothelioma, |
| | non-small cell lung cancer |
| Cobomarsen | Cutaneous T-cell lymphoma, |
| (MRG-106) | colorectal cancer |

Accordingly, in some embodiments, the present disclosure relates to compositions and methods for treating a cancer in a subject by administering to the subject a therapeutically effective amount of a composition including a complex formed between (i) an miRNA listed in Table 3, and (ii) a 3E10 antibody or variant thereof, or antigen-binding fragment thereof, as described herein.

In some embodiments, the present disclosure relates to compositions and methods for treating a cancer in a subject by administering to the subject a therapeutically effective amount of a composition including a complex formed between (i) a respective miRNA listed in Table 3, and (ii) a 3E10 antibody or variant thereof, or antigen-binding fragment thereof, as described herein, where the cancer is a cancer associated with the respective miRNA in Table 3.

saRNAs

In some embodiments, the composition for treatment of cancer described herein includes a complex formed between (i) an saRNA, and (ii) a 3E10 antibody or variant thereof, or antigen-binding fragment thereof, as described herein. Small-activating RNA (saRNA) is a class of noncoding dsRNA about 21 nt in length with 2 nt overhangs at both end (Zhou et al., Delivery of nucleic acid therapeutics for cancer immunotherapy, Medicine in Drug Discovery, Mar. 24, 2020; Kwok et al., Developing small activating RNA as a therapeutic: current challenges and promises. Ther. Deliv. 2019; 10(3):151-64). Although it shares similar structure with siRNA, it has the opposite mechanism of gene regulation. saRNA in the cytoplasm is specifically loaded to an AGO2 protein and this RNA-AGO2 complex is transported to the nucleus to induce targeted gene promoters for gene activation (Li et al., Small DsRNAs induce transcriptional activation in human cells. Proc. Natl. Acad. Sci. U.S.A 2006; 103(46): 17337-42). It has been reported that saRNA-AGO2 complex in the nucleus recruits essential protein for transcription initiation such as RNA helicase A, RNA polymerase-associated protein CTR9 homolog (CTR9) and RNA polymerase II-associated factor 1 homolog (PAF1) (Portnoy et al., SaRNA-guided Ago2 targets the RITA complex to promoters to stimulate transcription. Cell Res. 2016; 26(3): 320-35). Due to its ability of gene upregulation, saRNA shows the potential for applications such as cancer immunotherapy. Accordingly, in some embodiments, the composition for treatment of cancer described herein includes a complex formed between (i) an saRNA inducing activation of a gene encoding an immune checkpoint molecules, and (ii) a 3E10 antibody or variant thereof, or antigen-binding fragment thereof, as described herein.

In one example, an saRNA can upregulate the transcription factor CCATT/enhancer binding protein alpha (CEBPA) which leads to an increase in functional C/EBP protein and albumin and inhibits growth of liver cancer in a rat model. Other non-limiting examples of saRNAs being studied for treating cancer are listed in Table 4.

TABLE 4
saRNAs being studied for treatment of cancers

| saRNA | Cancer |
|---|---|
| C/EBPa-saRNA | Hepatocellular carcinoma, |
| | pancreatic ductal adenocarcinoma |
| dsP21-322 | Bladder tumor |

### Antagomirs

In some embodiments, the composition for treatment of cancer described herein includes a complex formed between (i) an antagomir, and (ii) a 3E10 antibody or variant thereof, or antigen-binding fragment thereof, as described herein. An antagomir is a small synthetic RNA that is complementary to the specific miRNA target with either mispairing at the cleavage site of Ago2 or some sort of base modification to inhibit Ago2 cleavage. Antagomirs are sequestered specific endogenous microRNA in competition with cellular target mRNAs, inducing miRNA repression and preventing mRNA target degradation via RISC. Thus, antagomirs can be used in treatments where miRNA loss of function is advantageous.

An example of an antagomir is anti-miR21. Studies have shown that silencing of miR21 through use of anti-miR21 affected viability, apoptosis and the cell cycle in colon cancer cells (Song et al., “The anti-miR21 antagomir, a therapeutic tool for colorectal cancer, has a potential synergistic effect by perturbing an angiogenesis-associated miR30,” Front. Genet., January 2014).

Another example of an antagomir is antagomir-221. Studies have shown that antagomir-221 was able to reduce cellular proliferation by suppressing the function of miR-221 which plays an important role in HCC as it inhibits tumor-suppressive target proteins such as P27KIP1, P57KIP2, and phosphatase and tensin homolog (PTEN). Likewise, several studies have shown that antagomir-21 reversed epitheliam-mesenchymal transition (EMT) through inactivation of AKT serine/threonine kinase 1 (AKT) and ERK1/2 pathways by targeting PTEN. This action of antagomir-21 can potentially be used to target the causal mechanism of the malignant propensity of breast cancers. See, e.g., Atri, et al., AGO-Driven Non-Coding RNAs (2019).

AntagomiR that targets miR-155, is in phase 1 (NCT02580552) and phase 2 clinical trials (NCT03713320). miR-155 regulates differentiation and proliferation of blood and lymphoid cells and is a suitable target for treating certain kinds of lymphoma and leukemia. See, e.g., “RNA-Based Therapeutics: From Antisense Oligonucleotides to miRNAs,” 2020 Jan 7; 9(1):137.

Accordingly, in some embodiments, the composition for treatment of cancer described herein includes a complex formed between (i) an antagomir targeting microRNA modulating translation of a tumor-associated mRNA, and (ii) a 3E10 antibody or variant thereof, or antigen-binding fragment thereof, as described herein. Non-limiting examples of antagomirs include antagomir-221, antagomir-21 and antagomir-155.

### Antisense Oligonucleotides (ASOs)

In some embodiments, the composition for treatment of cancer described herein includes a complex formed between (i) an antisense oligonucleotide, and (ii) a 3E10 antibody or variant thereof, or antigen-binding fragment thereof, as described herein. Antisense oligonucleotides (ASOs) are short, synthetic, chemically modified chains of nucleotides that have the potential to target any gene product of interest. Typically, an ASO is a single-stranded sequence complementary to the sequence of the target gene's transcribed messenger RNA (mRNA) within a cell (Rinaldi et al., “Antisense oligonucleotides: the next frontier for treatment of neurological disorders,” Nat. Rev. Neurol. 2018; 14(1):9-21; Bennett, Therapeutic Antisense Oligonucleotides Are Coming of Age. Ann. Rev. Med. 2019; 70:307-321). An ASO targets the corresponding mRNA to degrade the targeted complex by mechanisms such as endogenous cellular RNase H.

One example of an ASO being used for cancer therapy is an ASO targeting CD39 mRNA so as to improve CD8+ T cell proliferation, thereby improving antitumor immune responses. Zhou, et al. Delivery of nucleic acid therapeutics for cancer immunotherapy. Medicine in Drug Discovery, 6(2020) 100023.

Other non-limiting Examples of ASO and cancer they are associated with are given in Table 5.

TABLE 5
Example ASOs being studied for treatment of cancers (see, e.g., Int. J. Mol.
Sci 22 (2021) 3295):

| Drug/Therapeutic | Target | Cancer |
|---|---|---|
| AEG35156 | AEG35156 | Hepatocellular Cancer, Pancreatic Cancer, |
| | | Breast Cancer, Non-Small Cell Lung Cancer, |
| | | Leukemia, Lymphoma |
| Apatorsen | HSP27 | Urologic Cancer, Bladder Cancer, Prostate |
| (OGX-427) | | Cancer, Urothelial Cancer, Non-Small Cell |
| | | Lung Cancer |
| ARRx (AZD5312) | AR | Prostate cancer |
| AZD4785 | KRAS | Non-Small Cell Lung Cancer |
| AZD8701 | FOXP3 | Advanced cancer |
| AZD9150 | STAT3 | Bladder cancer, lymphoma, malignancies |
| BP1001 | GRB2 | Ph1 Positive Leukemia, Acute Myeloid |
| | | Leukemia, Chronic Myelogenous Leukemia |
| Cenersen (EL625) | TP53 | Acute Myleogenous Leukemia, lymphoma |
| CpG 7909 | TLR9 | Melanoma, Breast Cancer, Renal Cancer, |
| (PF03512676) | | Lymphoma, Non-Small Cell Lung Cancer, |
| | | Esophageal Cancer, Prostate Cancer |
| CpG ODN | TLR9 | Leukemia |
| (GNKG168) | | |
| CpG | TLR9 | Breast cancer |
| Oligonucleotide | | |
| CpG-ODN | TLR9 | Glioblastoma |
| Custirsen | ApoJ | Prostate cancer, breast cancer, non-small |
| (OGX-011) | | cell lung cancer |
| Danvatirsen | STAT3 | Advanced cancers |
| (AZD9150, | | |
| ISIS STAT3Rx) | | |
| EGFR Antisense | EGFR | Head and Neck Squamous Cell Cancer, |
| DNA | | Gastric Cancer, Ovarian Cancer, Prostate |
| | | Cancer |
| EZN-2968 | HIF1A | Hepatocellular cancer, lymphoma |
| (RO7070179, | | |
| SPC2968) | | |
| G4460 | CMYB | Leukemia, hematologic malignancies |
| IGF-1R/AS ODN | IGF1 | Glioma |
| IGV-001 containing | IGF1R | Glioblastoma |
| autologous GBM | | |
| cells treated with | | |
| antisense | | |
| oligonucleotide | | |
| (IMV-001) | | |
| IMO-2055 (EMD | TLR9 | Renal cell cancer, colorectal cancer, |
| 1201081) | | non-small cell lunch cancer, head and |
| | | neck cancer |
| ION251 | IRF4 | Myeloma |
| ION537 | YAP1 | Advanced solid tumors |
| ISIS 183750 | EIF4E | Castrate-resistant prostate cancer, non- |
| (ISIS-EIF4ERx, | | small cell lung cancer, colorectal cancer |
| LY2275796) | | |
| ISIS 2503 | HRAS | Colorectal cancer, pancreatic cancer |
| ISIS 5132 | CRAF | Ovarian cancer |
| L-Bcl-2 antisense | BCL2 | Advanced lymphoid malignancies |
| LErafAON | CRAF | Cancers |
| Lucanix | TGFB2 | Non-small cell lung cancer |
| LY2181308 | BIRC5 | Non-small cell lung cancer |
| LY900003 | PKCA | Melanoma, lung cancer, non-small cell |
| (ISIS 3521, | | lung cancer, breast cancer |
| Affinitak) | | |
| MTL-CEBPA | CEBPA | Hepatocellular cancer |
| Oblimersen, | Blocks | Antisense oligodeoxyribonucleotide; |
| Genasense ® | production | several types of cancer, including chronic |
| (Oblimersen | of the Bcl-2 | lymphocytic leukemia, B-cell lymphoma, |
| Sodium) (G3139) | protein | and breast cancer and as described in |
| | | U.S. Pat. Nos. 7,314,927 and 7,795,232, |
| | | each of which are hereby incorporated by |
| | | reference in their entireties. |
| OGX-427 | HSP27 | Cancers |
| PNT2258 | BCL2 | Prostate cancer, lymphoma, melanoma |
| SPC2996 | BCL2 | Chronic lymphocytic leukemia |
| TGF 2 Antisense- | TGFB2 | Advanced cancers |
| GMCSF Gene | | |
| Modified Autologous | | |
| Tumor Cell (TAG) | | |
| Vaccine | | |
| SD-101 | TLR9 | Cancers |
| Trabedersen | TGFB2 | Glioblastoma, anaplastic astrocytoma, |
| (AP 12009, OT-101) | | pancreatic cancer, melanoma, colorectal |
| | | cancer |
| VEGF-Antisense | VEGF | Mesothelioma |
| Oligonucleotide | | |

Accordingly, in some embodiments, the present disclosure relates to compositions and methods for treating a cancer in a subject by administering to the subject a therapeutically effective amount of a composition including a complex formed between (i) an antisense oligonucleotide listed in Table 5, and (ii) a 3E10 antibody or variant thereof, or antigen-binding fragment thereof, as described herein.

In some embodiments, the present disclosure relates to compositions and methods for treating a cancer in a subject by administering to the subject a therapeutically effective amount of a composition including a complex formed between (i) a respective an antisense oligonucleotide listed in Table 5, and (ii) a 3E10 antibody or variant thereof, or antigen-binding fragment thereof, as described herein, where the cancer is a cancer associated with the respective an antisense oligonucleotide in Table 5.

In some embodiments, the present disclosure relates to compositions and methods for treating a cancer in a subject by administering to the subject a therapeutically effective amount of a composition including a complex formed between (i) an antisense oligonucleotide targeting a transcript from a gene listed in Table 5, and (ii) a 3E10 antibody or variant thereof, or antigen-binding fragment thereof, as described herein.

In some embodiments, the present disclosure relates to compositions and methods for treating a cancer in a subject by administering to the subject a therapeutically effective amount of a composition including a complex formed between (i) an antisense oligonucleotide targeting a transcript from a respective gene listed in Table 5, and (ii) a 3E10 antibody or variant thereof, or antigen-binding fragment thereof, as described herein, where the cancer is a cancer associated with the respective gene in Table 5.

### Decoy Oligonucleotides

In some embodiments, a 3E10 antibody or variant thereof, or antigen-binding fragment thereof described herein includes a polynucleotide that encodes a decoy-oligonucleotide. Transfection of cis-element double-stranded oligonucleotides, referred to as decoy oligodeoxynucleotides, has been reported to be a powerful tool that provides a new class of antigene strategies for gene therapy (Crinelli et al., Design and characterization of decoy oligonucleotides containing locked nucleic acids. Nucleic Acid Res. 2002; 30(11): 2435-2443.) One such example is STAT3 decoy oligonucleotide, which is a double-stranded 15-mer oligonucleotide, corresponding closely to the signal transducer and activator of transcription 3 (STAT3) response element within the c-fos promoter, with potential antineoplastic activity. STAT3 decoy oligonucleotide binds specifically to activated STAT3 and blocks binding of STAT3 to DNA sequences on a variety of STAT3-responsive promoters, which results in the inhibition of STAT3-mediated transcription and, potentially, the inhibition of tumor cell proliferation. STAT3 is constitutively activated in a variety of cancers including squamous cell carcinoma of the head and neck, contributing to the loss of cell growth control and neoplastic transformation.

### Genome Editing Polynucleotides

Examples of polynucleotides that encode complexes that can perform genome editing are described in the following sections.

### Zinc Finger Nucleases

In some embodiments, a 3E10 antibody or variant thereof, or antigen-binding fragment thereof described herein includes a polynucleotide that encodes a zinc-finger nuclease. Zinc-finger nucleases are genome editing nucleases. They are artificial restriction enzymes generated by fusing a zinc finger DNA-binding domain to a DNA-cleavage domain. The binding specificity of the designed zinc-finger domain directs the zinc-finger nuclease to a specific genomic site. Compared with other programmable nucleases, the use of zinc-finger nucleases is often limited by poor targeting density and relatively high levels of off-target effects, leading to cytotoxicity (Song et al., The Use of CRISPR/Cas9, ZFNs, and TALENs in Generating Site-Specific Genome Alterations, 2014, Methods in Enzymology). However, zinc-finger nucleases are also the smallest type of programmable nuclease, making it possible to express them using delivery vectors such as adeno-associated viral (AAV) vector.

### Transcription Activator-like Effector Nucleases (TALEN)

In some embodiments, a 3E10 antibody or variant thereof, or antigen-binding fragment thereof described herein includes a polynucleotide that encodes a Transcription Activator-like Effector Nucleases (TALEN). TALEN is a genome editing nuclease (Zhou et al., Delivery of nucleic acid therapeutics for cancer immunotherapy, Medicine in Drug Discovery, Mar. 24, 2020). TALEN are restriction enzymes that can be engineered to cut specific sequences of DNA. They are engineered by fusing a TAL effector DNA-binding domain to a DNA cleavage domain.

### CRISPR/Cas

In some embodiments, a 3E10 antibody or variant thereof, or antigen-binding fragment thereof described herein includes a polynucleotide that encodes CRISPER/Cas. Clustered regularly interspaced short palindromic repeats/CRISPR associated protein (CRISPR/Cas) system. CRISPR/Cas is a genome editing nuclease (Zhou et al., Delivery of nucleic acid therapeutics for cancer immunotherapy, Medicine in Drug Discovery, Mar. 24, 2020). The CRISPR/Cas9 system is currently one of the most comprehensively studied tools because of its simple utilities, programmability, cost effectiveness, and most importantly, the highly efficient multiplex genome engineering capability (Yin et al., CRISPR-Cas: a tool for cancer research and therapeutics. Nat Rev. Clin. Oncol. 2019; 16(5):281-95). For instance, CRISPR/Cas9 system contains two critical components, Cas9 nuclease, and a gRNA, the latter of which is a fusion of a crRNA and a constant tracrRNA (Li et al, Non-viral delivery systems for CRISPR/Cas9 based genome editing: challenges and opportunities. Biomaterials. 2018; 171:207-18).

### Aptamer

In some embodiments, a 3E10 antibody or variant thereof, or antigen-binding fragment thereof described herein includes a polynucleotide that encodes an aptamer. Nucleic acid aptamers are single-stranded (ss) oligonucleotide (DNA or RNA) molecules that fold into distinct secondary or tertiary structures, giving them high affinity and specific binding abilities toward their corresponding targets (Zhu et al., Nucleic Acid Aptamer-Mediated Drug Delivery for Targeted Cancer Therapy, ChemMedChem 2015, 10, 39-45). Aptamers are selected from a random library of 10¹³-10¹⁶ ssDNA or ssRNA molecules through an in vitro technology known as SELEX (systematic evolution of ligands by exponential enrichment) (Ellington et al., Nature. 1990, 346, 818-822; Tuerk et al., Science. 1990, 249, 505-510).

After an aptamer sequence is identified by SELEX, modified nucleotides may be incorporated into the sequence, e.g., to promote stability and/or resistance to nuclease degradation and/or to increase the efficiency of the aptamer. For instance, aptamer APTA-12 includes a gemcitabine residue, which is a 2′, 2′-difluoro analogue of 2′deoxycytidine. See, for example, Park J Y et al., Mol. Ther. Nucleic Acids 2018, 12, 543-553.

Generally, aptamers can be used in cancer therapy to either directly inhibit the activity of a target molecule (where the aptamer is acting as the functional therapeutic molecule), or to target a therapeutic molecule, e.g., a chemotherapeutic or other anti-cancer agent, to a cancerous tissue. In some embodiments, the aptamers used in the methods and compositions described herein directly inhibit the activity of a target molecule, rather than target a cancerous tissue. This is because the 3E10 molecule complexed with the aptamer already targets various cancerous tissues, as described herein. Commonly, therapeutic aptamers used for cancer therapy act as antagonists of oncoproteins or their ligands by binding to one of them, thereby blocking protein-protein or receptor-ligand interactions that promote cancer development and/or progression. For review of the use of aptamers for treatment of cancer see, for example. Han et al., Application and development of aptamer in cancer—from clinical diagnosis to cancer therapy, Journal of Cancer, 2020, 11, 6902-6915; Zhu et al., Nucleic Acid Aptamer-Mediated Drug Delivery for Targeted Cancer Therapy, ChemMedChem 2015, 10, 39-45; and Subjakova et al., Polymer Nanoparticles and Nanomotors Modified by DNA RNA Aptamers and Antibodies in Targeted Therapy of Cancer, Polymers, 2021, 13, 341; Morita Y et al., Cancers (Basel), 2018; 10(3):80, the disclosures of which are incorporated herein by reference.

In some embodiments, the present disclosure relates to compositions and methods for treating a cancer in a subject by administering to the subject a therapeutically effective amount of a composition including a complex formed between (i) an aptamer, and (ii) a 3E10 antibody or variant thereof, or antigen-binding fragment thereof, as described herein. Non-limiting examples of aptamers that have been studies for the treatment of cancer are presented in Table 6 below.

TABLE 6
Example aptamers studied for the treatment of cancer.

| Aptamer | Molecular Target | Cancer Type | Aptamer Sequence |
|---|---|---|---|
| Sgc8c | Protein tyrosine | Leukemia; | ATC TAA CTG CTG |
| | kinase 7 (PTK-7) | acute lymphoblastic | CGC CGC CGG GAA |
| | | leukemia | AAT ACT GTA CGG |
| | | | TTA GA (SEQ ID |
| | | | NO: XX) |
| AS1411 | Nucleolin; | Leukemia; | GGT GGT GGT GGT |
| | MCF7 cells | acute lymphoblastic | TGT GGT GGT GGT GG |
| | | leukemia; | (SEQ ID NO: XX) |
| | | breast cancer | |
| NOX-A12 | CXCL12, | Leukemia; | GCG UGG UGU GAU |
| | MS-5 cells | Chronic lymphocytic | CUA GAU GUA UUG |
| | | leukemia | GCU GAU CCU AGU |
| | | | CAG GUA CGC (SEQ |
| | | | ID NO: XX) |
| KHIC12 | HL60 cells | Leukemia; | ATC CAG AGT GAC |
| | | Acute myeloid | GCA GCA TGC CCT |
| | | leukemia | AGT TAC TAC TAC |
| | | | TCT TTT TAG CAA |
| | | | ACG CCC TCG CTT |
| | | | TGG ACA CGG TGG |
| | | | CTT AGT (SEQ ID |
| | | | NO: XX) |
| K19 | Sigles-5, | Leukemia; | AAG GGG TTG GGT |
| | NB4 cells | Acute myeloid | GGG TTT ATA CAA |
| | | leukemia | ATT AAT TAA TAT |
| | | | TGT ATG GTA TAT TT |
| | | | (SEQ ID NO: XX) |
| TD05 | IgM, | Leukemia; | ACC GGG AGG ATA |
| | Ramos cells | Burkitt's lymphoma | GTT CGG TGG CTG |
| | | | TTC AGG GTC TCC |
| | | | TCC CGG TG (SEQ ID |
| | | | NO: XX) |
| HB5 | HER2 | Breast cancer | AAC CGC CCA AAT |
| | | | CCC TAA GAG TCT |
| | | | GCA CTT GTC ATT |
| | | | TTG TAT ATG TAT |
| | | | TTG GTT TTT GGC |
| | | | TCT CAC AGA CAC |
| | | | ACT ACA CAC GCA |
| | | | CAT G (SEQ ID NO: XX) |
| HeA2_3 | HER2 | Breast cancer | TCT AAA AGG ATT |
| | | | CTT CCC AAG GGG |
| | | | ATC CAA TTC AAA |
| | | | CAG C (SEQ ID NO: XX) |
| H2 | Her2 | Breast cancer | GGG CCG TCG AAC |
| | | | ACG AGC ATG GTG |
| | | | CGT GGA CCT AGG |
| | | | ATG ACC TGA GTA |
| | | | CTG TCC (SEQ ID |
| | | | NO: XX) |
| S6 | SK-BR-3 cells | Breast cancer | TGG ATG GGG AGA |
| | | | TCC GTT GAG TAA |
| | | | GCG GGC GTG TCT |
| | | | CTC TGC CGC CTT |
| | | | GCT ATG GGG (SEQ ID |
| | | | NO: XX) |
| SYL3C | EpCAM | Breast cancer | CAC TAC AGA GGT |
| | | | TGC GTC TGT CCC |
| | | | ACG TTG TCA TGG |
| | | | GGG GTT GGC CTG |
| | | | (SEQ ID NO: XX) |
| APTA-12 | Nucleolin | Pancreatic cancer | GGT GGT GGT GGT |
| | | | TZ*T GGT GGT GGT |
| | | | GG (SEQ ID NO: XX) |
| | | | Z* = gemcitabine |
| M17 | MMP14 proteinase, | Pancreatic cancer | AGG GCC CGA CGT |
| | MIA PaCa-2 and | | GAC GGC ACG TCG |
| | PANC-1 cell lines | | GAT ATC TCA TGC |
| | | | GTG T (SEQ ID NO: XX) |
| S-1 | DHX9, RNA helicase | Colorectal cancer | GCC CAG CAT GCA |
| | | | TTA CTG ATC GTG |
| | | | GTG TTT GCT TAG |
| | | | CCC A (SEQ ID NO: XX) |
| SL2B | VEGF | Colorectal cancer | TTT TTT TTT ACA TTC |
| | | | CTA AGT CTG AAA |
| | | | CAT TAC AGC TTG |
| | | | CTA CAC GAG AAG |
| | | | AGC CGC CAT AGT A |
| | | | (SEQ ID NO: XX) |
| CAA01 | CEA | Colorectal cancer | GGG UCG UGU CGG |
| | | | AUC CAG GCA CGA |
| | | | CGC AUA GCC UUG |
| | | | GGA GCG AGG AAA |
| | | | GCU UCU AAG GUA |
| | | | ACG AU (SEQ ID |
| | | | NO: XX) |
| CA50 A02 | CA50 | Colorectal cancer | GGG UCG UGU CGG |
| | | | AUC CAG CUC GAA |
| | | | AGU GGG CUG GCG |
| | | | AUG UGU CCC GAA |
| | | | GCU UCU AAG GUA |
| | | | ACG AU (SEQ ID |
| | | | NO: XX) |
| CA72-4 A01 | CA72-4 | Colorectal cancer | GGG UCG UGU CGG |
| | | | AUC CUG CGA AGG |
| | | | GGG GCA GAG GUU |
| | | | UGA CGC GAG AAA |
| | | | GCU UCU AAG GUA |
| | | | ACG AU (SEQ ID |
| | | | NO: XX) |
| APT-43 | Lung cancer marker | Lung cancer | CTA TAG CAA TGG |
| | | | TAC GGT ACT TCC |
| | | | TCT CAG GTG GGT |
| | | | GTA TGT GGG CTC |
| | | | CCT TTA CTG ATT |
| | | | GGG TCA AAA GTG |
| | | | CAC GCT ACT TTG |
| | | | CTA A (SEQ ID NO: XX) |
| N/A | A549 cell line | Lung cancer | GGT TGC ATG CCG |
| | | | TGG GGA GGG GGG |
| | | | TGG GTT TTA TAG |
| | | | CGT ACT CAG (SEQ ID |
| | | | NO: XX) |
| TA6 | CD44, | Lung cancer | TTG GGA CGG TGT |
| | SKOV3, IGROV, and | | TAA ACGA AAG GGG |
| | A2780 cell lines | | ACG AC (SEQ ID |
| | | | NO: XX) |
| CA125.1 | CA125 | Lung cancer | AAA AUG CAU GGA |
| | | | GCG AAG GUG UGG |
| | | | GGG AUA CCA ACC |
| | | | GCG CCG UG (SEQ ID |
| | | | NO: XX) |
| Apt928 | CD70 | Lung cancer | GCT GTG TGA CTC |
| | | | CTG CAA GCG GGA |
| | | | AGA GGG CAG GGG |
| | | | AGG GAG GGT GAC |
| | | | GCG GAA GAG GCA |
| | | | AGC AGC TGT ATC |
| | | | TTG TCT CC (SEQ ID |
| | | | NO: XX) |
| R13 | A2780, | Lung cancer | CTC TAG TTA TTG |
| | SKOV3 cells | | AGT TTT CTT TTA |
| | | | TGG GTG GGT GGG |
| | | | GGG TTT TT (SEQ ID |
| | | | NO: XX) |
| HF3-58 | A2780T cells | Lung cancer | TTG GAG CAG CGT |
| | | | GGA GGA TAT GCT |
| | | | TTC CGA CCG TGT |
| | | | TCG TTT GTT ATA |
| | | | ACG CTG CTC C (SEQ |
| | | | ID NO: XX) |
| HA5-68 | A2780T cells | Lung cancer | TTA AGG AGC AGC |
| | | | GTG GAG GAT ATC |
| | | | GGT GTT TAT GGT |
| | | | GTC TGT CTT CCT |
| | | | CCA GTT TCC TTC |
| | | | TGC GCC TT (SEQ ID |
| | | | NO: XX) |
| ARC126 (RNA) | PDGF-B | | Akiyama, Kachi et al., |
| | | | 2006 |
| AX102 (RNA) | PDGF-B | | Sennino, Falcon et al., |
| | | | 2007 |
| SL (2)-B (DNA) | VEGF-165 | | Kaur, Li et al., 2013 |
| RNV66 (DNA) | VEGF-165 | | Gantenbein, Sarikaya et |
| | | | al., 2015 |
| FCL-II (DNA, | Nucleolin | | Fan, Sun et al., 2017 |
| modified form | | | |
| AS1411) | | | |
| NOX-A12 (RNA) | CXCL12 | | Vater, Sahlmann et al., |
| | | | 2013; Liu, Alomran et al., |
| | | | 2014; Hoellenriegel, |
| | | | Zboralski et al., 2014; |
| | | | Zboralski, Hoehlig et al., |
| | | | 2017 |
| E0727 (RNA) | EGFR | | Li, Nguyen et al., 2011 |
| CL428 (RNA) | EGFR | | [8], Esposito, Passaro et |
| KDI130 (RNA) | EGFR | | al., 2011 [7], Wan, |
| TuTu2231 (RNA) | EGFR | | Tamuly et al., 2013; |
| | | | Buerger, Nagel-Wolfrum |
| | | | et al., 2003; Wang, Song |
| | | | et al., 2014 |
| Trimeric apt (DNA) | HER2 | | Kim and Jeong 2011; |
| | | | Mahlknecht, Maron et al., |
| | | | 2013 |
| PNDA-3 (DNA) | Periostin | | Lee, Kim et al., 2013 |
| TTA140,41 (DNA) | TN-C | | Hicke, Stephens et al., |
| | | | 2006; Daniels, Chen et |
| | | | al., 2003; Hicke, Marion |
| | | | et al., 2001 |
| GBI-1042 (DNA) | TN-C | | Hicke, Stephens et al., |
| | | | 2006; Daniels, Chen et |
| | | | al., 2003; Hicke, Marion |
| | | | et al., 2001 |
| NAS-24 (DNA) | Vimentin | | Zamay, Kolovskaya et al., |
| | | | 2014 |
| YJ-1 (RNA) | CEA | | Lee, Han et al., 2012 |
| AGE-apt (DNA) | AGE | | Ojima, Matsui et al., 2014 |
| A-P50 (RNA) | NF-KB | | Mi, Zhang et al., 2008 |
| GL21.T (RNA) | Axl | | Cerchia, Esposito et al., |
| | | | 2012 |
| OPN-R3 (RNA) | OPN | | Mi, Guo et al., 2009; |
| | | | Talbot, Mi et al., 2011 |
| AGC03 (DNA) | HGC-27 | | Zhang, Zhang et al., 2014; |
| | | | Cao, Yuan et al., 2014 |
| cy-apt (DNA) | HGC-27 | | Zhang, Zhang et al., 2014; |
| | | | Cao, Yuan et al., 2014 |
| BC15 (DNA) | hnRNP A1 | | Li, Wang et al., 2012 |
| A9g (RNA) | PSMA | | Dassie, Hernandez et al., |
| | | | 2014 |
| ESTA (DNA) | E-selectin | | Mann, Somasunderam et |
| | | | al., 2010; Kang, Hasan et |
| | | | al., 2015; Kang, Blache et |
| | | | al., 2016; Morita, Kamal |
| | | | et al., 2016 |
| M12-23 (RNA) | 4-1 BB | | McNamara, Kolonias et |
| | | | al., 2008 |
| OX40-apt (RNA) | OX40 | | Dollins, Nair et al., 2008; |
| | | | Pratico, Sullenger et al., |
| | | | 2013 |
| CD28-apt (RNA) | CD28 | | Pastor, Soldevilla et al., |
| | | | 2013 |
| Del60 (RNA) | CTLA-4 | | Santulli-Marotto, Nair et |
| | | | al., 2003 |
| PSMA-4-1BB-apt | PSMA/4-1BB | | Pastor, Kolonias et al., |
| (RNA) | | | 2011 |
| CD16a/c-Met-apt | CD16a/c-Met | | Eder, Vande Woude et al., |
| (RNA) | | | 2009 |
| VEGF-4-1BB apt | VEGF/4-1BB | | Schrand, Berezhnoy et al., |
| (DNA) | | | 2014 |
| MP7 (DNA) | PD-1 | | Prodeus, Abdul-Wahid et |
| | | | al., 2015 |
| aptPD-L1 (DNA) | PD-L1 | | Lai, Huang et al., 2016 |
| R5A1 (RNA) | IL 10R | | Berezhnoy, Stewart et al., |
| | | | 2012 |
| CL-42 (RNA) | IL4R | | Roth, De La Fuente et al., |
| | | | 2012 |
| CD44-EpCAM | CD44/EpCAM | | Zheng, Zhao et al., 2017 |
| aptamer (RNA) | | | |
| TIM3 Apt (RNA) | TIM3 | | Hervas-Stubbs, Soldevilla |
| | | | et al., 2016 |
| CD40apt (RNA) | CD40 | | Soldevilla, Villanueva et |
| | | | al., 2015 |
| AptCTLA-4 (DNA) | CTLA-4 | | Huang, Lai et al., 2017 |
| AON-D21 1-Aptamer | C5a | | Ajona, Ortiz-Espinosa et |
| (RNA/DNA) | | | al., 2017 |

Accordingly, in some embodiments, the present disclosure relates to compositions and methods for treating a cancer in a subject by administering to the subject a therapeutically effective amount of a composition including a complex formed between (i) an aptamer selected from those aptamers listed in Table 6, and (ii) a 3E10 antibody or variant thereof, or antigen-binding fragment thereof, as described herein.

In some embodiments, the present disclosure relates to compositions and methods for treating a cancer in a subject by administering to the subject a therapeutically effective amount of a composition including a complex formed between (i) an aptamer that specifically binds to a molecular target selected from those molecular targets listed in Table 6, and (ii) a 3E10 antibody or variant thereof, or antigen-binding fragment thereof, as described herein.

In some embodiments, the present disclosure relates to compositions and methods for treating a cancer in a subject by administering to the subject a therapeutically effective amount of a composition including a complex formed between (i) an aptamer that specifically binds to a respective molecular target selected from those molecular targets listed in Table 6, and (ii) a 3E10 antibody or variant thereof, or antigen-binding fragment thereof, as described herein, where the cancer is a cancer associated with the molecular target within Table 6.

In some embodiments, the present disclosure relates to compositions and methods for treating a cancer in a subject by administering to the subject a therapeutically effective amount of a composition including a complex formed between (i) a respective aptamer selected from those aptamers listed in Table 6, and (ii) a 3E10 antibody or variant thereof, or antigen-binding fragment thereof, as described herein, where the cancer is a cancer associated with the respective aptamer within Table 6.

### Ribozyme

In some embodiments, a 3E10 antibody or variant thereof, or antigen-binding fragment thereof described herein includes a polynucleotide that encodes a ribozyme. Ribozymes are catalytically active RNA molecules. Ribozymes occur naturally in various sizes and shapes. They catalyze cleavage and ligation of specific phosphodiester bonds. Peptide bond formation during protein synthesis on the ribosome is catalyzed by ribosomal RNA. The biological functions of ribozymes are diverse and they play central roles during transfer RNA maturation, intron splicing, replication of RNA viruses or viroids, the regulation of messenger RNA stability, and protein synthesis (Westhof et al. in Encyclopedia of Virology (3^{rd} Edition), 2008).

### Treatment of Cancers

In one aspect, the present disclosure provides methods for treating a cancer in a subject by parenterally administering to the periphery of the subject a therapeutically effective amount of a composition including a complex formed between (i) a therapeutic polynucleotide and (ii) a 3E10 antibody or variant thereof, or antigen-binding fragment thereof, as described herein. In some embodiments, therapeutic polynucleotide is a polynucleotide immunostimulant capable of stimulating a pattern recognition receptor (PRR) that can stimulate any of the PRRs described herein and/or includes any of the polynucleotide ligands described herein.

In some embodiments, the cancer is a carcinoma, a sarcoma, a blastoma, a papilloma, or an adenoma. In other embodiments, the cancer is a metastatic cancer.

In some embodiments, methods are provided for treating a carcinoma, a sarcoma, a blastoma, a papilloma, or an adenoma in a subject by parenterally administering to the periphery of the subject a therapeutically effective amount of a composition including a complex formed between (i) a therapeutic polynucleotide and (ii) a 3E10 antibody or variant thereof, or antigen-binding fragment thereof, as described herein. In some embodiments, the therapeutic polynucleotide is a polynucleotide ligand capable of stimulating RIG-I. In some embodiments, the polynucleotide ligand has a sequence that is at least 80%, at least 85%, at least 90%, at least 95%, at least 96%, at least 97%, at least 98%, at least 99%, or 100% identical to the nucleotide sequence of 3p-hpRNA (SEQ ID NO:XX).

In some embodiments, methods are provided for treating a metastatic cancer in a subject by parenterally administering to the periphery of the subject a therapeutically effective amount of a composition including a complex formed between (i) a therapeutic polynucleotide and (ii) a 3E10 antibody or variant thereof, or antigen-binding fragment thereof, as described herein. In some embodiments, the therapeutic polynucleotide is a polynucleotide ligand capable of stimulating RIG-I. In some embodiments, the polynucleotide ligand has a sequence that is at least 80%, at least 85%, at least 90%, at least 95%, at least 96%, at least 97%, at least 98%, at least 99%, or 100% identical to the nucleotide sequence of 3p-hpRNA (SEQ ID NO:XX).

In some embodiments, the cancer is selected from the group consisting of bladder cancer, blood cancer, brain cancer, breast cancer, bone cancer, cervical cancer, colorectal cancer, endocrine cancer, esophageal cancer, gastric cancer, head and neck cancer, hepatobiliary cancer, leukemia, lung cancer, lymphoma, skin cancer, melanoma, myeloma, ovarian cancer, pancreatic cancer, prostate cancer, renal cancer, thyroid cancer, and uterine cancer.

In some embodiments, methods are provided for treating a subject suffering from a bladder cancer, blood cancer, brain cancer, breast cancer, bone cancer, cervical cancer, colorectal cancer, endocrine cancer, esophageal cancer, gastric cancer, head and neck cancer, hepatobiliary cancer, leukemia, lung cancer, lymphoma, skin cancer, melanoma, myeloma, ovarian cancer, pancreatic cancer, prostate cancer, renal cancer, thyroid cancer, and uterine cancer by parenterally administering to the periphery of the subject a therapeutically effective amount of a composition including a complex formed between (i) a therapeutic polynucleotide and (ii) a 3E10 antibody or variant thereof, or antigen-binding fragment thereof, as described herein. In some embodiments, the therapeutic polynucleotide is a polynucleotide ligand capable of stimulating RIG-I. In some embodiments, the polynucleotide ligand has a sequence that is at least 80%, at least 85%, at least 90%, at least 95%, at least 96%, at least 97%, at least 98%, at least 99%, or 100% identical to the nucleotide sequence of 3p-hpRNA (SEQ ID NO:XX).

In some embodiments, the cancer is a skin cancer selected from the group consisting of basal cell carcinoma, squamous cell carcinoma, and melanoma. In one embodiment, the cancer is melanoma.

In some embodiments, methods are provided for treating basal cell carcinoma, squamous cell carcinoma, or melanoma in a subject by parenterally administering to the periphery of the subject a therapeutically effective amount of a composition including a complex formed between (i) a therapeutic polynucleotide and (ii) a 3E10 antibody or variant thereof, or antigen-binding fragment thereof, as described herein. In some embodiments, the therapeutic polynucleotide is a polynucleotide ligand capable of stimulating RIG-I. In some embodiments, the polynucleotide ligand has a sequence that is at least 80%, at least 85%, at least 90%, at least 95%, at least 96%, at least 97%, at least 98%, at least 99%, or 100% identical to the nucleotide sequence of 3p-hpRNA (SEQ ID NO:XX).

In some embodiments, methods are provided for treating melanoma in a subject by parenterally administering to the periphery of the subject a therapeutically effective amount of a composition including a complex formed between (i) a therapeutic polynucleotide and (ii) a 3E10 antibody or variant thereof, or antigen-binding fragment thereof, as described herein. In some embodiments, the therapeutic polynucleotide is a polynucleotide ligand capable of stimulating RIG-I. In some embodiments, the polynucleotide ligand has a sequence that is at least 80%, at least 85%, at least 90%, at least 95%, at least 96%, at least 97%, at least 98%, at least 99%, or 100% identical to the nucleotide sequence of 3p-hpRNA (SEQ ID NO:XX).

### Cancer of the Haematopoietic System

In one embodiment, the cancer of the central nervous system is a cancer of the hematopoietic system. Accordingly, methods are provided for treating a cancer of the hematopoietic system in a subject by parenterally administering to the periphery of the subject a therapeutically effective amount of a composition including a complex formed between (i) a polynucleotide ligand capable of stimulating a pattern recognition receptor (PRR) and (ii) a 3E10 antibody or variant thereof, or antigen-binding fragment thereof, as described herein. In some embodiments, the cancer of the hematopoietic system is a malignant lymphoma, a plasmocytoma, or a granulocytic sarcoma. In some embodiments, the polynucleotide ligand is capable of stimulating RIG-I. In some embodiments, the polynucleotide ligand has a sequence that is at least 80%, at least 85%, at least 90%, at least 95%, at least 96%, at least 97%, at least 98%, at least 99%, or 100% identical to the nucleotide sequence of 3p-hpRNA (SEQ ID NO:XX).

In some embodiments, the treatment of the cancer of the hematopoietic system reduces the tumor burden of the subject. In some embodiments, the treatment reduces the tumor burden of the subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more.

In some embodiments, the cancer is a cancer of the hematopoietic system that has not metastasized to the spinal cord of the subject. In some embodiments, for example as demonstrated in Example 7, the treatment reduces the probability of the cancer metastasizing to the spinal cord. Accordingly, in some embodiments, a method is provided for reducing the risk of metastasis in a subject with a cancer of the hematopoietic system by parenterally administering to the periphery of the subject a therapeutically effective amount of a composition including a complex formed between (i) a polynucleotide ligand capable of stimulating a pattern recognition receptor (PRR) and (ii) a 3E10 antibody or variant thereof, or antigen-binding fragment thereof, as described herein. In some embodiments, the polynucleotide ligand is capable of stimulating RIG-I. In some embodiments, the polynucleotide ligand has a sequence that is at least 80%, at least 85%, at least 90%, at least 95%, at least 96%, at least 97%, at least 98%, at least 99%, or 100% identical to the nucleotide sequence of 3p-hpRNA (SEQ ID NO:XX).

### Germ Cell Tumors

In one embodiment, the cancer of the central nervous system is a germ cell tumor. Accordingly, methods are provided for treating a germ cell tumor in a subject by parenterally administering to the periphery of the subject a therapeutically effective amount of a composition including a complex formed between (i) a polynucleotide ligand capable of stimulating a pattern recognition receptor (PRR) and (ii) a 3E10 antibody or variant thereof, or antigen-binding fragment thereof, as described herein. In some embodiments, the germ cell tumor is a germinoma, an embryonal carcinoma, a yolk sac tumor, a Choriocarcinoma, a teratoma, or a mixed germ cell tumor. In some embodiments, the polynucleotide ligand is capable of stimulating RIG-I. In some embodiments, the polynucleotide ligand has a sequence that is at least 80%, at least 85%, at least 90%, at least 95%, at least 96%, at least 97%, at least 98%, at least 99%, or 100% identical to the nucleotide sequence of 3p-hpRNA (SEQ ID NO:XX).

In some embodiments, the treatment of the germ cell tumor reduces the tumor burden of the subject. In some embodiments, the treatment reduces the tumor burden of the subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more.

In some embodiments, the cancer is a germ cell tumor that has not metastasized to the spinal cord of the subject. In some embodiments, for example as demonstrated in Example 7, the treatment reduces the probability of the cancer metastasizing to the spinal cord. Accordingly, in some embodiments, a method is provided for reducing the risk of metastasis in a subject with a germ cell tumor by parenterally administering to the periphery of the subject a therapeutically effective amount of a composition including a complex formed between (i) a polynucleotide ligand capable of stimulating a pattern recognition receptor (PRR) and (ii) a 3E10 antibody or variant thereof, or antigen-binding fragment thereof, as described herein. In some embodiments, the polynucleotide ligand is capable of stimulating RIG-I. In some embodiments, the polynucleotide ligand has a sequence that is at least 80%, at least 85%, at least 90%, at least 95%, at least 96%, at least 97%, at least 98%, at least 99%, or 100% identical to the nucleotide sequence of 3p-hpRNA (SEQ ID NO:XX).

### Tumors of the Sellar Region

In one embodiment, the cancer of the central nervous system is a tumor of the sellar region. Accordingly, methods are provided for treating a tumor of the sellar region in a subject by parenterally administering to the periphery of the subject a therapeutically effective amount of a composition including a complex formed between (i) a polynucleotide ligand capable of stimulating a pattern recognition receptor (PRR) and (ii) a 3E10 antibody or variant thereof, or antigen-binding fragment thereof, as described herein. In some embodiments, the tumor of the sellar region is craniopharyngioma, a granular cell tumor, a pituicytoma, or a spindle cell oncocytoma of the adenohypophysis. In some embodiments, the polynucleotide ligand is capable of stimulating RIG-I. In some embodiments, the polynucleotide ligand has a sequence that is at least 80%, at least 85%, at least 90%, at least 95%, at least 96%, at least 97%, at least 98%, at least 99%, or 100% identical to the nucleotide sequence of 3p-hpRNA (SEQ ID NO:XX).

In some embodiments, the treatment of the tumor of the sellar region reduces the tumor burden of the subject. In some embodiments, the treatment reduces the tumor burden of the subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more.

In some embodiments, the cancer is a tumor of the sellar region that has not metastasized to the spinal cord of the subject. In some embodiments, for example as demonstrated in Example 7, the treatment reduces the probability of the cancer metastasizing to the spinal cord. Accordingly, in some embodiments, a method is provided for reducing the risk of metastasis in a subject with a tumor of the sellar region by parenterally administering to the periphery of the subject a therapeutically effective amount of a composition including a complex formed between (i) a polynucleotide ligand capable of stimulating a pattern recognition receptor (PRR) and (ii) a 3E10 antibody or variant thereof, or antigen-binding fragment thereof, as described herein. In some embodiments, the polynucleotide ligand is capable of stimulating RIG-I. In some embodiments, the polynucleotide ligand has a sequence that is at least 80%, at least 85%, at least 90%, at least 95%, at least 96%, at least 97%, at least 98%, at least 99%, or 100% identical to the nucleotide sequence of 3p-hpRNA (SEQ ID NO:XX).

### Compositions for Treating Cancers

In one aspect, the present disclosure provides pharmaceutical compositions including a complex formed between a therapeutic polynucleotide as described herein, and a 3E10 antibody or variant thereof, or antigen-binding fragment thereof, as described herein.

In some embodiments, therapeutic polynucleotide encodes a protein or peptide for cancer therapy. In some embodiments, the protein or peptide for cancer therapy is a tumor antigen.

In other embodiments, the tumor antigen is selected from the group consisting of a oncoviral protein antigen, a neoantigen, and an antigen derived from a cancer-germline gene. In some embodiments, the oncoviral protein antigen is at least one selected from the group consisting of folate receptor, HER2, papillomavirus oncoprotein E6 and papillomavirus oncoprotein E7 carcinoembryonic antigen (CEA), mucin 1, EGFR, cMyc, mutant TP53, squamous cell carcinoma antigen recognized by T cells 3 (SART3), beta-human chorionic gonadotropin (beta-hCG), Wilms' Tumor antigen 1 (WT1), Survivin, MAGE3, p53, ring finger protein 43 and translocase of the outer mitochondrial membrane 34 (TOMM34), prostate-specific antigen (PSA)-TRICOM, and KRAS.

In some embodiments, the neoantigen is at least one selected from the group consisting of BRCA1, BRCA2 BRAF, KRAS, EGFR, IDH1, PIK3CA, ROS1, HLA, JAK1, JAK2, PARK2, ATM, p53, TP53, erbb2 interacting protein (ERBB2IP), Beta-2-Microglobulin (β2m), cyclin-dependent kinase inhibitor 2A (CDKN2A), Claudin-18.2, alternate reading frame (ARF), and cyclin-dependent kinase 4 (CDK4).

In some embodiments, wherein the cancer germline gene is at least one selected from the group consisting of MAGEA1, MAGEA2, MAGEA3, MAGEA4, MAGEA5, MAGEA6, MAGEA8, MAGEA9, MAGEA10, MAGEA11, MAGEA12, BAGE, BAGE2, BAGE3, BAGE4, BAGE5, MAGEB1, MAGEB2, MAGEB5, MAGEB6, MAGEB3, MAGEB4, GAGE1, GAGE2A, GAGE3, GAGE4, GAGE5, GAGE6, GAGE7, GAGE8, SSX1, SSX2, SSX2b, SSX3, SSX4, CTAG1B, LAGE-1b, CTAG2, MAGEC1, MAGEC3, SYCP1, BRDT, MAGEC2, SPANXA1, SPANXB1, SPANXC, SPANXD, SPANXN1, SPANXN2, SPANXN3, SPANXN4, SPANXN5, XAGE1D, XAGE1C, XAGE1B, XAGE1, XAGE2, XAGE3, XAGE-3b, XAGE-4/RP11-167P23.2, XAGE5, DDX43, SAGE1, ADAM2, PAGE5, CT16.2, PAGE1, PAGE2, PAGE2B, PAGE3, PAGE4, LIPI, VENTXP1, IL13RA2, TSP50, CTAGE1, CTAGE-2, CTAGE5, SPA17, ACRBP, CSAG1, CSAG2, DSCR8, MMA1b, DDX53, CTCFL, LUZP4, CASC5, TFDP3, JARID1B, LDHC, MORC1, DKKL1, SPO11, CRISP2, FMR1NB, FTHL17, NXF2, TAF7L, TDRD1, TDRD6, TDRD4, TEX15, FATE1, TPTE, CT45A1, CT45A2, CT45A3, CT45A4, CT45A5, CT45A6, HORMAD1, HORMAD2, CT47A1, CT47A2, CT47A3, CT47A4, CT47A5, CT47A6, CT47A7, CT47A8, CT47A9, CT47A10, CT47A11, CT47B1, SLCO6A1, TAG, LEMD1, HSPB9, CCDC110, ZNF165, SPACA3, CXorf48, THEG, ACTL8, NLRP4, COX6B2, LOC348120, CCDC33, LOC196993, PASD1, LOC647107, TULP2, CT66/AA884595, PRSS54, RBM46, CT69/BC040308, CT70/BI818097, SPINLW1, TSSK6, ADAM29, CCDC36, LOC440934, SYCE1, CPXCR1, TSPY3, TSGA10, HIWI, MIWI, PIWI, PIWIL2, ARMC3, AKAP3, Cxorf61, PBK, C21orf99, OIP5, CEP290, CABYR, SPAG9, MPHOSPH1, ROPN1, PLAC1, CALR3, PRM1, PRM2, CAGE1, TTK, LY6K, IMP-3, AKAP4, DPPA2, KIAA0100, DCAF12, SEMG1, POTED, POTEE, POTEA, POTEG, POTEB, POTEC, POTEH, GOLGAGL2 FA, CDCA1, PEPP2, OTOA, CCDC62, GPATCH2, CEP55, FAM46D, TEX14, CTNNA2, FAM133A, LOC130576, ANKRD45, ELOVL4, IGSF11, TMEFF1, TMEFF2, ARX, SPEF2, GPAT2, TMEM108, NOL4, PTPN20A, SPAG4, MAEL, RQCD1, PRAME, TEX101, SPATA19, ODF1, ODF2, ODF3, ODF4, ATAD2, ZNF645, MCAK, SPAG1, SPAG6, SPAG8, SPAG17, FBXO39, RGS22, cyclin A1, C15orf60, CCDC83, TEKT5, NR6A1, TMPRSS12, TPPP2, PRSS55, DMRT1, EDAG, NDR, DNAJB8, CSAG3B, CTAG1A, GAGE12B, GAGE12C, GAGE12D, GAGE12E, GAGE12F, GAGE12G, GAGE12H, GAGE12I, GAGE12J, GAGE13, LOC728137, MAGEA2B, MAGEA9B/LOC728269, NXF2B, SPANXA2, SPANXB2, SPANXE, SSX4B, SSX5, SSX6, SSX7, SSX9, TSPY1D, TSPY1E, TSPY1F, TSPY1G, TSPY1H, TSPY1I, TSPY2, and XAGE1E.

In yet other embodiments, the peptide for cancer therapy is a proinflammatory cytokine.

In some embodiments, the proinflammatory cytokine is at least one selected from the group consisting of IL-1, IL-6, IL-8, IL-12, IFN-γ, IL-18, IL-15, IL-2, TNF-α, IL-10, TGF-β, CSF-1, CCL2, CCL3, CCL5, and VEGF.

In further embodiments, the therapeutic polynucleotide is non-replicating unmodified mRNA. In some embodiments, the therapeutic polynucleotide is non-replicating modified mRNA. In some embodiments, the therapeutic polynucleotide is a self-amplifying mRNA. In some embodiments, the therapeutic polynucleotide is a plasmid encoding the protein or peptide. In some embodiments, the therapeutic polynucleotide is a plasmid encoding an antisense sequence. In some embodiments, wherein the therapeutic polynucleotide is a gene-regulating polynucleotide.

In some embodiments, the gene-regulating polynucleotide is an siRNA. In some embodiments, the siRNA comprises a lipid-based nanoparticles (LNPs) siRNA, a KRAS siRNA, a Her2 siRNA, a VEGF siRNA, a EGFR siRNA, a SOSC1 siRNA, a ADAR1 siRNA, a PLK1 siRNA, cMyc siRNA, a TP53 siRNA, a HIF2a siRNA, or a Bcl2 siRNA.

In some embodiments, the gene-regulating polynucleotide is an miRNA. In some embodiments, the miRNA comprises miR-15a, miR-15b, miR-16, miR-20b, miR-21, miR-28, miR-34a, miR-34b, miR-34c, miR-125b, miR-130b, miR-138, miR-138-5p, miR-155, miR-195, miR-197, miR-200, miR-210, miR-221, miR-222, miR-424, miR-497, miR-503, or miR-513

In some embodiments, the gene-regulating polynucleotide is a small-activating RNA (saRNA). In some embodiments, the saRNA comprises an saRNA for the transcription factor CCATT/enhancer binding protein alpha (CEBPA).

In some embodiments, the gene-regulating polynucleotide is an antagomir.

In some embodiments, the gene-regulating polynucleotide is an antisense oligonucleotide.

In some embodiments, the gene-regulating polynucleotide is a decoy oligonucleotide

In some embodiments, the genome editing polynucleotide encodes a zinc-finger nuclease. In some embodiments, the genome editing polynucleotide encodes a transcription activator-like effector nuclease (TALEN). In some embodiments, the genome editing polynucleotide encodes a CRISPR system comprising a Cas protein and a guide RNA.

In some embodiments, the therapeutic polynucleotide is an effector polynucleotide. In some embodiments, the effector polynucleotide is an aptamer. In some embodiments, the aptamer comprises a PSMA aptamer, a HER2 aptamer, a MUC1 aptamer, a CD117 aptamer, a PTK7 aptamer, CTLA-4 aptamer, TLS11a aptamer, PD-1 aptamer, a PD-1 aptamer, a Macugen aptamer, AS1411, Sgc8, TD05, ARC1779, a-Thrombin (TBA), Macugen, E10030, AS1411, ARC1779, NU172, NOX-A12, NOX-E36, NOX-H94, ARC1905, REG1, ARC19499, AS1411, AS1411, EpCAM, A10-3-J1, Sgc8c, TSA14, 5TR1, Endo28, EGFR, A10, Sgc8c, AS1411, NOX-A12, KH1C12, K19, TD05, AS1411, HB5, HeA2_3, H2, S6, SYL3C, APTA-12, M17, S-1, SL2B, CAA01, CA50 A02, CA72-4 A01, APT-43, TA6, CA125.1, Apt928, R13, HF3-58, or HA5-68.

In some embodiments, the aptamer is an immune checkpoint inhibitor selected from B7-H3, B7-H4, BTLA, CD160, CTLA4, KIR, LAG3, PD-1, PD-L1, PD-L2, TIM3, and TIGIT, or combinations thereof.

In some embodiments, the effector polynucleotide is a ribozyme. In some embodiments, the ribozyme targets human telomerase reverse transcriptase (hTERT) RNA.

In some embodiments, a pharmaceutical composition described herein has a molar ratio of 3E10 antibody or variant thereof, or antigen-binding fragment thereof to therapeutic polynucleotide of at least 2:1. The use of molar ratios of 3E10 antibody or variant thereof, or antigen-binding fragment thereof to therapeutic polynucleotide in the compositions described herein protects the therapeutic polynucleotide from degradation. For instance, as illustrated in FIGS. 13A and 13B, while parental 3E10 and 3E10 (D31N) variant antibodies protected mRNA from RNAse A-mediated RNA degradation at molar ratios of 2:1 and 20:1, the protection afforded by the 20:1 molar ratio exceeded the protection afforded at 2:1.

Accordingly, in some embodiments, a pharmaceutical composition described herein has a molar ratio of 3E10 antibody or variant thereof, or antigen-binding fragment thereof to therapeutic polynucleotide that is greater than about 2:1. In some embodiments, a pharmaceutical composition described herein has a molar ratio of 3E10 antibody or variant thereof, or antigen-binding fragment thereof to therapeutic polynucleotide that is at least about 5:1. In some embodiments, a pharmaceutical composition described herein has a molar ratio of 3E10 antibody or variant thereof, or antigen-binding fragment thereof to therapeutic polynucleotide that is at least about 7.5:1. In some embodiments, a pharmaceutical composition described herein has a molar ratio of 3E10 antibody or variant thereof, or antigen-binding fragment thereof to therapeutic polynucleotide that is at least about 10:1. In some embodiments, a pharmaceutical composition described herein has a molar ratio of 3E10 antibody or variant thereof, or antigen-binding fragment thereof to therapeutic polynucleotide that is at least about 15:1. In some embodiments, a pharmaceutical composition described herein has a molar ratio of 3E10 antibody or variant thereof, or antigen-binding fragment thereof to therapeutic polynucleotide that is at least about 20:1. In some embodiments, a pharmaceutical composition described herein has a molar ratio of 3E10 antibody or variant thereof, or antigen-binding fragment thereof to therapeutic polynucleotide that is at least about 25:1. In some embodiments, a pharmaceutical composition described herein has a molar ratio of 3E10 antibody or variant thereof, or antigen-binding fragment thereof to therapeutic polynucleotide that is at least about 30:1. In some embodiments, a pharmaceutical composition described herein has a molar ratio of 3E10 antibody or variant thereof, or antigen-binding fragment thereof to therapeutic polynucleotide that is at least about 40:1.

In some embodiments, a pharmaceutical composition described herein has a molar ratio of 3E10 antibody or variant thereof, or antigen-binding fragment thereof to therapeutic polynucleotide that is at least about 1:1, at least about 2:1, at least about 3:1, at least about 4:1, at least about 5:1, at least about 6:1, at least about 7:1, at least about 8:1, at least about 9:1, at least about 10:1, at least about 15:1, at least about 20:1, at least about 25:1, at least about 30:1, at least about 35:1, at least about 40:1, at least about 45:1, at least about 50:1, at least about 55:1, at least about 60:1, at least about 65:1, at least about 70:1, at least about 75:1, at least about 80:1, at least about 85:1, at least about 90:1, at least about 95:1, at least about 100:1, at least about 105:1, at least about 110:1, at least about 115:1, at least about 120:1, at least about 125:1, at least about 130:1, at least about 135:1, at least about 140:1, at least about 145:1, at least about 150:1, at least about 155:1, at least about 160:1, at least about 165:1, at least about 170:1, at least about 175:1, at least about 180:1, at least about 185:1, at least about 190:1, at least about 195:1, at least about 200:1, at least about 205:1, at least about 210:1, at least about 215:1, at least about 220:1, at least about 225:1, at least about 230:1, at least about 235:1, at least about 240:1, at least about 245:1, at least about 250:1, or greater and any ranges in between.

In some embodiments, a pharmaceutical composition described herein has a molar ratio of 3E10 antibody or variant thereof, or antigen-binding fragment thereof to therapeutic polynucleotide that is 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, 200:1, 205:1, 210:1, 215:1, 220:1, 225:1, 230:1, 235:1, 240:1, 245:1, 250:1, or greater and any ranges in between.

In some embodiments, a pharmaceutical composition described herein has a molar ratio of 3E10 antibody or variant thereof, or antigen-binding fragment thereof to therapeutic polynucleotide that is less or no more than 1:1, no more than 2:1, no more than 3:1, no more than 4:1, no more than 5:1, no more than 6:1, no more than 7:1, no more than 8:1, no more than 9:1, no more than 10:1, no more than 15:1, no more than 20:1, no more than 25:1, no more than 30:1, no more than 35:1, no more than 40:1, no more than 45:1, no more than 50:1, no more than 55:1, no more than 60:1, no more than 65:1, no more than 70:1, no more than 75:1, no more than 80:1, no more than 85:1, no more than 90:1, no more than 95:1, no more than 100:1, no more than 105:1, no more than 110:1, no more than 115:1, no more than 120:1, no more than 125:1, no more than 130:1, no more than 135:1, no more than 140:1, no more than 145:1, no more than 150:1, no more than 155:1, no more than 160:1, no more than 165:1, no more than 170:1, no more than 175:1, no more than 180:1, no more than 185:1, no more than 190:1, no more than 195:1, no more than 200:1, no more than 205:1, no more than 210:1, no more than 215:1, no more than 220:1, no more than 225:1, no more than 230:1, no more than 235:1, no more than 240:1, no more than 245:1, no more than 250:1 and any ranges in between.

In some embodiments, a pharmaceutical composition described herein has a molar ratio of 3E10 antibody or variant thereof, or antigen-binding fragment thereof to therapeutic polynucleotide that is at least 10:1. In some embodiments, a pharmaceutical composition described herein has a molar ratio of 3E10 antibody or variant thereof, or antigen-binding fragment thereof to therapeutic polynucleotide that is at least 5:1. In some embodiments, a pharmaceutical composition described herein has a molar ratio of 3E10 antibody or variant thereof, or antigen-binding fragment thereof to therapeutic polynucleotide that is at least 7.5:1. In some embodiments, a pharmaceutical composition described herein has a molar ratio of 3E10 antibody or variant thereof, or antigen-binding fragment thereof to therapeutic polynucleotide that is at least 10:1. In some embodiments, a pharmaceutical composition described herein has a molar ratio of 3E10 antibody or variant thereof, or antigen-binding fragment thereof to therapeutic polynucleotide that is at least 15:1. In some embodiments, a pharmaceutical composition described herein has a molar ratio of 3E10 antibody or variant thereof, or antigen-binding fragment thereof to therapeutic polynucleotide that is at least 20:1. In some embodiments, a pharmaceutical composition described herein has a molar ratio of 3E10 antibody or variant thereof, or antigen-binding fragment thereof to therapeutic polynucleotide that is at least 25:1. In some embodiments, a pharmaceutical composition described herein has a molar ratio of 3E10 antibody or variant thereof, or antigen-binding fragment thereof to therapeutic polynucleotide that is at least 30:1. In some embodiments, a pharmaceutical composition described herein has a molar ratio of 3E10 antibody or variant thereof, or antigen-binding fragment thereof to therapeutic polynucleotide that is at least 40:1.

In some embodiments, a pharmaceutical composition described herein has a molar ratio of 3E10 antibody or variant thereof, or antigen-binding fragment thereof to therapeutic polynucleotide that is at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, at least 20:1, at least 21:1, at least 22:1, at least 23:1, at least 24:1, at least 25:1, at least 26:1, at least 27:1, at least 28:1, at least 29:1, at least 30:1, at least 31:1, at least 32:1, at least 33:1, at least 34:1, at least 35:1, at least 36:1, at least 37:1, at least 38:1, at least 39:1, at least 40:1, at least 41:1, at least 42:1, at least 43:1, at least 44:1, at least 45:1, or greater.

In some embodiments, a pharmaceutical composition described herein has a molar ratio of 3E10 antibody or variant thereof, or antigen-binding fragment thereof to therapeutic polynucleotide that is 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, or greater.

In some embodiments, a pharmaceutical composition described herein has a molar ratio of 3E10 antibody or variant thereof, or antigen-binding fragment thereof to therapeutic polynucleotide that is no more than about 50:1. In some embodiments, a pharmaceutical composition described herein has a molar ratio of 3E10 antibody or variant thereof, or antigen-binding fragment thereof to therapeutic polynucleotide that is no more than about 40:1. In some embodiments, a pharmaceutical composition described herein has a molar ratio of 3E10 antibody or variant thereof, or antigen-binding fragment thereof to therapeutic polynucleotide that is no more than about 30:1. In some embodiments, a pharmaceutical composition described herein has a molar ratio of 3E10 antibody or variant thereof, or antigen-binding fragment thereof to therapeutic polynucleotide that is no more than about 25:1. In some embodiments, a pharmaceutical composition described herein has a molar ratio of 3E10 antibody or variant thereof, or antigen-binding fragment thereof to therapeutic polynucleotide that is no more than about 20:1. In some embodiments, a pharmaceutical composition described herein has a molar ratio of 3E10 antibody or variant thereof, or antigen-binding fragment thereof to therapeutic polynucleotide that is no more than about 15:1. In some embodiments, a pharmaceutical composition described herein has a molar ratio of 3E10 antibody or variant thereof, or antigen-binding fragment thereof to therapeutic polynucleotide that is no more than about 10:1.

In some embodiments, a pharmaceutical composition described herein has a molar ratio of 3E10 antibody or variant thereof, or antigen-binding fragment thereof to therapeutic polynucleotide that is no more than about 50:1, no more than about 49:1, no more than about 48:1, no more than about 47:1, no more than about 46:1, no more than about 45:1, no more than about 44:1, no more than about 43:1, no more than about 42:1, no more than about 41:1, no more than about 40:1, no more than about 39:1, no more than about 38:1, no more than about 37:1, no more than about 36:1, no more than about 35:1, no more than about 34:1, no more than about 33:1, no more than about 32:1, no more than about 31:1, no more than about 30:1, no more than about 29:1, no more than about 28:1, no more than about 27:1, no more than about 26:1, no more than about 25:1, no more than about 24:1, no more than about 23:1, no more than about 22:1, no more than about 21:1, no more than about 20:1, no more than about 19:1, no more than about 18:1, no more than about 17:1, no more than about 16:1, no more than about 15:1, no more than about 14:1, no more than about 13:1, no more than about 12:1, no more than about 11:1, no more than about 10:1, no more than about 9:1, no more than about 8:1, no more than about 7:1, no more than about 6:1, no more than about 5:1, or less.

In some embodiments, a pharmaceutical composition described herein has a molar ratio of 3E10 antibody or variant thereof, or antigen-binding fragment thereof to therapeutic polynucleotide that is no more than 50:1, no more than 49:1, no more than 48:1, no more than 47:1, no more than 46:1, no more than 45:1, no more than 44:1, no more than 43:1, no more than 42:1, no more than 41:1, no more than 40:1, no more than 39:1, no more than 38:1, no more than 37:1, no more than 36:1, no more than 35:1, no more than 34:1, no more than 33:1, no more than 32:1, no more than 31:1, no more than 30:1, no more than 29:1, no more than 28:1, no more than 27:1, no more than 26:1, no more than 25:1, no more than 24:1, no more than 23:1, no more than 22:1, no more than 21:1, no more than 20:1, no more than 19:1, no more than 18:1, no more than 17:1, no more than 16:1, no more than 15:1, no more than 14:1, no more than 13:1, no more than 12:1, no more than 11:1, no more than 10:1, no more than 9:1, no more than 8:1, no more than 7:1, no more than 6:1, no more than 5:1, or less.

In some embodiments, a pharmaceutical composition described herein has a molar ratio of 3E10 antibody or variant thereof, or antigen-binding fragment thereof to therapeutic polynucleotide that is of from about 2:1 to about 50:1. In some embodiments, a pharmaceutical composition described herein has a molar ratio of 3E10 antibody or variant thereof, or antigen-binding fragment thereof to therapeutic polynucleotide that is of from about 2:1 to about 40:1. In some embodiments, a pharmaceutical composition described herein has a molar ratio of 3E10 antibody or variant thereof, or antigen-binding fragment thereof to therapeutic polynucleotide that is of from about 2:1 to about 30:1. In some embodiments, a pharmaceutical composition described herein has a molar ratio of 3E10 antibody or variant thereof, or antigen-binding fragment thereof to therapeutic polynucleotide that is of from about 2:1 to about 25:1. In some embodiments, a pharmaceutical composition described herein has a molar ratio of 3E10 antibody or variant thereof, or antigen-binding fragment thereof to therapeutic polynucleotide that is of from about 2:1 to about 20:1. In some embodiments, a pharmaceutical composition described herein has a molar ratio of 3E10 antibody or variant thereof, or antigen-binding fragment thereof to therapeutic polynucleotide that is of from about 2:1 to about 15:1. In some embodiments, a pharmaceutical composition described herein has a molar ratio of 3E10 antibody or variant thereof, or antigen-binding fragment thereof to therapeutic polynucleotide that is of from about 2:1 to about 10:1. In some embodiments, a pharmaceutical composition described herein has a molar ratio of 3E10 antibody or variant thereof, or antigen-binding fragment thereof to therapeutic polynucleotide that is of from about 2:1 to about 7.5:1. In some embodiments, a pharmaceutical composition described herein has a molar ratio of 3E10 antibody or variant thereof, or antigen-binding fragment thereof to therapeutic polynucleotide that is of from about 2:1 to about 5:1. In some embodiments, a pharmaceutical composition described herein has a molar ratio of 3E10 antibody or variant thereof, or antigen-binding fragment thereof to therapeutic polynucleotide that is of from about 2:1 to about 5:1. In some embodiments, a pharmaceutical composition described herein has a molar ratio of 3E10 antibody or variant thereof, or antigen-binding fragment thereof to therapeutic polynucleotide that is of from about 2:1 to about 3:1.

In some embodiments, a pharmaceutical composition described herein has a molar ratio of 3E10 antibody or variant thereof, or antigen-binding fragment thereof to therapeutic polynucleotide that is of from about 5:1 to about 50:1. In some embodiments, a pharmaceutical composition described herein has a molar ratio of 3E10 antibody or variant thereof, or antigen-binding fragment thereof to therapeutic polynucleotide that is of from about 5:1 to about 40:1. In some embodiments, a pharmaceutical composition described herein has a molar ratio of 3E10 antibody or variant thereof, or antigen-binding fragment thereof to therapeutic polynucleotide that is of from about 5:1 to about 30:1. In some embodiments, a pharmaceutical composition described herein has a molar ratio of 3E10 antibody or variant thereof, or antigen-binding fragment thereof to therapeutic polynucleotide that is of from about 5:1 to about 25:1. In some embodiments, a pharmaceutical composition described herein has a molar ratio of 3E10 antibody or variant thereof, or antigen-binding fragment thereof to therapeutic polynucleotide that is of from about 5:1 to about 20:1. In some embodiments, a pharmaceutical composition described herein has a molar ratio of 3E10 antibody or variant thereof, or antigen-binding fragment thereof to therapeutic polynucleotide that is of from about 5:1 to about 15:1. In some embodiments, a pharmaceutical composition described herein has a molar ratio of 3E10 antibody or variant thereof, or antigen-binding fragment thereof to therapeutic polynucleotide that is of from about 5:1 to about 10:1. In some embodiments, a pharmaceutical composition described herein has a molar ratio of 3E10 antibody or variant thereof, or antigen-binding fragment thereof to therapeutic polynucleotide that is of from about 5:1 to about 7.5:1.

In some embodiments, a pharmaceutical composition described herein has a molar ratio of 3E10 antibody or variant thereof, or antigen-binding fragment thereof to therapeutic polynucleotide that is of from about 10:1 to about 50:1. In some embodiments, a pharmaceutical composition described herein has a molar ratio of 3E10 antibody or variant thereof, or antigen-binding fragment thereof to therapeutic polynucleotide that is of from about 10:1 to about 40:1. In some embodiments, a pharmaceutical composition described herein has a molar ratio of 3E10 antibody or variant thereof, or antigen-binding fragment thereof to therapeutic polynucleotide that is of from about 10:1 to about 30:1. In some embodiments, a pharmaceutical composition described herein has a molar ratio of 3E10 antibody or variant thereof, or antigen-binding fragment thereof to therapeutic polynucleotide that is of from about 10:1 to about 25:1. In some embodiments, a pharmaceutical composition described herein has a molar ratio of 3E10 antibody or variant thereof, or antigen-binding fragment thereof to therapeutic polynucleotide that is of from about 10:1 to about 20:1. In some embodiments, a pharmaceutical composition described herein has a molar ratio of 3E10 antibody or variant thereof, or antigen-binding fragment thereof to therapeutic polynucleotide that is of from about 10:1 to about 15:1.

In some embodiments, a pharmaceutical composition described herein has a molar ratio of 3E10 antibody or variant thereof, or antigen-binding fragment thereof to therapeutic polynucleotide that is of from about 15:1 to about 50:1. In some embodiments, a pharmaceutical composition described herein has a molar ratio of 3E10 antibody or variant thereof, or antigen-binding fragment thereof to therapeutic polynucleotide that is of from about 15:1 to about 40:1. In some embodiments, a pharmaceutical composition described herein has a molar ratio of 3E10 antibody or variant thereof, or antigen-binding fragment thereof to therapeutic polynucleotide that is of from about 15:1 to about 30:1. In some embodiments, a pharmaceutical composition described herein has a molar ratio of 3E10 antibody or variant thereof, or antigen-binding fragment thereof to therapeutic polynucleotide that is of from about 15:1 to about 25:1. In some embodiments, a pharmaceutical composition described herein has a molar ratio of 3E10 antibody or variant thereof, or antigen-binding fragment thereof to therapeutic polynucleotide that is of from about 15:1 to about 20:1.

In some embodiments, a pharmaceutical composition described herein has a molar ratio of 3E10 antibody or variant thereof, or antigen-binding fragment thereof to therapeutic polynucleotide that is of from about 20:1 to about 50:1. In some embodiments, a pharmaceutical composition described herein has a molar ratio of 3E10 antibody or variant thereof, or antigen-binding fragment thereof to therapeutic polynucleotide that is of from about 20:1 to about 40:1. In some embodiments, a pharmaceutical composition described herein has a molar ratio of 3E10 antibody or variant thereof, or antigen-binding fragment thereof to therapeutic polynucleotide that is of from about 20:1 to about 30:1. In some embodiments, a pharmaceutical composition described herein has a molar ratio of 3E10 antibody or variant thereof, or antigen-binding fragment thereof to therapeutic polynucleotide that is of from about 20:1 to about 25:1.

In some embodiments, a pharmaceutical composition described herein has a molar ratio of 3E10 antibody or variant thereof, or antigen-binding fragment thereof to therapeutic polynucleotide that is of from about 25:1 to about 50:1. In some embodiments, a pharmaceutical composition described herein has a molar ratio of 3E10 antibody or variant thereof, or antigen-binding fragment thereof to therapeutic polynucleotide that is of from about 25:1 to about 40:1. In some embodiments, a pharmaceutical composition described herein has a molar ratio of 3E10 antibody or variant thereof, or antigen-binding fragment thereof to therapeutic polynucleotide that is of from about 25:1 to about 30:1. In some embodiments, a pharmaceutical composition described herein has a molar ratio of 3E10 antibody or variant thereof, or antigen-binding fragment thereof to therapeutic polynucleotide that is of from about 30:1 to about 50:1. In some embodiments, a pharmaceutical composition described herein has a molar ratio of 3E10 antibody or variant thereof, or antigen-binding fragment thereof to therapeutic polynucleotide that is of from about 30:1 to about 40:1. In yet other embodiments, other ranges falling with the range of about 2:1 to about 50:1 are contemplated.

In some embodiments, a pharmaceutical composition described herein has a molar ratio of 3E10 antibody or variant thereof, or antigen-binding fragment thereof to therapeutic polynucleotide that is of from 2:1 to 50:1, from 2:1 to 40:1, from 2:1 to 30:1, from 2:1 to 25:1, from 2:1 to 20:1, from 2:1 to 15:1, from 2:1 to 10:1, from 2:1 to 7.5:1, from 2:1 to 5:1, from 5:1 to 50:1, from 5:1 to 40:1, from 5:1 to 30:1, from 5:1 to 25:1, from 5:1 to 20:1, from 5:1 to 15:1, from 5:1 to 10:1, from 5:1 to 7.5:1, from 10:1 to 50:1, from 10:1 to 40:1, from 10:1 to 30:1, from 10:1 to 25:1, from 10:1 to 20:1, from 10:1 to 15:1, from 15:1 to 50:1, from 15:1 to 40:1, from 15:1 to 30:1, from 15:1 to 25:1, from 15:1 to 20:1, from 20:1 to 50:1, from 20:1 to 40:1, from 20:1 to 30:1, from 20:1 to 25:1, from 25:1 to 50:1, from 25:1 to 40:1, from 25:1 to 30:1, from 30: to 50:1, from 30:1 to 40:1, or from 40:1 to 50:1. In yet other embodiments, other ranges falling with the range of from 2:1 to 50:1 are contemplated.

In some embodiments, a pharmaceutical composition described herein has a molar ratio of 3E10 antibody or variant thereof, or antigen-binding fragment thereof to therapeutic polynucleotide that is of from about 1:1 to about 50:1. In some embodiments, a pharmaceutical composition described herein has a molar ratio of 3E10 antibody or variant thereof, or antigen-binding fragment thereof to therapeutic polynucleotide that is of from about 1:1 to about 30:1. In some embodiments, a pharmaceutical composition described herein has a molar ratio of 3E10 antibody or variant thereof, or antigen-binding fragment thereof to therapeutic polynucleotide that is of from about 1:1 to about 20:1. In some embodiments, a pharmaceutical composition described herein has a molar ratio of 3E10 antibody or variant thereof, or antigen-binding fragment thereof to therapeutic polynucleotide that is of from about 1:1 to about 10:1. In some embodiments, a pharmaceutical composition described herein has a molar ratio of 3E10 antibody or variant thereof, or antigen-binding fragment thereof to therapeutic polynucleotide that is of from about 1:1 to about 5:1.

In some embodiments, the molar ratio is related to the size of the nucleic acid (i.e., the therapeutic polynucleotide). For instance, longer polynucleotides are complexed at higher molar ratios and shorter polynucleotides are complexed at lower molar ratios.

In some embodiments, the size of the therapeutic polynucleotide is about 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 105 bp, 110 bp, 115 bp, 120 bp, 125 bp, 130 bp, 135 bp, 140 bp, 145 bp, 150 bp, 155 bp, 160 bp, 165 bp, 170 bp, 175 bp, 180 bp, 185 bp, 190 bp, 195 bp, 200 bp, 205 bp, 210 bp, 215 bp, 220 bp, 225 bp, 230 bp, 235 bp, 240 bp, 245 bp, 250 bp, 255 bp, 260 bp, 265 bp, 270 bp, 275 bp, 280 bp, 285 bp, 290 bp, 295 bp, 300 bp, 305 bp, 310 bp, 315 bp, 320 bp, 325 bp, 330 bp, 335 bp, 340 bp, 345 bp, 350 bp, 355 bp, 360 bp, 365 bp, 370 bp, 375 bp, 380 bp, 385 bp, 390 bp, 395 bp, 400 bp, 405 bp, 410 bp, 415 bp, 420 bp, 425 bp, 430 bp, 435 bp, 440 bp, 445 bp, 450 bp, 455 bp, 460 bp, 465 bp, 470 bp, 475 bp, 480 bp, 485 bp, 490 bp, 495 bp, 500 bp, 505 bp, 510 bp, 515 bp, 520 bp, 525 bp, 530 bp, 535 bp, 540 bp, 545 bp, 550 bp, or more and any range in between.

In some embodiments, the molar ratios disclosed herein are related to the size of the therapeutic polynucleotide as disclosed herein. For instance, longer polynucleotides are complexed at higher molar ratios and shorter polynucleotides are complexed at lower molar ratios.

In some embodiments, any molar ratio disclosed herein can be combined with any size of the therapeutic polynucleotide. Nonlimiting examples, include a pharmaceutical composition that has a molar ratio of 3E10 antibody to therapeutic polynucleotide that is 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, 200:1, 205:1, 210:1, 215:1, 220:1, 225:1, 230:1, 235:1, 240:1, 245:1, 250:1, or greater and any ranges in between, wherein the size therapeutic polynucleotide is about 10 pb, 15 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 105 bp, 110 bp, 115 bp, 120 bp, 125 bp, 130 bp, 135 bp, 140 bp, 145 bp, 150 bp, 155 bp, 160 bp, 165 bp, 170 bp, 175 bp, 180 bp, 185 bp, 190 bp, 195 bp, 200 bp, 205 bp, 210 bp, 215 bp, 220 bp, 225 bp, 230 bp, 235 bp, 240 bp, 245 bp, 250 bp, 255 bp, 260 bp, 265 bp, 270 bp, 275 bp, 280 bp, 285 bp, 290 bp, 295 bp, 300 bp, 305 bp, 310 bp, 315 bp, 320 bp, 325 bp, 330 bp, 335 bp, 340 bp, 345 bp, 350 bp, 355 bp, 360 bp, 365 bp, 370 bp, 375 bp, 380 bp, 385 bp, 390 bp, 395 bp, 400 bp, 405 bp, 410 bp, 415 bp, 420 bp, 425 bp, 430 bp, 435 bp, 440 bp, 445 bp, 450 bp, 455 bp, 460 bp, 465 bp, 470 bp, 475 bp, 480 bp, 485 bp, 490 bp, 495 bp, 500 bp, 505 bp, 510 bp, 515 bp, 520 bp, 525 bp, 530 bp, 535 bp, 540 bp, 545 bp, 550 bp, or more and any ranges in between.

The compositions of the present disclosure can be formulated for, and subsequently administered by, one of many common administrative routes. In some embodiments, the pharmaceutical composition is formulated for parenteral administration. In some embodiments, the parenteral administration is intramuscular administration, intravenous administration, or subcutaneous administration.

However, in other embodiments, the compositions described herein containing a complex formed between (i) a therapeutic polynucleotide, and (ii) a 3E10 antibody or variant thereof, or antigen-binding fragment thereof are administered by direct injection into the tumor. Accordingly, in some embodiments, the pharmaceutical composition is formulated for direct injection into the tumor. In some embodiments, the pharmaceutical composition is formulated for intrathecal administration (e.g., lumbar intrathecal administration or cisternal intrathecal administration), intracerebroventricular administration, or intraparenchymal administration.

In some embodiments, the therapeutic polynucleotides of the compositions described herein include one or more non-canonical nucleotides, e.g., to improve the stability and/or half-life of the mRNA in vivo. Examples of non-canonical nucleotides suitable for inclusion in the molecules described herein are described in U.S. Pat. No. 9,181,319, the content of which is incorporated herein by reference.

### Co-Therapies

In some embodiments, one or more of the following therapies can be used together with the foregoing antibody/polynucleotide treatment.

### Chemotherapy

Chemotherapy refers to the use of any drug to treat any disease. Chemotherapy is considered a systemic treatment because the drugs typically travel throughout the body, and can kill, for example, cancer cells that have spread (metastasized) to parts of the body far away from the original (primary) tumor or other source of the disease.

### Hormone Therapy

Hormone therapy is a cancer treatment that slows or stops the growth of cancer that uses hormones to grow. Certain cancers rely on hormones to grow. In these cases, hormone therapy may slow or stop their spread by blocking the body's ability to produce these particular hormones or changing how hormone receptors behave in the body. Hormone therapy is available via pills, injection or surgery that removes hormone-producing organs, for example, the ovaries in women and the testicles in men. It is typically recommended along with other cancer treatments.

### Immunotherapy

Immunotherapy is the treatment of disease by activating or suppressing the immune system. Immunotherapies designed to elicit or amplify an immune response are classified as activation immunotherapies, while immunotherapies that reduce or suppress are classified as suppression immunotherapies. Immunotherapy is a treatment that uses a person's own immune system to fight, for example cancer.

In some embodiments, the administered immunotherapy may suppress PD-1 protein. It has been shown that expression of PD-1 and PD-L1 was enhanced in cancer cells and related to defective immune responses. Furthermore, have suggested that two key immune checkpoint molecules are important therapeutic targets for cancer and infectious disease treatment.

In some embodiments, the antagonist monoclonal antibodies against PD-1 are OPDIVO® (nivolumab) and KEYTRUDA® (pembrolizumab) and are described in U.S. Pat. Nos. 7,595,048 and 8,952,136, each of which are hereby incorporated by reference in their entireties.

In some embodiments, the antagonist monoclonal antibodies against PD-L1 are BAVENCIO® (avelumab), TECENTRIQ® (atezolizumab), or Imfinzi® (durvalumab) and are described in U.S. Pat. Nos. 11,058,769, 8,952,136, and 8,779,108, each of which are hereby incorporated by reference in their entireties.

### Adoptive Cell Therapy

Adoptive cell therapy, also known as cellular immunotherapy, is a form of treatment that uses the cells of a person's immune system to eliminate cancer. Some of these approaches involve directly isolating a person's own immune cells and simply expanding their numbers, whereas other approaches involve genetically engineering a person's immune cells (via gene therapy) to enhance their cancer-fighting capabilities. Examples of adoptive-cell therapy include Tumor-Infiltrating Lymphocyte (TIL) Therapy, Engineered T Cell Receptor (TCR) Therapy, Chimeric Antigen Receptor (CAR) T Cell Therapy and Natural Killer (NK) Cell Therapy.

### Radiation Therapy

Radiation therapy is a type of cancer treatment that uses beams of intense energy to kill cancer cells. Radiation therapy frequently uses X-rays, but protons or other types of energy can also be used. While both healthy and cancerous cells are damaged by radiation therapy, the goal of radiation therapy is to destroy as few normal cells as possible.

### Surgery

Surgery, when used to treat cancer, is a procedure in which cancer is physically removed from the body. Frequently, a tumor and some nearby lymph nodes are removed.

Unless defined otherwise, all technical and scientific terms used herein have the same meanings as commonly understood by one of skill in the art to which the disclosed invention belongs. Publications cited herein and the materials for which they are cited are specifically incorporated by reference.

### EXAMPLES

The present invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

With respect to the experiments below, standard 3E10 sequence was used except wherein noted to be the D31N variant. Both standard 3E10 and the D31N variant were used as full length antibodies.

### Example 1: 3E10 Mediates mRNA Delivery to Tumors In Vivo

10 ug of mRNA encoding GFP was mixed with 0.1 mg of 3E10 for 15 minutes at room temperature. mRNA complexed to 3E10 was injected systemically to BALB/c mice bearing EMT6 flank tumors measuring 100 mm³. 20 hours after treatment, tumors were harvested and analyzed for mRNA expression (GFP) using IVIS imaging.

3E10-mediated delivery of mRNA resulted in significantly higher levels of GFP expression in the tumor compared to freely injected mRNA, which did not yield any GFP expression in the tumor. There was no detectable expression of GFP in any of the normal tissues examined with either treatment, including liver, spleen, heart, and kidney. The results indicate robust delivery of mRNA into tumors, with functional translation and expression.

### Example 2: 3E10 Mediates siRNA Delivery In Vivo

40 ug of fluorescently labeled siRNA was mixed with increased doses of 3E10 (0.25, 0.5, and 1 mg) for 15 minutes at room temperature. siRNA complexed to 3E10 was injected systemically to BALB/c mice bearing EMT6 flank tumors measuring 100 mm³. 20 hours after treatment, tumors were harvested and analyzed for siRNA delivery using IVIS imaging.

40 ug of fluorescently labeled siRNA was mixed with 1 mg 3E10 or 0.1 mg of the D31N variant of 3E10 for 15 minutes at room temperature. siRNA complexed to 3E10 was injected systemically to BALB/c mice bearing EMT6 flank tumors measuring 100 mm³. 20 hours after treatment, tumors were harvested and analyzed for siRNA delivery using IVIS imaging.

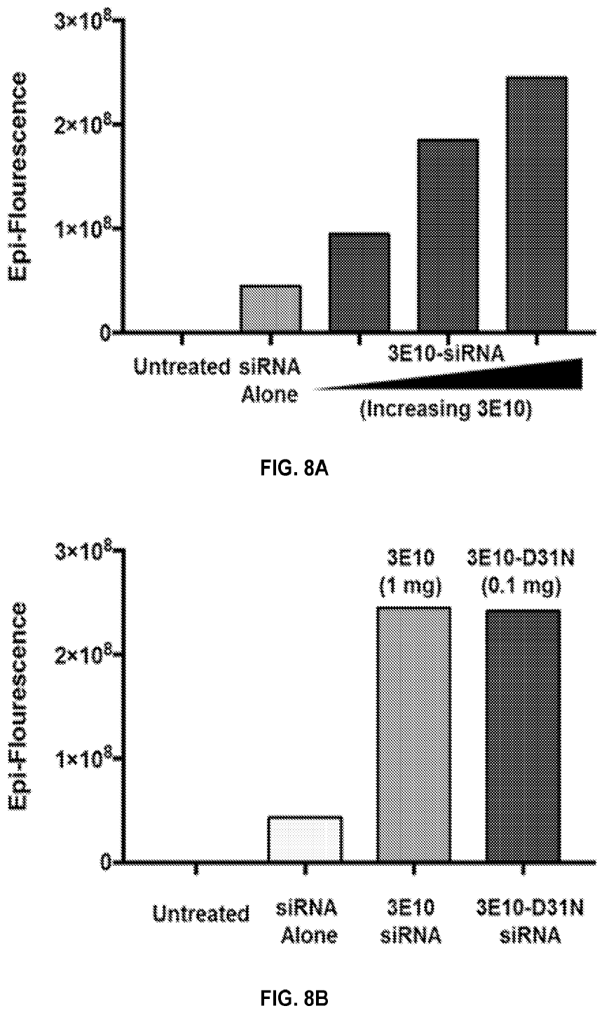

As shown in FIG. 8A, increasing doses of 3E10 result in higher accumulation of siRNA in tumors. As shown in FIG. 8B, a tenfold lower dose of D31N 3E10 resulted in similar levels of siRNA delivery as 3E10.

### Example 3: Localization of 3E10 to Tumors In Vivo Following Intravenous Administration

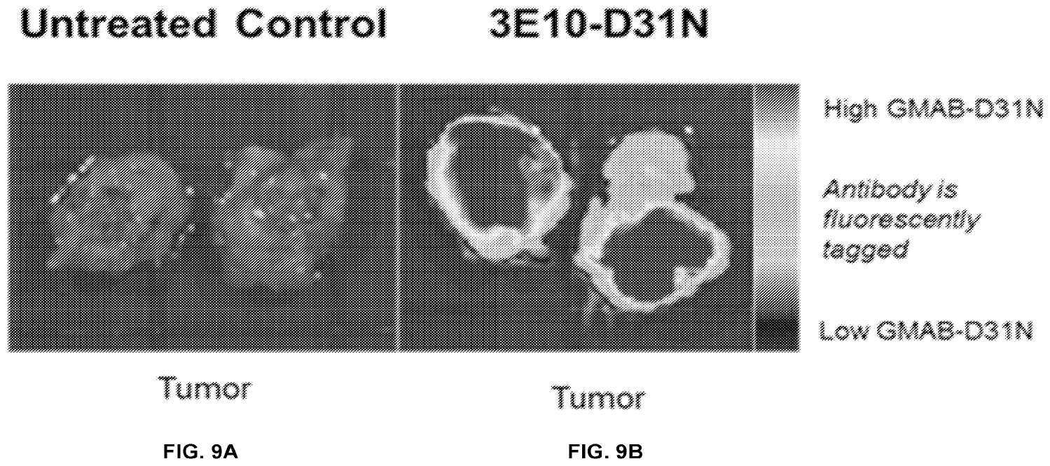

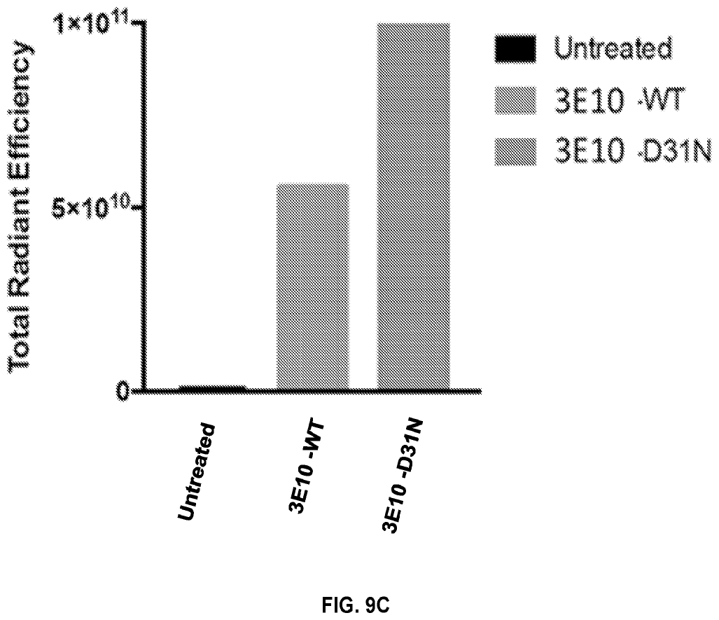

Mice bearing flank syngeneic colon tumors (CT26) were injected intravenously with 200 μg of 3E10, WT or D31N, labeled with VivoTag680 (Perkin Elmer). 24 hours after injection, tumors were harvested and imaged by IVIS (Perkin Elmer) (FIG. 9A-9B). Quantification of tumor distribution demonstrated that 3E10-D31N had higher accumulation in tumors when compared to 3E10-WT (FIG. 9C).

Distribution of ssDNA non-covalently associated with 3E10 was investigated. Mice bearing flank syngeneic colon tumors (CT26) were injected intravenously with 200 ug of 3E10, WT or D31N, mixed with 40 ug of labeled ssDNA (IR680). 24 hours after injection, tumors were harvested and imaged by IVIS (Perkin Elmer) (FIG. 10A-10C). Quantification of tissue distribution demonstrated that delivery of ssDNA by 3E10-D31N resulted in higher tumor accumulation when compared to 3E10-WT (FIG. 10D).

### Example 4: 3E10-Mediates Delivery of RIG-I Ligand, and Stimulation of RIG-I Activity

RIG-I reporter cells (HEK-Lucia RIG-I, Invivogen) were seeded at 50,000 cells per well and treated with RIG-I ligands (1 ug) or ligands complexed to 3E10-D31N (20 ug). This assay uses a cell line with a luciferase reporter that is activated when there is induction of interferons.

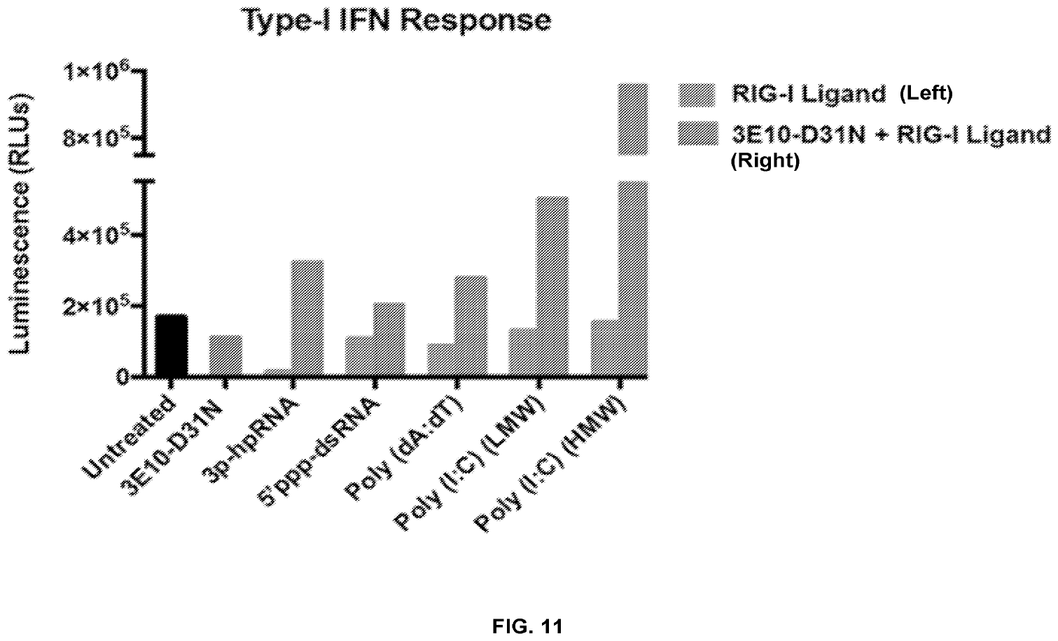

In all cases, RIG-I ligands alone did not stimulate IFN-γ secretion. Delivery of RIG-ligands with 3E10-D31N, however, stimulated IFN-γ secretion above controls, with the highest secretion observed for poly (I:C), both low and high molecular weight (LMW and HMW), as illustrated in FIG. 11.

### Example 5: 3E10-D31N+Poly (I:C) Mediates Melanoma Cell Death

Mouse melanoma cells (B16) were treated with buffer control, poly (I:C), 3E10 (WT or D31N), or poly (I:C) complexed with 3E10 (WT or D31N). 24 hours after treatment, cell viability was measured by CellTiter-Glo. In all cases, complexing of poly (I:C) with 3E10 (WT or D31N) reduced cell viability relative to controls (FIGS. 12A-12B).

### Example 6: Localization of 3E10-D31N to a Medulloblastoma Tumor Model In Vivo

To test whether 3E10-D31N (GMAB^{D31N}) can cross the blood-brain barrier and localize to a tumor in the central nervous system, mice bearing human models of a medulloblastoma tumor were intravenously administered fluorescently-labeled 3E10-D31N. Briefly, nude mice were implanted with a human model of medulloblastoma (DAYO) via intracisternal injection. Tumor growth was measured prior to start of treatment via luciferase expression. Mice were subsequently injected intravenously with a fluorescently labeled 3E10-D31N (200 ug). Tumor accumulation of labeled 3E10-D31N was monitored by IVIS imaging (FIG. 13A) and quantified (FIG. 13B).

As shown in FIG. 13A, 3E10-D31N accumulated and was retained in the CNS of mice for at least 8 days, both in the brain and at the spinal cord. No fluorescence was observed in control mice not administered the labeled 3E10-D31N. This is further shown quantitatively in FIG. 13B. These results demonstrate that 3E10-D31N is capable of crossing the blood-brain barrier (BBB) and localizing to CNS tumors and sites of possible metastasis following systemic administration.

### Example 7: Treatment of Medulloblastoma Tumor with a RIG-I Polynucleotide Agonist and 3E10-D31N Complex

To test whether 3E10-D31N can deliver therapeutically effective amounts of a RIG-I polynucleotide agonist to a cancer of the central nervous system, mice bearing human models of a medulloblastoma tumor were intravenously administered a complex of a 5′ triphosphate hairpin RNA agonist of RIG-I derived from the influenza A (H1N1) virus (3p-hpRNA; Invitrogen having the sequence:

(SEQ ID NO: XX)
5′ -pppGGAGCAAAAGCAGGGUGACAAAGACAUAAUGGAUCCAAACACU
GUGUCAAGCUUUCAGGUAGAUUGCUUUCUUUGGCAUGUCCGCAAAC-3′.

Briefly, nude mice were implanted with a human model of medulloblastoma (DAYO) via intracisternal injection. Tumor growth was measured prior to start of treatment via luciferase expression. Mice were subsequently treated with PBS control (200 ul), 3E10-D31N (GMAB^{D31N}) (550 ug), or 3E10-D31N (550 ug) complexed to the 3p-hpRNA RIG-I agonist (25 ug) (GMAB^{D31N}/3pRNA). At days 1, 3, 4, 8, 9, and 14, tumor growth was monitored by luciferase expression of the DAYO cells, as illustrated in FIGS. 14A and 14B, and as quantitated in FIG. 14C. As shown in FIG. 14, the tumor burden was significantly reduced at all time points measured in the GMAB^{D31N}/3pRNA treated mice, compared to control mice administered PBS or GMAB^{D31N} alone.

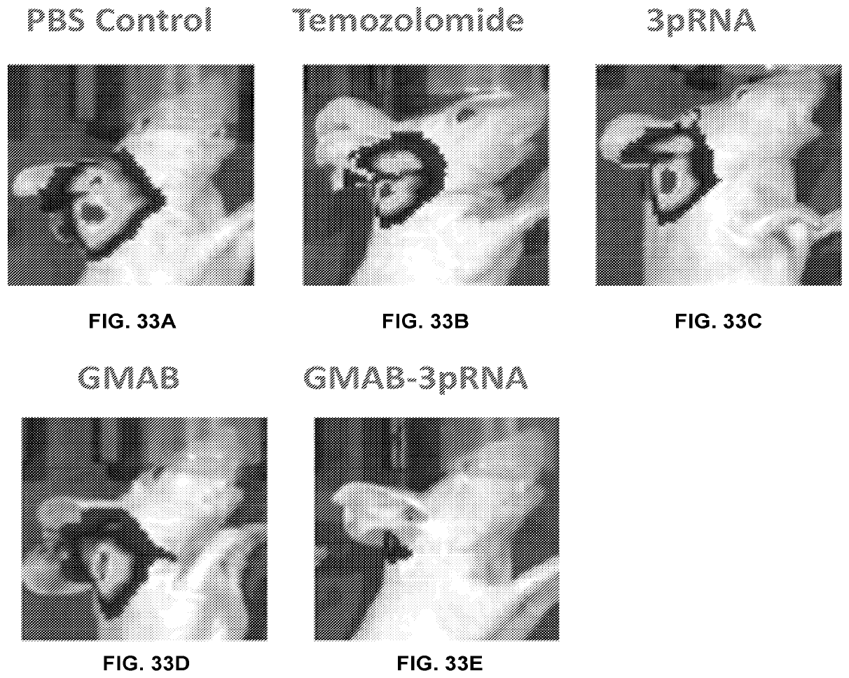

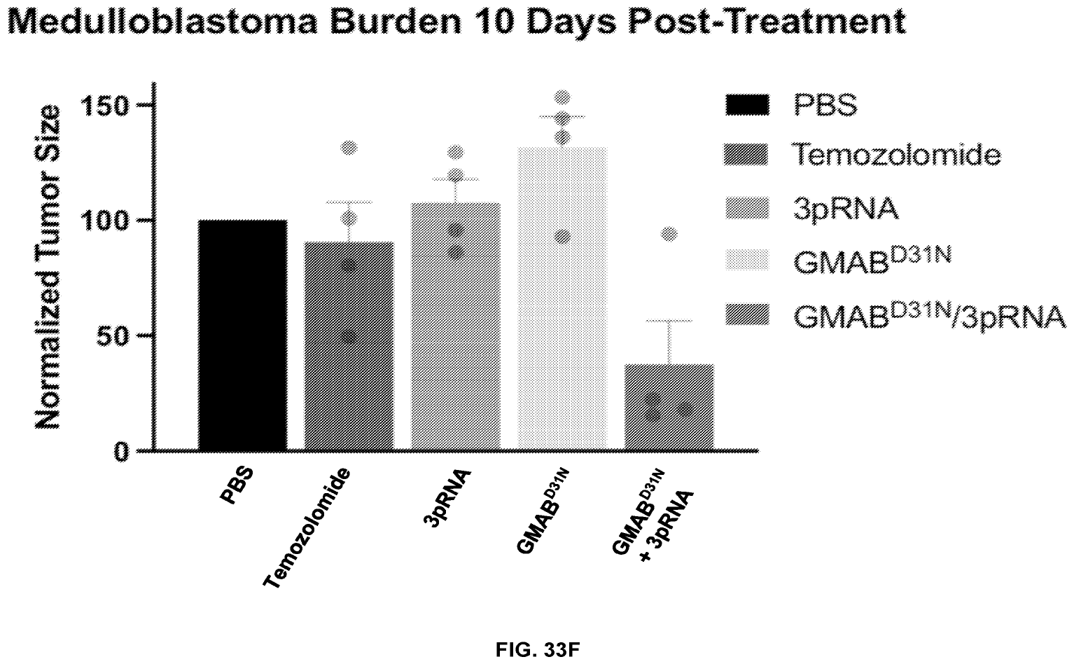

The tumor was imaged and tumor volume determined at day 10, following administration of a single dose of PBS control, temozolomide, 3p-hpRNA, 3E10-D31N alone, and 3E10-D31N/3p-hpRNA complex. Representative images are shown in FIGS. 33A-33E, while average tumor burden across each cohort is illustrated in FIG. 33F.

Upon termination of the mice at day 14 post administration, the brain and spinal cords of control and treated mice were imaged for luciferase expression in DAYO cells. As shown in FIG. 14D, a single dose of GMAB^{D31N}/3pRNA, but not the PBS control, significantly reduced tumor burden in the brain of the mice. Further, as illustrated in FIG. 14E, a single dose of GMAB^{D31N}/3pRNA, but not the PBS control, significantly prevents metastasis of the cancer to the spinal cord.

### Example 8: Molecular Modeling of 3E10 and Engineered Variants Thereof

WT HEAVY CHAIN scFv SEQUENCE
(SEQ ID NO: XX)
E VQLVESGGGL VKPGGSRKLS CAASGFTFSD YGMHWVRQAP
EKGLEWVAYI SSGSSTIYYA DTVKGRFTIS RDNAKNTLFL
QMTSLRSEDT AMYYCARRGL LLDYWGQGTT LTVS
LIGHT CHAIN scFv SEQUENCE
(SEQ ID NO: XX)
D IVLTQSPASL AVSLGQRATI SCRASKSVST SSYSYMHWYQ
QKPGQPPKLL IKYASYLESG VPARFSGSGS GTDFTLNIHP
VEEEDAATYY CQHSREFPWT FGGGTKLEIK RADAAPGGGG
SGGGGSGGGGS

Molecular modeling of 3E10 (Pymol) revealed a putative Nucleic Acid Binding pocket (NAB1) (FIGS. 15A-15B). Mutation of aspartic acid at residue 31 of CDR1 to asparagine increased the cationic charge of this residue and enhanced nucleic acid binding and delivery in vivo (3E10-D31N). Mutation of aspartic acid at residue 31 of CDR1 to arginine (3E10-D31R), further expanded the cationic charge while mutation to lysine (3E10-D31K) changed charge orientation (FIG. 15A).

NAB1 amino acids predicted from molecular modeling have been underlined in the heavy and light chain sequences above. FIG. 15B is an illustration showing molecular modeling of 3E10-scFv (Pymol) with NAB1 amino acid residues illustrated with punctate dots.

### Example 9: 3E10 (D31N) Protects mRNA Against RNA Degradation

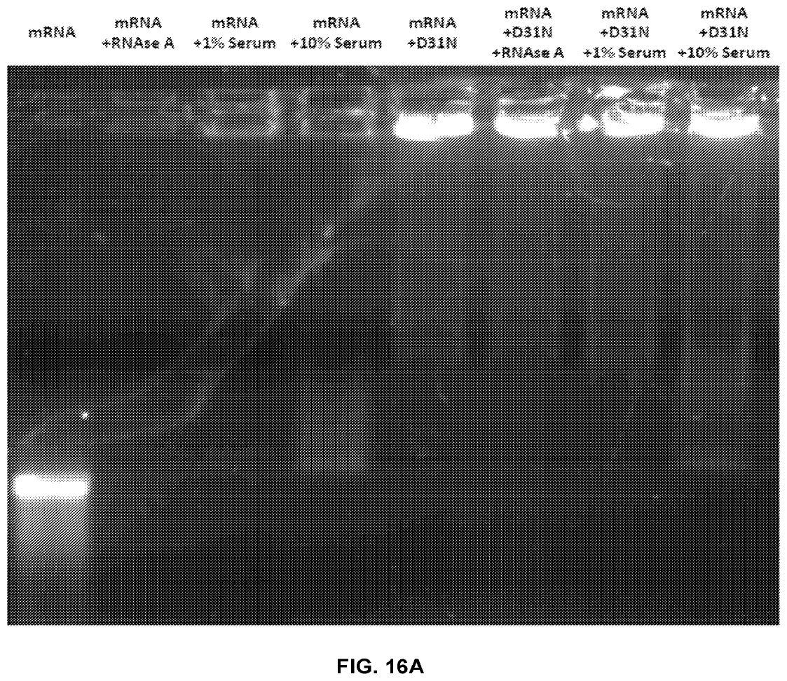

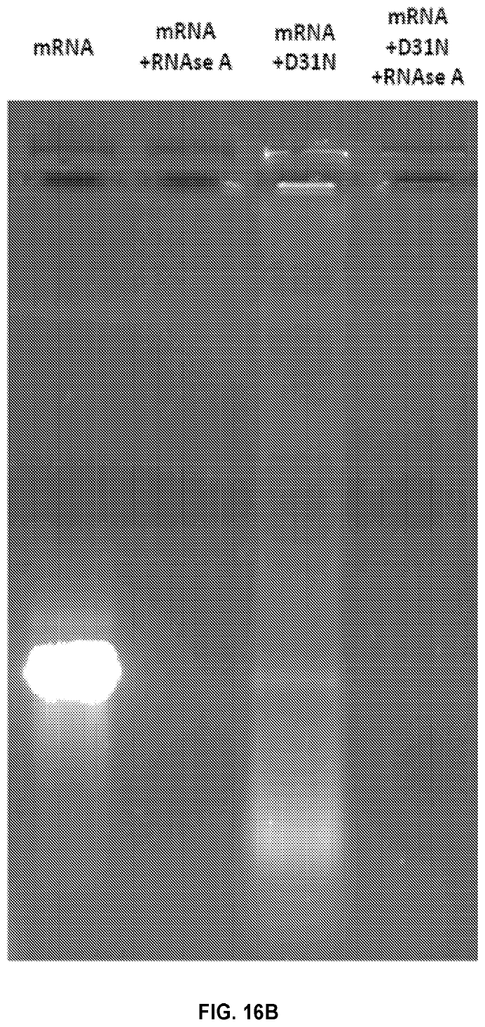

It was next investigated whether complexing mRNA with 3E10 (D31N) would protect the mRNA from degradation. Briefly, complexes of 3E10 (D31N) and mRNA encoding green fluorescent protein, a luciferase, having the sequence GFP_mRNA shown below as (SEQ ID NO:XX), were formed by mixing 3E10 (D31N) and mRNA at a 20:1 molar ratio. The free mRNA and the 3E10-mRNA complex were then incubated with 1% serum, 10% serum, or 16 μg/mL RNAse A for 10 minutes at 37° C. Gel electrophoresis analysis of the reactions was performed (FIG. 16A). As shown in FIG. 16A, free mRNA was degraded by incubation with each of 1% serum, 10% serum, and RNAse A. However, no apparent RNA degradation was observed when the complexed mRNA was incubated with any of 1% serum, 10% serum, or RNAse A, suggesting that 3E10 (D31N) protects mRNA from degradation.

(SEQ ID NO: XX)
GFP_mRNA -
AUGGUGAGCAAGGGCGAGGAGCUGUUCACCGGGGUGGUGCCCAUCCUGGU
CGAGCUGGACGGCGACGUAAACGGCCACAAGUUCAGCGUGUCCGGCGAGG
GCGAGGGCGAUGCCACCUACGGCAAGCUGACCCUGAAGUUCAUCUGCACC
ACCGGCAAGCUGCCCGUGCCCUGGCCCACCCUCGUGACCACCCUGACCUA
CGGCGUGCAGUGCUUCAGCCGCUACCCCGACCACAUGAAGCAGCACGACU
UCUUCAAGUCCGCCAUGCCCGAAGGCUACGUCCAGGAGCGCACCAUCUUC
UUCAAGGACGACGGCAACUACAAGACCCGCGCCGAGGUGAAGUUCGAGGG
CGACACCCUGGUGAACCGCAUCGAGCUGAAGGGCAUCGACUUCAAGGAGG
ACGGCAACAUCCUGGGGCACAAGCUGGAGUACAACUACAACAGCCACAAC
GUCUAUAUCAUGGCCGACAAGCAGAAGAACGGCAUCAAGGUGAACUUCAA
GAUCCGCCACAACAUCGAGGACGGCAGCGUGCAGCUCGCCGACCACUACC
AGCAGAACACCCCCAUCGGCGACGGCCCCGUGCUGCUGCCCGACAACCAC
UACCUGAGCACCCAGUCCGCCCUGAGCAAAGACCCCAACGAGAAGCGCGA
UCACAUGGUCCUGCUGGAGUUCGUGACCGCCGCCGGGAUCACUCUCGGCA
UGGACGAGCUGUACAAGUAA.

Next, it was investigated whether mRNA complexed at lower molar ratios were also protected against RNA degradation. Briefly, complexes of 3E10 (D31N) and mRNA encoding green fluorescent protein (GFP_mRNA; SEQ ID NO:XX) were formed by mixing 3E10 (D31N) and mRNA at a 2:1 molar ratio. The free mRNA and the 3E10-mRNA complex were then incubated with RNAse A under the conditions described above. Gel electrophoresis analysis of the reactions was performed (FIG. 16B). As shown in FIG. 16B, free mRNA was completely degraded by incubation with RNAse A. However, complexing the mRNA with 3E10 (D31N) at a 2:1 molar ratio resulted in some protection of the mRNA against degradation, as indicated by the presence of an RNA signal in the well, indicating the presence of intact 3E10 (D31N)-mRNA complex. The protection provided at a 2:1 molar ratio appears to be less than the protection afforded the mRNA when complexed at a 20:1 molar ratio.

### Example 10: 3E10 (D31N) Protects mRNA Against RNA Degradation in a Size-Dependent Fashion

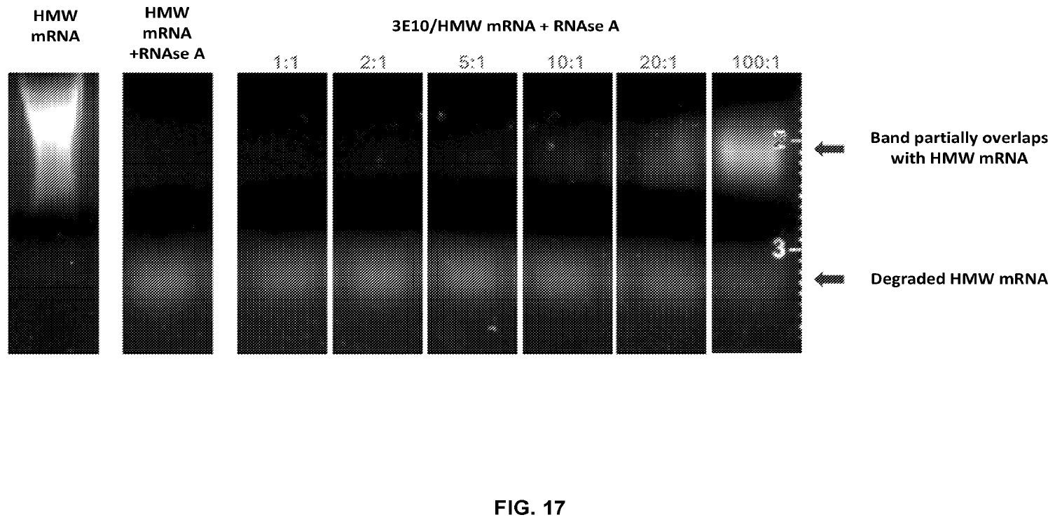

It was investigated whether 3E10-D31N would also protect larger mRNA molecules from enzymatic degradation when complexed, and whether larger stochiometric amounts of 3E10-D31N were necessary. Briefly, complexes of 3E10-D31N and a 14 kb mRNA (HMW mRNA) encoding a large protein, were formed by mixing 3E10-D31N and mRNA at 1:1, 2:1, 5:1, 10:1, 20:1, and 100:1 molar ratios. The free mRNA and the 3E10-mRNA complexes were then incubated with 6 μg/mL RNAse A for 10 minutes at 37° C. with the addition proteinase K to facilitate protein degradation. FIG. 17 shows agarose gel electrophoresis analysis of the protection assays. As shown in FIG. 17, free HMW mRNA, as well as HMW mRNA complexed at 1:1, 2:1, 5:1, and 10:1 molar ratios (3E10:mRNA) was completely degraded by incubation with RNAse A. However, as shown in FIG. 17, complexing the HMW mRNA with 3E10 at a molar ratio of 20:1 and 100:1 afforded increasing protection of the mRNA from degradation by RNAse A, as indicated by bands migrating at a similar distance as undegraded mRNA on the gel. These results, coupled with those of Example 9, suggest that 3E10 protects polynucleotides in a size-dependent manner.

### Example 11: 3E10-D31N Mediated Delivery of RIG-I Ligand Induces Type-I IFN Response in THP-1 Monocytes

It was investigated whether 3E10-D31N mediated delivery of a RIG-I stimulating ligand into monocytic cells derived from an acute monocytic leukemia effectively induces a Type-I IFN response characteristic of immunotherapy. Briefly, THP-1 monocytes were seeded into wells and incubated in DMEM supplemented with 20% FBS and 1% P/S at 20,000 cells/well. Cells were then treated with PBS (control), the 3p-hpRNA RIG-I agonist alone (1 ug/well), increasing amounts of 3E10-D31N (GMAB) alone, and 3E10-D31N/3p-hpRNA (1 ug 3p-hpRNA/well) complexes prepared in increasing molar ratios (3E10:3p-hpRNA), for 10 minutes, as indicated in FIG. 18. Sample media was sampled at indicated timepoints and measured for luciferase activity (reporter for type-I IFN). IFN response was monitored for three consecutive days, as shown in FIG. 18A (day 1), FIG. 18B (day 2), and FIG. 18C (day 3).

As shown in FIG. 18, exposure of the THP-1 monocytes with 3p-hpRNA RIG-I agonist alone resulted in a small (˜2-fold) increase in type-1 IFN response over the three days, consistent with poor cellular internalization of the naked polynucleotide agonist. Exposure of the THP-1 monocytes with 3E10-D31N alone resulted in an approximately 5-fold increase in type-1 IFN response in a dose independent fashion. In contrast, exposure of THP-1 monocytes with the 3E10-D31N/3p-hpRNA complexes increased type-1 IFN response 15-fold to 25-fold at day 2 and 10-fold to 20-fold at day three. These results are consistent with 3E10-D31N's ability to transport polynucleotides into cells. That the response was maintained into day 3 suggests that the 3p-hpRNA RIG-I agonist was released over time from 3E10-D31N. As shown in FIG. 18, the use of 3p-hpRNA complexes formed at higher molar ratios with 3E10-D31N, e.g., 3:1, 4:1, and 5:1, resulted in greater stimulation of the type-1 IFN response.

### Example 12: Exposure to Complexes Formed Between 3E10-D31N and a RIG-I Agonist Cause Cell Death in Human Brain Tumor Cells as Determined by Loss of Cell Viability

It was investigated whether 3E10-D31N mediated delivery of a RIG-I stimulating ligand into human brain tumor cells induces cellular death. Briefly, two cell lines derived from human brain tumors—U87 (primary glioblastoma) and U251 (glioblastoma multiforme)—were seeded into wells and then treated with PBS (control), the 3p-hpRNA RIG-I agonist alone (25 ug/well),3E10-D31N (GMAB) alone, and 3E10-D31N/3p-hpRNA complexes prepared at a molar ratio of 4:1 (3E10:3p-hpRNA).

Cell necrosis was measured after treatment using a CellTiter-Glo® Luminescent Cell Viability Assay (Promega), according to manufacture instructions (see, Technical Bulletin available online at the URL promega.com/resources/protocols/technical-bulletins/0/celltiter-glo-luminescent-cell-viability-assay-protocol/). As shown in FIG. 19, treatment with the 3p-hpRNA RIG-I agonist alone reduced cell viability slightly, while treatment with PBS or 3E10-D31N alone resulted in no detectable decrease in cell viability. In contrast, treatment with the 3E10-D31N3p-hpRNA complexes resulted in a significant reduction of cell viability in both brain tumor cell lines (approximately 15% and 20%, respectively), suggesting that 3E10-D31N-mediated delivery of a RIG-I agonist can be used to treat brain tumors.

### Example 13: Exposure to Complexes Formed Between 3E10-D31N and a RIG-I Agonist Cause Cell Death in Human Brain Tumor Cells as Determined by Loss of Cell Membrane Integrity

It was further investigated whether 3E10-D31N mediated delivery of a RIG-I stimulating ligand into human brain tumor cells induces cellular death. Briefly, two cell lines derived from human brain tumors—U87 (primary glioblastoma) and U251 (glioblastoma multiforme)—were seeded into wells and then treated with PBS (control), the 3p-hpRNA RIG-I agonist alone (1 ug/well), 3E10-D31N (GMAB) alone, and 3E10-D31N/3p-hpRNA complexes prepared at a molar ratio of 5:1 (3E10:3p-hpRNA).

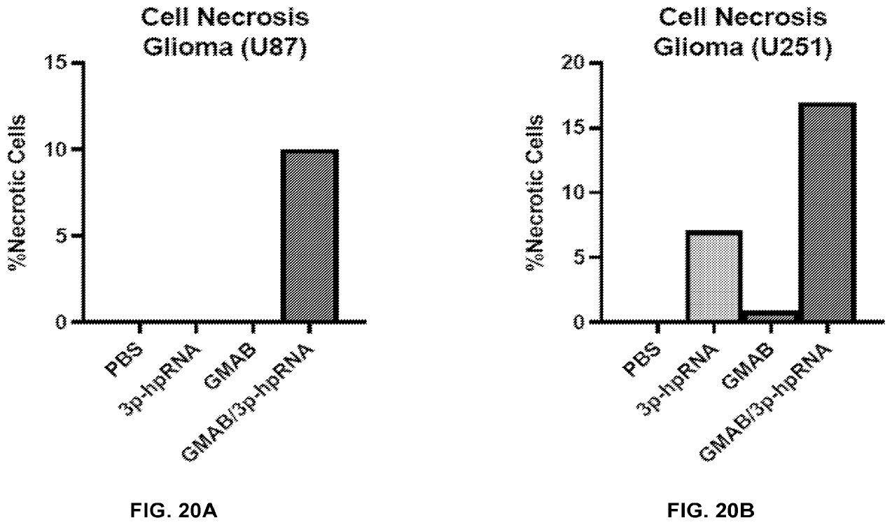

Cell necrosis was measured after treatment using a CellTox™ Green Cytotoxicity Assay (Promega), according to manufacture instructions (see, Technical Bulletin available online at the URL promega.com/-/media/files/resources/protocols/technical-manuals/101/celltox-green-cytotoxicity-assay-protocol.pdf). As shown in FIG. 20, treatment with the 3p-hpRNA RIG-I agonist alone induced cell death in a small percentage (approximately 7%) of U251 cells, but not in U87 cells. Similarly, treatment with PBS or 3E10-D31N alone resulted in no cell death. In contrast, treatment with the 3E10-D31N/3p-hpRNA complexes resulted in significant cell death in both brain tumor cell lines (approximately 10% and 17%, respectively), further suggesting that 3E10-D31N-mediated delivery of a RIG-I agonist can be used to treat brain tumors.

### Example 14: Exposure to Complexes Formed Between 3E10-D31N and a RIG-I Agonist Cause Cell Death in Colorectal Tumor Cells as Determined by Loss of Cell Membrane Integrity

It was further investigated whether 3E10-D31N mediated delivery of a RIG-I stimulating ligand into colorectal cancer cells induces cellular death. Briefly, two cell lines derived from human colorectal cancers—DLD1 (Dukes' type C colorectal adenocarcinoma) and DLD1 BRCA KO (Dukes' type C colorectal adenocarcinoma with a biallelic BRCA1 knock-out)—and two cell lines derived from murine colorectal cancers—MC38 (colon adenocarcinoma) and CT26 (colon adenocarcinoma)—were seeded into wells and then treated with PBS (control), the 3p-hpRNA RIG-I agonist alone (1 ug/well), 3E10-D31N (GMAB) alone, and 3E10-D31N/3p-hpRNA complexes prepared at a molar ratio of 5.1 (3E10:3p-hpRNA).

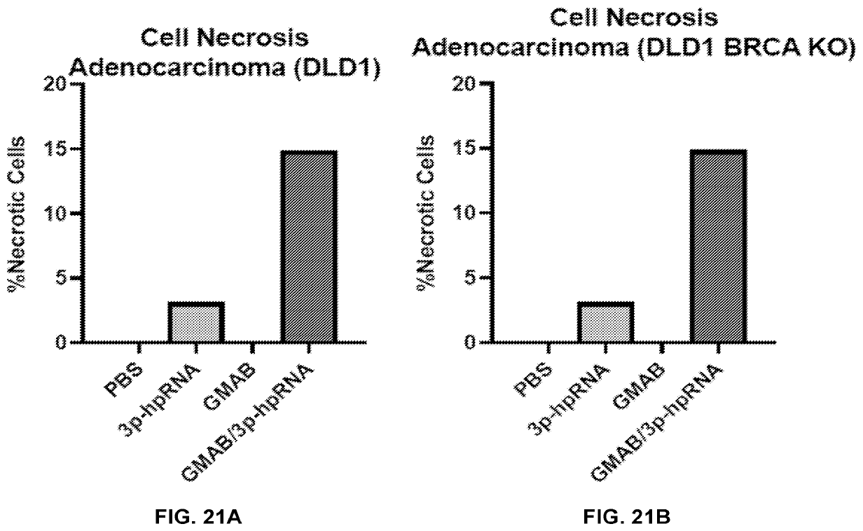

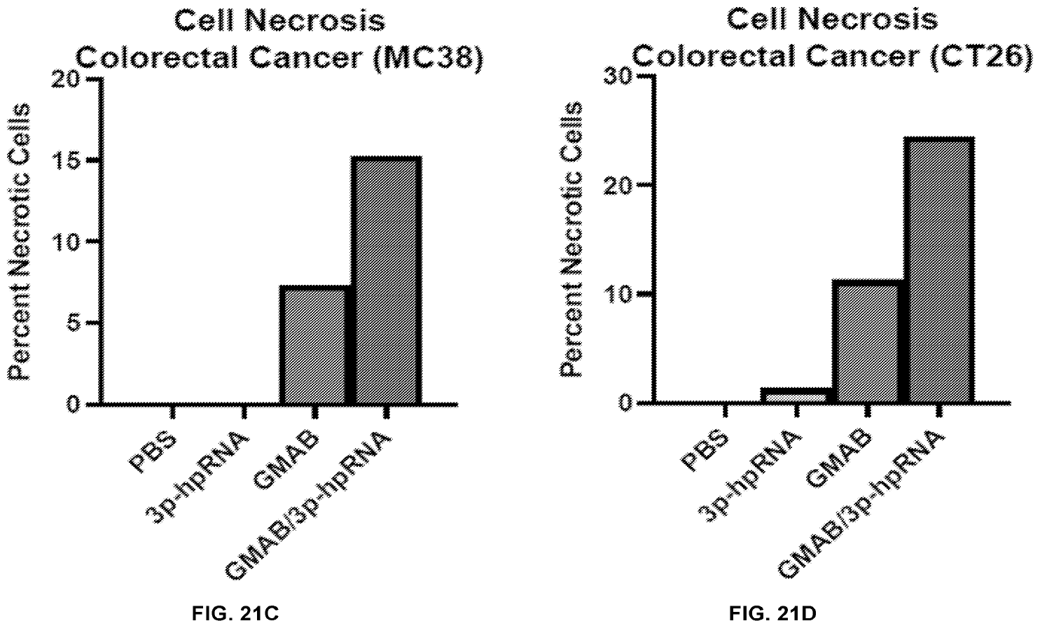

Cell necrosis was measured after treatment using a CellTox™ Green Cytotoxicity Assay (Promega), according to manufacture instructions (see, Technical Bulletin available online at the URL promega.com/-/media/files/resources/protocols/technical-manuals/101/celltox-green-cytotoxicity-assay-protocol.pdf). As shown in FIG. 21, treatment with the 3p-hpRNA RIG-I agonist alone induced cell death in a small percentage (approximately 3%) of the human cell lines, but not in the murine cell lines. Similarly, treatment with PBS alone resulted in no cell death. Treatment with 3E10-D31N alone induced cell death in moderate percentages (approximately 7% and 10%, respectively) of the murine cell lines, but not the human cell lines. In contrast, treatment with the 3E10-D31N/3p-hpRNA complexes resulted in significant cell death in both human (approximately 15% in FIGS. 21A and 21B) and murine (approximately 15% and 25% in FIGS. 21C and 21D, respectively) colorectal cancer cell lines, suggesting that 3E10-D31N-mediated delivery of a RIG-I agonist can be used to treat colorectal cancers.

### Example 15: Exposure to Complexes Formed Between 3E10-D31N and a RIG-I Agonist Cause Cell Death in Human Breast Cancer Cells as Determined by Loss of Cell Membrane Integrity

It was further investigated whether 3E10-D31N mediated delivery of a RIG-I stimulating ligand into human breast cancer cells induces cellular death. Briefly, a cell line derived from a human mammary triple-negative breast cancer (TNBC) adenocarcinoma (MDA-MB-231) was seeded into wells and then treated with PBS (control), the 3p-hpRNA RIG-I agonist alone, 3E10-D31N (GMAB) alone, or 3E10-D31N/3p-hpRNA complexes (3E10:3p-hpRNA).

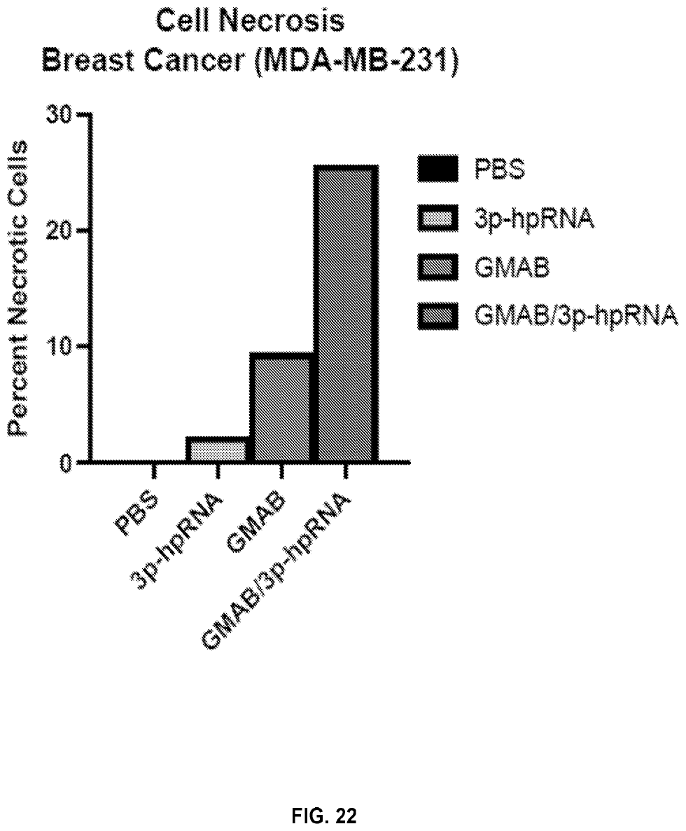

Cell necrosis was measured after treatment using a CellTox™ Green Cytotoxicity Assay (Promega), according to manufacture instructions (see, Technical Bulletin available online at the URL promega.com/-/media/files/resources/protocols/technical-manuals/101/celltox-green-cytotoxicity-assay-protocol.pdf). As shown in FIG. 22, treatment with the 3p-hpRNA RIG-I agonist alone induced cell death in a small percentage (approximately 2%) of breast cancer cells. Treatment with 3E10-D31N alone induced cell death in a moderate percentage (approximately 10%) of the breast cancer cells. In contrast, treatment with the 3E10-D31N/3p-hpRNA complexes resulted in significant cell death in the breast cancer cells (approximately 25%), suggesting that 3E10-D31N-mediated delivery of a RIG-I agonist can be used to treat breast cancers.

### Example 16: 3E10-D31N Mediated Delivery of RIG-I Ligand Induces an Increase in Proinflammatory IL-10 Production and a Decrease in Pro-Tumor IL-6 in Melanoma Cells

The mechanism of 3E10-D31N/3p-hpRNA RIG-I agonist induced cell death of melanoma cells was further investigated by determining changes in inflammatory cytokines produced in such cells. Briefly, B16 cells—a murine model of melanoma—were injected into mice, who were treated with PBS (control), the 3p-hpRNA RIG-I agonist alone, 3E10-D31N (GMAB) alone, 3E10-D31N/3p-hpRNA complexes, or an anti-CTLA-4 antibody.

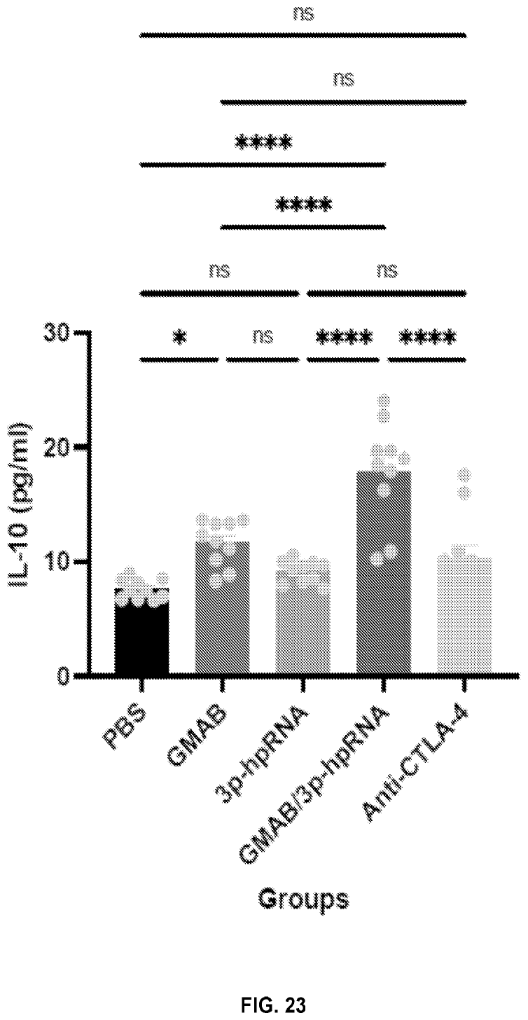

The levels of pro-inflammatory IL-10 and pro-tumor IL-6 were then determined. Given the known mechanism of action for RIG-I induced cell death, it was expected that if cell death was occurring as a result of delivery of the RIG-I agonist to the cells such that the agonist was released following delivery, IL-10 production would be increased and IL-6 production would be decreased. Briefly, following treatment, IL-10 and IL-6 levels in the tumor were determined by ELISA. As shown in FIG. 23, treatment with 3E10-D31N alone (*p<0.05), but not 3p-hpRNA alone (ns=not significant) induced a small increase in IL-10 production relative to PBS treatment. However, treatment with the 3E10-D31N/3p-hpRNA complexes resulted in significantly increased IL-10 production relative to all other cohorts (****p<0.00005). These results are consistent with RIG-I-induced mechanism of cell death. No increase in IL-10 production was observed following treatment with the anti-CLTA-4 antibody, consistent with a different mechanism of action for cell death mediated by the antibody.

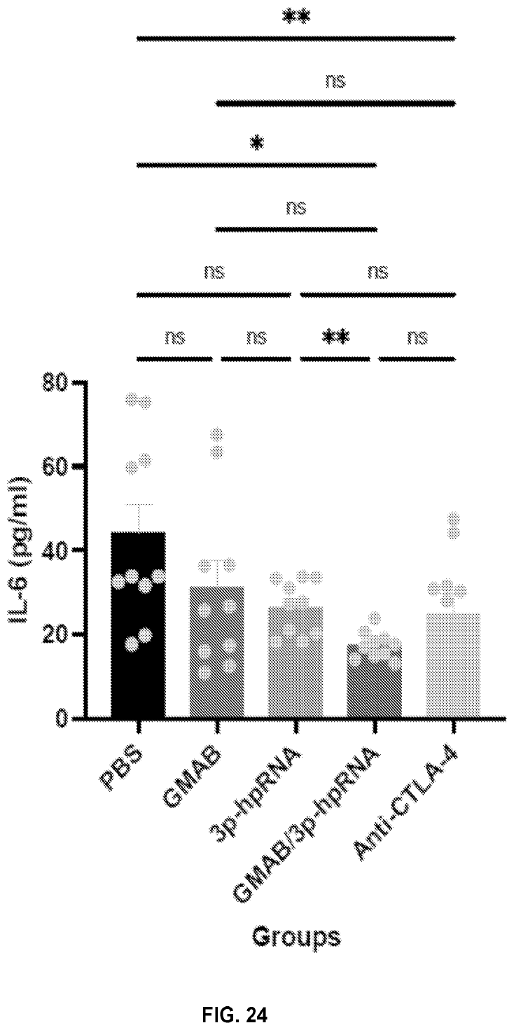

Similarly, as illustrated in FIG. 24, treatment with the 3E10-D31N/3p-hpRNA complexes resulted in significantly decreased IL-6 production relative to treatment with the PBS control (*p<0.05) and 3p-hpRNA treatment alone (**p<0.005), while treatment with 3E10-D31N or 3p-hpRNA alone resulted in no significant decrease in IL-6 production (ns=not significant). These results are again consistent with a RIG-I-induced mechanism of cell death. A decrease in IL-6 production was also observed following treatment with the anti-CTLA-4 antibody, consistent with inducement of cell death.

### Example 17: Localization of 3E10-D31N to Orthotopic Pancreatic Tumors In Vivo Following Intravenous Administration

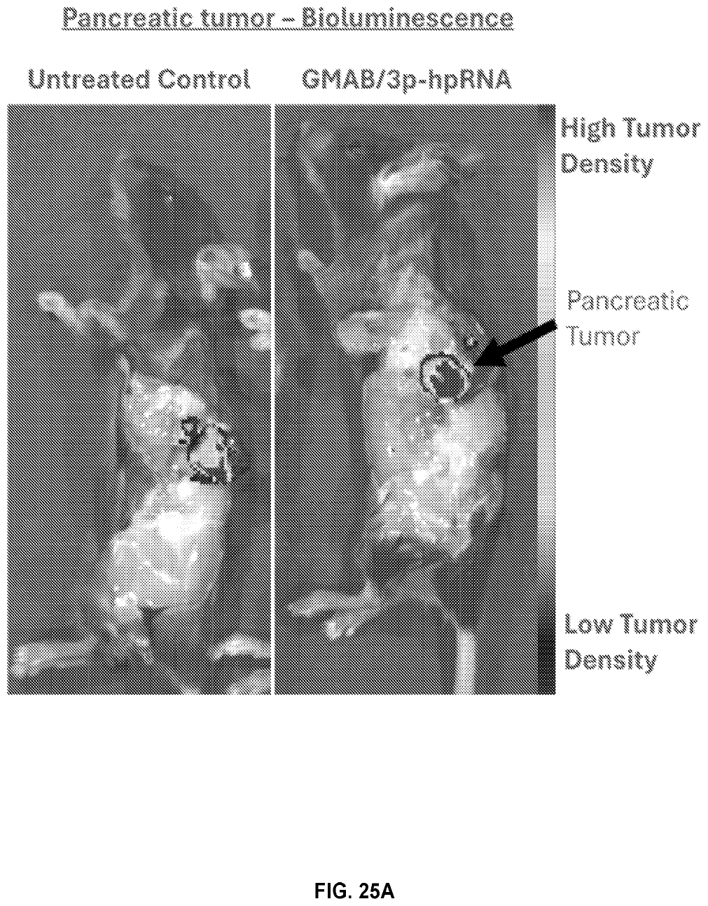

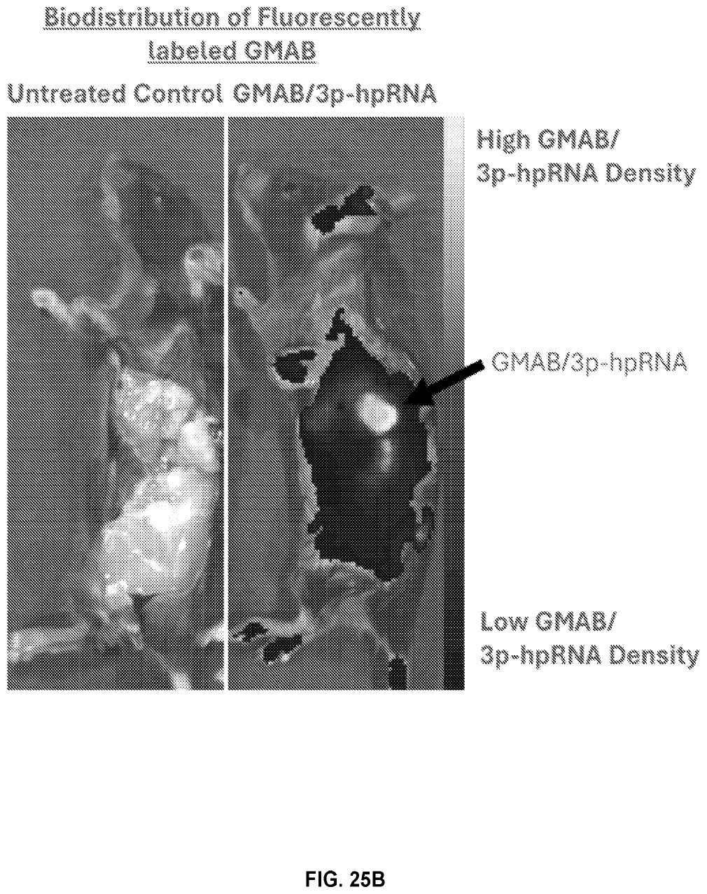

Pancreatic tumors are difficult to target for therapy in vivo. To investigate whether 3E10-D31N was capable of targeting and delivering a therapeutic polynucleotide to pancreatic cancer in vivo, mice bearing orthotopic pancreatic tumors were intravenously administered PBS (control), fluorescently-labeled 3E10-D31N (GMAB) alone, or fluorescently-labeled 3E10-D31N complexed with 3p-hpRNA (GMAB/3p-hpRNA; 5 mice per cohort). The orthotopic pancreatic tumors were visualized by luciferase expression in vivo (FIG. 25A; representative mice). As shown in FIG. 25B, fluorescently labeled 3E10-D31N complexed with 3p-hpRNA localized to the tumors in vivo, as indicated by the high density of fluorescence at the same location as the reporter luciferase expression in FIG. 25A.

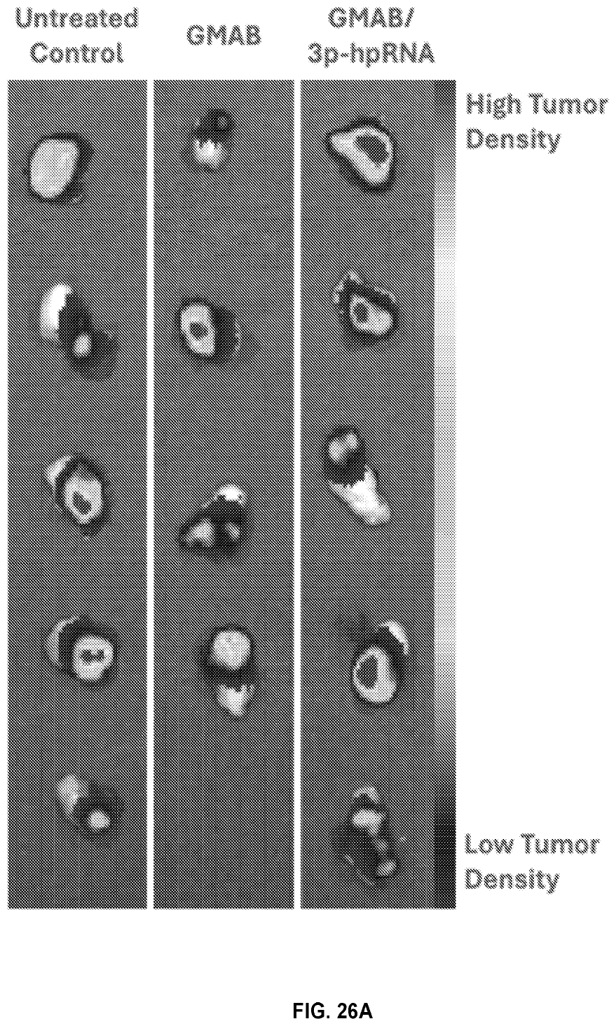

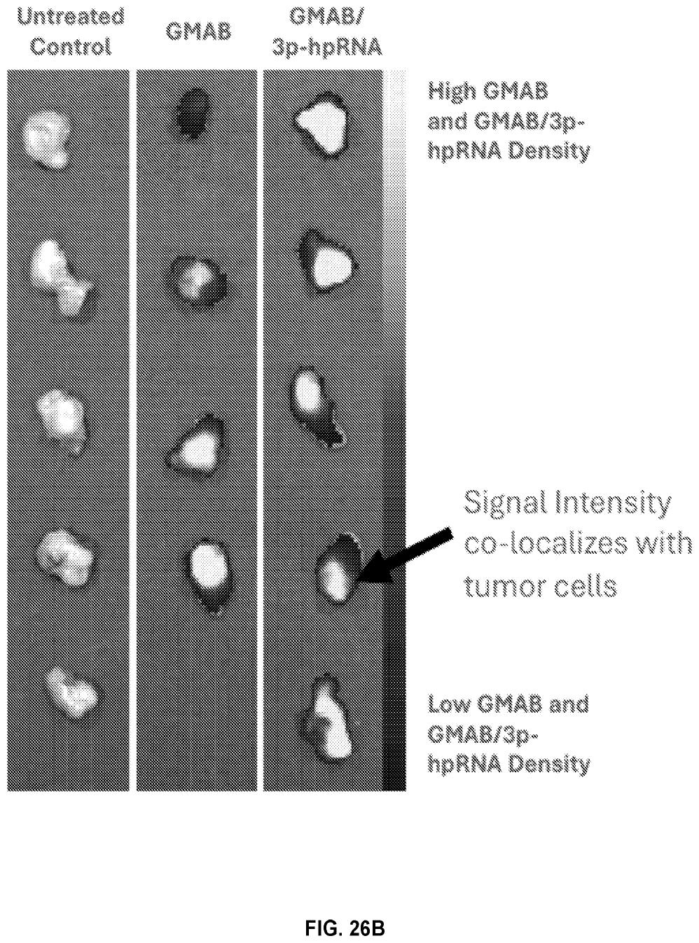

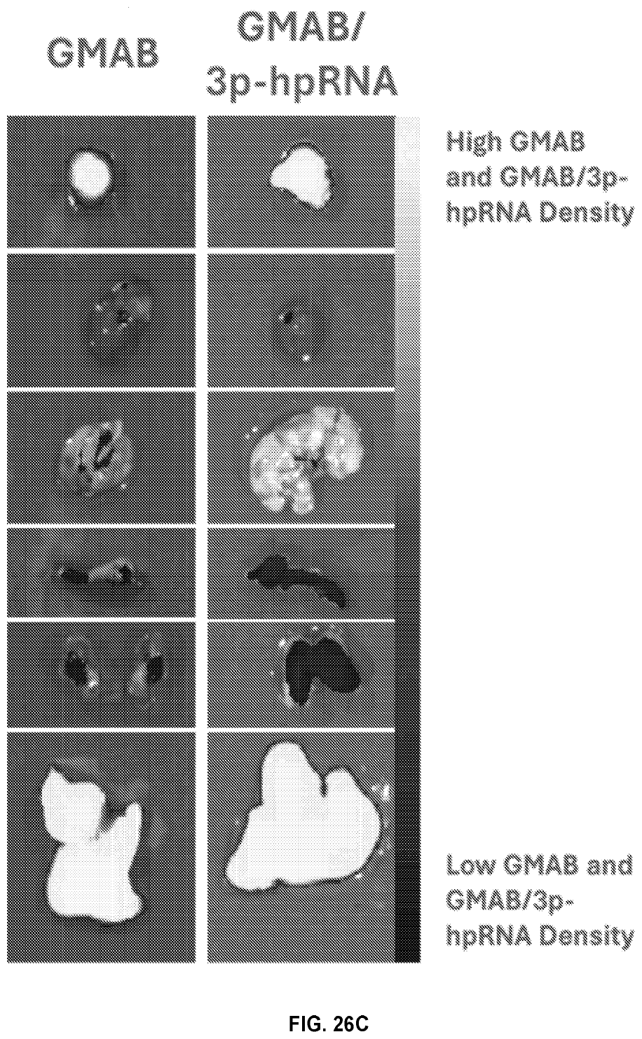

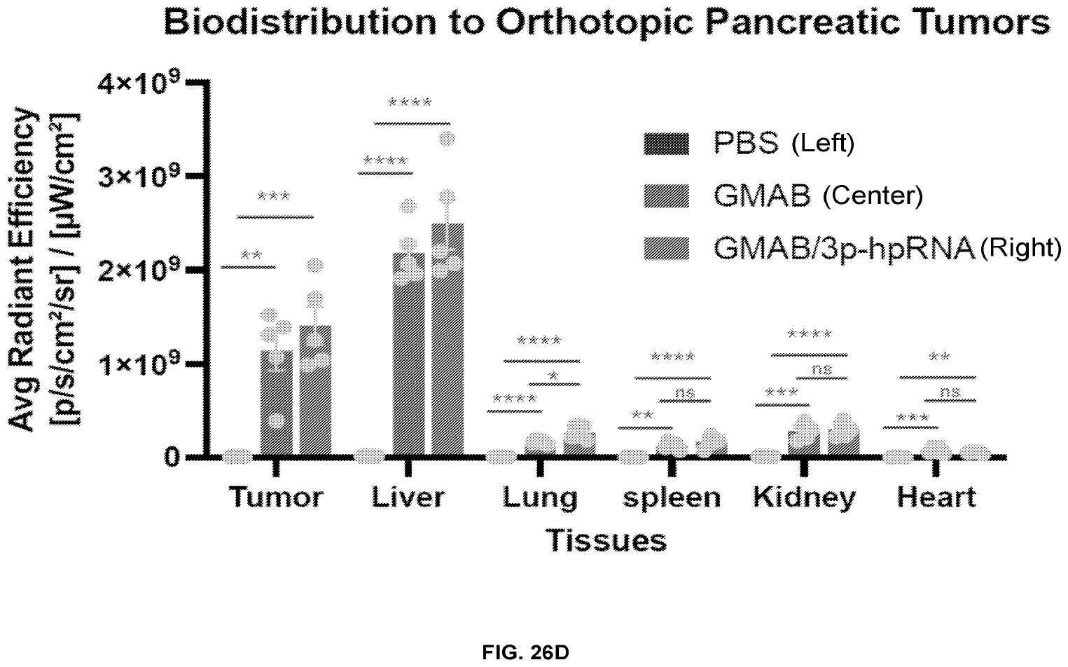

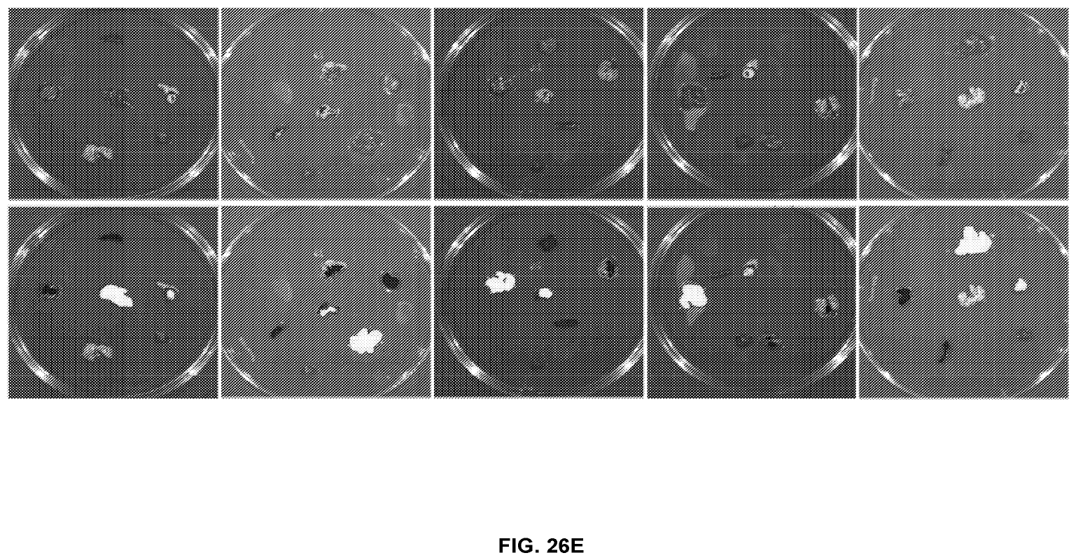

To further investigate whether 3E10-D31N was able to mediate delivery of the 3p-hpRNA into the cancerous tissue, the orthotopic pancreatic tumors were resected from each mouse and imaged for luciferase expression (confirming tumor tissue) and fluorescence (visualizing 3E10). FIG. 26A shows that tumor tissue was resected from each mouse. As shown in FIG. 26B, the resected tumor from mice administered fluorescently labeled 3E10-D31N alone (middle column) and 3E10-D31N complexed with 3p-hpRNA (right column), was internalized into the cancerous tissue. As expected, tumors from mice administered PBS showed no fluorescence.

To further investigate the biodistribution of 3E10-D31N following intravenous administration, the liver, lung, spleen, kidney, and heart of each mouse was dissected after sacrifice, and imaged for luciferase expression (indicating cancerous tissue) and fluorescence (visualizing 3E10). Examples of fluorescence images of each organ from representative mice administered 3E10-D31N alone (left column) and 3E10-D31N complexed with 3p-hpRNA (right column) are shown in FIG. 26C (top to bottom: tumor, liver, lung, spleen, kidney, and heart). The average radiant efficiency (fluorescence) measured for each tissue across each cohort of mice is illustrated in FIG. 26D. As can be seen, 3E10 specifically targets the tumor, in vivo. Fluorescence in the liver is a consequence of drug metabolism (*p<0.05; **p<0.005; ***p<0.0005; ****p<0.00005). Luciferase (top row) and fluorescent (bottom row) images the organs from each mouse administered 3E10-D31N complexed with 3p-hpRNA are shown in FIG. 26E. These results demonstrate that 3E10-D31N is capable of localizing and delivering therapeutic polynucleotides to pancreatic cancer in a tissue-specific fashion in vivo.

### Example 18: 3E10-D31N Mediated Delivery of RIG-I Ligand Induces Cytokine Expression Consistent with RIG-I Mediated Cell Death in Melanoma Cells

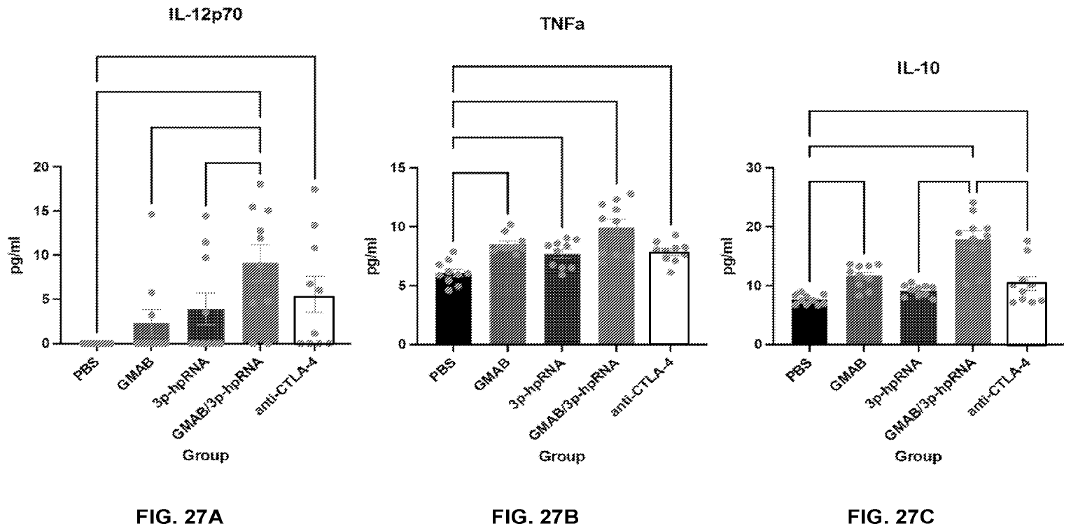

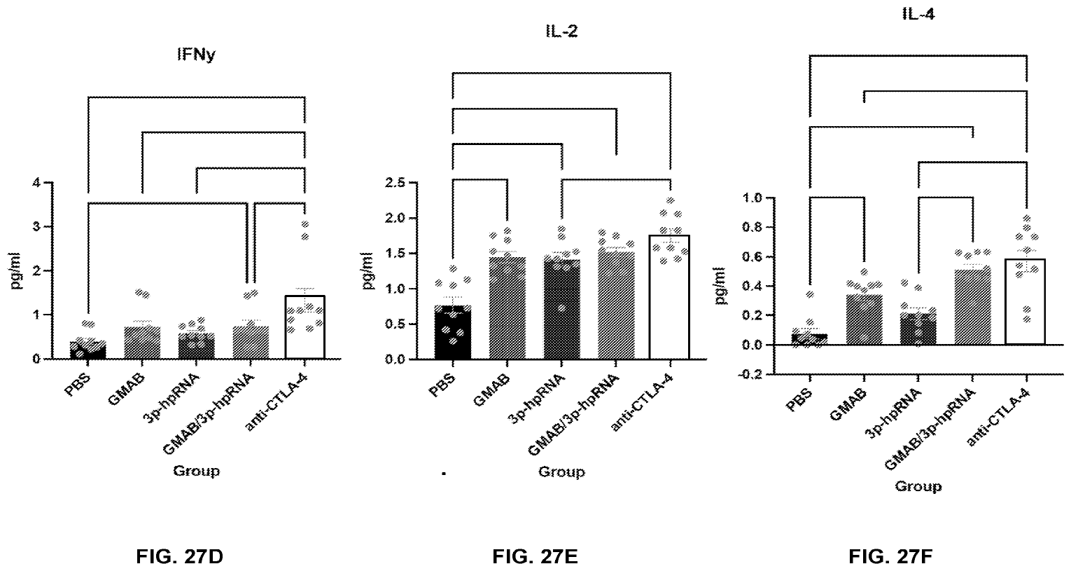

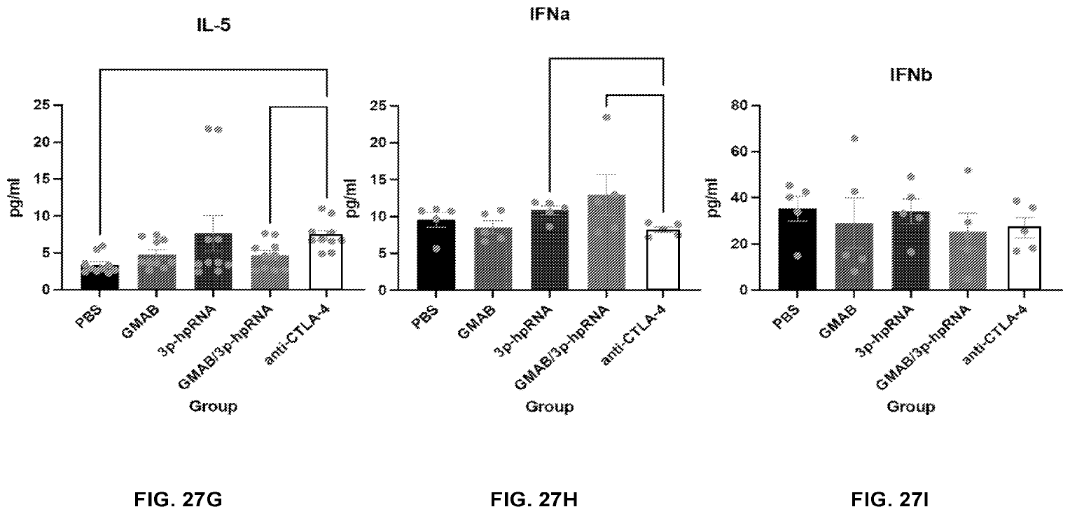

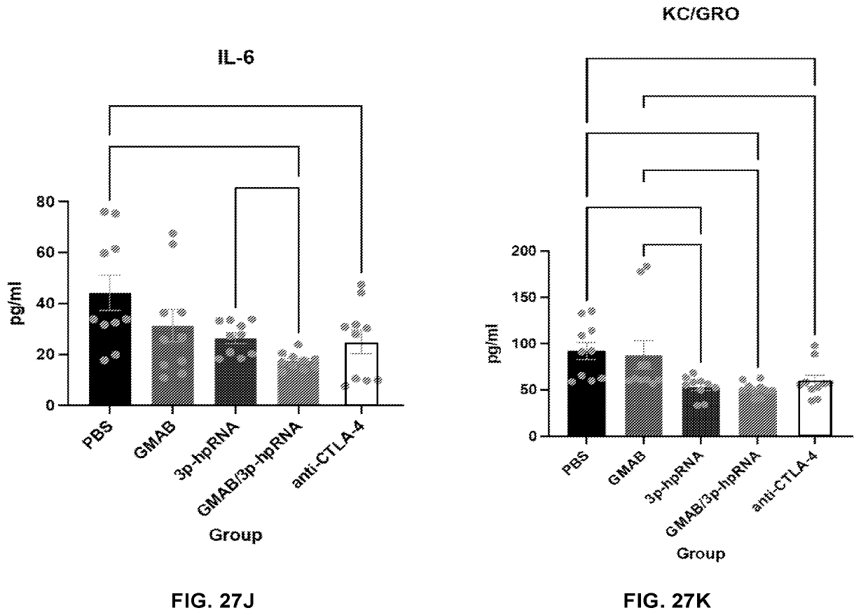

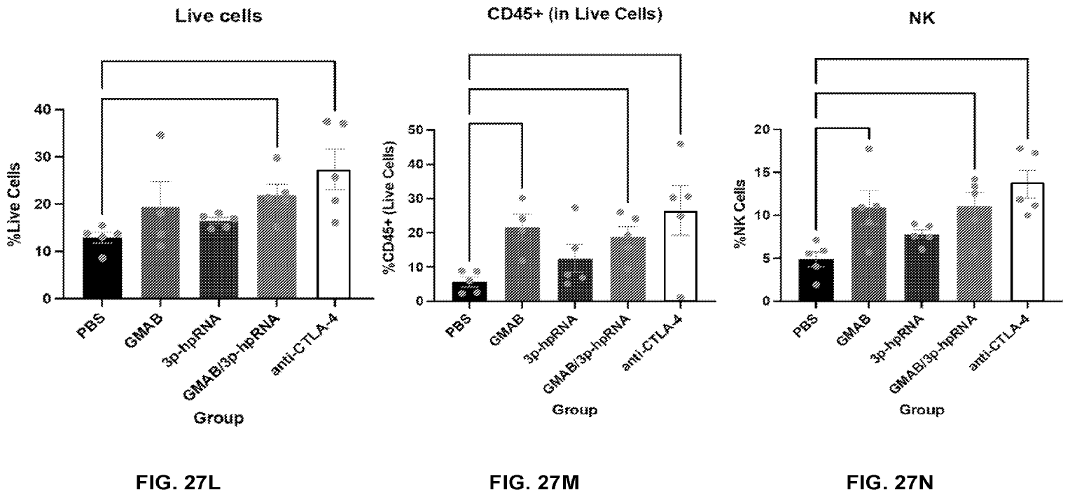

The mechanism of 3E10-D31N/3p-hpRNA RIG-I agonist induced cell death of melanoma cells was further investigated by determining changes in inflammatory cytokines produced in such cells. Briefly, B16 cells—a murine model of melanoma—were injected into mice to generate melanoma tumors. At days 7 and 11 post-injection, the mice were treated with PBS (control), the 3p-hpRNA RIG-I agonist alone, 3E10-D31N (GMAB) alone, 3E10-D31N/3p-hpRNA complexes (3E10:3p-hpRNA), or an anti-CTLA-4 antibody. At day 12, tumors were resected from the mice and analyzed for infiltration of natural killer (NK) cells and tumor infiltrating leukocytes (TILs) by flow cytometry, as well as for the levels of various cytokines, as indicated in FIG. 27.

As shown in FIG. 27L, the percentage of live cells in tumors from mice treated with the 3E10-D31N/3p-hpRNA complexes and anti-CTLA-4 antibody was significantly higher than the percentage of live cells in tumors from the negative control mice treated with PBS, indicative of the presence of a greater number of TILs. Consistent with this result, the proportion of the live cells that were CD45+ or NK cells was significantly higher in tumors from mice treated with the 3E10-D31N/3p-hpRNA complexes and anti-CTLA-4 antibody than in tumors from the negative control mice treated with PBS. Further, the levels of IL-12p70 (FIG. 27A), TNFα (FIG. 27B), IL-10 (FIG. 27C), IFNγ (FIG. 27D), IL-2 (FIG. 27E), IL-4 (FIG. 27F), IL-5 (FIG. 27G), IFNα (FIG. 27H), IFNβ (FIG. 27I), IL-6 (FIG. 27J) and KC/GRO (FIG. 27K) in the tumors were also determined. As shown collectively in FIG. 27, only those mice treated with the 3E10-D31N/3p-hpRNA complexes promoted tumor infiltration by CD45+ TILs and induced IL-12, IL-10, and TNFα production.

### Example 19: 3E10-D31N Mediated Delivery of RIG-I Ligand Induces Type-I IFN Response in THP-1 Monocytes

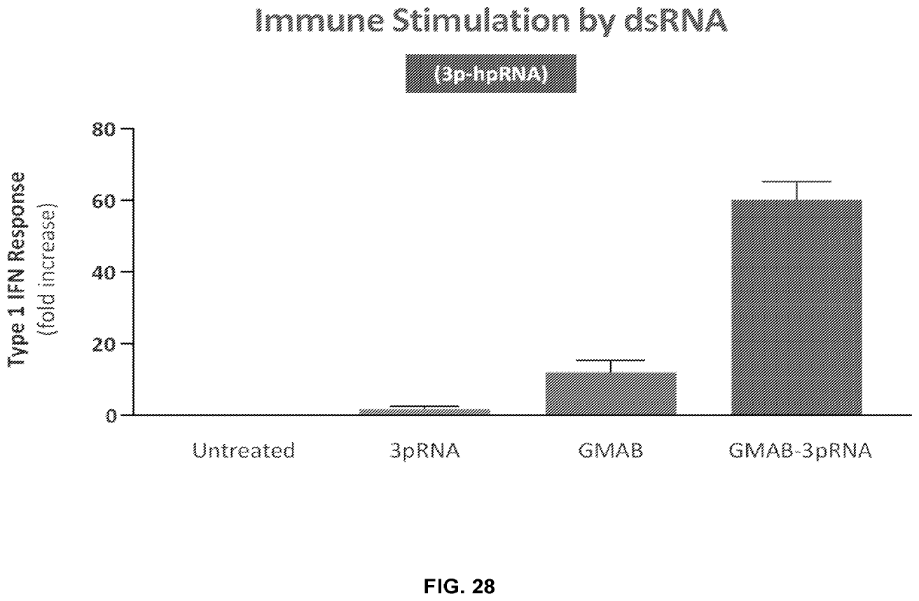

It was investigated whether 3E10-D31N mediated delivery of a RIG-I stimulating ligand into monocytic cells derived from an acute monocytic leukemia effectively induces a Type-I IFN response characteristic of immunotherapy. Briefly, THP-1 monocytes were seeded into wells and incubated in DMEM supplemented with 20% FBS and 1% P/S at 20,000 cells/well. Cells were then treated with PBS (control), the 3p-hpRNA RIG-I agonist alone (1 ug/well), increasing amounts of 3E10-D31N (GMAB) alone, and 3E10-D31N/3p-hpRNA (1 ug 3p-hpRNA/well) complexes, for 10 minutes. Sample media was sampled at indicated timepoints and measured for luciferase activity (reporter for type-I IFN). The cell line includes a luciferase reporter that is expressed when the IFN pathway is induced. As shown in FIG. 28, treatment of the cells with 3E10-D31N/3p-hpRNA complexes elicited a significantly greater Type I IFN response than treatment with E10-D31N alone. Treatment with PBS and 3p-hpRNA RIG-I agonist alone did not result in a Type I IFN response.

### Example 20: Exposure to Complexes Formed Between 3E10-D31N and a RIG-I Agonist Cause Cell Death in Colorectal Tumor Cells

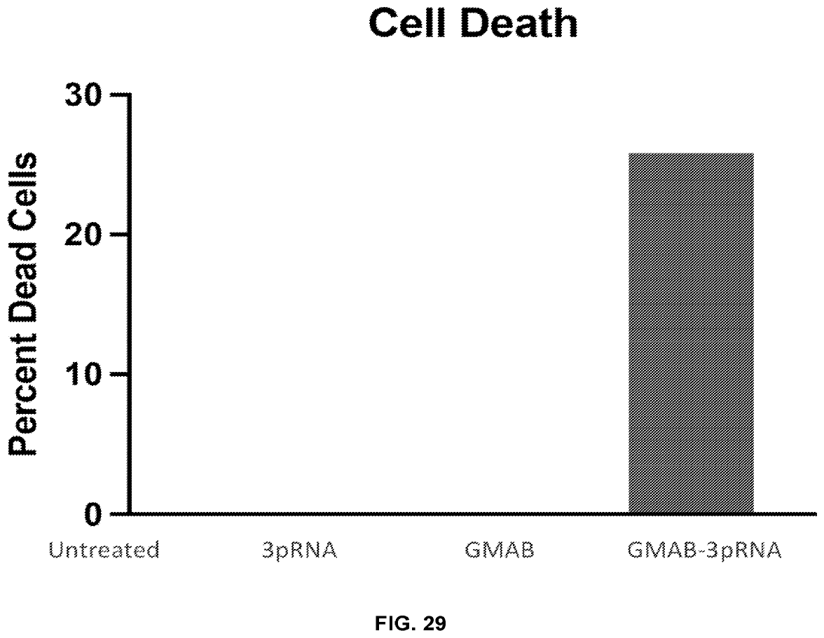

It was further investigated whether 3E10-D31N mediated delivery of a RIG-I stimulating ligand into colorectal cancer cells induces cellular death. Briefly, cells from a MC38 mouse colon cancer cell line, which is syngeneic with C57Bl/6 mice, were seeded into wells and then treated with PBS (control), the 3p-hpRNA RIG-I agonist alone, 3E10-D31N (GMAB) alone, and 3E10-D31N/3p-hpRNA complexes (3E10:3p-hpRNA). Cell death was then measured. As shown in FIG. 29, treatment with the 3E10-D31N/3p-hpRNA complexes resulted in significant cell death, while all other treatments did not induce cell death.

### Example 21: 3E10-D31N Mediated Delivery of RIG-I Ligand Suppresses Tumor Growth in a Murine Model of Melanoma

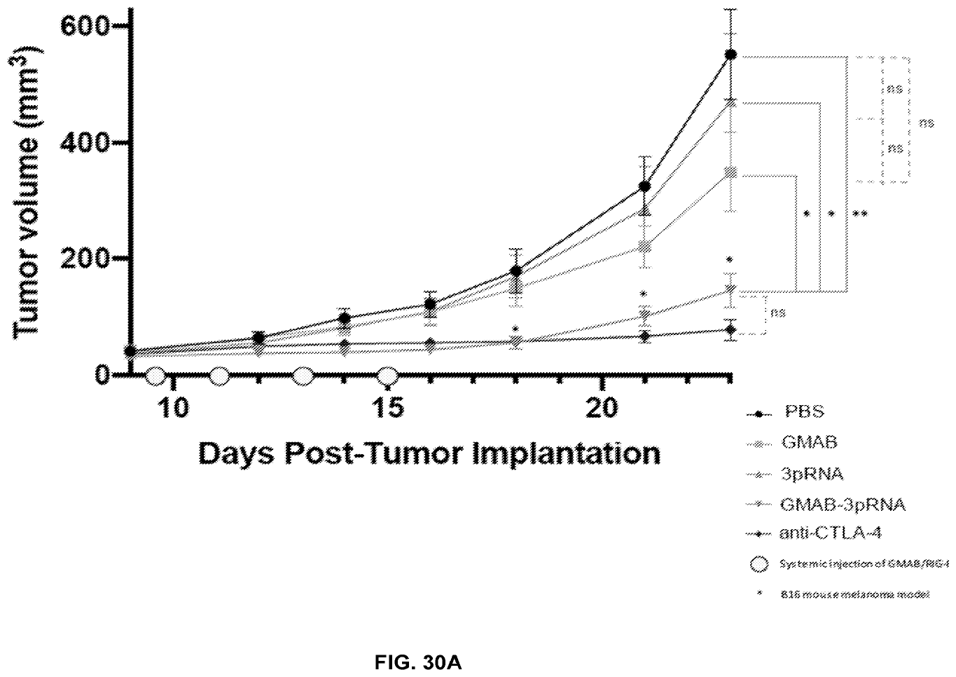

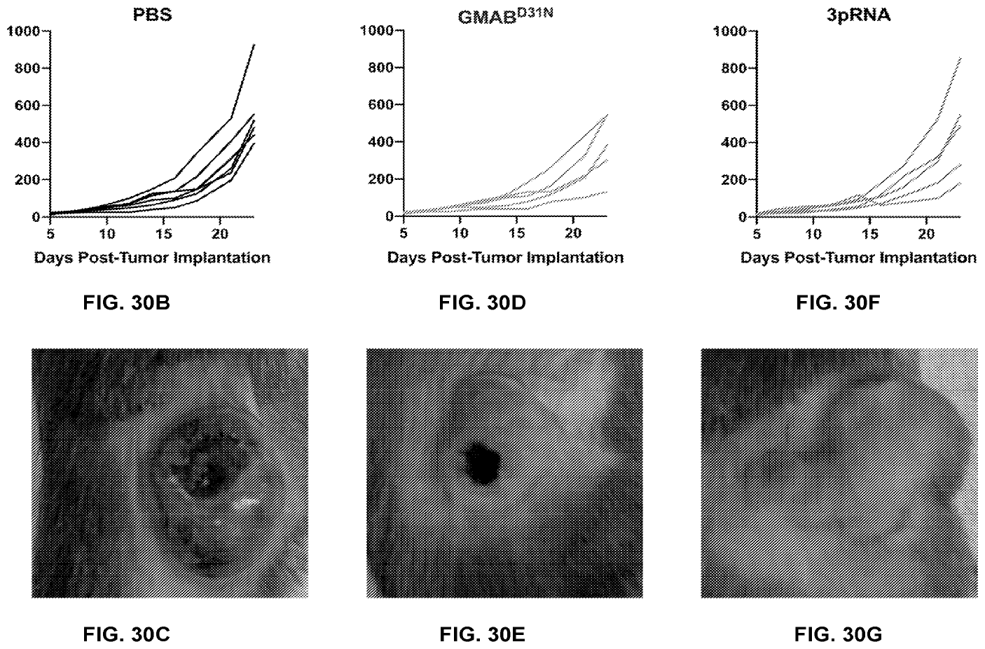

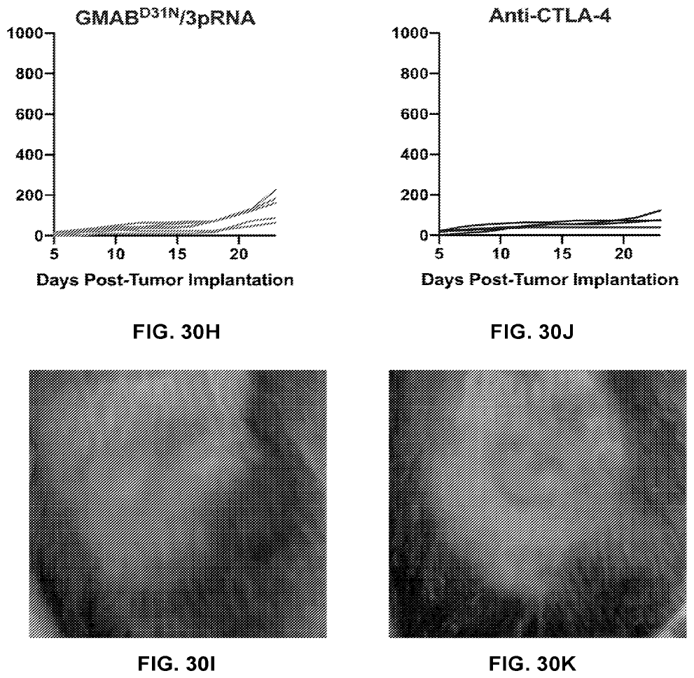

It was investigated whether complexes of 3E10-D31N/3p-hpRNA RIG-I agonist would suppress melanoma tumor growth in vivo. Briefly, B16 cells—a murine model of melanoma—were injected into mice to generate melanoma tumors. At days 9, 11, 13, and 15 post-injection, the mice were treated with PBS (control; ●), 3E10-D31N (GMAB; ▪) alone, 3p-hpRNA alone (▴), 3E10-D31N/3p-hpRNA complexes (▾), or an anti-CTLA-4 antibody (♦). Tumor volumes in each of the mice where measured overtime. As shown in FIG. 30A, treatment with 3E10-D31N/3p-hpRNA complexes and the anti-CTLA-4 antibody significantly suppressed tumor growth, relative to treatment with PBS, 3E10-D31N alone, and 3p-hpRNA alone (*p<0.05; **p<0.005). No significant difference was seen between treatment with 3E10-D31N/3p-hpRNA complexes and the anti-CTLA-4 antibody (ns=not significant) or between treatment with 3E10-D31N alone or 3p-hpRNA alone and the negative control (ns=not significant).

FIGS. 30B-30G illustrate the tumor volume (mm³) for each individual mouse in each cohort and representative images of the tumors at day 23 post-transplantation, following systemic administration of PBS (control; 30B and 30C), 3E10-D31N (GMAB; 30D and 30E) alone, 3p-hpRNA alone (30F and 30G), 3E10-D31N/3p-hpRNA complexes (30H and 30I), or an anti-CTLA-4 antibody (30J and 30K) at days 9, 11, 13, and 15 post tumor-transplantation.

### Example 22: Co-Therapy with 3E10-D31N/RIG-I Ligand Complexes and Anti-PD-1 Antibodies Synergizes to Suppress Tumor Growth in a Murine Model of Colon Cancer

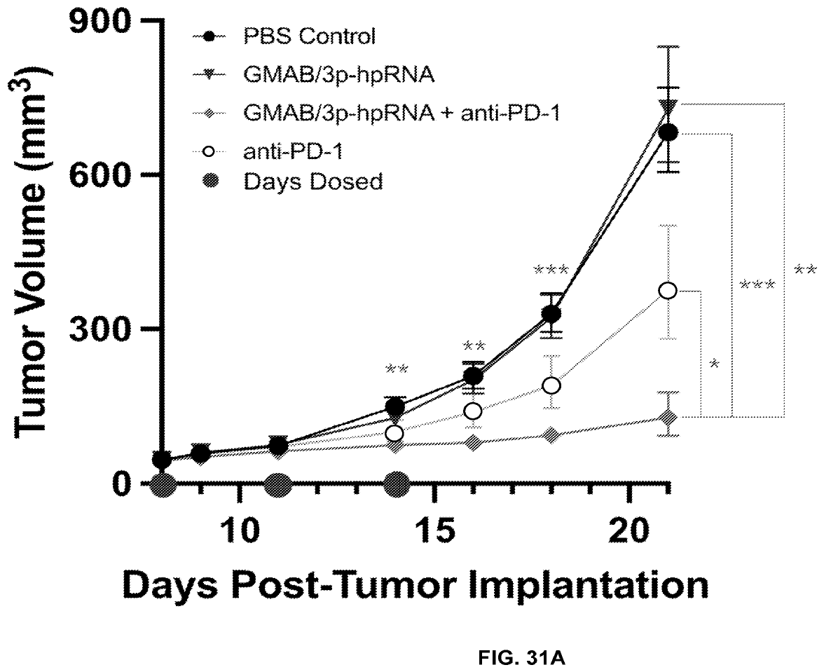

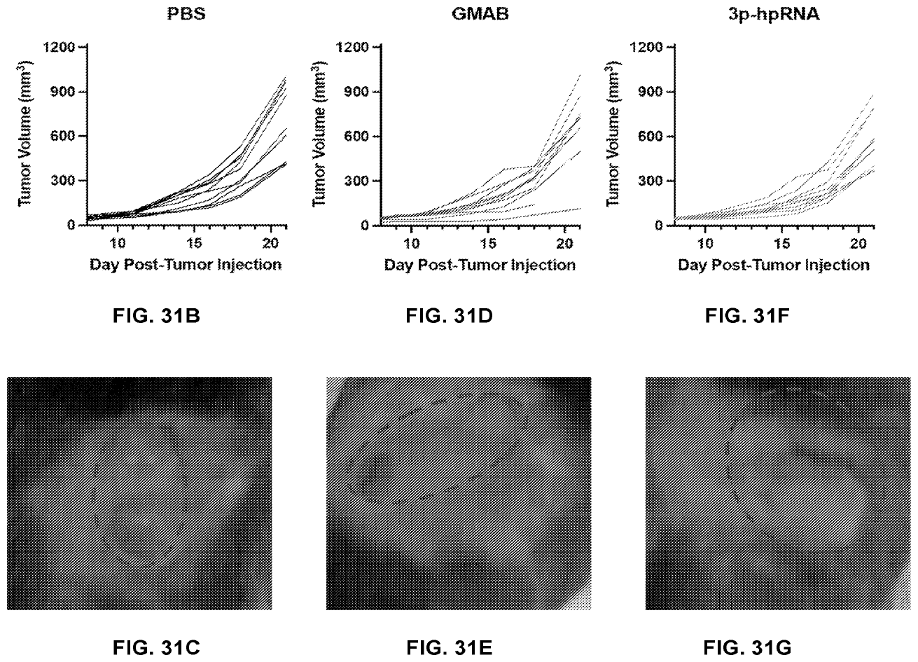

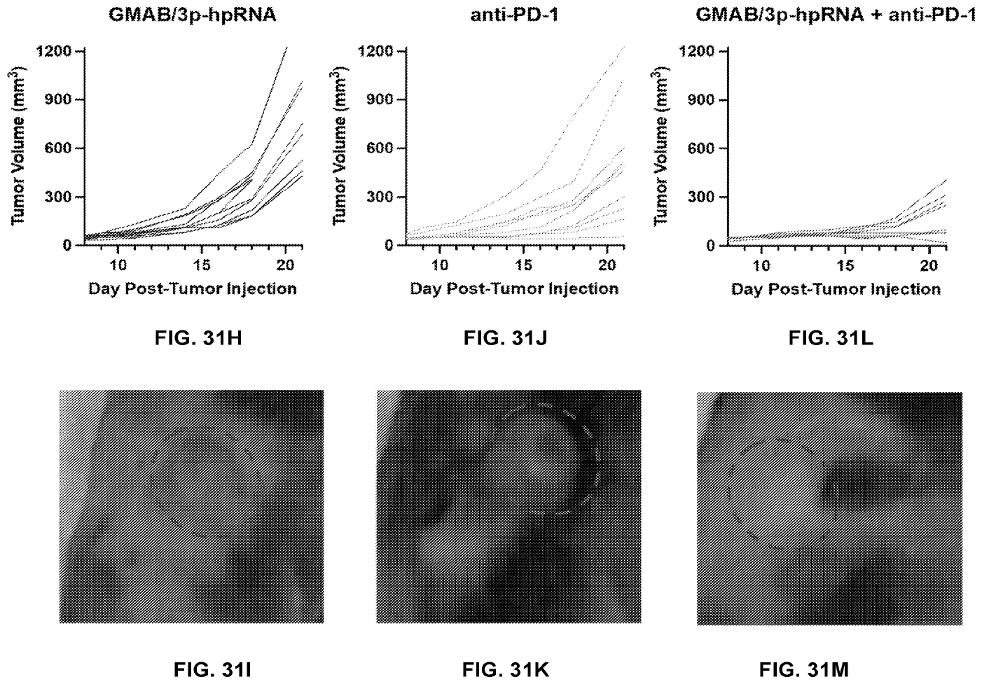

It was investigated whether complexes of 3E10-D31N/3p-hpRNA RIG-I agonist would suppress colon cancer tumor growth in vivo. Briefly, MC38 cells—a mouse colon cancer cell line—were injected into mice to generate colon tumors. At days 8, 11, and 14 post-injection, the mice were treated with PBS (control; ●), 3E10-D31N/3p-hpRNA complexes (▾), 3E10-D31N/3p-hpRNA complexes+anti-PD-1 (♦), or anti-PD-1 alone (∘). Tumor volumes were measured overtime. As shown in FIG. 31A, treatment with 3E10-D31N/3p-hpRNA complexes alone failed to suppress tumor growth, relative to the negative control (PBS). Treatment with the anti-PD-1 antibody alone moderately suppressed tumor growth, relative to the negative control (PBS). However, co-treatment with 3E10-D31N/3p-hpRNA complexes and the anti-PD-1 antibody significantly suppressed tumor growth relative to all other treatments (*p<0.05; **p<0.005; ***p<0.0005).

FIGS. 31B-31M illustrate the tumor volume (mm³) for each individual mouse in each cohort and representative images of the tumors at day 21 post-injection, following systemic administration of PBS (control; 31B and 31C), 3E10-D31N alone (GMAB; 31D and 31E), 3p-hpRNA alone (31F and 31G), 3E10-D31N/3p-hpRNA complexes (31H and 31I), an anti-PD-1 antibody (31J and 31K), or 3E10-D31N/3p-hpRNA complexes+anti-PD-1 antibody (31L and 31M).

### Example 23: Co-Therapy with 3E10-D31N/RIG-I Ligand Complexes and Anti-PD-1 Antibodies Synergizes to Suppress Tumor Growth in a Murine Model of Breast Cancer

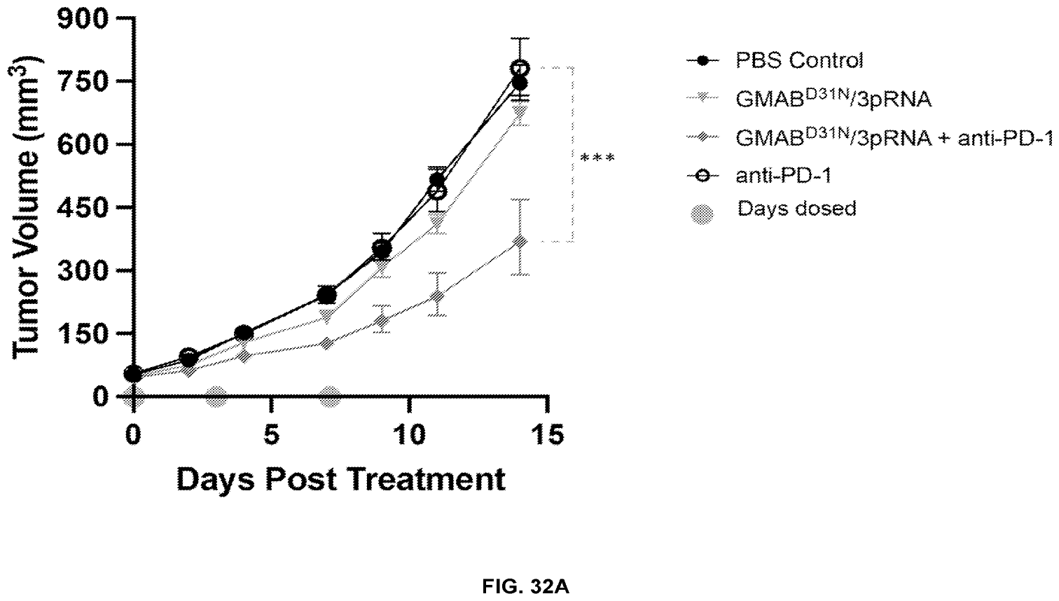

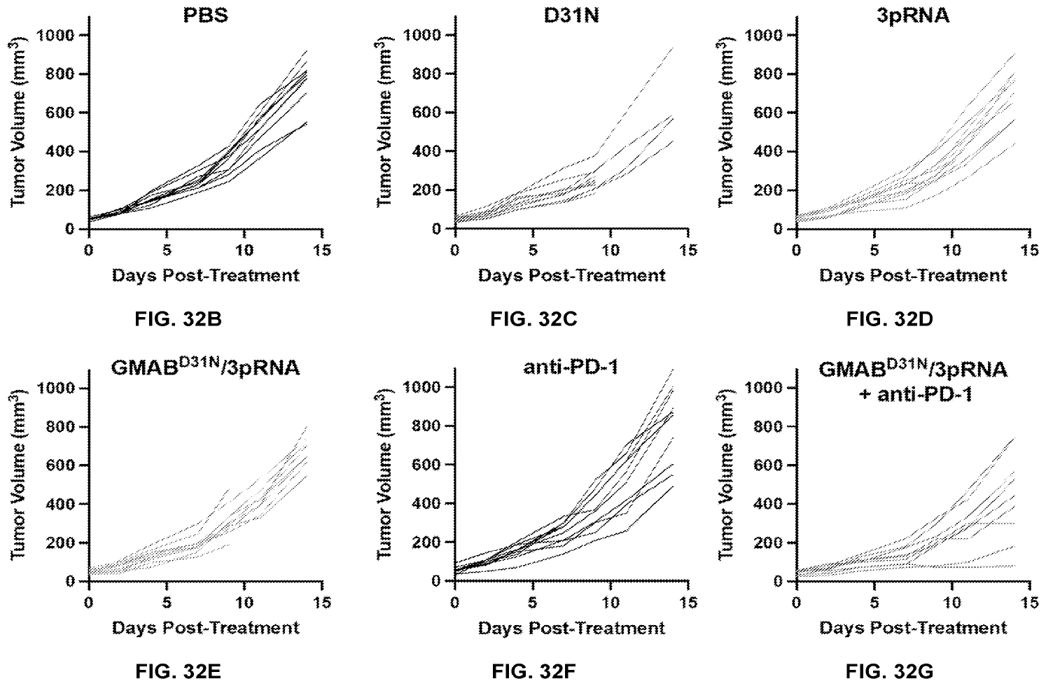

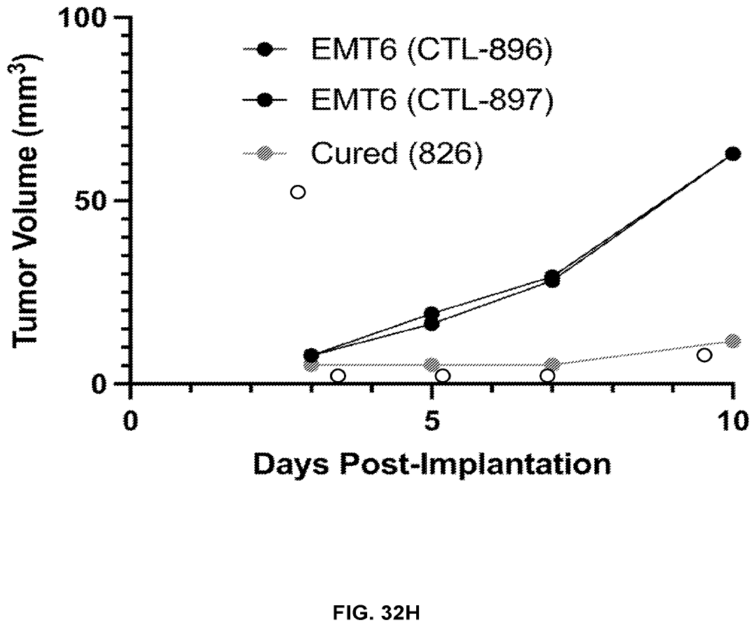

It was investigated whether complexes of 3E10-D31N/3p-hpRNA RIG-I agonist would suppress breast cancer tumor growth in vivo. EMT6 is a murine mammary carcinoma cell line derived from a transplanted hyperplastic alveolar nodule of a BALB/c mouse. In syngeneic mice, EMT6 cells form tumors and spontaneous metastases, primarily to the lungs. Accordingly, EMT6 cells were injected into mice to generate lung cancer tumors. At days 8, 11, and 14 post-injection, the mice were treated with PBS (control; ●), 3E10-D31N/3p-hpRNA complexes (▾), 3E10-D31N/3p-hpRNA complexes+anti-PD-1 (♦), or anti-PD-1 alone (∘). Tumor volumes were measured over time. As shown in FIG. 32A, treatment with 3E10-D31N/3p-hpRNA complexes alone or the anti-PD-1 antibody alone failed to suppress tumor growth, relative to the negative control (PBS). However, co-treatment with 3E10-D31N/3p-hpRNA complexes and the anti-PD-1 antibody significantly suppressed tumor growth relative to all other treatments (***p<0.0005).

FIGS. 32B-32G illustrate the tumor volume (mm³) for each individual mouse in each cohort following systemic administration of PBS (control 32B), 3E10(D31N) alone (GMAB; 32C), 3p-hpRNA alone (32D), 3E10(D31N)/3p-hpRNA complexes (32E), an anti-PD-1 antibody (32F), or 3E10(D31N)/3p-hpRNA complexes+anti-PD-1 antibody (32G). FIG. 32H illustrates that co-treatment with 3E10-D31N/3p-hpRNA complexes and the anti-PD-1 antibody generates an immunological memory. Specifically, FIG. 32H demonstrates that a mouse rechallenged with tumor cells does not grow a tumor once immunological memory has formed post treatment.

### Example 24: 3E10-D31N is Internalized and Associates with gDNA In Vivo

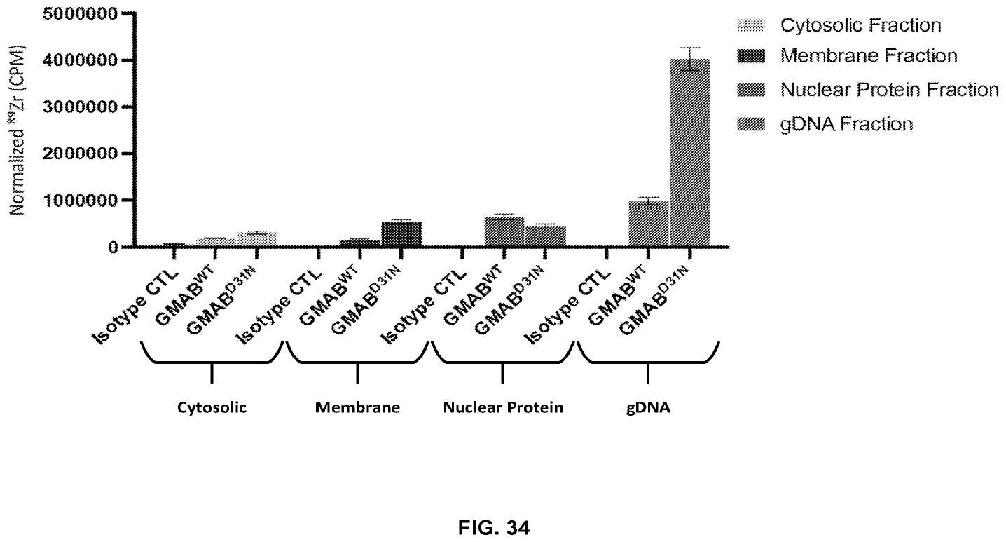

Internalization and cellular location experiments for 3E10-D31N were investigated. Isotype control, 3E10-WT (GMAB^{WT}), and 3E10-D31N (GMAB^{D3}1N) antibodies were labeled with ⁸⁹Zr and administered to cells in vivo. After an amount of time, cellular components (cytosol, membrane, nuclear protein, and gDNA) were fractionated and assayed for ⁸⁹Zr signal (Counts per Minute, CPM). As shown in FIG. 34, the majority of the internalized GMAB^{WT} and GMAB^{D31N} antibodies localized to the nucleus and were found associated with both the nuclear protein fraction of the nucleus and the gDNA fraction. However, a larger portion of GMAB^{D31N} associated with the gDNA fraction than did GMAB^{WT}, suggesting that GMAB^{D31N} localizes more readily to chromatin than does GMAB^{WT}.

### Example 25: Chimeric 3E10-D31N (cD31N) Non-Covalently Binds to RNA and Distributes to Tumors In Vivo

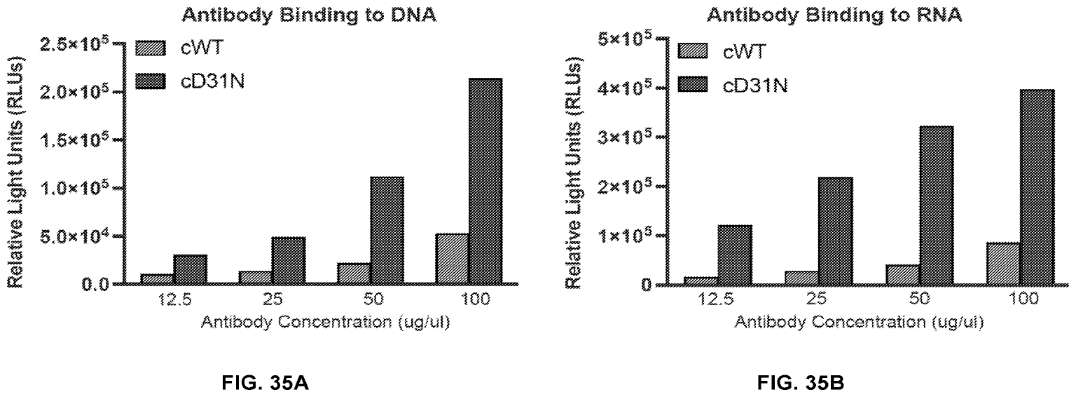

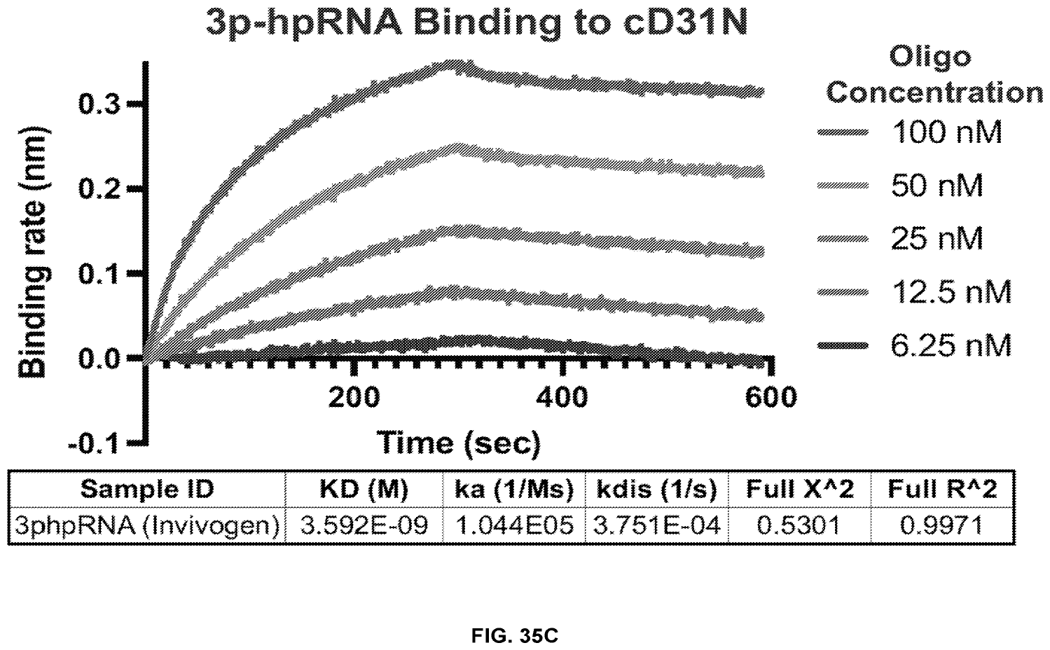

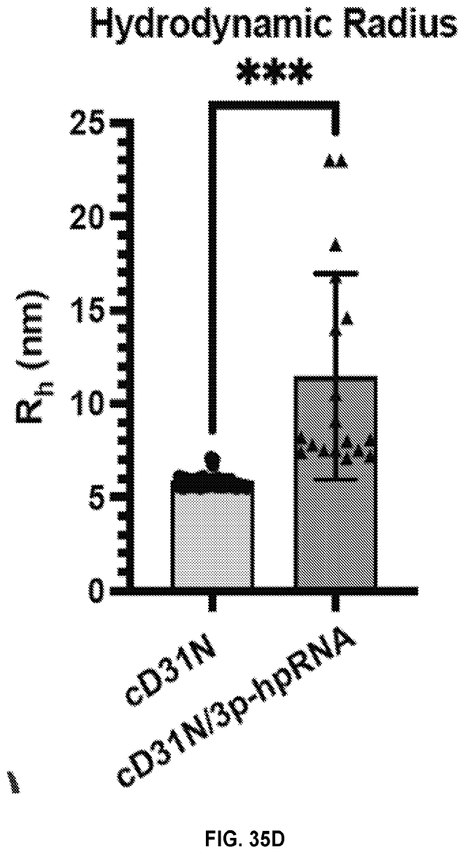

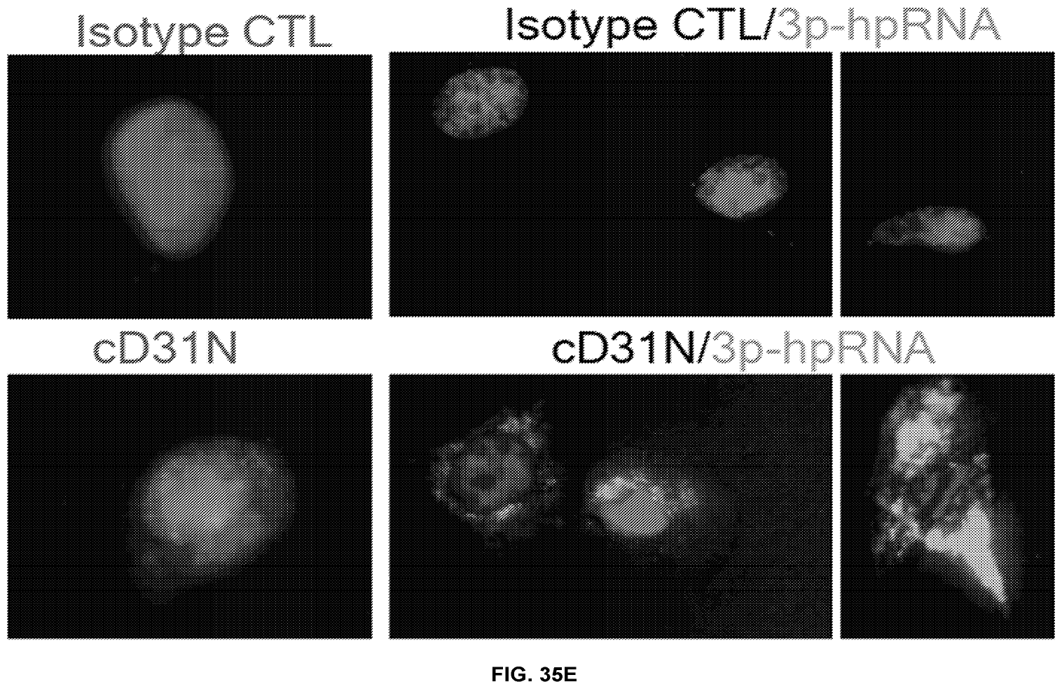

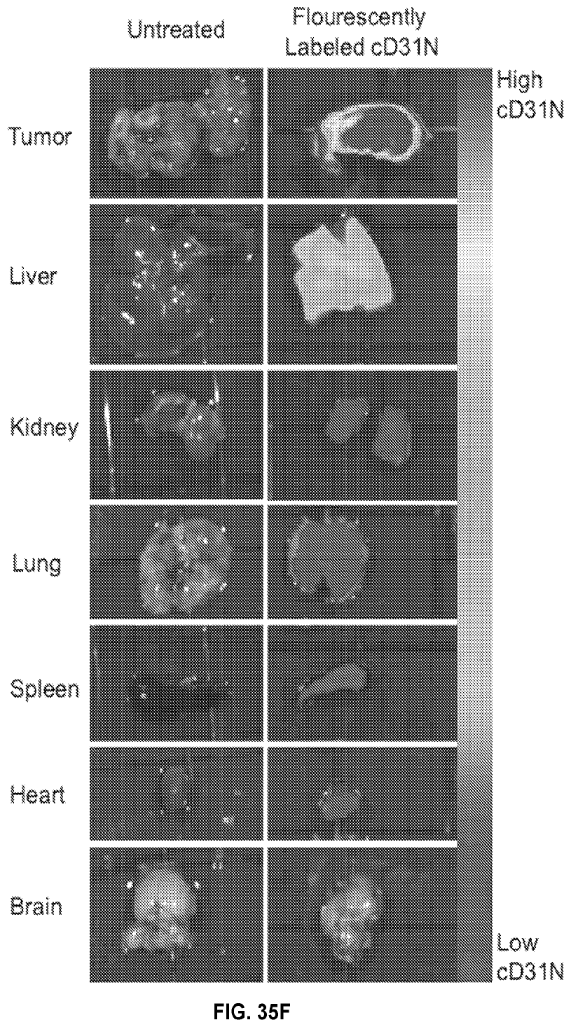

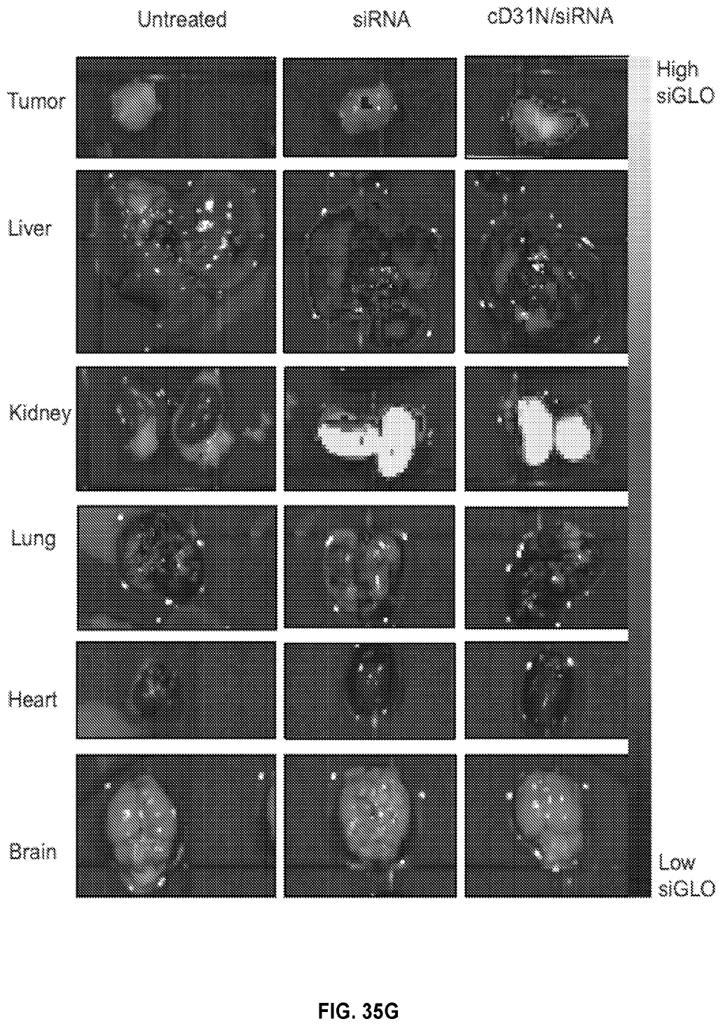

Nucleic acid binding for chimeric 3E10-D31N (cD31N) were investigated. At selected antibodies concentrations, cD31N and the related chimeric 3E10 without the D31N substitution (cWT) were assayed by ELISA to determine binding kinetics to nucleic acids. As shown in FIG. 35A and FIG. 35B, cD31N shows a greater than 2-fold increase preferential binding to RNA versus DNA at all antibody concentrations tested (12.5, 25, 50 and 100 μg/l) measured by relative light units (RLUs). In another experiment, the cD31N affinity for 89 nucleotide 3p-hpRNA cD31N was measured by Bio-Layer Interferometry (BLI) as a function of increasing RNA concentration. As shown in FIG. 35C, the binding rates (nm) increased precipitously and remained the greatest over time (sec) for the highest oligo concentration (100 nM) versus oligo concentrations of 6.25 nM, 12.5 nM, 25 nM, and 50 nM. Further shown in FIG. 35C, are the derived values for the dissociation constant (K_{D}) of 3p-hpRNA and related parameters. In another experiment, the hydrodynamic radii of cD31N/3p-hpRNA antibody RNA complexes (3p-ARCs) were compared and measured against to cD31N alone using dynamic light scattering. As shown in FIG. 35D, the dynamic light scattering was statistically greater for 3p-ARCs when compared to cD31N alone. The data are mean s.e.m. from n=16 and p values were determined by two-tailed unpaired t-test.

The penetration and distribution of cD31N into U2OS human osteosarcoma cells was investigated. Isotype control and cD31N were incubated with a fluorescently labeled 3p-hpRNA and administered to U2OS human osteosarcoma cells. As shown in FIG. 35E, the delivery of fluorescently labeled 3p-hpRNA into U2OS cells was greater when cells are treated with 3p-hpRNA complexed with cD31N compared to labeled 3p-hpRNA incubated with an isotype control. IVIS visualization of tumor and normal tissue distribution was investigated 24 hours after administration of fluorescently labeled cD31N in mice (n=1) bearing EMT6 flank tumors. FIG. 35F shows increased tissue distribution of fluorescently labeled cD31N in the tumor and liver versus several other tissues, e.g., spleen, heart and brain. IVIS visualization of tumor and normal tissue distribution was investigated 24 hours after administration of siGLO (fluorescently labeled siRNA) complexed to cD31N in mice (n=1) bearing EMT6 flank tumors. FIG. 35G shows increased tissue distribution of siGLO in the tumor and liver versus several other tissues, e.g., spleen, heart and brain.

### Example 26: Chimeric 3E10-D31N Antibody (cD31N)/3p-hpRNA Antibody/RNA Complexes Induce a RIG-I-Dependent Interferon Response

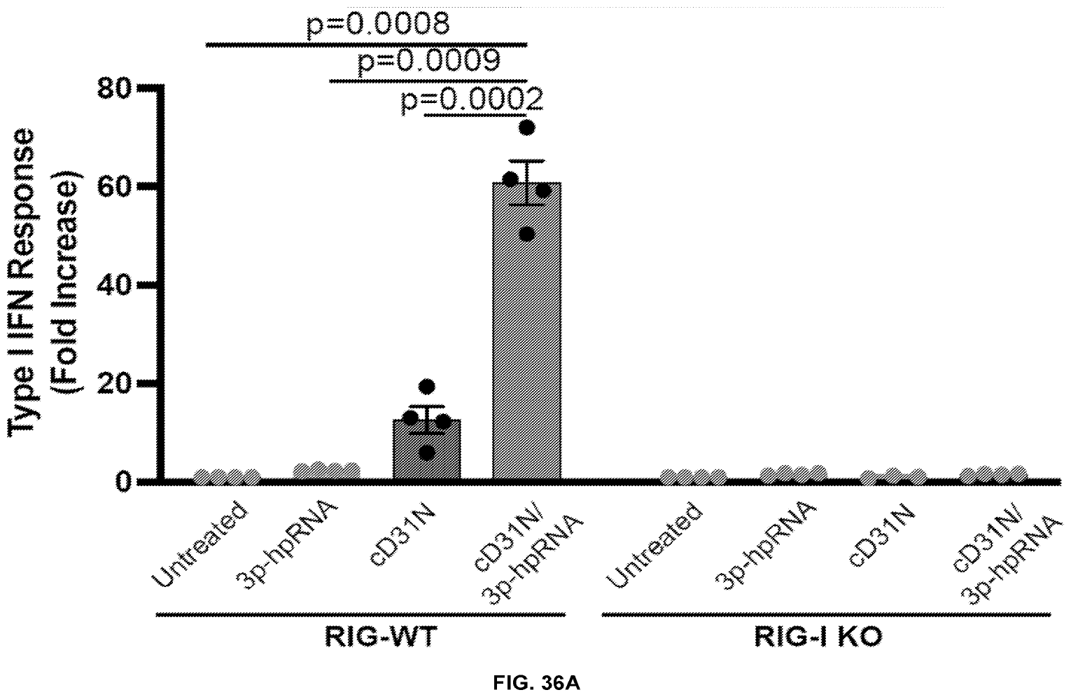

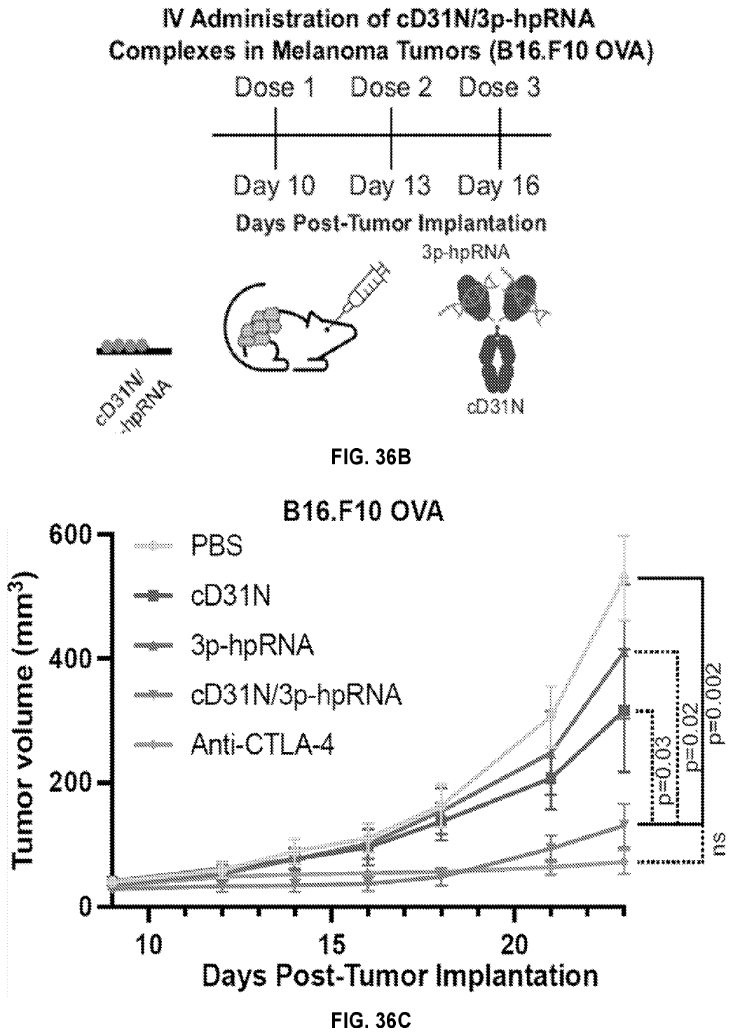

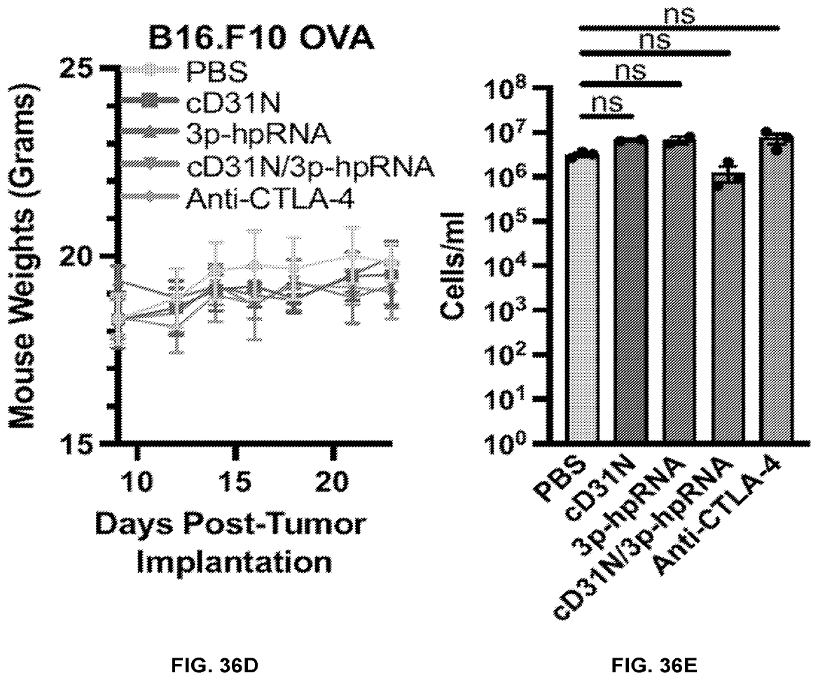

Chimeric 3E10-D31N antibody (cD31N)/3p-hpRNA antibody/RNA complexes were investigated for their ability to induce a RIG-I-dependent interferon response and mediate tumor growth suppression in a mouse model of melanoma. THP-1 monocytes or THP-1 RIG-I KO monocytes containing an IFN-response luciferase reporter constructed were used to test a type I IFN induction response and these cells were treated with vehicle control (PBS), 3p-hpRNA, cD31N, or cD31N/3p-hpRNA. As shown FIG. 36A, the 3E10-D31N antibody (cD31N)/3p-hpRNA antibody/RNA complex showed greatest ability to induce a RIG-I-dependent interferon response (˜60-fold increase). For the tumor growth study, an in vivo dosing schedule was outlined for IV administration of cD31N/3p-hpRNA complexes by retro-orbital IV injection in immune competent C57Bl/6 mice bearing B16.F10.OVA flank tumors as shown in FIG. 36B. Tumor growth curves in mice bearing B16.F10.OVA flank tumors treated with vehicle control (PBS), cD31N, 3p-hpRNA, cD31N/3p-hpRNA, or anti-CTLA-4 was measured. Tumor volume data (mm³) from n=5-6 per group was recovered and tumors volumes were significantly less in mice treated with cD31N/3p-hpRNA versus cD31N alone or 3p-hpRNA alone. FIG. 36D illustrates body weights of treated mice bearing B16.F10.OVA flank tumors over the course of the experiment. FIG. 36E illustrates terminal bone marrow cell counts following treatment in B16.F10.OVA flank tumor bearing mice.

### Example 27: cD31N/3p-hpRNA Antibody/RNA Complexes Induce Tumor Infiltrating Leukocytes in Melanoma Flank Tumors

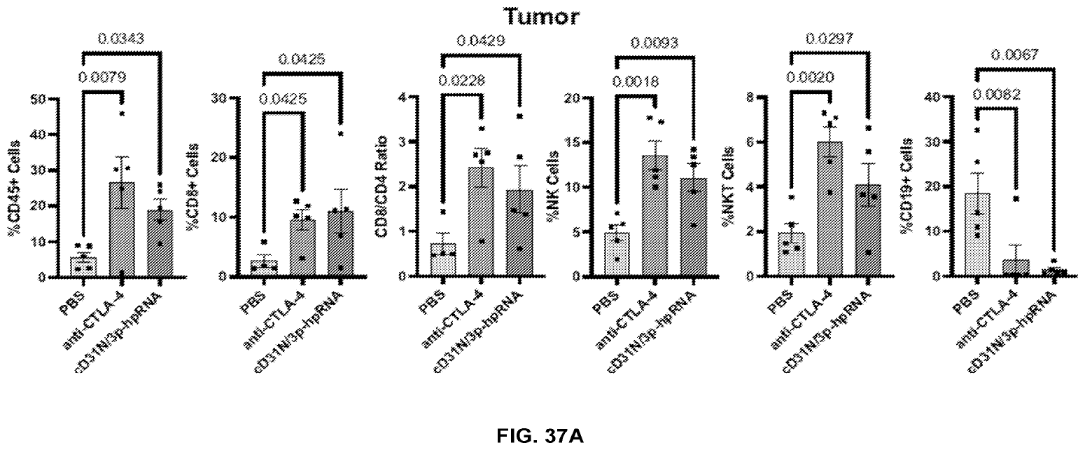

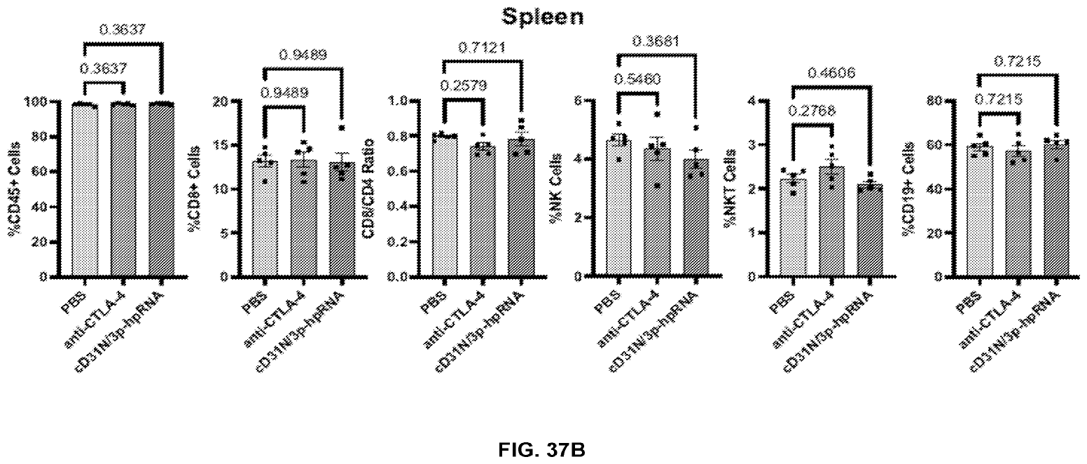

The cD31N/3p-hpRNA antibody/RNA complexes were investigated to determine whether they can induce tumor infiltrating leukocytes in melanoma flank tumors. As shown in FIG. 37A and FIG. 37B, the percentages of CD45+, CD8+, NK, NKT, and CD19+ cells were measured in tumors and in splenic controls (FIG. 37B) harvested from treated C57Bl/6 mice bearing B16.F10.OVA tumors 24 hours after two doses of indicated treatments were administered (on days 10 and 13 post-implantation).

### Example 28: cD31N/3p-hpRNA Complexes Penetrate Orthotopic Pancreatic Tumors, Enhance Survival, Induce Tumor Necrosis, and Promote Immunogenicity

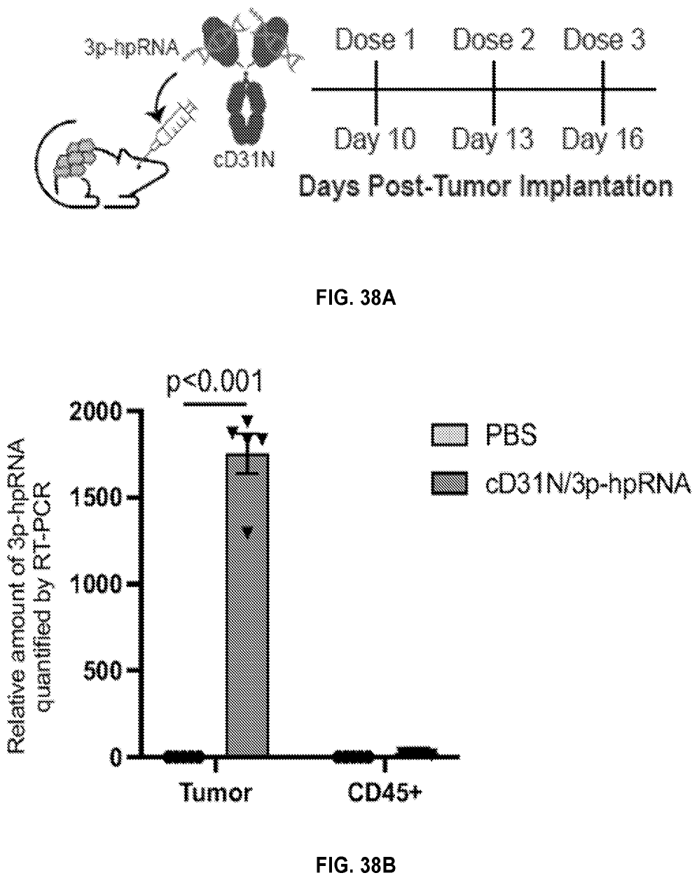

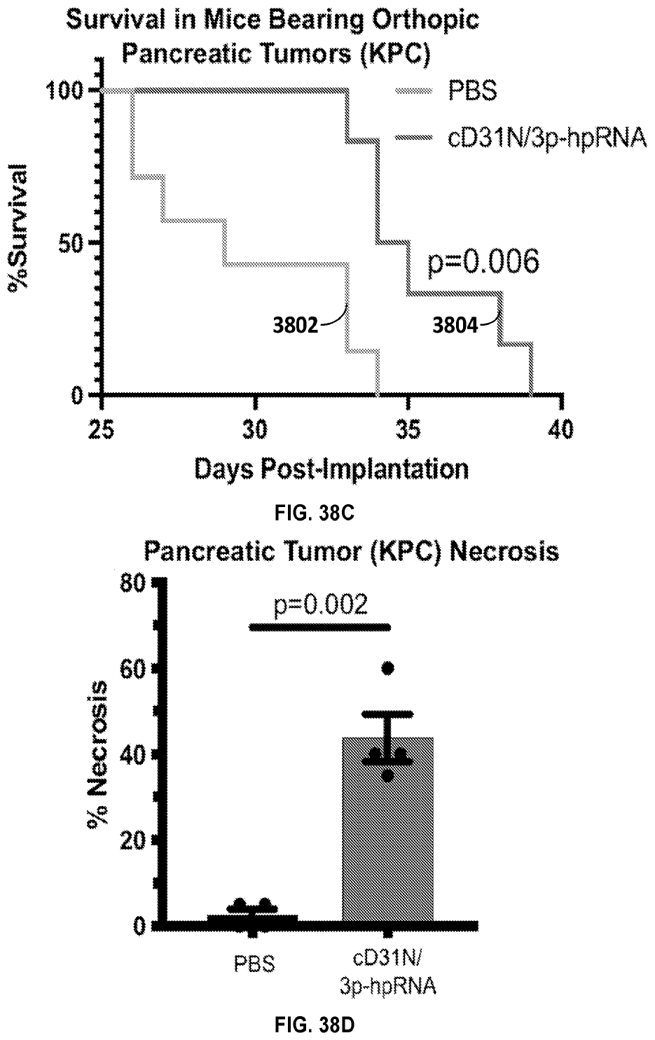

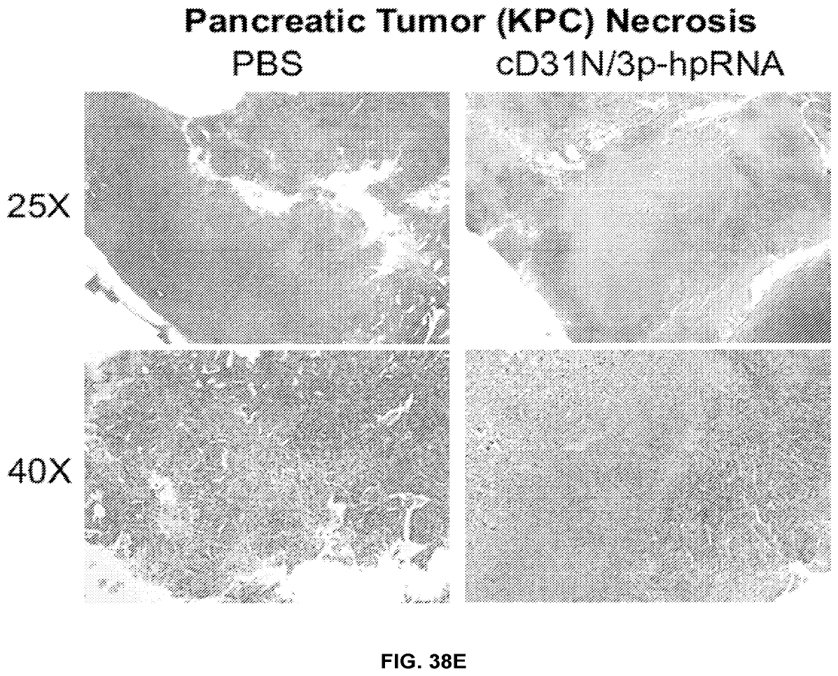

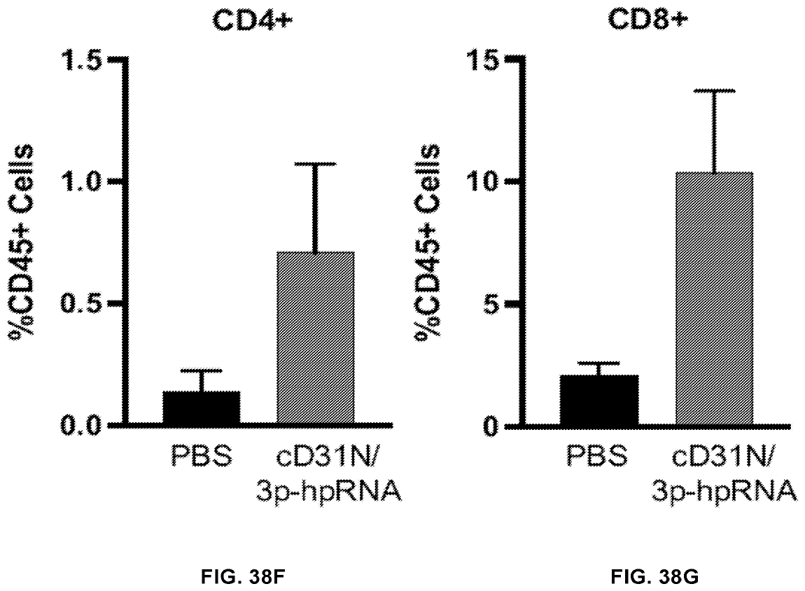

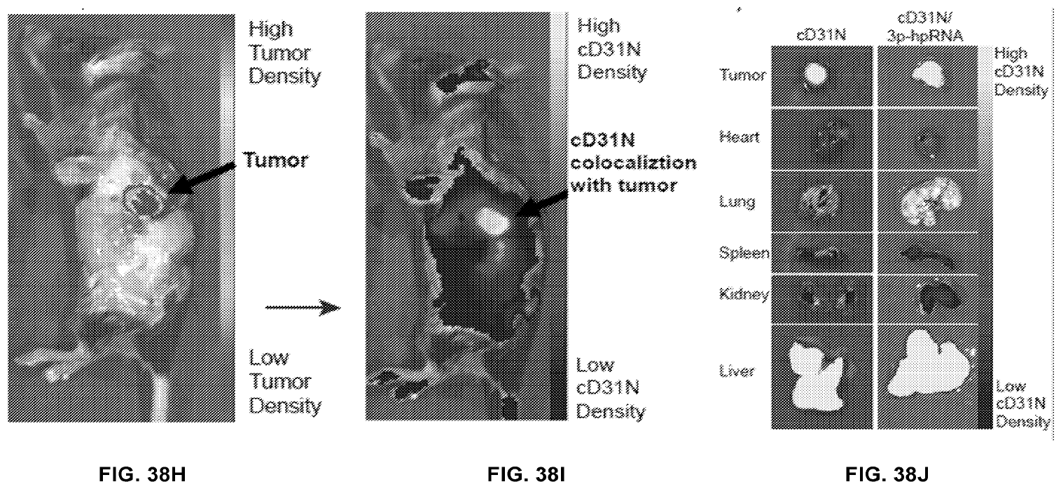

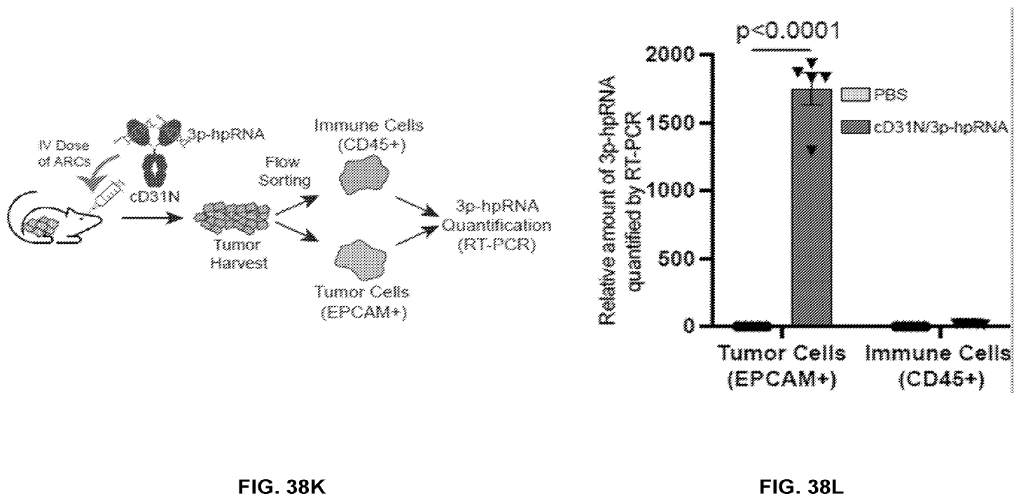

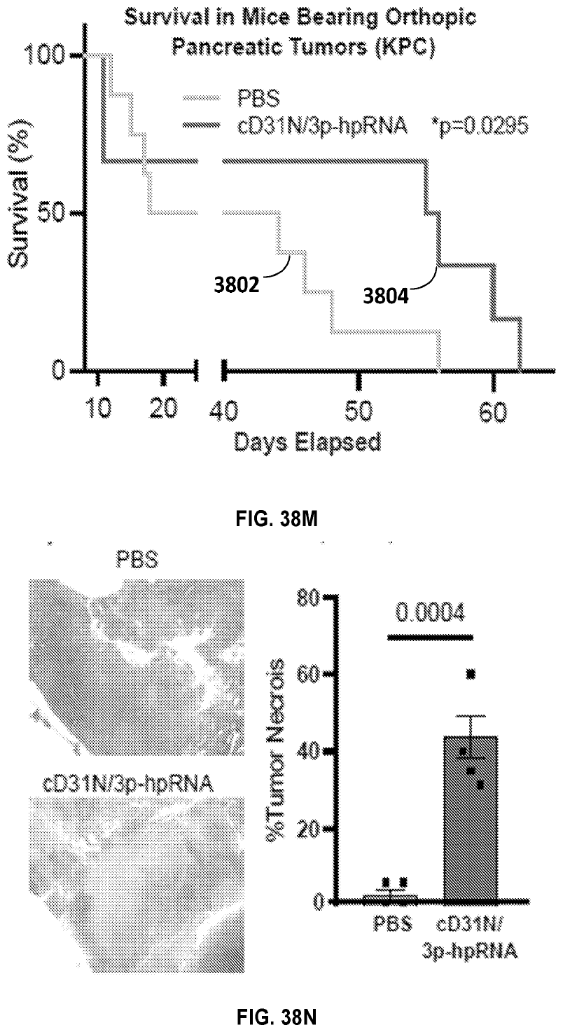

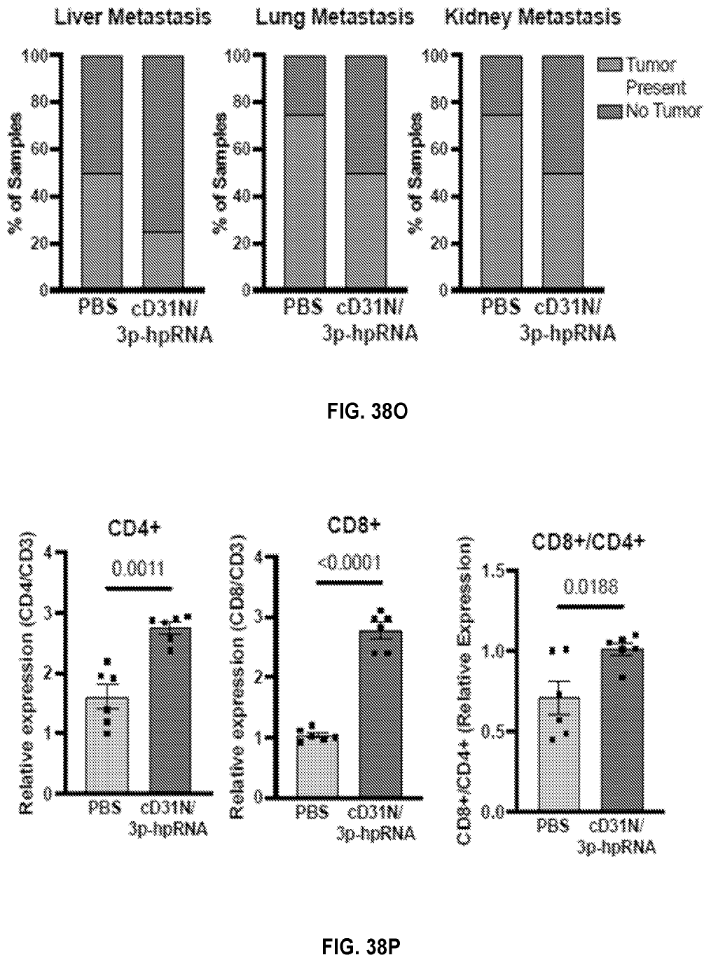

It was investigated whether complexes of 3E10-D31N/3p-hpRNA RIG-I agonist could treat a pancreatic cancer model in vivo. The murine KPC model of pancreatic ductal adenocarcinoma develops many key features observed in human PDA including pancreatic intraepithelial neoplasia and a robust inflammatory reaction including exclusion of effector T cells. Hingorani S. R., et al., Cancer Cell, 7:469 (2005) and Olive K. P., et al., Science, 324:1457 (2009). Accordingly, KPC mice were administered three doses of 3E10-D31N/3p-hpRNA by retro-orbital IV injection at 10, 13, and 16 days post tumor implantation, respectively, as shown in FIG. 38A.

To investigate whether the complexes were delivering the 3p-hpRNA RIG-I agonist to tumor tissue, RT-PCR quantification of 3p-hpRNA was performed from tumor and CD45+ cells isolated from KPC orthotopic tumors by flow sorting one day following the last of three doses of 3E10-D31N/3p-hpRNA RIG-I agonist. As shown in FIG. 38B, 3p-hpRNA was present in tumor cells of mice administered the complex, but not in mice administered PBS control. 3p-hpRNA was not observed in CD45+ cells. Data from n=5 per group with statistical analysis performed by student's t-test for individual comparison, with p values indicated.

A survival study was performed in the KPC model to evaluate functional delivery of the 3p-hpRNA by 3E10-D31N. Following three doses of systemically administered 3E10-D31N/3p-hpRNA (FIG. 38A), mice administered 3p-hpRNA (3804) had significant improvements in survival relative to the mice administered PBS buffer control (3802), as shown in FIG. 38C (p=0.006) and in FIG. 38M (p=0.0295). Data is from n=6-7 with statistical analysis performed by Log-rank (Mantel-Cox) test, with indicated p values.

Further, approximately half of KPC tumor cells in mice administered 3p-hpRNA showed a necrotic histology, while KPC tumor cells in mice administered the PBS control did not. FIG. 38D illustrates quantification of histological analysis of tumor cell necrosis. Treatment with 3E10-D31N/3p-hpRNA RIG-I agonist led to robust increases in tumor cell necrosis when examined 7 days after the last treatment compared to controls (FIG. 38D and FIG. 38N, respectively), consistent with immunogenic cell death via RIG-I stimulation. Data with statistical analysis was performed by student's t-test for individual comparison, with p values indicated. Consistent with this finding, H&E staining images as shown in FIG. 38E, were performed from tumor sections from control and cD31N/3p-hpRNA treated mice at two magnifications (25× and 40×) and illustrate tumor cell necrosis in cD31N/3p-hpRNA treated mice relative to PBS buffer controls. Moreover, much higher percentages of CD45+ cells in mice treated with 3p-hpRNA were also CD4+ (FIG. 38F) and/or CD8+ (FIG. 38G).

It was investigated whether complexes of 3E10-D31N/3p-hpRNA RIG-I agonist could be used to treat pancreatic cancer, by evaluating the ability of 3E10-D31N to penetrate orthotopic pancreatic tumors generated from KPC cells surgically implanted into pancreata of syngeneic mice (FIG. 38H). We fluorescently labeled 3E10-D31N for in vivo imaging and found that IV-administered 3E10-D31N efficiently targeted orthotopic KPC tumors in mice, as demonstrated by robust co-localization of the labeled antibody with the luciferase-expressing tumors (FIG. 38H and FIG. 38I), despite these tumors being poorly vascularized and difficult to penetrate. Furthermore, complexation of labeled 3E10-D31N with 3p-hpRNA did not compromise the antibody's ability to target these orthotopic pancreatic tumors (FIG. 38J) and there was almost no localization to non-malignant tissues except for liver.

The delivery of intact 3p-hpRNA to the pancreatic tumors was investigated following IV administration of the 3E10-D31N/3p-hpRNA RIG-I agonist. Three IV doses of 3E10-D31N/3p-hpRNA RIG-I agonist were given on days 10, 13, and 16 post-implantation, and on the day following the last dose, the tumors were isolated and single cell suspensions from the tumor masses were flow-sorted into EPCAM+ epithelial (tumor) or CD45+ populations (FIG. 38K). Using an RT-PCR assay to quantify the full length 3p-hpRNA, we observed a more than 1000-fold difference in 3p-hpRNA delivery specifically into KPC pancreatic tumor cells relative to CD45+ immune cells within the same tumors (p<0.0001) (FIG. 38L), highlighting the tumor cell targeting specificity of 3E10-D31N antibody-RNA complexes in vivo.

As another measure of efficacy, sections of liver, lung, and kidney (obtained on day 7 after the last treatment) were investigated for evidence of metastasis. In FIG. 38O it is shown that 3E10-D31N/3p-hpRNA RIG-I agonist treatment reduced the proportion of mice with detectable metastases in these organs.

Given the immunologically cold phenotype of KPC pancreatic tumors, it was investigated whether 3E10-D31N/3p-hpRNA RIG-I agonist promoted accumulation of tumor infiltrating lymphocytes (TILs). In a parallel experiment, on day 1 following the last of the 3 doses of 3E10-D31N/3p-hpRNA RIG-I agonist, tumors were isolated and total RNA was analyzed by RT-PCR to quantify CD3, CD4 and CD8 mRNA expression. In FIG. 38P, significant increases were observed in the proportion of CD4 and CD8 cells, as well as an increase in the CD8/CD4 ratio, consistent with RIG-I mediated stimulation8

Taken together, these data indicate that a complex of 3E10-D31N/3p-hpRNA RIG-I agonist is effective for treatment of pancreatic cancer in vivo.

### Example 29: cD31N Binds Multiple Species of RNA

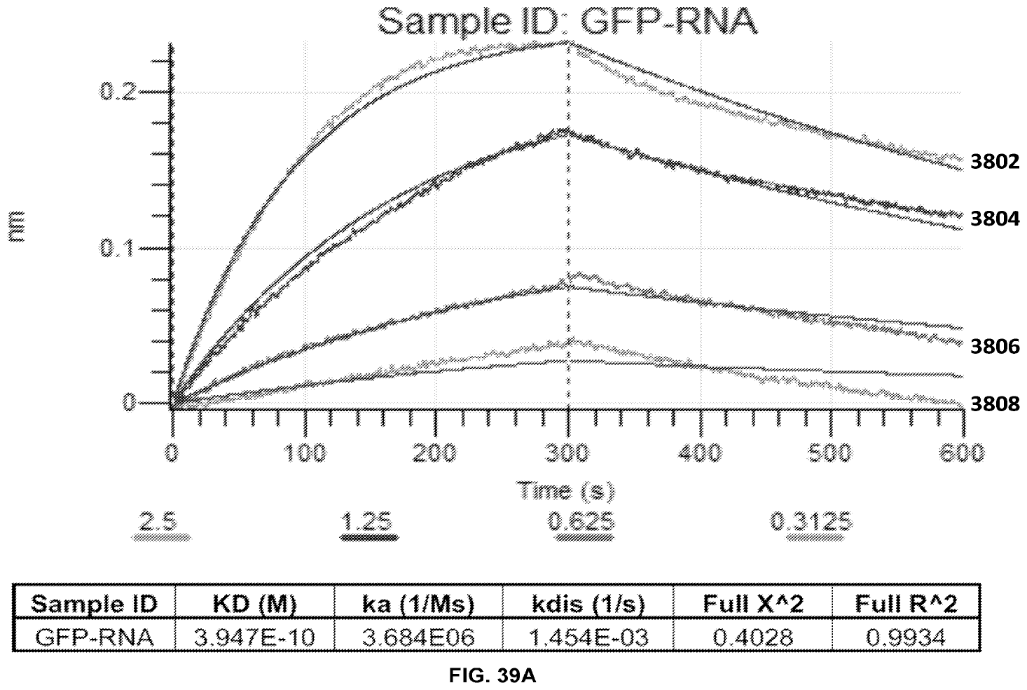

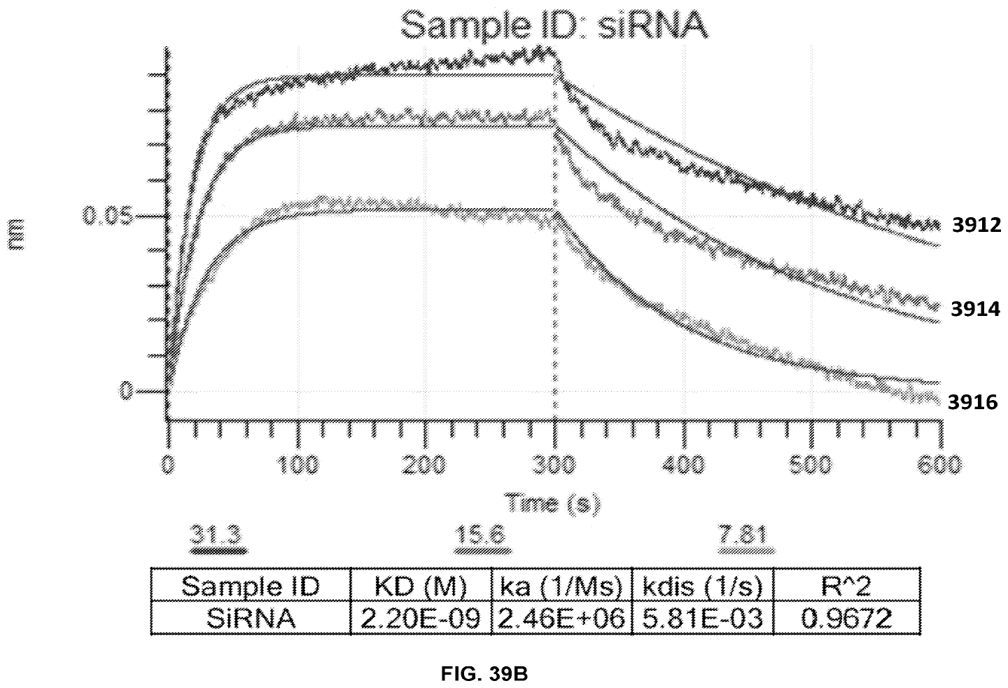

FIGS. 39A and 39B collectively illustrate that cD31N binds multiple species of RNA. Bio-Layer Interferometry (BLI) measurement of cD31N affinity for (FIG. 39A) GFP-mRNA and (FIG. 39B) a KRAS-targeting siRNA. In FIG. 39A, 3902 shows collected and fitted curves for 2.5 s, 3904 shows collected and fitted curves for 1.25 s, 3906 shows collected and fitted curves for 0.625 s, and 3908 shows collected and fitted curves for 0.3125 s. In FIG. 39B, 3912 shows collected and fitted curves for 31.3 s, 3914 shows collected and fitted curves for 15.6 s, and 3916 shows collected and fitted curves for 7.81 s. Derived values for the dissociation constants (KD) and related parameters are indicated in the corresponding tables.

### Example 30: cD31N Cellular Penetration is ENT2 Dependent

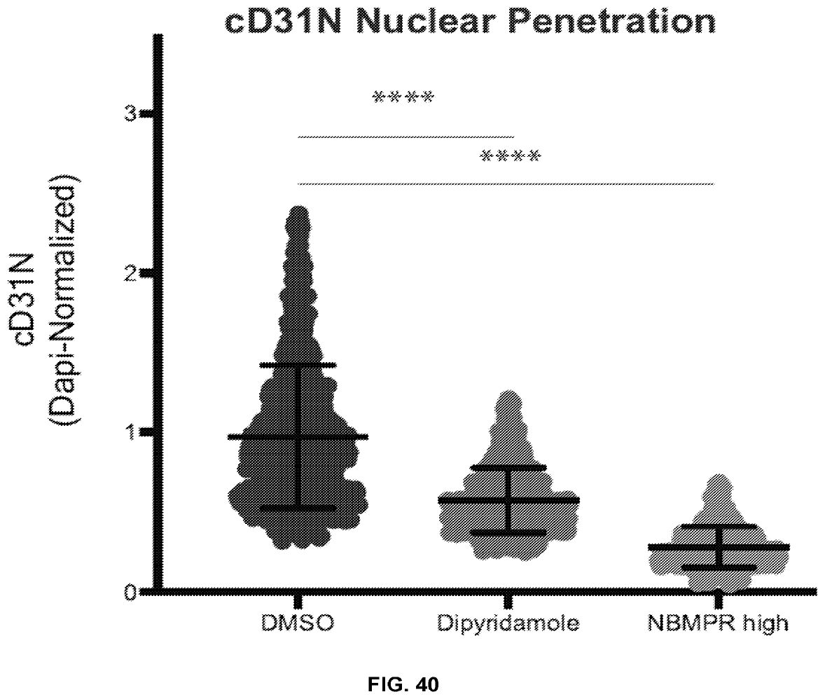

FIG. 40 illustrates that cD31N cellular penetration is ENT2 dependent. U2OS human osteosarcoma cells were pre-treated with dipyridamole or NBMPR (6-S-[(4-Nitrophenyl)methyl]-6-thioinosine) for 30 minutes prior to incubation with cD31N. Following 30 minutes of treatment with cD31N, uptake was quantified by immunofluorescence normalized to DAPI (4′,6-diamidino-2-phenylindole) staining of nuclei. Statistical analysis performed by student's t-test for individual comparison, with p values indicated.

### Example 31: ENT2 is Highly Expressed in Multiple Tumor Models

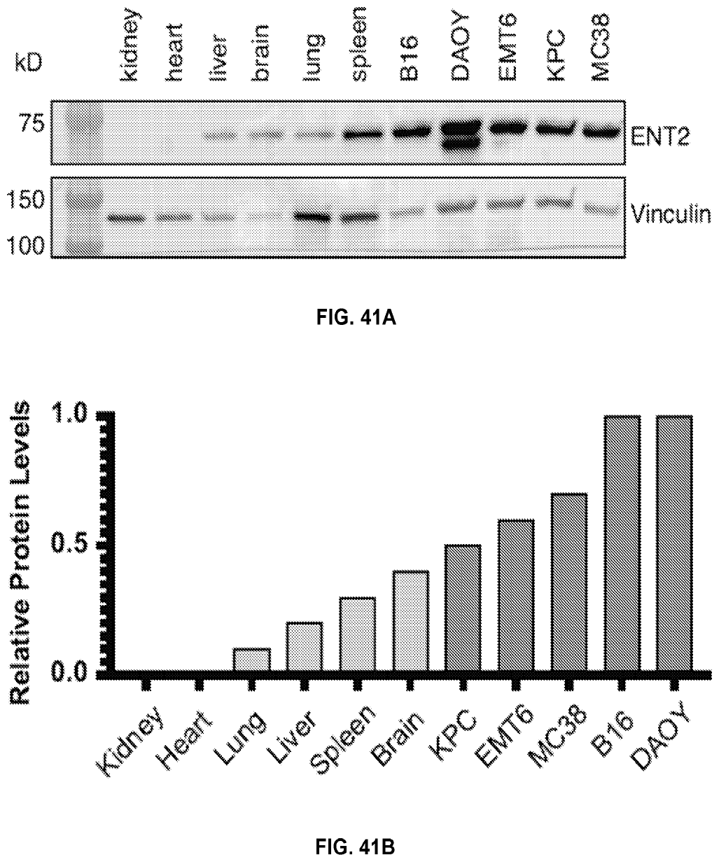

FIGS. 41A and 41B collectively illustrate that ENT2 is highly expressed in multiple tumor models. Western blot analysis (FIG. 41A) and quantification (FIG. 41B) of ENT2 protein expression in cell lysates prepared from murine tissues (C57Bl/6) and indicated tumor cell lines.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A method for treating a cancer in a subject in need thereof, the method comprising:
administering to the subject a therapeutically effective amount of a composition comprising a complex formed between (i) a therapeutic polynucleotide, and (ii) a 3E10 antibody or variant thereof, or antigen-binding fragment thereof;
wherein the 3E10 antibody or antigen-binding fragment thereof comprises:
(a) a light chain variable region (VL) complementarity determining region (CDR) 1 comprising the amino acid sequence of 3E10-VL-CDR1m,
(b) a VL CDR2 comprising the amino acid sequence of 3E10-VL-CDR2m,
(c) a VL CDR3 comprising the amino acid sequence of 3E10-VL-CDR3m,
(d) a heavy chain variable region (VH) CDR1 comprising the amino acid sequence of 3E10-VH-CDR1m,
(e) a VH CDR2 comprising the amino acid sequence of 3E10-VH-CDR2m, and
(f) a VH CDR3 comprising the amino acid sequence of 3E10-VH-CDR3m;
wherein the cancer is selected from the group consisting of endocrine cancer, head and neck cancer, hepatobiliary cancer, ovarian cancer, renal cancer, and thyroid cancer.

2. The method of claim 1, wherein the cancer is a carcinoma, a sarcoma, a blastoma, a papilloma, or an adenoma.

3. The method of claim 1, wherein the cancer is metastatic cancer.

4.-8. (canceled)

9. The method of claim 1, wherein the administering is by parenteral administration.

10.-11. (canceled)

12. The method of claim 1, wherein the composition comprises a molar ratio of (i) 3E10 antibody or variant thereof, or antigen-binding fragment thereof to (ii) therapeutic polynucleotide of at least about 2:1.

13.-14. (canceled)

15. The method of claim 1, wherein the composition comprises a molar ratio of (i) 3E10 antibody or variant thereof, or antigen-binding fragment thereof to (ii) therapeutic polynucleotide of no more than about 200:1.

16.-20. (canceled)

21. The method of claim 1, wherein the therapeutic polynucleotide is a polynucleotide immunostimulant.

22.-33. (canceled)

34. The method of claim 1, wherein the therapeutic polynucleotide encodes a protein or peptide for cancer therapy.

35. The method of claim 34, wherein the protein or peptide for cancer therapy is a tumor antigen.

36.-39. (canceled)

40. The method of claim 34, wherein the protein or peptide for cancer therapy is a proinflammatory cytokine.

41. (canceled)

42. The method of claim 1, wherein the therapeutic polynucleotide is non-replicating unmodified mRNA.

43. The method of claim 1, wherein the therapeutic polynucleotide is non-replicating modified mRNA.

44. The method of claim 1, wherein the therapeutic polynucleotide is a self-amplifying mRNA.

45. The method of claim 1, wherein the therapeutic polynucleotide is a plasmid encoding the protein or peptide.

46. The method of claim 1, wherein the therapeutic polynucleotide is a plasmid encoding an antisense sequence.

47. The method of claim 1, wherein the therapeutic polynucleotide is a gene-regulating polynucleotide.

48.-56. (canceled)

57. The method of claim 1, wherein the therapeutic polynucleotide encodes a genome editing effector.

58.-60. (canceled)

61. The method of claim 1, wherein the therapeutic polynucleotide is an effector polynucleotide.

62. The method of claim 61, wherein the effector polynucleotide is an aptamer.

63. (canceled)

64. The method of claim 61, wherein the effector polynucleotide is a ribozyme.

65.-255. (canceled)


SEQUENCE LISTING (152 sequences)

SEQ ID NO: 1 - protein, 8 aa, synthetic construct
GFTFSDYG

SEQ ID NO: 2 - protein, 8 aa, synthetic construct
GFTFSNYG

SEQ ID NO: 3 - protein, 8 aa, synthetic construct
ISSGSSTI

SEQ ID NO: 4 - protein, 8 aa, synthetic construct
ISSSSSTI

SEQ ID NO: 5 - protein, 9 aa, synthetic construct
ARRGLLLDY

SEQ ID NO: 6 - protein, 10 aa, synthetic construct
KSVSTSSYSY

SEQ ID NO: 7 - protein, 10 aa, synthetic construct
KTVSTSSYSY

SEQ ID NO: 8 - skipped

SEQ ID NO: 9 - protein, 9 aa, synthetic construct
QHSREFPWT

SEQ ID NO: 10 - DNA, 24 bp, synthetic construct
  source 1..24: mol_type = other DNA
  modified_base 7: mod_base = OTHER; note = 2-prime-fluoro guanosine
  modified_base 15: mod_base = OTHER; note = 2-prime-O-methylated adenosine
  modified_base 23: mod_base = OTHER; note = 2-prime-fluoro uridine
  modified_base 22^23: mod_base = OTHER; note = Linked by a phosphorothioate bond
  modified_base 23^24: mod_base = OTHER; note = Linked by a phosphorothioate bond
gacgctgaccctgaagttcatctt

SEQ ID NO: 11 - DNA, 24 bp, synthetic construct
  source 1..24: mol_type = other DNA
  modified_base 21: mod_base = OTHER; note = 2-prime-O-methylated cytidine
  modified_base 11: mod_base = OTHER; note = 2-prime-fluoro uridine
  modified_base 1^2: mod_base = OTHER; note = Linked by a phosphorothioate bond
  modified_base 2^3: mod_base = OTHER; note = Linked by a phosphorothioate bond
aagatgaacttcagggtcagcgtc

SEQ ID NO: 12 - DNA, 24 bp, synthetic construct
  source 1..24: mol_type = other DNA
  modified_base 7: mod_base = OTHER; note = 2-prime-fluoro guanosine
  modified_base 15: mod_base = OTHER; note = 2-prime-O-methylated adenosine
  modified_base 23: mod_base = OTHER; note = 2-prime-fluoro uridine
  modified_base 1^2: mod_base = OTHER; note = Linked by a phosphorothioate bond
  modified_base 2^3: mod_base = OTHER; note = Linked by a phosphorothioate bond
  modified_base 22^23: mod_base = OTHER; note = Linked by a phosphorothioate bond
  modified_base 23^24: mod_base = OTHER; note = Linked by a phosphorothioate bond
gacgctgaccctgaagttcatctt

SEQ ID NO: 13 - DNA, 24 bp, synthetic construct
  source 1..24: mol_type = other DNA
  modified_base 22: mod_base = p
  modified_base 11: mod_base = OTHER; note = 2-prime-fluorouridine
  modified_base 21: mod_base = OTHER; note = 2-prime-O-methylated cytidine
  modified_base 1^2: mod_base = OTHER; note = Linked by a phosphorothioate bond
  modified_base 2^3: mod_base = OTHER; note = Linked by a phosphorothioate bond
aagatgaacttcagggtcagcgtc

SEQ ID NO: 14-23 - skipped (10 entries)

SEQ ID NO: 24 - RNA, 89 nt, synthetic construct
  source 1..89: mol_type = other RNA
ggagcaaaagcagggtgacaaagacataatggatccaaacactgtgtcaagctttcaggtagattgctttctttggcatgtccgcaaac

SEQ ID NO: 25 - DNA, 41 bp, synthetic construct
  source 1..41: mol_type = other DNA
atctaactgctgcgccgccgggaaaatactgtacggttaga

SEQ ID NO: 26 - DNA, 26 bp, synthetic construct
  source 1..26: mol_type = other DNA
ggtggtggtggttgtggtggtggtgg

SEQ ID NO: 27 - RNA, 45 nt, synthetic construct
  source 1..45: mol_type = other RNA
gcgtggtgtgatctagatgtattggctgatcctagtcaggtacgc

SEQ ID NO: 28 - DNA, 78 bp, synthetic construct
  source 1..78: mol_type = other DNA
atccagagtgacgcagcatgccctagttactactactctttttagcaaacgccctcgctttggacacggtggcttagt

SEQ ID NO: 29 - DNA, 50 bp, synthetic construct
  source 1..50: mol_type = other DNA
aaggggttgggtgggtttatacaaattaattaatattgtatggtatattt

SEQ ID NO: 30 - DNA, 44 bp, synthetic construct
  source 1..44: mol_type = other DNA
accgggaggatagttcggtggctgttcagggtctcctcccggtg

SEQ ID NO: 31 - DNA, 88 bp, synthetic construct
  source 1..88: mol_type = other DNA
aaccgcccaaatccctaagagtctgcacttgtcattttgtatatgtatttggtttttggctctcacagacacactacacacgcacatg

SEQ ID NO: 32 - DNA, 40 bp, synthetic construct
  source 1..40: mol_type = other DNA
tctaaaaggattcttcccaaggggatccaattcaaacagc

SEQ ID NO: 33 - DNA, 54 bp, synthetic construct
  source 1..54: mol_type = other DNA
gggccgtcgaacacgagcatggtgcgtggacctaggatgacctgagtactgtcc

SEQ ID NO: 34 - DNA, 57 bp, synthetic construct
  source 1..57: mol_type = other DNA
tggatggggagatccgttgagtaagcgggcgtgtctctctgccgccttgctatgggg

SEQ ID NO: 35 - DNA, 48 bp, synthetic construct
  source 1..48: mol_type = other DNA
cactacagaggttgcgtctgtcccacgttgtcatggggggttggcctg

SEQ ID NO: 36 - DNA, 26 bp, synthetic construct
  source 1..26: mol_type = other DNA
  modified_base 14: mod_base = OTHER; note = n is gemcitabine
ggtggtggtggttntggtggtggtgg

SEQ ID NO: 37 - DNA, 40 bp, synthetic construct
  source 1..40: mol_type = other DNA
agggcccgacgtgacggcacgtcggatatctcatgcgtgt

SEQ ID NO: 38 - DNA, 40 bp, synthetic construct
  source 1..40: mol_type = other DNA
gcccagcatgcattactgatcgtggtgtttgcttagccca

SEQ ID NO: 39 - DNA, 64 bp, synthetic construct
  source 1..64: mol_type = other DNA
tttttttttacattcctaagtctgaaacattacagcttgctacacgagaagagccgccatagta

SEQ ID NO: 40 - RNA, 65 nt, synthetic construct
  source 1..65: mol_type = other RNA
gggtcgtgtcggatccaggcacgacgcatagccttgggagcgaggaaagcttctaaggtaacgat

SEQ ID NO: 41 - RNA, 65 nt, synthetic construct
  source 1..65: mol_type = other RNA
gggtcgtgtcggatccagctcgaaagtgggctggcgatgtgtcccgaagcttctaaggtaacgat

SEQ ID NO: 42 - RNA, 65 nt, synthetic construct
  source 1..65: mol_type = other RNA
gggtcgtgtcggatcctgcgaaggggggcagaggtttgacgcgagaaagcttctaaggtaacgat

SEQ ID NO: 43 - DNA, 88 bp, synthetic construct
  source 1..88: mol_type = other DNA
ctatagcaatggtacggtacttcctctcaggtgggtgtatgtgggctccctttactgattgggtcaaaagtgcacgctactttgctaa

SEQ ID NO: 44 - DNA, 45 bp, synthetic construct
  source 1..45: mol_type = other DNA
ggttgcatgccgtggggaggggggtgggttttatagcgtactcag

SEQ ID NO: 45 - DNA, 30 bp, synthetic construct
  source 1..30: mol_type = other DNA
ttgggacggtgttaaacgaaaggggacgac

SEQ ID NO: 46 - RNA, 44 nt, synthetic construct
  source 1..44: mol_type = other RNA
aaaatgcatggagcgaaggtgtgggggataccaaccgcgccgtg

SEQ ID NO: 47 - DNA, 80 bp, synthetic construct
  source 1..80: mol_type = other DNA
gctgtgtgactcctgcaagcgggaagagggcaggggagggagggtgacgcggaagaggcaagcagctgtatcttgtctcc

SEQ ID NO: 48 - DNA, 44 bp, synthetic construct
  source 1..44: mol_type = other DNA
ctctagttattgagttttcttttatgggtgggtggggggttttt

SEQ ID NO: 49 - DNA, 58 bp, synthetic construct
  source 1..58: mol_type = other DNA
ttggagcagcgtggaggatatgctttccgaccgtgttcgtttgttataacgctgctcc

SEQ ID NO: 50 - DNA, 68 bp, synthetic construct
  source 1..68: mol_type = other DNA
ttaaggagcagcgtggaggatatcggtgtttatggtgtctgtcttcctccagtttccttctgcgcctt

SEQ ID NO: 51 - protein, 115 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVS

SEQ ID NO: 52 - protein, 132 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGS

SEQ ID NO: 53 - RNA, 720 nt, synthetic construct
  source 1..720: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 54 - protein, 446 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 116 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 56 - protein, 5 aa, synthetic construct
DYGMH

SEQ ID NO: 57 - protein, 17 aa, synthetic construct
YISSGSSTIYYADTVKG

SEQ ID NO: 58 - protein, 7 aa, synthetic construct
RGLLLDY

SEQ ID NO: 59 - protein, 19 aa, synthetic construct
MGWSCIILFLVATATGVHS

SEQ ID NO: 60 - protein, 218 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 111 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 62 - protein, 15 aa, synthetic construct
RASKSVSTSSYSYMH

SEQ ID NO: 63 - protein, 7 aa, synthetic construct
YASYLES

SEQ ID NO: 64-65 - skipped (2 entries)

SEQ ID NO: 66 - protein, 446 aa, synthetic construct; identical to SEQ ID NO: 54

SEQ ID NO: 67 - protein, 116 aa, synthetic construct; identical to SEQ ID NO: 55

SEQ ID NO: 68 - protein, 5 aa, synthetic construct
NYGMH

SEQ ID NO: 69 - protein, 5 aa, synthetic construct
  VARIANT 1: note = where X is D or N
XYGMH

SEQ ID NO: 70-72 - skipped (3 entries)

SEQ ID NO: 73 - protein, 218 aa, synthetic construct; identical to SEQ ID NO: 60

SEQ ID NO: 74-78 - skipped (5 entries)

SEQ ID NO: 79 - protein, 17 aa, synthetic construct
YISSGSSTIYYADSVKG

SEQ ID NO: 80 - protein, 17 aa, synthetic construct
YISSSSSTIYYADSVKG

SEQ ID NO: 81 - protein, 15 aa, synthetic construct
RASKSVSTSSYSYLA

SEQ ID NO: 82 - protein, 15 aa, synthetic construct
RASKTVSTSSYSYMH

SEQ ID NO: 83 - protein, 7 aa, synthetic construct
YASYLQS

SEQ ID NO: 84 - protein, 17 aa, synthetic construct
  VARIANT 5: note = X are separately any amino acid
  VARIANT 14: note = X are separately any amino acid
YISSXSSTIYYADXVKG

SEQ ID NO: 85 - protein, 15 aa, synthetic construct
  VARIANT 5: note = X are separately any amino acid
  VARIANT 14: note = X are separately any amino acid
  VARIANT 15: note = X are separately any amino acid
RASKXVSTSSYSYXX

SEQ ID NO: 86 - protein, 7 aa, synthetic construct
  VARIANT 6: note = X are separately any amino acid
YASYLXS

SEQ ID NO: 87 - protein, 5 aa, synthetic construct
QYGMH

SEQ ID NO: 88 - protein, 5 aa, synthetic construct
EYGMH

SEQ ID NO: 89 - skipped

SEQ ID NO: 90 - protein, 5 aa, synthetic construct
  VARIANT 5: note = X is K or R
QYGMX

SEQ ID NO: 91 - protein, 5 aa, synthetic construct
  VARIANT 5: note = X is K or R
EYGMX

SEQ ID NO: 92 - protein, 17 aa, synthetic construct
YISSGSSTIYYAETVKG

SEQ ID NO: 93 - protein, 17 aa, synthetic construct
  VARIANT 16: note = X is H or R
YISSGSSTIYYADTVXG

SEQ ID NO: 94 - protein, 17 aa, synthetic construct
  VARIANT 16: note = X is H or R
YISSGSSTIYYAETVXG

SEQ ID NO: 95 - protein, 7 aa, synthetic construct
  VARIANT 1: note = X is K or H
XGLLLDY

SEQ ID NO: 96 - protein, 8 aa, synthetic construct
RGLLLBEY

SEQ ID NO: 97 - protein, 7 aa, synthetic construct
  VARIANT 1: note = X is K or H
XGLLLEY

SEQ ID NO: 98 - protein, 15 aa, synthetic construct
  VARIANT 1: note = X is K or H
XASKSVSTSSYSYMH

SEQ ID NO: 99 - protein, 15 aa, synthetic construct
  VARIANT 1: note = X is R or H
RASXSVSTSSYSYMH

SEQ ID NO: 100 - protein, 15 aa, synthetic construct
  VARIANT 15: note = X is K or R
RASKSVSTSSYSYMX

SEQ ID NO: 101 - protein, 15 aa, synthetic construct
  VARIANT 1: note = X is K or H
  VARIANT 4: note = X is H or R
XASXSVSTSSYSYMH

SEQ ID NO: 102 - protein, 15 aa, synthetic construct
  VARIANT 1: note = X is K or H
  VARIANT 15: note = X is K or R
XASKSVSTSSYSYMX

SEQ ID NO: 103 - protein, 15 aa, synthetic construct
  VARIANT 4: note = X is R or H
  VARIANT 15: note = X is K or R
RASXSVSTSSYSYMX

SEQ ID NO: 104 - protein, 7 aa, synthetic construct
YASYLDS

SEQ ID NO: 105 - protein, 9 aa, synthetic construct
  VARIANT 2: note = X is K or R
QXSREFPWT

SEQ ID NO: 106 - protein, 9 aa, synthetic construct
  VARIANT 4: note = X is K or H
QHSXEFPWT

SEQ ID NO: 107 - protein, 9 aa, synthetic construct
QHSRDFPWT

SEQ ID NO: 108 - protein, 9 aa, synthetic construct
  VARIANT 2: note = X is K or R
  VARIANT 4: note = X is K or H
QXSXEFPWT

SEQ ID NO: 109 - protein, 9 aa, synthetic construct
  VARIANT 2: note = X is K or R
QXSRDFPWT

SEQ ID NO: 110 - protein, 9 aa, synthetic construct
  VARIANT 4: note = X is K or H
QHSXDFPWT

SEQ ID NO: 111 - skipped

SEQ ID NO: 112 - protein, 17 aa, synthetic construct
  VARIANT 5: note = X is G or S
  VARIANT 13: note = X is D or E
  VARIANT 14: note = X is T or S
  VARIANT 16: note = X is K, R or H
YISSXSSTIYYAXXVXG

SEQ ID NO: 113 - protein, 7 aa, synthetic construct
  VARIANT 1: note = X can be R,K or H
  VARIANT 6: note = X can be D or E
XGLLLXY

SEQ ID NO: 114 - protein, 15 aa, synthetic construct
  VARIANT 1: note = X is K, R or H
  VARIANT 4: note = X is K, R or H
  VARIANT 5: note = X is T or S
  VARIANT 14: note = X is M or L
  VARIANT 15: note = X is K, R, A or H
XASXXVSTSSYSYXX

SEQ ID NO: 115 - protein, 7 aa, synthetic construct
  VARIANT 6: note = X can be D,E,N or Q
YASYLXS

SEQ ID NO: 116 - protein, 9 aa, synthetic construct
  VARIANT 2: note = X is K, R or H
  VARIANT 4: note = X is K, R or H
  VARIANT 5: note = X is D or E
QXSXXFPWT

SEQ ID NO: 117 - protein, 116 aa, synthetic construct
EVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 118 - protein, 116 aa, synthetic construct
EVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 119 - protein, 116 aa, synthetic construct
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 120 - protein, 116 aa, synthetic construct
EVQLVQSGGGLIQPGGSLRLSCAASGFTFSNYGMHWVRQAPGKGLEWVSYISSGSSTIYYADSVKGRFTISRDNSKNTLYLQMNSLRAEDTAVYYCARRGLLLDYWGQGTTVTVSS

SEQ ID NO: 121 - protein, 116 aa, synthetic construct
EVQLVESGGGLIQPGGSLRLSCAASGFTFSNYGMHWVRQAPGKGLEWVSYISSGSSTIYYADSVKGRFTISRDNSKNTLYLQMTSLRAEDTAVYYCARRGLLLDYWGQGTTLTVSS

SEQ ID NO: 122 - protein, 116 aa, synthetic construct
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 123 - protein, 116 aa, synthetic construct
EVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 124 - protein, 116 aa, synthetic construct
EVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 125 - protein, 116 aa, synthetic construct
EVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 126 - protein, 116 aa, synthetic construct
EVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 127 - protein, 111 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIK

SEQ ID NO: 128 - protein, 111 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIK

SEQ ID NO: 129 - protein, 111 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIK

SEQ ID NO: 130 - protein, 111 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIK

SEQ ID NO: 131 - protein, 111 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 132 - protein, 111 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIK

SEQ ID NO: 133 - protein, 111 aa, synthetic construct
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIK

SEQ ID NO: 134 - protein, 111 aa, synthetic construct
DIQMTQSPSSLSASVGDRVTITCRASKSVSTSSYSYLAWYQQKPEKAPKLLIKYASYLQSGVPSRFSGSGSGTDFTLTISSLQPEDFATYYCQHSREFPWTFGAGTKLELK

SEQ ID NO: 135 - protein, 111 aa, synthetic construct
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELK

SEQ ID NO: 136 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 137 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 138 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 139 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 140 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 141 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 142 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 143 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 144 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 145 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 146 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 147 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 148 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 149 - protein, 515 aa, synthetic construct
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 150 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 151 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 152 - protein, 446 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK